SEQUENCE LISTING (155 sequences)

SEQ ID NO: 1 - DNA, 31 bp, synthetic construct
  misc_feature 1..31: note = Description of Artificial Sequence: Synthetic oligonucleotide
  source 1..31: mol_type = other DNA
agagggtatataatggaagctcgacttccag

SEQ ID NO: 2 - DNA, 52 bp, synthetic construct
  misc_feature 1..52: note = Description of Artificial Sequence: Synthetic oligonucleotide
  source 1..52: mol_type = other DNA
gggaatttccggggactttccgggaatttccggggactttccgggaatttcc

SEQ ID NO: 3 - DNA, 85 bp, synthetic construct
  misc_feature 1..85: note = Description of Artificial Sequence: Synthetic oligonucleotide
  source 1..85: mol_type = other DNA
caccagacagtgacgtcagctgccagatcccatggccgtcatactgtgacgtctttcagacaccccattgacgtcaatgggagaa

SEQ ID NO: 4 - DNA, 90 bp, synthetic construct
  misc_feature 1..90: note = Description of Artificial Sequence: Synthetic oligonucleotide
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 5 - DNA, 115 bp, synthetic construct
  misc_feature 1..115: note = Description of Artificial Sequence: Synthetic polynucleotide
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 6 - DNA, 115 bp, synthetic construct
  misc_feature 1..115: note = Description of Artificial Sequence: Synthetic polynucleotide
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 7 - DNA, 48 bp, synthetic construct
  misc_feature 1..48: note = Description of Artificial Sequence: Synthetic oligonucleotide
  source 1..48: mol_type = other DNA
tgagtcagtgactcagtgagtcagtgactcagtgagtcagtgactcag

SEQ ID NO: 8 - DNA, 120 bp, synthetic construct
  misc_feature 1..120: note = Description of Artificial Sequence: Synthetic polynucleotide
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 9 - DNA, 32 bp, synthetic construct
  misc_feature 1..32: note = Description of Artificial Sequence: Synthetic oligonucleotide
  source 1..32: mol_type = other DNA
gtctagacgtctagacgtctagacgtctagac

SEQ ID NO: 10 - DNA, 24 bp, synthetic construct
  misc_feature 1..24: note = Description of Artificial Sequence: Synthetic oligonucleotide
  source 1..24: mol_type = other DNA
cagacacagacacagacacagaca

SEQ ID NO: 11 - DNA, 65 bp, synthetic construct
  misc_feature 1..65: note = Description of Artificial Sequence: Synthetic oligonucleotide
  source 1..65: mol_type = other DNA
ggatccggtactcgagatctgcgatctaagtaagcttggcattccggtactgttggtaaagccac

SEQ ID NO: 12 - DNA, 588 bp, unidentified
  misc_feature 1..588: note = Description of Artificial Sequence: Synthetic polynucleotide
  source 1..588: mol_type = other DNA; note = Cytomegalovirus
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 13 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 544 bp, synthetic construct
  misc_feature 1..544: note = Description of Artificial Sequence: Synthetic polynucleotide
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 15 - DNA, 399 bp, synthetic construct
  misc_feature 1..399: note = Description of Artificial Sequence: Synthetic polynucleotide
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 16 - DNA, 511 bp, synthetic construct
  misc_feature 1..511: note = Description of Artificial Sequence: Synthetic polynucleotide
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 17 - DNA, 408 bp, synthetic construct
  misc_feature 1..408: note = Description of Artificial Sequence: Synthetic polynucleotide
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 18 - DNA, 344 bp, unidentified
  misc_feature 1..344: note = Description of Artificial Sequence: Synthetic polynucleotide
  source 1..344: mol_type = other DNA; note = Simian virus 40
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 19 - DNA, 1229 bp, synthetic construct
  misc_feature 1..1229: note = Description of Artificial Sequence: Synthetic polynucleotide
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 20 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 1718 bp, synthetic construct
  misc_feature 1..1718: note = Description of Artificial Sequence: Synthetic polynucleotide
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 212 bp, synthetic construct
  misc_feature 1..212: note = Description of Artificial Sequence: Synthetic polynucleotide
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 23 - DNA, 2379 bp, synthetic construct
  misc_feature 1..2379: note = Description of Artificial Sequence: Synthetic polynucleotide
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 24 - DNA, 529 bp, synthetic construct
  misc_feature 1..529: note = Description of Artificial Sequence: Synthetic polynucleotide
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 25 - DNA, 794 bp, synthetic construct
  misc_feature 1..794: note = Description of Artificial Sequence: Synthetic polynucleotide
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 26 - DNA, 532 bp, synthetic construct
  misc_feature 1..532: note = Description of Artificial Sequence: Synthetic polynucleotide
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 27 - DNA, 701 bp, synthetic construct
  misc_feature 1..701: note = Description of Artificial Sequence: Synthetic polynucleotide
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 28 - DNA, 465 bp, synthetic construct
  misc_feature 1..465: note = Description of Artificial Sequence: Synthetic polynucleotide
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 29 - DNA, 538 bp, synthetic construct
  misc_feature 1..538: note = Description of Artificial Sequence: Synthetic polynucleotide
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 30 - DNA, 1091 bp, synthetic construct
  misc_feature 1..1091: note = Description of Artificial Sequence: Synthetic polynucleotide
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 31 - protein, 298 aa, unidentified
  REGION 1..298: note = Description of Unknown: ABI sequence
VPLYGFTSICGRRPEMEAAVSTIPRFLQSSSGSMLDGRFDPQSAAHFFGVYDGHGGSQVANYCRERMHLALAEEIAKEKPMLCDGDTWLEKWKKALFNSFLRVDSEIESVAPETVGSTSVVAVVFPSHIFVANCGDSRAVLCRGKTALPLSVDHKPDREDEAARIEAAGGKVIQWNGARVFGVLAMSRSIGDRYLKPSIIPDPEVTAVKRVKEDDCLILASDGVWDVMTDEEACEMARKRILLWHKKNAVAGDASLLADERRKEGKDPAAMSAAEYLSKLAIQRGSKDNISVVVVDLK

SEQ ID NO: 32 - protein, 191 aa, Homo sapiens
DGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 33 - protein, 121 aa, synthetic construct
  REGION 1..121: note = Description of Artificial Sequence: Synthetic polypeptide
GSQVQLVESGGGLVQAGGSLRLSCTASGRTGTIYSMAWFRQAPGKEREFLATVGWSSGITYYMDSVKGRFTISRDKGKNTVYLQMDSLKPEDTAVYYCTATRAYSVGYDYWGQGTQVTVSS

SEQ ID NO: 34 - protein, 119 aa, synthetic construct
  REGION 1..119: note = Description of Artificial Sequence: Synthetic polypeptide
EVQLQASGGGFVQPGGSLRLSCAASGSTSRQYDMGWFRQAPGKEREFVSAISSNQDQPPYYADSVKGRFTISRDNSKNTVYLQMNSLRAEDTATYYCAFKQHHANGAYWGQGTQVTVSS

SEQ ID NO: 35 - protein, 119 aa, synthetic construct
  REGION 1..119: note = Description of Artificial Sequence: Synthetic polypeptide
EVQLQASGGGFVQPGGSLRLSCAASGRFSWGEEMGWFRQAPGKEREFVSAISWAATPWQYYADSVKGRFTISRDNSKNTVYLQMNSLRAEDTATYYCADEWHIGHVSYWGQGTQVTVSS

SEQ ID NO: 36 - protein, 119 aa, synthetic construct
  REGION 1..119: note = Description of Artificial Sequence: Synthetic polypeptide
EVQLQASGGGFVQPGGSLRLSCAASGTTSDNDTMGWFRQAPGKEREFVSAISWNGGRDEYYADSVKGRFTISRDNSKNTVYLQMNSLRAEDTATYYCAYQDNRSWQEYWGQGTQVTVSS

SEQ ID NO: 37 - protein, 119 aa, synthetic construct
  REGION 1..119: note = Description of Artificial Sequence: Synthetic polypeptide
EVQLQASGGGFVQPGGSLRLSCAASGGYSRADDMGWFRQAPGKEREFVSAISFGETDSFYYADSVKGRFTISRDNSKNTVYLQMNSLRAEDTATYYCAYHNYTNMFEYWGQGTQVTVSS

SEQ ID NO: 38 - protein, 119 aa, synthetic construct
  REGION 1..119: note = Description of Artificial Sequence: Synthetic polypeptide
EVQLQASGGGFVQPGGSLRLSCAASGTTYGQTNMGWFRQAPGKEREFVSAISGLQGRDLYYADSVKGRFTISRDNSKNTVYLQMNSLRAEDTATYYCAFHDFLRMWEYWGQGTQVTVSS

SEQ ID NO: 39 - protein, 415 aa, unidentified
  REGION 1..415: note = Description of Unknown: Caspase 9 sequence
DEADRRLLRRCRLRLVEELQVDQLWDVLLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 40 - protein, 60 aa, synthetic construct
  REGION 1..60: note = Description of Artificial Sequence: Synthetic polypeptide
FNVLMVHKRSHTGERPLQCEICGFTCRQKGNLLRHIKLHTGEKPFKCHLCNYACQRRDAL

SEQ ID NO: 41 - protein, 217 aa, synthetic construct
  REGION 1..217: note = Description of Artificial Sequence: Synthetic polypeptide
DPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 42 - protein, 312 aa, synthetic construct
  REGION 1..312: note = Description of Artificial Sequence: Synthetic polypeptide
AGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEAADAHRLHAPTSRGGASVEETDQSHLATAGSTSSHSLQKYYITGEAEGFPAT

SEQ ID NO: 43 - protein, 107 aa, unidentified
  REGION 1..107: note = Description of Unknown: FKBP sequence
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 44 - protein, 93 aa, unidentified [sequence omitted]

SEQ ID NO: 45 - protein, 19 aa, synthetic construct
  REGION 1..19: note = Description of Artificial Sequence: Synthetic peptide
GGGGSGGGGSGGGGSVDGF

SEQ ID NO: 46 - protein, 9 aa, synthetic construct
  REGION 1..9: note = Description of Artificial Sequence: Synthetic peptide
ASGGGGSAS

SEQ ID NO: 47 - protein, 246 aa, unidentified
  REGION 1..246: note = Description of Unknown: Granzyme B sequence
QPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRH

SEQ ID NO: 48 - protein, 279 aa, unidentified
  REGION 1..279: note = Description of Unknown: iCasp9 sequence
DVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 49 - protein, 65 aa, unidentified
  REGION 1..65: note = Description of Unknown: KRAB sequence
RTLVTFKDVFVDFTREEWKLLDTAQQIVYRNVMLENYKNLVSLGYQLTKPDVILRLEKGEEPWLV

SEQ ID NO: 50 - protein, 116 aa, synthetic construct
  REGION 1..116: note = Description of Artificial Sequence: Synthetic polypeptide
QMQLLESGPGLVKPSETLSLTCTVSGGSIWGWIRQPPGKGLEWIGSIYSSGSTYYNPSLKSRVTTSVDTSKNQFSLRLSSVTAADTAVYYCVAWFGDLLSLKGVELWGQGTLVTVS

SEQ ID NO: 51 - protein, 111 aa, synthetic construct
  REGION 1..111: note = Description of Artificial Sequence: Synthetic polypeptide
QSELTQPPSASGTPGQRVTISCSGSSSNIGSNYVYWYQQLPGTAPKLLIYRNNQRPSGVPDRFSGSKSGTSASLAISGLRSEDEADYYCAAWDDSLSAWVFGGGTQLDILG

SEQ ID NO: 52 - protein, 299 aa, unidentified
  REGION 1..299: note = Description of Unknown: PR domain sequence
GIRKFKKFNKVRVVRALDAVALPQPLGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 53 - protein, 177 aa, unidentified
  REGION 1..177: note = Description of Unknown: PYL sequence
TQDEFTQLSQSIAEFHTYQLGNGRCSSLLAQRIHAPPETVWSVVRRFDRPQIYKHFIKSCNVSEDFEMRVGCTRDVNVISGLPANTSRERLDLLDDDRRVTGFSITGGEHRLRNYKSVTTVHRFEKEEEEERIWTVVLESYVVDVPEGNSEEDTRLFADTVIRLNLQKLASITEAMN

SEQ ID NO: 54 - protein, 194 aa, synthetic construct
  REGION 1..194: note = Description of Artificial Sequence: Synthetic polypeptide
DCEVNNGSSLRDECITNLLVFGFLQSCSDNSFRRELDALGHELPVLAPQWEGYDELQTDGNRSSHSRLGRIEADSESQEDIIRNIARHLAQVGDSMDRSIPPGLVNGLALQLRNTSRSEEDRNRDLATALEQLLQAYPRDMEKEKTMLVLALLLAKKVASHTPSLLRDVFHTTVNFINQNLRTYVRSLARNGMD

SEQ ID NO: 55 - protein, 531 aa, unidentified
  REGION 1..531: note = Description of Unknown: VPR sequence
EASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 56 - protein, 503 aa, synthetic construct
  REGION 1..503: note = Description of Artificial Sequence: Synthetic polypeptide
SRGSEFMTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 57 - protein, 176 aa, unidentified
  REGION 1..176: note = Description of Unknown: ZF10-1 sequence
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 58 - DNA, 894 bp, unidentified
  misc_feature 1..894: note = Description of Unknown: ABI sequence
  source 1..894: mol_type = other DNA
gtgcccctgtatggcttcacttccatttgtggccgacggcctgaaatggaagccgcggtgtcaaccataccacggtttctgcagagctcatcaggctccatgctggacggacgctttgatccacagtctgccgcacatttctttggagtctacgacggccacgggggcagccaggtcgccaactactgcagggaaaggatgcatttggcacttgccgaagagatcgccaaagagaagcccatgttgtgtgatggggatacctggctggagaagtggaagaaagcgctttttaactcttttctgagagtggattctgagatagaatctgtcgcacccgagaccgtgggcagcacatccgtcgtagccgtagtgtttccctcccacatattcgtcgccaactgcggcgacagtcgagccgtcctctgccgaggtaagaccgccctgcctctgagtgttgaccataagcccgaccgggaggatgaggccgcccgaatcgaggccgccggtggaaaagtcatccaatggaacggcgcaagagtgttcggcgtgctggcgatgtccaggagcattggagaccggtacctgaagcccagcataatcccagatcccgaagtgaccgcagtcaagagggtgaaagaggacgattgtctgatcctggctagcgatggcgtatgggacgtgatgactgatgaggaggcgtgtgaaatggcccgcaagcgaatcctgctgtggcataaaaaaaacgcagtcgcgggggacgcttctcttctggcagacgaaaggcgcaaagaaggtaaagacccggctgctatgagcgccgccgaatatctcagtaagctggcaattcagcgagggtccaaagacaacatttccgtggtcgtggtagacctcaaa

SEQ ID NO: 59 - DNA, 212 bp, unidentified
  misc_feature 1..212: note = Description of Unknown: 4x ZF10-1 binding site sequence
  source 1..212: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggata

SEQ ID NO: 60 - DNA, 573 bp, Homo sapiens
  source 1..573: mol_type = other DNA
gacgggtccggggagcagcccagaggcggggggcccaccagctctgagcagatcatgaagacaggggcccttttgcttcagggtttcatccaggatcgagcagggcgaatgggtggagaggcacccgagctggccctggacccggtgcctcaggatgcgtccaccaagaagctgagcgagtgtctcaagcgcatcggggacgaactggacagtaacatggagctgcagaggatgattgccgccgtggacacagactccccccgagaggtctttttccgagtggcagctgacatgttttctgacggcaacttcaactggggccgggttgtcgcccttttctactttgccagcaaactggtgctcaaggccctgtgcaccaaggtgccggaactgatcagaaccatcatgggctggacattggacttcctccgggagcggctgttgggctggatccaagaccagggtggttgggacggcctcctctcctactttgggacgcccacgtggcagaccgtgaccatctttgtggcgggagtgctcaccgcctcactcaccatctggaagaagatgggc

SEQ ID NO: 61 - DNA, 363 bp, synthetic construct
  misc_feature 1..363: note = Description of Artificial Sequence: Synthetic polynucleotide
  source 1..363: mol_type = other DNA
ggctctcaggttcaattggtggaatctggagggggtctcgtacaggcaggcggttctctccgactgagttgcacagcctccggtaggactgggaccatctactcaatggcctggtttcgccaggccccaggcaaagaaagagagtttcttgccactgtaggttggagttctgggatcacatactacatggattcagttaaaggaagattcactatcagccgagataaagggaaaaatactgtgtacctccagatggactctctgaaaccggaggacacggctgtctactactgtacagccacccgcgcctactccgtagggtatgactactgggggcaaggaacacaggtaaccgtctctagc

SEQ ID NO: 62 - DNA, 357 bp, synthetic construct
  misc_feature 1..357: note = Description of Artificial Sequence: Synthetic polynucleotide
  source 1..357: mol_type = other DNA
gaggtgcagctgcaggccagcggtggcggattcgtgcagcccggaggttcactgaggctgagttgcgccgccagcggctctacgagtcgacaatatgacatgggctggttcaggcaggcccccggcaaggagagggagttcgtgagcgccatcagctctaaccaagatcagcctccctactatgccgactcagtgaagggcaggttcaccatcagcagggacaacagcaagaacaccgtgtacctgcagatgaactctctgagggccgaggacaccgccacctattactgcgccttcaagcagcaccatgcaaatggcgcatactggggacagggaacccaggtgaccgtgtctagc

SEQ ID NO: 63 - DNA, 357 bp, synthetic construct
  misc_feature 1..357: note = Description of Artificial Sequence: Synthetic polynucleotide
  source 1..357: mol_type = other DNA
gaggtgcagctgcaggccagcggtggcggattcgtgcagcccggaggttcactgaggctgagttgcgccgccagcggccggttctcctggggtgaagagatgggctggttcaggcaggcccccggcaaggagagggagttcgtgagcgccatcagctgggccgctaccccctggcagtactatgccgactcagtgaagggcaggttcaccatcagcagggacaacagcaagaacaccgtgtacctgcagatgaactctctgagggccgaggacaccgccacctattactgcgccgatgagtggcacataggccacgtcagttactggggacagggaacccaggtgaccgtgtctagc

SEQ ID NO: 64 - DNA, 357 bp, synthetic construct
  misc_feature 1..357: note = Description of Artificial Sequence: Synthetic polynucleotide
  source 1..357: mol_type = other DNA
gaggtgcagctgcaggccagcggtggcggattcgtgcagcccggaggttcactgaggctgagttgcgccgccagcggcaccacttcagataatgataccatgggctggttcaggcaggcccccggcaaggagagggagttcgtgagcgccatcagctggaacggcgggcgggacgaatactatgccgactcagtgaagggcaggttcaccatcagcagggacaacagcaagaacaccgtgtacctgcagatgaactctctgagggccgaggacaccgccacctattactgcgcctaccaagacaacaggagctggcaagaatactggggacagggaacccaggtgaccgtgtctagc

SEQ ID NO: 65 - DNA, 357 bp, synthetic construct
  misc_feature 1..357: note = Description of Artificial Sequence: Synthetic polynucleotide
  source 1..357: mol_type = other DNA
gaggtgcagctgcaggccagcggtggcggattcgtgcagcccggaggttcactgaggctgagttgcgccgccagcggcggttattctcgcgccgatgatatgggctggttcaggcaggcccccggcaaggagagggagttcgtgagcgccatcagcttcggagagacggacagcttttactatgccgactcagtgaagggcaggttcaccatcagcagggacaacagcaagaacaccgtgtacctgcagatgaactctctgagggccgaggacaccgccacctattactgcgcctaccacaattacactaatatgtttgagtactggggacagggaacccaggtgaccgtgtctagc

SEQ ID NO: 66 - DNA, 357 bp, synthetic construct
  misc_feature 1..357: note = Description of Artificial Sequence: Synthetic polynucleotide
  source 1..357: mol_type = other DNA
gaggtgcagctgcaggccagcggtggcggattcgtgcagcccggaggttcactgaggctgagttgcgccgccagcggcaccacttacggacaaaccaatatgggctggttcaggcaggcccccggcaaggagagggagttcgtgagcgccatcagcggacttcaaggcagggatctttactatgccgactcagtgaagggcaggttcaccatcagcagggacaacagcaagaacaccgtgtacctgcagatgaactctctgagggccgaggacaccgccacctattactgcgccttccacgatttccttaggatgtgggaatactggggacagggaacccaggtgaccgtgtctagc

SEQ ID NO: 67 - DNA, 1245 bp, unidentified
  misc_feature 1..1245: note = Description of Unknown: Caspase 9 sequence
  source 1..1245: mol_type = other DNA
gacgaagcggatcggcggctcctgcggcggtgccggctgcggctggtggaagagctgcaggtggaccagctctgggacgtcctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggattcggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 68 - DNA, 180 bp, synthetic construct
  misc_feature 1..180: note = Description of Artificial Sequence: Synthetic polynucleotide
  source 1..180: mol_type = other DNA
ttcaatgtcttaatggttcataagcgaagccatactggtgaacgcccattgcagtgcgaaatatgcggctttacctgccgccagaaaggtaacctcctccgccacattaaactgcacacaggggaaaaaccttttaagtgtcacctctgcaactatgcatgccaaagaagagatgcgctc

SEQ ID NO: 69 - DNA, 651 bp, synthetic construct
  misc_feature 1..651: note = Description of Artificial Sequence: Synthetic polynucleotide
  source 1..651: mol_type = other DNA
gaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctc

SEQ ID NO: 70 - DNA, 936 bp, synthetic construct
  misc_feature 1..936: note = Description of Artificial Sequence: Synthetic polynucleotide
  source 1..936: mol_type = other DNA
gccggcgatatgagagctgctaacctgtggccttctccactgatgatcaagcggtccaagaagaacagtctggccctgagcctgaccgccgaccagatggtttcagcactgctggatgccgagcctcctatcctgtacagcgagtacgaccccaccagaccttttagcgaggccagcatgatgggcctgctgaccaatctggccgacagagaactggtgcacatgatcaactgggccaagcgcgtgcccggctttgtggatctgacactgcacgaccaagtgcatctgctcgagtgcgcctggctggaaatcctgatgatcggactcgtgtggcggagcatggaacaccctgtgaagctgctgttcgcccctaacctgctgctggacagaaaccagggcaaatgcgtggaaggcatggtggaaatcttcgatatgctgctggccacctccagccggttccggatgatgaatctgcagggcgaagagttcgtgtgcctgaagtccattatcctgctgaacagcggcgtgtacacctttctgagcagcaccctgaagtctctggaagagaaggaccacatccacagagtgctggacaagatcaccgacacactgatccacctgatggccaaggccggactgactctgcagcagcagcatcaaagactggcccagctgctgctcatcctgagccacatcagacacatgagcaacaaaggcatggaacatctgtacagcatgaagtgcaagaacgtggtgcccctgtacgatctgctgctcgaagccgctgatgcccacagactgcatgcccctacatctagaggcggagcctccgtggaagagacagatcagtctcatctggccaccgccggcagcacaagctctcattctctgcagaagtactacatcaccggcgaggccgagggatttcctgccaca

SEQ ID NO: 71 - DNA, 321 bp, unidentified
  misc_feature 1..321: note = Description of Unknown: FKBP sequence
  source 1..321: mol_type = other DNA
ggagtgcaggtggaaaccatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 72 - DNA, 279 bp, unidentified
  misc_feature 1..279: note = Description of Unknown: FRB sequence
  source 1..279: mol_type = other DNA
atcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaag

SEQ ID NO: 73 - DNA, 57 bp, synthetic construct
  misc_feature 1..57: note = Description of Artificial Sequence: Synthetic oligonucleotide
  source 1..57: mol_type = other DNA
gggggtggaggttcagggggtggaggttcaggtggtggcggtagtgtcgatggcttc

SEQ ID NO: 74 - DNA, 27 bp, synthetic construct
  misc_feature 1..27: note = Description of Artificial Sequence: Synthetic oligonucleotide
  source 1..27: mol_type = other DNA
gctagtggcggcggcggcagtgctagt

SEQ ID NO: 75 - DNA, 738 bp, unidentified
  misc_feature 1..738: note = Description of Unknown: Granzyme B sequence
  source 1..738: mol_type = other DNA
caaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacaagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgccac

SEQ ID NO: 76 - DNA, 837 bp, unidentified
  misc_feature 1..837: note = Description of Unknown: iCasp9 sequence
  source 1..837: mol_type = other DNA
gatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 77 - DNA, 195 bp, unidentified
  misc_feature 1..195: note = Description of Unknown: KRAB sequence
  source 1..195: mol_type = other DNA
agaacactggttacgttcaaggacgtgtttgtggactttacacgtgaggagtggaaattgctggatactgcgcaacaaattgtgtatcgaaatgtcatgcttgagaattacaagaacctcgtcagtctcggataccagttgacgaaaccggatgtgatccttaggctcgaaaagggggaagaaccttggctggta

SEQ ID NO: 78 - DNA, 348 bp, synthetic construct
  misc_feature 1..348: note = Description of Artificial Sequence: Synthetic polynucleotide
  source 1..348: mol_type = other DNA
cagatgcaactgttggaatcgggaccgggtttggtcaagccgagcgagacattgagtttaacgtgcacggtttcaggcggatcaatttggggttggattcgccagccgccaggaaagggactggagtggattggatctatttactcctccggttccacctactataatccgagcttgaagtcccgtgtgacaacaagcgtggatacatccaaaaaccagttttcattgcgcctgtcctcagtaaccgcagccgacacagccgtatattattgcgttgcttggtttggcgacttattaagtcttaaaggggtagagctttggggccagggaactcttgtgacggtatcg

SEQ ID NO: 79 - DNA, 333 bp, synthetic construct
  misc_feature 1..333: note = Description of Artificial Sequence: Synthetic polynucleotide
  source 1..333: mol_type = other DNA
caatccgagttgacccagccgcctagtgctagtggaacaccgggacagcgcgtgacaatttcatgctccggttcaagctcaaatatcggctctaattatgtttactggtatcagcagcttccaggtactgcgcctaagctcttaatctaccgtaacaatcaacgtccgtccggtgtgcccgaccgcttctcaggcagtaaaagtggtacttccgcatcccttgcaatttcgggacttcgtagcgaagatgaggcagactattactgtgcagcatgggatgattccttatcagcttgggtattcggtggcggaactcaattagacattttgggg

SEQ ID NO: 80 - DNA, 897 bp, unidentified
  misc_feature 1..897: note = Description of Unknown: PR domain sequence
  source 1..897: mol_type = other DNA
gggatccgaaaatttaaaaagttcaataaagtcagagttgtgagagcactggatgctgttgctctcccacagccattgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacatgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacagcttcatctgtactgcttgaatacatttatccagtcccgggcactgagtgttgaatttccagaaatgatgtctgaagttattgctgcacaattacccaagatattggcagggatggtgaaaccccttctctttcataaaaag

SEQ ID NO: 81 - DNA, 531 bp, unidentified
  misc_feature 1..531: note = Description of Unknown: PYL sequence
  source 1..531: mol_type = other DNA
actcaagacgaattcacccaactctcccaatcaatcgccgagttccacacgtaccaactcggtaacggccgttgctcatctctcctagctcagcgaatccacgcgccgccggaaacagtatggtccgtggtgagacgtttcgataggccacagatttacaaacacttcatcaaaagctgtaacgtgagtgaagatttcgagatgcgagtgggatgcacgcgcgacgtgaacgtgataagtggattaccggcgaatacgtctcgagagagattagatctgttggacgatgatcggagagtgactgggtttagtataaccggtggtgaacataggctgaggaattataaatcggttacgacggttcatagatttgagaaagaagaagaagaagaaaggatctggaccgttgttttggaatcttatgttgttgatgtaccggaaggtaattcggaggaagatacgagattgtttgctgatacggttattagattgaatcttcagaaacttgcttcgatcactgaagctatgaac

SEQ ID NO: 82 - DNA, 1593 bp, unidentified
  misc_feature 1..1593: note = Description of Unknown: VPR sequence
  source 1..1593: mol_type = other DNA
gaagcctctggaagcggcagagctgacgccctggatgacttcgacctggatatgctgggcagcgacgctctggacgattttgacctcgacatgctgggatctgatgcactcgacgatttcgatttggacatgctcggcagtgatgccttggacgactttgatcttgatatgctcatcaacagccggtccagcggcagccccaagaaaaaaagaaaagtgggctcccagtacctgcctgacaccgacgacagacaccggatcgaggaaaagcggaagcggacctacgagacattcaagagcatcatgaagaagtccccattcagcggccccaccgatcctagacctccacctagaagaatcgccgtgcctagcagatctagcgcctccgtgcctaaacctgctcctcagccttatcctttcaccagcagcctgagcaccatcaactacgacgagttccctaccatggtgttccccagcggccagatctctcaggcttctgctcttgctccagctcctcctcaggttctgcctcaagctcctgcaccagcaccggctccagctatggtttctgctttggctcaggcccctgctcctgtgcctgttcttgctcctggaccacctcaggctgttgctcctcctgctccaaaacctacacaggccggcgaaggcacactgtctgaagctctgctgcagctccagttcgatgacgaagatctgggcgccctgctgggcaattctacagatcctgccgtgtttaccgatctggccagcgtggacaacagcgagtttcagcagctcctgaatcagggcatccctgtggctcctcacaccaccgaacctatgctgatggaataccccgaggccatcaccagactggtcaccggtgctcaaagaccacctgatccagctccagcaccactgggagcacctggactgcctaatggactgctgtctggcgacgaggacttcagctctatcgccgacatggatttctctgccctgctcggctctggcagcggctctagagatagcagagaaggcatgttcctgcctaagcctgaggccggctctgccatctccgatgtgttcgagggaagagaagtgtgccagcctaagcggatccggccttttcaccctcctggaagcccttgggccaacagacctctgcctgcttctctggcccctacaccaacaggacctgtgcacgaacctgtgggcagtctgaccccagctcctgttcctcaacctctggatcccgctcctgctgtgacacctgaagcctctcatctgctggaagatcccgacgaagagacaagccaggccgtgaaggccctgagagaaatggccgacacagtgatccctcagaaagaggaagccgccatctgcggacagatggacctgtctcatcctccaccaagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttt

SEQ ID NO: 83 - DNA, 1509 bp, synthetic construct
  misc_feature 1..1509: note = Description of Artificial Sequence: Synthetic polynucleotide
  source 1..1509: mol_type = other DNA
tctagaggatccgaattcatgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 84 - DNA, 528 bp, unidentified
  misc_feature 1..528: note = Description of Unknown: ZF10-1 sequence
  source 1..528: mol_type = other DNA
tctagacccggcgaaagacccttccagtgccggatctgcatgcggaacttcagcagaaggcacggcctggacagacacaccagaacacacacaggcgagaagcctttccagtgtagaatctgtatgcgcaatttcagcgaccacagcagcctgaagcggcacctgagaacccataccggcagccagaaaccatttcaatgccgcatctgtatgagaaacttctccgtgcggcacaacctgaccagacacctgaggacacacaccggggagaaaccctttcagtgcagaatatgcatgaggaatttctccgaccactccaacctgagccgccacctgaaaactcacaccggctctcaaaagccatttcagtgtcgtatatgtatgcggaatttttcccagcggagcagcctcgtgcgccatctgaggactcatactggcgaaaagcccttccaatgtcgcatatgcatgcgcaactttagcgagtccggccacctgaagagacatctgcggacacacctgagaggctct

SEQ ID NO: 85 - protein, 19 aa, synthetic construct
  REGION 1..19: note = Description of Artificial Sequence: Synthetic peptide
GGGGSGGGGSGGGGSVDGF

SEQ ID NO: 86 - protein, 9 aa, synthetic construct
  REGION 1..9: note = Description of Artificial Sequence: Synthetic peptide
ASGGGGSAS

SEQ ID NO: 87 - protein, 60 aa, synthetic construct; identical to SEQ ID NO: 40
  REGION 1..60: note = Description of Artificial Sequence: Synthetic polypeptide

SEQ ID NO: 88 - protein, 19 aa, synthetic construct
  REGION 1..19: note = Description of Artificial Sequence: Synthetic peptide
GGGGSGGGGSGGGGSVDGF

SEQ ID NO: 89 - protein, 9 aa, synthetic construct
  REGION 1..9: note = Description of Artificial Sequence: Synthetic peptide
ASGGGGSAS

SEQ ID NO: 90 - protein, 60 aa, synthetic construct; identical to SEQ ID NO: 40
  REGION 1..60: note = Description of Artificial Sequence: Synthetic polypeptide

SEQ ID NO: 91 - protein, 19 aa, synthetic construct
  REGION 1..19: note = Description of Artificial Sequence: Synthetic peptide
GGGGSGGGGSGGGGSVDGF

SEQ ID NO: 92 - protein, 9 aa, synthetic construct
  REGION 1..9: note = Description of Artificial Sequence: Synthetic peptide
ASGGGGSAS

SEQ ID NO: 93 - protein, 60 aa, synthetic construct; identical to SEQ ID NO: 40
  REGION 1..60: note = Description of Artificial Sequence: Synthetic polypeptide

SEQ ID NO: 94 - protein, 176 aa, unidentified; identical to SEQ ID NO: 57
  REGION 1..176: note = Description of Unknown: ZF domain sequence

SEQ ID NO: 95 - protein, 65 aa, synthetic construct; identical to SEQ ID NO: 49
  REGION 1..65: note = Description of Artificial Sequence: Synthetic polypeptide

SEQ ID NO: 96 - protein, 45 aa, synthetic construct
  REGION 1..45: note = Description of Artificial Sequence: Synthetic polypeptide
RTLVTFKDVFVDFTREEWKLLDTAQQIVYRNVMLENYKNLVSLGY

SEQ ID NO: 97 - protein, 531 aa, synthetic construct; identical to SEQ ID NO: 55
  REGION 1..531: note = Description of Artificial Sequence: Synthetic polypeptide

SEQ ID NO: 98 - DNA, 195 bp, synthetic construct
  misc_feature 1..195: note = Description of Artificial Sequence: Synthetic polynucleotide
  source 1..195: mol_type = other DNA
agaaccctggtcaccttcaaggacgtgttcgtggacttcacccgggaagagtggaagctgctggatacagcccagcagatcgtgtaccggaacgtgatgctggaaaactacaagaatctggtgtccctgggctaccagctgaccaagcctgacgtgatcctgcggctggaaaagggcgaagaaccttggctggtg

SEQ ID NO: 99 - DNA, 135 bp, synthetic construct
  misc_feature 1..135: note = Description of Artificial Sequence: Synthetic polynucleotide
  source 1..135: mol_type = other DNA
agaaccctggtcaccttcaaggacgtgttcgtggacttcacccgggaagagtggaagctgctggatacagcccagcagatcgtgtaccggaacgtgatgctggaaaactacaagaatctggtgtccctgggctac

SEQ ID NO: 100 - DNA, 243 bp, unidentified
  misc_feature 1..243: note = Description of Unknown: ACP binding domain sequence
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 101 - protein, 19 aa, synthetic construct
  REGION 1..19: note = Description of Artificial Sequence: Synthetic peptide
GGGGSGGGGSGGGGSVDGF

SEQ ID NO: 102 - protein, 9 aa, synthetic construct
  REGION 1..9: note = Description of Artificial Sequence: Synthetic peptide
ASGGGGSAS

SEQ ID NO: 103 - protein, 60 aa, synthetic construct; identical to SEQ ID NO: 40
  REGION 1..60: note = Description of Artificial Sequence: Synthetic polypeptide

SEQ ID NO: 104 - protein, 19 aa, synthetic construct
  REGION 1..19: note = Description of Artificial Sequence: Synthetic peptide
GGGGSGGGGSGGGGSVDGF

SEQ ID NO: 105 - protein, 9 aa, synthetic construct
  REGION 1..9: note = Description of Artificial Sequence: Synthetic peptide
ASGGGGSAS

SEQ ID NO: 106 - protein, 60 aa, synthetic construct; identical to SEQ ID NO: 40
  REGION 1..60: note = Description of Artificial Sequence: Synthetic polypeptide

SEQ ID NO: 107 - protein, 497 aa, unidentified
  REGION 1..497: note = Description of Unknown: XIAP sequence
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 108 - DNA, 1491 bp, unidentified
  misc_feature 1..1491: note = Description of Unknown: XIAP sequence
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 109 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMSLSDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 110 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatgagcctaagcgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 111 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMSLDDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 112 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatgagcctagacgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 113 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLSDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 114 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaagcgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 115 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLDDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 116 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctagacgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 117 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGSLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 118 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggaagcctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 119 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGMGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 120 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaatggggctaactgattggaagcccagtgaagacccttgggaacaacatgctaaatggtatccagggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 121 - protein, 497 aa, synthetic construct
  REGION 1..497: note = Description of Artificial Sequence: Synthetic polypeptide
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYSGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 122 - DNA, 1491 bp, synthetic construct
  misc_feature 1..1491: note = Description of Artificial Sequence: Synthetic polynucleotide
  source 1..1491: mol_type = other DNA
atgacttttaacagttttgaaggatctaaaacttgtgtacctgcagacatcaataaggaagaagaatttgtagaagagtttaatagattaaaaacttttgctaattttccaagtggtagtcctgtttcagcatcaacactggcacgagcagggtttctttatactggtgaaggagataccgtgcggtgctttagttgtcatgcagctgtagataggtggcaatatggagactcagcagttggaagacacaggaaagtatccccaaattgcagatttatcaacggcttttatcttgaaaatagtgccacgcagtctacaaattctggtatccagaatggtcagtacaaagttgaaaactatctgggaagcagagatcattttgccttagacaggccatctgagacacatgcagactatcttttgagaactgggcaggttgtagatatatcagacaccatatacccgaggaaccctgccatgtatagtgaagaagctagattaaagtcctttcagaactggccagactatgctcacctaaccccaagagagttagcaagtgctggactctactacacaggtattggtgaccaagtgcagtgcttttgttgtggtggaaaactgaaaaattgggaaccttgtgatcgtgcctggtcagaacacaggcgacactttcctaattgcttctttgttttgggccggaatcttaatattcgaagtgaatctgatgctgtgagttctgataggaatttcccaaattcaacaaatcttccaagaaatccatccatggcagattatgaagcacggatctttacttttgggacatggatatactcagttaacaaggagcagcttgcaagagctggattttatgctttaggtgaaggtgataaagtaaagtgctttcactgtggaggagggctaactgattggaagcccagtgaagacccttgggaacaacatgctaaatggtatagcgggtgcaaatatctgttagaacagaagggacaagaatatataaacaatattcatttaactcattcacttgaggagtgtctggtaagaactactgagaaaacaccatcactaactagaagaattgatgataccatcttccaaaatcctatggtacaagaagctatacgaatggggttcagtttcaaggacattaagaaaataatggaggaaaaaattcagatatctgggagcaactataaatcacttgaggttctggttgcagatctagtgaatgctcagaaagacagtatgcaagatgagtcaagtcagacttcattacagaaagagattagtactgaagagcagctaaggcgcctgcaagaggagaagctttgcaaaatctgtatggatagaaatattgctatcgtttttgttccttgtggacatctagtcacttgtaaacaatgtgctgaagcagttgacaagtgtcccatgtgctacacagtcattactttcaagcaaaaaatttttatgtct

SEQ ID NO: 123 - protein, 422 aa, synthetic construct
  REGION 1..422: note = Description of Artificial Sequence: Synthetic polypeptide
MDEADRRLLRRCRLRLVEELQVDQLWDVLLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSHHHHHH

SEQ ID NO: 124 - DNA, 1266 bp, synthetic construct
  misc_feature 1..1266: note = Description of Artificial Sequence: Synthetic polynucleotide
  source 1..1266: mol_type = other DNA
atggacgaagcggatcggcggctcctgcggcggtgccggctgcggctggtggaagagctgcaggtggaccagctctgggacgtcctgctgagccgcgagctgttcaggccccatatgatcgaggacatccagcgggcaggctctggatctcggcgggatcaggccaggcagctgatcatagatctggagactcgagggagtcaggctcttcctttgttcatctcctgcttagaggacacaggccaggacatgctggcttcgtttctgcgaactaacaggcaagcagcaaagttgtcgaagccaaccctagaaaaccttaccccagtggtgctcagaccagagattcgcaaaccagaggttctcagaccggaaacacccagaccagtggacattggttctggaggattcggtgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcacaccaccaccaccaccac

SEQ ID NO: 125 - protein, 280 aa, unidentified
  REGION 1..280: note = Description of Unknown: iCasp9 sequence
MDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 126 - DNA, 840 bp, unidentified
  misc_feature 1..840: note = Description of Unknown: iCasp9 sequence
  source 1..840: mol_type = other DNA
atggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 127 - protein, 442 aa, unidentified [sequence omitted]

SEQ ID NO: 128 - DNA, 1326 bp, unidentified
  misc_feature 1..1326: note = Description of Unknown: CRBN sequence
  source 1..1326: mol_type = other DNA
atggctggggagggagatcaacaggacgcagcgcacaacatgggtaaccatcttccactcctcccggctgagagcgaggaggaggatgaaatggaggttgaggatcaggattccaaagaggcaaagaagccaaatatcatcaactttgacacatccctccccacttcacatacataccttggggctgatatggaggagtttcatggccgcactctccacgacgatgactcatgtcaagtaatcccggtcctcccacaagtgatgatgattctcatccccgggcaaacactcccgctccagctgttccacccccaagaagtaagtatggtccgaaaccttatccaaaaagatcggacgttcgccgttctggcgtactccaacgtacaagaacgcgaagctcagtttggcacgaccgcagaaatatacgcctaccgggaggagcaagatttcggaatagaaatcgtgaaagtgaaggcaattgggagacagcgatttaaagtattggaattgcgaacgcagagcgatggtattcaacaggccaaagtccaaatactgccggaatgcgtgcttccgtctactatgagcgcagtgcagttggaatctttgaacaagtgccagatatttccaagcaagccagtatctcgggaggaccaatgtagctataagtggtggcaaaagtaccaaaagcgcaaattccactgtgcaaacctgacttcttggcccagatggctgtattccttgtacgatgcggagactttgatggatagaattaaaaagcaactgcgagaatgggacgaaaacctgaaagacgactcacttccgagtaatccaatcgatttctcataccgggtggctgcttgtttgcccatagatgacgtgttgcggatccagctgcttaaaataggatccgccatacaacggcttcggtgtgagctggatataatgaataagtgtacaagcctctgttgtaagcaatgtcaagagaccgaaattactactaagaatgagatattctctttgtcactttgcggacctatggctgcctacgttaatccacatgggtacgtgcacgagactcttaccgtgtataaagcctgtaatcttaaccttataggcaggccatccactgaacacagttggttccctggttatgcgtggacggtagcacagtgtaaaatttgtgcatctcacatcggctggaagtttacggcaactaaaaaggacatgagtccccagaagttttggggtctcacccgatccgcccttctgccgaccatcccagataccgaagacgaaatctctccagataaagtgatactgtgtttg

SEQ ID NO: 129 - protein, 388 aa, synthetic construct
  REGION 1..388: note = Description of Artificial Sequence: Synthetic polypeptide
MAGEGDQQDAAHNMGNHLPLLPAESEEEDEMEVEDQDSKEAKKPNIINFDTSLPTSHTYLGADMEEFHGRTLHDDDSCQVIPVLPQVMMILIPGQTLPLQLFHPQEVSMVRNLIQKDRTFAVLAYSNVQEREAQFGTTAEIYAYREEQDFGIEIVKVKAIGRQRFKVLELRTQSDGIQQAKVQILPECVLPSTYDAETLMDRIKKQLREWDENLKDDSLPSNPIDFSYRVAACLPIDDVLRIQLLKIGSAIQRLRCELDIMNKCTSLCCKQCQETEITTKNEIFSLSLCGPMAAYVNPHGYVHETLTVYKACNLNLIGRPSTEHSWFPGYAWTVAQCKICASHIGWKFTATKKDMSPQKFWGLTRSALLPTIPDTEDEISPDKVILCL

SEQ ID NO: 130 - DNA, 1164 bp, synthetic construct
  misc_feature 1..1164: note = Description of Artificial Sequence: Synthetic polynucleotide
  source 1..1164: mol_type = other DNA
atggcgggcgagggcgatcagcaagacgcggctcacaacatgggaaatcacttacccttacttcccgcagaatctgaagaggaagatgagatggaggtagaggatcaggactctaaagaagcgaaaaagcctaatatcatcaactttgacacctctcttccaaccagtcacacttacctgggggcagacatggaggaattccatggtcgaactctccacgacgacgatagctgccaagtgataccagtgctccctcaggtaatgatgattcttattcctggacagaccctgcctctacagctgttccaccctcaagaggtgagcatggtcaggaatttgatccagaaggacagaacatttgcggtcctggcctactcaaacgtacaggagcgagaggctcagttcgggaccactgccgaaatatacgcgtaccgggaggagcaagacttcgggatcgaaatcgtgaaggtaaaggccattggcagacaacggtttaaggtccttgagctccggacgcagagtgatgggatacaacaggctaaagtgcagatcctaccagagtgtgtattaccatctacctatgatgcagagactctcatggaccgcataaaaaagcaattaagggaatgggacgaaaacctcaaggacgattcactcccatccaaccccattgacttctcatatagggtcgctgcttgtttgcctatcgacgacgtccttaggatacagctcctgaaaatcggaagcgcaatacaaagattgcgctgtgagctggacattatgaataagtgcacttcactgtgttgtaagcagtgtcaagagaccgaaatcactacgaagaacgagatcttctctctctccctctgcgggccaatggcagcatatgtaaatccgcatgggtatgttcatgagacactaactgtgtacaaggcctgcaatttaaacttgatcggccggccatctacagaacacagttggttcccgggttatgcctggacagtggcacagtgcaaaatttgtgcttcccacattggatggaaatttacagccacaaagaaggatatgagtccccaaaagttctggggactgaccaggagcgctttattgcccaccatcccggacacagaggacgaaatctctccggacaaggtgattctctgtctg

SEQ ID NO: 131 - protein, 61 aa, unidentified
  REGION 1..61: note = Description of Unknown: d913 sequence
MFNVLMVHKRSHTGERPLQCEICGFTCRQKGNLLRHIKLHTGEKPFKCHLCNYACQRRDAL

SEQ ID NO: 132 - DNA, 183 bp, unidentified
  misc_feature 1..183: note = Description of Unknown: d913 sequence
  source 1..183: mol_type = other DNA
atgttcaacgtgctgatggtgcacaagcggagccacaccggcgaaagacctctgcagtgtgaaatctgcggcttcacctgtcggcagaagggcaacctgctgcggcacatcaaactgcacacaggcgagaagcccttcaagtgccacctgtgcaattacgcctgccagagaagagatgccctg

SEQ ID NO: 133 - protein, 61 aa, synthetic construct
  REGION 1..61: note = Description of Artificial Sequence: Synthetic polypeptide
MFNVLMVHRRSHTGERPLQCEICGFTCRQRGNLLRHIRLHTGERPFRCHLCNYACQRRDAL

SEQ ID NO: 134 - DNA, 183 bp, synthetic construct
  misc_feature 1..183: note = Description of Artificial Sequence: Synthetic polynucleotide
  source 1..183: mol_type = other DNA
atgttcaacgtgctgatggtgcacaggcggagccacaccggcgaaagacctctgcagtgtgaaatctgcggcttcacctgtcggcagaggggcaacctgctgcggcacatcagactgcacacaggcgagaggcccttcaggtgccacctgtgcaattacgcctgccagagaagagatgccctg

SEQ ID NO: 135 - protein, 239 aa, unidentified
  REGION 1..239: note = Description of Unknown: Smac/DIABLO sequence
MAALKSWLSRSVTSFFRYRQCLCVPVVANFKKRCFSELIRPWHKTVTIGFGVTLCAVPIAQKSEPHSLSSEALMRRAVSLVTDSTSTFLSQTTYALIEAITEYTKAVYTLTSLYRQYTSLLGKMNSEEEDEVWQVIIGARAEMTSKHQEYLKLETTWMTAVGLSEMAAEAAYQTGADQASITARNHIQLVKLQVEEVHQLSRKAETKLAEAQIEELRQKTQEEGEERAESEQEAYLRED

SEQ ID NO: 136 - DNA, 717 bp, unidentified
  misc_feature 1..717: note = Description of Unknown: Smac/DIABLO sequence
  source 1..717: mol_type = other DNA
atggccgctctgaagtcctggctgagcagaagcgtgaccagcttcttccggtacagacagtgcctgtgcgtgcccgtggtggccaacttcaagaagagatgcttcagcgagctgatcagaccctggcacaagaccgtgaccatcggctttggcgtgaccctgtgtgccgtgcctatcgctcagaagtctgagcctcacagcctgtctagcgaggcccttatgagaagggccgtgtctctggtcaccgacagcaccagcacatttctgagccagaccacatacgccctgatcgaggccatcaccgagtacaccaaggccgtgtacaccctgaccagcctgtaccggcagtacacatctctgctgggcaagatgaacagcgaggaagaggacgaagtctggcaagtgatcatcggcgccagagccgagatgaccagcaagcaccaagagtacctgaagctggaaaccacctggatgacagccgtgggcctgtctgaaatggccgccgaagctgcttatcagaccggcgctgatcaggccagcatcaccgccagaaatcacatccagctggtcaagctgcaggtcgaggaagtgcaccagctgtccagaaaggccgagacaaagctggctgaggcccagatcgaggaactgcggcagaaaacccaagaggaaggcgaggaaagagccgagtctgagcaagaggcctacctgagagaggac

SEQ ID NO: 137 - protein, 45 aa, unidentified; identical to SEQ ID NO: 96
  REGION 1..45: note = Description of Unknown: minKRAB sequence

SEQ ID NO: 138 - DNA, 65 bp, synthetic construct
  misc_feature 1..65: note = Description of Artificial Sequence: Synthetic oligonucleotide
  source 1..65: mol_type = other DNA
tccaggcgatctgacggttcactaaacgagctctgcttatataggcctcccaccgtacacgccta

SEQ ID NO: 139 - DNA, 1491 bp, synthetic construct; identical to SEQ ID NO: 110
  misc_feature 1..1491: note = Description of Artificial Sequence: Synthetic polynucleotide
  source 1..1491: mol_type = other DNA

SEQ ID NO: 140 - DNA, 1491 bp, synthetic construct; identical to SEQ ID NO: 112
  misc_feature 1..1491: note = Description of Artificial Sequence: Synthetic polynucleotide
  source 1..1491: mol_type = other DNA

SEQ ID NO: 141 - DNA, 1491 bp, synthetic construct; identical to SEQ ID NO: 114
  misc_feature 1..1491: note = Description of Artificial Sequence: Synthetic polynucleotide
  source 1..1491: mol_type = other DNA

SEQ ID NO: 142 - DNA, 1491 bp, synthetic construct; identical to SEQ ID NO: 116
  misc_feature 1..1491: note = Description of Artificial Sequence: Synthetic polynucleotide
  source 1..1491: mol_type = other DNA

SEQ ID NO: 143 - DNA, 1491 bp, synthetic construct; identical to SEQ ID NO: 118
  misc_feature 1..1491: note = Description of Artificial Sequence: Synthetic polynucleotide
  source 1..1491: mol_type = other DNA

SEQ ID NO: 144 - DNA, 1491 bp, synthetic construct; identical to SEQ ID NO: 120
  misc_feature 1..1491: note = Description of Artificial Sequence: Synthetic polynucleotide
  source 1..1491: mol_type = other DNA

SEQ ID NO: 145 - DNA, 1491 bp, synthetic construct; identical to SEQ ID NO: 122
  misc_feature 1..1491: note = Description of Artificial Sequence: Synthetic polynucleotide
  source 1..1491: mol_type = other DNA

SEQ ID NO: 146 - DNA, 2061 bp, synthetic construct
  misc_feature 1..2061: note = Description of Artificial Sequence: Synthetic polynucleotide
  source 1..2061: mol_type = other DNA
atggcgggcgagggcgatcagcaagacgcggctcacaacatgggaaatcacttacccttacttcccgcagaatctgaagaggaagatgagatggaggtagaggatcaggactctaaagaagcgaaaaagcctaatatcatcaactttgacacctctcttccaaccagtcacacttacctgggggcagacatggaggaattccatggtcgaactctccacgacgacgatagctgccaagtgataccagtgctccctcaggtaatgatgattcttattcctggacagaccctgcctctacagctgttccaccctcaagaggtgagcatggtcaggaatttgatccagaaggacagaacatttgcggtcctggcctactcaaacgtacaggagcgagaggctcagttcgggaccactgccgaaatatacgcgtaccgggaggagcaagacttcgggatcgaaatcgtgaaggtaaaggccattggcagacaacggtttaaggtccttgagctccggacgcagagtgatgggatacaacaggctaaagtgcagatcctaccagagtgtgtattaccatctacctatgatgcagagactctcatggaccgcataaaaaagcaattaagggaatgggacgaaaacctcaaggacgattcactcccatccaaccccattgacttctcatatagggtcgctgcttgtttgcctatcgacgacgtccttaggatacagctcctgaaaatcggaagcgcaatacaaagattgcgctgtgagctggacattatgaataagtgcacttcactgtgttgtaagcagtgtcaagagaccgaaatcactacgaagaacgagatcttctctctctccctctgcgggccaatggcagcatatgtaaatccgcatgggtatgttcatgagacactaactgtgtacaaggcctgcaatttaaacttgatcggccggccatctacagaacacagttggttcccgggttatgcctggacagtggcacagtgcaaaatttgtgcttcccacattggatggaaatttacagccacaaagaaggatatgagtccccaaaagttctggggactgaccaggagcgctttattgcccaccatcccggacacagaggacgaaatctctccggacaaggtgattctctgtctggggggtggaggttcagggggtggaggttcaggtggtggcggtagtgtcgatggcttcatggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 147 - DNA, 1080 bp, synthetic construct
  misc_feature 1..1080: note = Description of Artificial Sequence: Synthetic polynucleotide
  source 1..1080: mol_type = other DNA
atgttcaacgtgctgatggtgcacaagcggagccacaccggcgaaagacctctgcagtgtgaaatctgcggcttcacctgtcggcagaagggcaacctgctgcggcacatcaaactgcacacaggcgagaagcccttcaagtgccacctgtgcaattacgcctgccagagaagagatgccctggggggtggaggttcagggggtggaggttcaggtggtggcggtagtgtcgatggcttcatggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatca

SEQ ID NO: 148 - DNA, 1077 bp, synthetic construct
  misc_feature 1..1077: note = Description of Artificial Sequence: Synthetic polynucleotide
  source 1..1077: mol_type = other DNA
atgttcaacgtgctgatggtgcacaggcggagccacaccggcgaaagacctctgcagtgtgaaatctgcggcttcacctgtcggcagaggggcaacctgctgcggcacatcagactgcacacaggcgagaggcccttcaggtgccacctgtgcaattacgcctgccagagaagagatgccctggggggtggaggttcagggggtggaggttcaggtggtggcggtagtgtcgatggcttcgatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaggttgcggcgtcgcttctcctcgctgcatttcatggtggaggtgaggggcgacctgactgccaggagaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaggattgtgaacatcttcaatgggaccagctgccccagcctgggagggaggcccaggctctttttcatccaggcctgtggtggggagcagagagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaggagtggctcctggtacgttgagaccctggacgacatctttgagcagtgggctcactctgaagacctgcagtccctcctgcttagggtcgctaatgctgtttcggtgagagggatttatagacagatgcctggttgctttaatttcctccggagaggacttttctttagaacatca

SEQ ID NO: 149 - DNA, 2061 bp, synthetic construct
  misc_feature 1..2061: note = Description of Artificial Sequence: Synthetic polynucleotide
  source 1..2061: mol_type = other DNA
atggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaagttgcggcgtcgcttctcctcgctgcatttcatggtggaggtgaagggcgacctgactgccaagaaaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaagattgtgaacatcttcaatgggaccagctgccccagcctgggagggaagcccaagctctttttcatccaggcctgtggtggggagcagaaagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaagagtggctcctggtacgttgagaccctggacgacatctttgagcagtgggctcactctgaagacctgcagtccctcctgcttagggtcgctaatgctgtttcggtgaaagggatttataaacagatgcctggttgctttaatttcctccggaaaaaacttttctttaaaacatcagggggtggaggttcagggggtggaggttcaggtggtggcggtagtgtcgatggcttcatggcgggcgagggcgatcagcaagacgcggctcacaacatgggaaatcacttacccttacttcccgcagaatctgaagaggaagatgagatggaggtagaggatcaggactctaaagaagcgaaaaagcctaatatcatcaactttgacacctctcttccaaccagtcacacttacctgggggcagacatggaggaattccatggtcgaactctccacgacgacgatagctgccaagtgataccagtgctccctcaggtaatgatgattcttattcctggacagaccctgcctctacagctgttccaccctcaagaggtgagcatggtcaggaatttgatccagaaggacagaacatttgcggtcctggcctactcaaacgtacaggagcgagaggctcagttcgggaccactgccgaaatatacgcgtaccgggaggagcaagacttcgggatcgaaatcgtgaaggtaaaggccattggcagacaacggtttaaggtccttgagctccggacgcagagtgatgggatacaacaggctaaagtgcagatcctaccagagtgtgtattaccatctacctatgatgcagagactctcatggaccgcataaaaaagcaattaagggaatgggacgaaaacctcaaggacgattcactcccatccaaccccattgacttctcatatagggtcgctgcttgtttgcctatcgacgacgtccttaggatacagctcctgaaaatcggaagcgcaatacaaagattgcgctgtgagctggacattatgaataagtgcacttcactgtgttgtaagcagtgtcaagagaccgaaatcactacgaagaacgagatcttctctctctccctctgcgggccaatggcagcatatgtaaatccgcatgggtatgttcatgagacactaactgtgtacaaggcctgcaatttaaacttgatcggccggccatctacagaacacagttggttcccgggttatgcctggacagtggcacagtgcaaaatttgtgcttcccacattggatggaaatttacagccacaaagaaggatatgagtccccaaaagttctggggactgaccaggagcgctttattgcccaccatcccggacacagaggacgaaatctctccggacaaggtgattctctgtctg

SEQ ID NO: 150 - DNA, 1077 bp, synthetic construct; identical to SEQ ID NO: 148
  misc_feature 1..1077: note = Description of Artificial Sequence: Synthetic polynucleotide
  source 1..1077: mol_type = other DNA

SEQ ID NO: 151 - DNA, 1077 bp, synthetic construct
  misc_feature 1..1077: note = Description of Artificial Sequence: Synthetic polynucleotide
  source 1..1077: mol_type = other DNA
atggatgtcggtgctcttgagagtttgaggggaaatgcagatttggcttacatcctgagcatggagccctgtggccactgcctcattatcaacaatgtgaacttctgccgtgagtccgggctccgcacccgcactggctccaacatcgactgtgagaggttgcggcgtcgcttctcctcgctgcatttcatggtggaggtgaggggcgacctgactgccaggagaatggtgctggctttgctggagctggcgcggcaggaccacggtgctctggactgctgcgtggtggtcattctctctcacggctgtcaggccagccacctgcagttcccaggggctgtctacggcacagatggatgccctgtgtcggtcgagaggattgtgaacatcttcaatgggaccagctgccccagcctgggagggaggcccaggctctttttcatccaggcctgtggtggggagcagagagaccatgggtttgaggtggcctccacttcccctgaagacgagtcccctggcagtaaccccgagccagatgccaccccgttccaggaaggtttgaggaccttcgaccagctggacgccatatctagtttgcccacacccagtgacatctttgtgtcctactctactttcccaggttttgtttcctggagggaccccaggagtggctcctggtacgttgagaccctggacgacatctttgagcagtgggctcactctgaagacctgcagtccctcctgcttagggtcgctaatgctgtttcggtgagagggatttatagacagatgcctggttgctttaatttcctccggagaggacttttctttagaacatcagggggtggaggttcagggggtggaggttcaggtggtggcggtagtgtcgatggcttcttcaacgtgctgatggtgcacaggcggagccacaccggcgaaagacctctgcagtgtgaaatctgcggcttcacctgtcggcagaggggcaacctgctgcggcacatcagactgcacacaggcgagaggcccttcaggtgccacctgtgcaattacgcctgccagagaagagatgccctg

SEQ ID NO: 152 - protein, 4 aa, unidentified
  REGION 1..4: note = Description of Unknown: hairy-related basic helix-loop-helix repressor domain sequence
WRPW

SEQ ID NO: 153 - DNA, 582 bp, synthetic construct
  misc_feature 1..582: note = Description of Artificial Sequence: Synthetic polynucleotide
  source 1..582: mol_type = other DNA
gactgtgaggtcaacaacggttccagcctcagggatgagtgcatcacaaacctactggtgtttggcttcctccaaagctgttctgacaacagcttccgcagagagctggacgcactgggccacgagctgccagtgctggctccccagtgggagggctacgatgagctgcagactgatggcaaccgcagcagccactcccgcttgggaagaatagaggcagattctgaaagtcaagaagacatcatccggaatattgccaggcacctcgcccaggtcggggacagcatggaccgtagcatccctccgggcctggtgaacggcctggccctgcagctcaggaacaccagccggtcggaggaggaccggaacagggacctggccactgccctggagcagctgctgcaggcctaccctagagacatggagaaggagaagaccatgctggtgctggccctgctgctggccaagaaggtggccagtcacacgccgtccttgctccgtgatgtctttcacacaacagtgaattttattaaccagaacctacgcacctacgtgaggagcttagccagaaatgggatggac

SEQ ID NO: 154 - protein, 6 aa, synthetic construct
  REGION 1..6: note = Description of Artificial Sequence: Synthetic peptide
  VARIANT 4..5: note = X can be any amino acid
DSGXXS

SEQ ID NO: 155 - protein, 6 aa, synthetic construct
  REGION 1..6: note = Description of Artificial Sequence: Synthetic 6xHis tag
HHHHHH

(19) United States
(12) Patent Application Publication
(10) Pub. No.: US 2023/0405144 A1
(43) Pub. Date: Dec. 21, 2023

(54) INDUCIBLE CELL DEATH SYSTEMS

(71) Applicant: Senti Biosciences, Inc., South San Francisco, CA (US)

(72) Inventors: Rocky CHEUNG, South San Francisco, CA (US); Russell Morrison GORDLEY, San Francisco, CA (US); Timothy Kuan-Ta LU, Cambridge, MA (US); Michelle Elizabeth HUNG, South San Francisco, CA (US); Rebecca Tayler COTTMAN, San Carlos, CA (US)

(21) Appl. No.: 18/199,154

(22) Filed: May 18, 2023

Related U.S. Application Data
(63) Continuation of application No. PCT/US2021/060397, filed on Nov. 22, 2021.
(60) Provisional application No. 63/116,433, filed on Nov. 20, 2020.

(51) Int. Cl. A61K 47/68; A61K 47/64; A61K 47/66

(52) U.S. Cl. CPC A61K 47/6897; A61K 47/64; A61K 47/6425; A61K 47/67; A61K 47/6815; A61K 47/6813; A61K 47/6829

(57) ABSTRACT

Provided herein are compositions and methods for inducing cell death in a regulated manner, for example in a safety switch system. Inducible cell death can be triggered by ligand binding to a one or more ligand binding domains. Cell death can include induction of apoptosis.

### CROSS REFERENCE TO RELATED APPLICATIONS

This application is a continuation of International Application No. PCT/US2021/060397, filed Nov. 22, 2021, which claims the benefit of U.S. Provisional Application No. 63/116,433 filed Nov. 20, 2020, each of which are hereby incorporated in their entirety by reference for all purposes.

### SEQUENCE LISTING

The instant application contains a Sequence Listing which has been submitted electronically in XML format and is hereby incorporated by reference in its entirety. Said Sequence Listing XML, created on Aug. 31, 2023, is named STB-026WOC1.xml, and is 280,103 bytes in size.

### BACKGROUND

Currently available cell and gene therapy products can lack control, which can lead to safety concerns such as toxicity in subjects that receive the therapies. Thus, additional methods of controlling and regulating these therapies are needed.

### SUMMARY

Disclosed herein are inducible cell death systems that can be used for inducing cell death in a regulated manner, for example in a safety switch system.

In some aspects, the present disclosure provides an inducible cell death system comprising two or more polypeptide monomers, wherein each polypeptide monomer comprises one or more ligand binding domains and a cell death-inducing domain, wherein the polypeptide monomers are configured to oligomerize upon contacting the polypeptide monomers with a cognate ligand of the one or more ligand binding domains and generate a cell-death inducing signal in a cell in which the polypeptide monomers are expressed, and wherein:
the one or more ligand binding domains of each of the two or more polypeptide monomers comprise a domain or functional fragment thereof selected from the group consisting of:
an ABI domain, optionally comprising the amino sequence of SEQ ID NO: 31; a PYL domain, optionally comprising the amino acid sequence of SEQ ID NO: 53; a caffeine-binding single-domain antibody, optionally comprising the amino acid sequence of SEQ ID NO: 33; a cannabidiol binding domain, optionally comprising an amino acid sequence selected from the group consisting of SEQ ID NO: 34, 35, 36, 37, and 38; a hormone-binding domain of estrogen receptor (ER) domain, optionally comprising the amino acid sequence of SEQ ID NO: 42; a heavy chain variable region (VH) of an anti-nicotine antibody, optionally comprising the amino acid sequence of SEQ ID NO: 50, and/or the light chain variable region (VL) of an anti-nicotine antibody, optionally comprising the amino acid sequence of SEQ ID NO: 51; an FKBP domain, optionally comprising the amino acid sequence of SEQ ID NO: 43; and a progesterone receptor domain, optionally comprising the amino acid sequence of SEQ ID NO: 52; or
the one or more ligand binding domains of a first of the two or more polypeptide monomers comprise an FKBP domain, optionally comprising the amino acid sequence of SEQ ID NO: 43, and the one or more ligand binding domains of a second of the two or more polypeptide monomers comprise an FRB domain, optionally comprising the amino acid sequence of SEQ ID NO: 44; or
the one or more ligand binding domains of a first of the two or more polypeptide monomers comprise a cereblon domain, optionally comprising the amino acid sequence set forth in one of SEQ ID NOs: 127 and 129, and the one or more ligand binding domains of a second of the two or more polypeptide monomers comprise a degron, optionally comprising the amino acid sequence set forth in one of SEQ ID NOs: 131 and 133, and
optionally wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32.

In some aspects, each polypeptide monomer comprises the same ligand binding domain, optionally wherein each polypeptide monomer comprises:
an FKBP domain, optionally wherein the cognate ligand is FK1012, a derivative thereof, or an analog thereof; or
an ABI domain and a PYL domain, optionally wherein the cognate ligand is abscisic acid; or
a first cannabidiol binding domain comprising the amino acid sequence of SEQ ID NO: 34, and a second cannabidiol binding domain comprising an amino acid sequence selected from the group consisting of SEQ ID NO: 35, 36, 37, and 38, optionally wherein the cognate ligand is a phytocannabinoid, optionally the phytocannabinoid is cannabidiol; or
a hormone-binding domain of estrogen receptor (ER) domain and an FKBP domain, optionally wherein the cognate ligand is rapamycin or a derivative thereof or an analog thereof and/or tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen;
two caffeine-binding single-domain antibodies, optionally wherein each caffeine-binding single-domain antibody comprises the amino acid sequence of SEQ ID NO: 33, optionally wherein the cognate ligand is caffeine or a derivative thereof; or
a first progesterone receptor domain comprising the amino acid sequence of SEQ ID NO: 52, and a second first progesterone receptor domain comprising the amino acid sequence of SEQ ID NO: 52, optionally wherein the cognate ligand is mifepristone or a derivative thereof, optionally wherein the polypeptide monomers are configured to form homooligomers upon contact with the cognate ligand, and optionally wherein the homooligomers comprise homodimers.

In some aspects, a first polypeptide monomer comprises a first ligand binding domain and a second polypeptide monomer comprises a second ligand binding domain, optionally wherein:
the first monomer comprises an FKBP domain and the second monomer comprises an FRB domain, optionally wherein the cognate ligand is rapamycin or a derivative thereof; or
the first polypeptide monomer comprises a hormone-binding domain of estrogen receptor (ER) domain and the second polypeptide monomer comprises an FKBP domain, optionally wherein the cognate ligand is rapamycin or a derivative thereof and/or tamoxifen or a metabolite thereof; or
the first polypeptide monomer comprises an FRB domain and the second polypeptide monomer comprises a hormone-binding domain of estrogen receptor (ER) domain, optionally wherein the cognate ligand is rapamycin or a derivative thereof and/or tamoxifen or a metabolite thereof; or wherein the first polypeptide monomer comprises a hormone-binding domain of estrogen receptor (ER) domain and an FKBP domain, and the second polypeptide monomer comprises an FRB domain and a hormone-binding domain of estrogen receptor (ER) domain, optionally wherein the cognate ligand is rapamycin or a derivative thereof and/or tamoxifen or a metabolite thereof; or
the first polypeptide monomer comprises an ABI domain and the second polypeptide monomer comprises a PYL domain, optionally wherein the cognate ligand comprises abscisic acid; or
the first polypeptide monomer comprises a heavy chain variable region (VH) of an anti-nicotine antibody and the second polypeptide monomer comprises a light chain variable region (VL) of an anti-nicotine antibody, optionally wherein the anti-nicotine antibody is a Nic12 antibody, optionally wherein the VH comprises the amino acid sequence of SEQ ID NO: 50, and optionally wherein the VL comprises the amino acid sequence of SEQ ID NO: 51, and optionally wherein the cognate ligand is nicotine or a derivative thereof; or
the first polypeptide monomer comprises a cannabidiol binding domain comprising an amino acid sequence selected from the group consisting of SEQ ID NO: 35, 36, 37, and 38 and the second polypeptide monomer comprises a cannabidiol binding domain comprising the amino acid sequence of SEQ ID NO: 34. optionally wherein the cognate ligand is a phytocannabinoid, optionally wherein the phytocannabinoid is cannabidiol; or
the first polypeptide monomer comprises a cereblon domain comprising the amino acid sequence set forth in one of SEQ ID NOs: 127 and 129 and the second polypeptide monomer comprises a degron domain comprising the amino acid sequence set forth in one of SEQ ID NOs: 131 and 133, optionally wherein the cognate ligand is an IMiD, optionally wherein the IMiD is an FDA-approved drug, and optionally wherein the IMiD is selected from the group consisting of: thalidomide, lenalidomide, and pomalidomide,
optionally wherein the polypeptide monomers are configured to form heterooligomers upon contact with the cognate ligand, and optionally wherein the heterooligomers comprise heterodimers.

In some aspects, the at least one of the two or more polypeptide monomers further comprises a linker localized between each ligand binding domain and cell death-inducing domain, optionally wherein the linker comprises an amino acid sequence selected from the group consisting of: GGGGSGGGGSGGGGSVDGF (SEQ ID NO: 101) and ASGGGGSAS (SEQ ID NO: 102), optionally wherein each polypeptide monomer further comprises a linker localized between each ligand binding domain and cell death-inducing domain.

In some aspects, the present disclosure provides an inducible cell death system comprising an activation-conditional control polypeptide (ACP), wherein the ACP comprises a ligand binding domain and a transcriptional effector domain, and
wherein upon binding of the ligand binding domain to a cognate ligand, the ACP is capable of modulating transcriptional expression of a gene of interest operably linked to an ACP-responsive promoter,
wherein the gene of interest comprises a cell death inducing polypeptide, and
optionally wherein the ligand binding domain comprises a degron, optionally wherein the degron is capable of inducing degradation of the ACP, and
optionally wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box, or the degron comprises a cereblon (CRBN) polypeptide substrate domain capable of binding CRBN in response to an immunomodulatory drug (IMiD) thereby promoting ubiquitin pathway-mediated degradation of the regulatable polypeptide, optionally wherein the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN, optionally wherein the CRBN polypeptide substrate domain is a chimeric fusion product of native CRBN polypeptide sequences, optionally wherein the CRBN polypeptide substrate domain is a IKZF3/ZFP91/IKZF3 chimeric fusion product having the amino acid sequence of

(SEQ ID NO: 103)
FNVLMVHKRSHTGERPLQCEICGFTCRQKGNLLRHIKLHTGEKPFKCHL
CNYACQRRDAL.

In some aspects, the present disclosure provides an inducible cell death system PGP-21,DNA comprising an activation-conditional control polypeptide (ACP), wherein the ACP comprises one or more ligand binding domains and a transcription factor comprising a nucleic acid-binding domain and a transcriptional effector domain,
wherein the ACP undergoes nuclear localization upon binding of the ligand binding domain to a cognate ligand, and
wherein when localized to a cell nucleus, the ACP is capable of inducing transcriptional expression of a gene of interest operably linked to an ACP-responsive promoter,
wherein the gene of interest comprises a cell death-inducing domain,
optionally wherein the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, and
optionally wherein each of the one or more ligand binding domains comprises: a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen; or a progesterone receptor domain optionally comprising the amino acid sequence of SEQ ID NO: 52, and optionally wherein the cognate ligand is mifepristone or a derivative thereof, or optionally wherein each of the one or more ligand binding domain comprises a domain or functional fragment thereof selected from the group consisting of:
an ABI domain, optionally comprising the amino acid sequence of SEQ ID NO: 31, and optionally wherein the cognate ligand is abscisic acid;
a PYL domain, optionally comprising the amino acid sequence of SEQ ID NO: 53, and optionally wherein the cognate ligand is abscisic acid;
a caffeine-binding single-domain antibody optionally comprising the amino acid sequence of SEQ ID NO: 33, and optionally wherein the cognate ligand is caffeine or a derivative thereof;
a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol;
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen;
a heavy chain variable region (VH) of an anti-nicotine antibody optionally comprising the amino acid sequence of SEQ ID NO: 50, and optionally wherein the cognate ligand is nicotine or a derivative thereof;
a light chain variable region (VL) of an anti-nicotine antibody optionally comprising the amino acid sequence of SEQ ID NO: 51, and optionally wherein the cognate ligand is nicotine or a derivative thereof;
a progesterone receptor domain optionally comprising the amino acid sequence of SEQ ID NO: 52, and optionally wherein the cognate ligand is mifepristone or a derivative thereof;
an FKBP domain optionally comprising the amino acid sequence of SEQ ID NO: 43, and optionally wherein the cognate ligand is rapamycin, AP1903, AP20187, FK1012, derivatives thereof, or analogs thereof; and
an FRB domain optionally comprising the amino acid sequence of SEQ ID NO: 44, and optionally wherein the cognate ligand is rapamycin, AP1903, AP20187, FK1012, derivatives thereof, or analogs thereof,
optionally wherein the nucleic acid-binding domain comprises a DNA-binding zinc finger protein domain (ZF protein domain), optionally wherein the ZF protein domain is modular in design and is composed of an array of zinc finger motifs, optionally wherein the ZF-protein domain comprises one to ten zinc finger motifs,
optionally wherein the gene of interest is a cell death-inducing polypeptide, optionally wherein the cell death-inducing domain is derived from a protein selected from the group consisting of:
caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32.

An inducible cell death system comprising an engineered regulatable cell survival polypeptide, the cell survival polypeptide comprising a pro-survival polypeptide and a heterologous ligand binding domain,
wherein upon binding of the ligand binding domain to a cognate ligand, the cognate ligand inhibits the pro-survival polypeptide,
optionally wherein the pro-survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein, optionally wherein the ligand binding domain is localized at the N-terminal region of the pro-survival polypeptide or at the C-terminal region of the pro-survival polypeptide.

In some aspects the present disclosure provides an inducible cell death system comprising a regulatable cell survival polypeptide and a cell death-inducing polypeptide, wherein the cell-survival polypeptide comprises a pro-survival polypeptide and a heterologous ligand binding domain,
wherein when expressed the cell survival polypeptide is capable of inhibiting the cell death-inducing polypeptide, and
wherein upon binding to a cognate ligand, the cognate ligand inhibits the pro-survival polypeptide, optionally wherein the cell survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein, optionally wherein the ligand binding domain is localized at the N-terminal region of the pro-survival polypeptide or at the C-terminal region of the pro-survival polypeptide.

In some aspects, the XIAP comprises the amino acid sequence of SEQ ID NO: 107, or the modified XIAP comprises one or more amino acid substitutions within positions 305-325 of SEQ ID NO: 107, optionally wherein the one or more amino acid substitutions are at one or more positions of SEQ ID NO: 107 selected from the group consisting of: 305, 306, 308, and 325, optionally wherein the amino acid substitution at position 305 of SEQ ID NO: 107 is G305M, optionally wherein the amino acid substitution at position 306 of SEQ ID NO: 107 is G3065, optionally wherein the amino acid substitution at position 308 of SEQ ID NO: 107 is selected from the group consisting of T3085 and T308D, optionally wherein the amino acid substitution at position 325 of SEQ ID NO: 107 is P325S.

In some aspects the ligand binding domain comprises a domain, or functional fragment thereof, selected from the group consisting of:
an ABI domain, optionally comprising the amino acid sequence of SEQ ID NO: 31, and optionally wherein the cognate ligand is abscisic acid;
a PYL domain, optionally comprising the amino acid sequence of SEQ ID NO: 53, and optionally wherein the cognate ligand is abscisic acid;
a caffeine-binding single-domain antibody optionally comprising the amino acid sequence of SEQ ID NO: 33, and optionally wherein the cognate ligand is caffeine or a derivative thereof;
a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol;
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen;
a heavy chain variable region (VH) of an anti-nicotine antibody optionally comprising the amino acid sequence of SEQ ID NO: 50, and optionally wherein the cognate ligand is nicotine or a derivative thereof;
a light chain variable region (VL) of an anti-nicotine antibody optionally comprising the amino acid sequence of SEQ ID NO: 51, and optionally wherein the cognate ligand is nicotine or a derivative thereof;
a progesterone receptor domain optionally comprising the amino acid sequence of SEQ ID NO: 52, and optionally wherein the cognate ligand is mifepristone or a derivative thereof;
an FKBP domain optionally comprising the amino acid sequence of SEQ ID NO: 43, and optionally wherein the cognate ligand is rapamycin, AP1903, AP20187, FK1012, derivatives thereof, or analogs thereof; and
an FRB domain optionally comprising the amino acid sequence of SEQ ID NO: 44, and optionally wherein the cognate ligand is rapamycin, AP1903, AP20187, FK1012, derivatives thereof, or analogs thereof.

In some aspects, the ligand binding domain comprises a degron, optionally wherein the degron is capable of inducing degradation of the regulatable cell survival polypeptide, and optionally wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box, optionally wherein the degron comprises a cereblon (CRBN) polypeptide substrate domain capable of binding CRBN in response to an immunomodulatory drug (IMiD) thereby promoting ubiquitin pathway-mediated degradation of the regulatable polypeptide, optionally wherein the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN, optionally wherein the CRBN polypeptide substrate domain is a chimeric fusion product of native CRBN polypeptide sequences, optionally wherein the CRBN polypeptide substrate domain is a IKZF3/ZFP91/IKZF3 chimeric fusion product having the amino acid sequence of FNVLMVHKRSHTGERPLQCEICGFTCRQKGNLLRHIKLHTGEKPFKCHLCNYACQRRD AL (SEQ ID NO: 103), optionally wherein the cognate ligand is an IMiD, optionally wherein the IMiD is an FDA-approved drug, and optionally wherein the IMiD is selected from the group consisting of: thalidomide, lenalidomide, and pomalidomide.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B 1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the Bax comprises the amino acid sequence of SEQ ID NO: 32.

In some aspects, the present disclosure provides an activation-conditional control polypeptide (ACP) comprising:
a) a first chimeric polypeptide, wherein the first chimeric polypeptide comprises a first ligand binding domain and a transcriptional activation domain; and
b) a second chimeric polypeptide, wherein the second chimeric polypeptide comprises a second ligand binding domain and a nucleic acid-binding domain,
wherein the first chimeric polypeptide and the second chimeric polypeptide oligomerize to form the multimeric ACP via a cognate ligand that binds to each ligand binding domain, and
wherein the multimeric ACP is capable of inducing transcriptional expression of a gene of interest operably linked to an ACP-responsive promoter, and
optionally wherein the transcriptional activation domain is selected from the group consisting of:
a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain),
optionally wherein each of the one or more ligand binding domains comprises a domain, or functional fragment thereof, selected from the group consisting of:
an ABI domain, optionally comprising the amino acid sequence of SEQ ID NO: 31, and optionally wherein the cognate ligand is abscisic acid;
a PYL domain, optionally comprising the amino acid sequence of SEQ ID NO: 53, and optionally wherein the cognate ligand is abscisic acid;
a caffeine-binding single-domain antibody optionally comprising the amino acid sequence of SEQ ID NO: 33, and optionally wherein the cognate ligand is caffeine or a derivative thereof; a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol;
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen;
a heavy chain variable region (VH) of an anti-nicotine antibody optionally comprising the amino acid sequence of SEQ ID NO: 50, and optionally wherein the cognate ligand is nicotine or a derivative thereof;
a light chain variable region (VL) of an anti-nicotine antibody optionally comprising the amino acid sequence of SEQ ID NO: 51, and optionally wherein the cognate ligand is nicotine or a derivative thereof;
a progesterone receptor domain optionally comprising the amino acid sequence of SEQ ID NO: 52, and optionally wherein the cognate ligand is mifepristone or a derivative thereof;
an FKBP domain optionally comprising the amino acid sequence of SEQ ID NO: 43, and optionally wherein the cognate ligand is rapamycin, AP1903, AP20187, FK1012, derivatives thereof, or analogs thereof; and
an FRB domain optionally comprising the amino acid sequence of SEQ ID NO: 44, and optionally wherein the cognate ligand is rapamycin, AP1903, AP20187, FK1012, derivatives thereof, or analogs thereof,
optionally wherein the gene of interest is a cell death-inducing polypeptide, optionally wherein the cell death-inducing domain is derived from a protein selected from the group consisting of:
caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32.

The ACP of claim 13, wherein the nucleic acid-binding domain comprises a DNA-binding zinc finger protein domain (ZF protein domain), optionally wherein the ZF protein domain is modular in design and is composed of an array of zinc finger motifs, optionally wherein the ZF-protein domain comprises one to ten zinc finger motifs.

In some aspects, the chimeric polypeptide further comprises a linker localized between the nucleic acid-binding domain and the transcriptional effector domain, optionally wherein the linker comprises one or more 2A ribosome skipping tags, optionally wherein each 2A ribosome skipping tag is selected from the group consisting of: P2A, T2A, E2A, and F2A.

In some aspects, the chimeric polypeptide comprises a first ligand binding domain operably linked to the nucleic acid-binding domain and a second ligand binding domain operably linked to the transcriptional effector domain; optionally wherein:
each of the first and second ligand binding domains comprises a hormone-binding domain of estrogen receptor (ER) domain, optionally wherein the cognate ligand is tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen; or
each of the first and second ligand binding domains comprises a progesterone receptor domain., optionally wherein the cognate ligand is mifepristone or a derivative thereof, and optionally wherein when the ligand binding domain comprises an ABI domain or a PYL domain, the cognate ligand is abscisic acid; or
each of the first and second ligand binding domains comprises a caffeine-binding single-domain antibody, optionally wherein the cognate ligand is caffeine or a derivative thereof; or
each of the first and the second ligand binding domains comprises a cannabidiol binding domain, optionally wherein the cognate ligand is a cannabidiol or a phytocannabinoid,
optionally wherein the cannabidiol binding domain comprises a single-domain antibody or a nanobody, and optionally wherein the cannabidiol binding domain comprises an amino acid sequence selected from the group consisting of SEQ ID NO: 34, 35, 36, 37, and 38.

In some aspects, the nucleic acid-binding domain binds to the ACP-responsive promoter, optionally wherein the ACP-responsive promoter comprises an ACP-binding domain sequence and a promoter sequence, optionally wherein the promoter sequence comprises a minimal promoter, optionally wherein the promoter sequence is an inducible promoter and further comprises a responsive element selected from the group consisting of: NFκB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule-responsive promoters, and tandem repeats thereof, and optionally wherein the ACP-responsive promoter comprises a synthetic promoter, and optionally wherein the ACP-binding domain comprises one or more zinc finger binding sites.

In some aspects, the ligand binding domain is localized N-terminal to the transcriptional effector domain or C-terminal to the transcriptional effector domain.

In some aspects, the present disclosure provides an isolated cell comprising an inducible cell death system as previously described or an ACP as previously described.

In some aspects, the present disclosure provides an engineered nucleic acid encoding an inducible cell death system as previously described or an ACP as previously described.

In some aspects, provided herein is an isolated cell comprising an inducible cell death polypeptide comprising two or more monomers, wherein each monomer comprises one or more ligand binding domains and a cell death-inducing domain, wherein each of the one or more ligand binding domains comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain, wherein each monomer is oligomerizable via a cognate ligand that binds to the ligand binding domain, and wherein when the ligand oligomerizes each monomer, a cell death-inducing signal is generated in the cell.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

In some aspects, the cell death-inducing domain comprises caspase 9, or a functional truncation thereof. In some aspects, the cell death-inducing domain comprises the caspase 9 amino acid sequence of Table D.

In some aspects, the cell death-inducing domain comprises Bid, or a functional truncation thereof. In some aspects, the cell death-inducing domain comprises the Bid amino acid sequence of Table D.

In some aspects, the ABI domain comprises the amino acid sequence of Table D. In some aspects, the PYL domain comprises the amino acid sequence of Table D.

In some aspects, the caffeine-binding single-domain antibody comprises the amino acid sequence of Table D.

In some aspects, the cannabidiol binding domain comprises an amino acid sequence selected from the group consisting of the sequences for CA14, DB6, DB11, DB18, and DB21 as shown in Table D.

In some aspects, the hormone-binding domain of estrogen receptor (ER) domain comprises the amino acid sequence of Table D.

In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D.

In some aspects, the progesterone receptor domain comprises the amino acid sequence of Table D.

In some aspects, the FKBP domain comprises the amino acid sequence of Table D.

In some aspects, the FRB domain comprises the amino acid sequence of Table D.

In some aspects, each monomer comprises the same ligand binding domain. In some aspects, the inducible cell death polypeptide comprises homooligomers. In some aspects, the homooligomers comprise homodimers. In some aspects, each monomer comprises an FKBP domain. In some aspects, the ligand is FK1012, a derivative thereof, or an analog thereof.

In some aspects, the cell death-inducing domain comprises Bid, or a functional truncation thereof. In some aspects, the cell death-inducing domain comprises the Bid amino acid sequence of Table D.

In some aspects, each monomer comprises an ABI domain and a PYL domain. In some aspects, the ligand is abscisic acid. In some aspects, the cell death-inducing domain comprises caspase 9, or a functional truncation thereof. In some aspects, the cell death-inducing comprises the caspase 9 amino acid sequence of Table D.

In some aspects, each monomer comprises a cannabidiol binding domain comprising the CA14 amino acid sequence of Table D and a cannabidiol binding domain comprising an amino acid sequence selected from the group consisting of the sequences of DB6, DB11, DB18, and DB21 of Table D.

In some aspects, each monomer comprises a hormone-binding domain of estrogen receptor (ER) domain and an FKBP domain. In some aspects, each monomer comprises an FRB domain and a hormone-binding domain of estrogen receptor (ER) domain. In some aspects, the cell death-inducing domain comprises caspase 9, or a functional truncation thereof. In some aspects, the cell death-inducing domain comprises the caspase 9 amino acid sequence of Table D. In some aspects, the ligand is rapamycin, a derivative thereof, or an analog thereof. In some aspects, the ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, each monomer comprises two caffeine-binding single-domain antibodies. In some aspects, each caffeine-binding single-domain antibody comprises the amino acid sequence of Table D. In some aspects, the ligand is caffeine or a derivative thereof.

In some aspects, a first monomer comprises a first ligand binding domain and a second monomer comprises a second ligand binding domain. In some aspects, the inducible cell death polypeptide comprises heterooligomers. In some aspects, the heterooligomers comprise heterodimers. In some aspects, the first monomer comprises an FKBP domain and the second monomer comprises an FRB domain. In some aspects, the cell death-inducing domain comprises Bid, or a functional truncation thereof. In some aspects, the cell death-inducing domain comprises the Bid amino acid sequence of Table D.

In some aspects, the first monomer comprises a hormone-binding domain of estrogen receptor (ER) domain and the second monomer comprises an FKBP domain. In some aspects, the first monomer comprises an FRB domain and the second monomer comprises a hormone-binding domain of estrogen receptor (ER) domain. In some aspects, the first monomer comprises a hormone-binding domain of estrogen receptor (ER) domain and an FKBP domain, and the second monomer comprises an FRB domain and the second monomer comprises a hormone-binding domain of estrogen receptor (ER) domain. In some aspects, the cell death-inducing domain comprises caspase 9, or a functional truncation thereof. In some aspects, the cell death-inducing domain comprises the caspase 9 amino acid sequence of Table D. In some aspects, the ligand is rapamycin, a derivative thereof, or an analog thereof. In some aspects, the ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, the first monomer comprises an ABI domain and the second monomer comprises a PYL domain. In some aspects, the ligand is abscisic acid.

In some aspects, the first monomer comprises a heavy chain variable region (VH) of an anti-nicotine antibody and the second monomer comprises a light chain variable region (VL) of an anti-nicotine antibody. In some aspects, the anti-nicotine antibody is a Nic12 antibody. In some aspects, the VH comprises the VH amino acid sequence of Table D. In some aspects, the VL comprises the VL amino acid sequence of Table D. In some aspects, the ligand is nicotine or a derivative thereof.

In some aspects, the first monomer comprises a cannabidiol binding domain comprising an amino acid sequence selected from the group consisting of the sequences of DB6, DB11, DB18, and DB21 of Table D and the second monomer comprises a cannabidiol binding domain comprising the amino acid sequence of CA14 of Table D. In some aspects, the ligand is a cannabidiol or a phytocannabinoid.

In some aspects, each monomer further comprises a linker localized between each ligand binding domain and cell death-inducing domain. In some aspects, the linker comprises an amino acid sequence selected from the group consisting of: GGGGSGGGGSGGGGSVDGF (SEQ ID NO: 85) and ASGGGGSAS (SEQ ID NO: 86).

Also disclosed herein is an isolated cell comprising an activation-conditional control polypeptide (ACP), wherein the ACP comprises one or more ligand binding domains and a transcription factor comprising a nucleic acid-binding domain and a transcriptional effector domain, wherein the ACP undergoes nuclear localization upon binding of the ligand binding domain to a cognate ligand, and wherein when localized to a cell nucleus, the ACP is capable of inducing transcriptional expression of a gene of interest operably linked to an ACP-responsive promoter.

Also disclosed herein is an isolated cell comprising a multimeric activation-conditional control polypeptide (ACP), wherein the multimeric ACP comprises: (a) a first chimeric polypeptide, wherein the first chimeric polypeptide comprises a first ligand binding domain and a transcriptional activation domain; and (b) a second chimeric polypeptide, wherein the second chimeric polypeptide comprises a second ligand binding domain and a nucleic acid-binding domain, wherein the first chimeric polypeptide and the second chimeric polypeptide multimerize to form the multimeric ACP via a cognate ligand that binds to each ligand binding domain, and wherein the multimeric ACP is capable of inducing transcriptional expression of a gene of interest operably linked to an ACP-responsive promoter.

In some aspects, each ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain.

In some aspects, the ABI domain comprises the ABI amino acid sequence of Table D. In some aspects, the PYL domain comprises the PYL amino acid sequence of Table D. In some aspects, the caffeine-binding single-domain antibody comprises the amino acid sequence of Table D. In some aspects, the cannabidiol binding domain comprises an amino acid sequence selected from the group consisting of the sequences of CA14, DB6, DB11, DB18, and DB21 of Table D. In some aspects, the hormone-binding domain of estrogen receptor (ER) domain comprises the amino acid sequence of Table D. In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D. In some aspects, the progesterone receptor domain comprises the amino acid sequence of Table D. In some aspects, the FKBP domain comprises the amino acid sequence of Table D. In some aspects, the FRB domain comprises the amino acid sequence of Table D.

In some aspects, the nucleic acid-binding domain comprises a DNA-binding zinc finger protein domain (ZF protein domain). In some aspects, the ZF protein domain is modular in design and is composed of zinc finger arrays (ZFA). In some aspects, the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the chimeric polypeptide further comprises a linker localized between the nucleic acid-binding domain and the transcriptional effector domain. In some aspects, the linker comprises one or more 2A ribosome skipping tags. In some aspects, each 2A ribosome skipping tag is selected from the group consisting of: P2A, T2A, E2A, and F2A.

In some aspects, the chimeric polypeptide comprises a first ligand binding domain operably linked to the nucleic acid-binding domain and a second ligand binding domain operably linked to the transcriptional effector domain.

In some aspects, each of the first and second ligand binding domains comprises a hormone-binding domain of estrogen receptor (ER) domain. In some aspects, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, each of the first and second ligand binding domains comprises a progesterone receptor domain. In some aspects, the cognate ligand is mifepristone or a derivative thereof.

In some aspects, when the ligand binding domain comprises an ABI domain or a PYL domain, the cognate ligand is abscisic acid.

In some aspects, when the ligand binding domain comprises a caffeine-binding single-domain antibody, the cognate ligand is caffeine or a derivative thereof.

In some aspects, when the ligand binding domain comprises a cannabidiol binding domain, the cognate ligand is a cannabidiol or a phytocannabinoid. In some aspects, the cannabidiol binding domain comprises a single-domain antibody or a nanobody. In some aspects, the cannabidiol binding domain comprises an amino acid sequence selected from the group consisting of the sequence of CA14, DB6, DB11, DB18, and DB21 of Table D.

In some aspects, when the ligand binding domain comprises a hormone-binding domain of estrogen receptor (ER) domain, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, when the ligand binding domain comprises a heavy chain variable region (VH) of an anti-nicotine antibody or a light chain variable region (VL) of an anti-nicotine antibody, the cognate ligand is nicotine or a derivative thereof.

In some aspects, when the ligand binding domain is a progesterone receptor domain, the cognate ligand is mifepristone or a derivative thereof.

In some aspects, when the ligand binding domain comprises an FKBP domain or an FRB domain, the cognate ligand is rapamycin, AP1903, AP20187, FK1012, derivatives thereof, or analogs thereof.

In some aspects, the nucleic acid-binding domain comprises a DNA-binding zinc finger protein domain (ZF protein domain).

In some aspects, the ZF protein domain is modular in design and is composed of zinc finger arrays (ZFA). In some aspects, the ZF protein domain comprises one to ten ZFA.

In some aspects, the nucleic acid-binding domain binds to the ACP-responsive promoter. In some aspects, the ACP-responsive promoter comprises an ACP-binding domain sequence and a promoter sequence. In some aspects, the promoter sequence is derived from a promoter selected from the group consisting of: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof. In some aspects, the ACP-responsive promoter comprises a synthetic promoter. In some aspects, the ACP-responsive promoter comprises a minimal promoter.

In some aspects, the ACP-binding domain comprises one or more zinc finger binding sites.

In some aspects, the transcriptional effector domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

Also disclosed herein is an isolated cell comprising an activation-conditional control polypeptide (ACP), wherein the ACP comprises a ligand binding domain and a transcriptional effector domain, and wherein upon binding of the ligand binding domain to a cognate ligand, the ACP is capable of modulating transcriptional expression of a gene of interest operably linked to an ACP-responsive promoter.

In some aspects, ligand binding domain is localized 5′ of the transcriptional effector domain or 3′ of the transcriptional effector domain. In some aspects, the transcriptional effector domain comprises a transcriptional repressor domain. In some aspects, the transcriptional repressor domain is selected from the group consisting of: a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the transcriptional effector domain comprises a transcriptional activator domain. In some aspects, the transcriptional activator domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

In some aspects, the ACP is a transcription factor. In some aspects, the ACP is a zinc-finger-containing transcription factor. In some aspects, the zinc finger-containing transcription factor comprises a DNA-binding zinc finger protein domain (ZF protein domain) and the transcriptional repressor domain or the transcriptional activator domain. In some aspects, the ZF protein domain is modular in design and is composed of a zinc finger array (ZFA). In some aspects, the ZFA comprises one to ten ZF motifs.

In some aspects, the DNA-binding zinc finger protein domain binds to the ACP-responsive promoter. In some aspects, the ACP-responsive promoter comprises an ACP-binding domain and a promoter sequence. In some aspects, the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof. In some aspects, the ACP-responsive promoter is a synthetic promoter. In some aspects, the ACP-responsive promoter comprises a minimal promoter. In some aspects, the ACP-binding domain comprises one or more zinc finger binding sites.

In some aspects, the gene of interest is a cell death-inducing polypeptide.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

In some aspects, the cell death-inducing polypeptide is caspase 9 or a functional truncation thereof. In some aspects, the cell death-inducing domain comprises the caspase 9 amino acid sequence of Table D. In some aspects, the cell death-inducing polypeptide is Diphtheria toxin fragment A (DTA). In some aspects, the cell death-inducing domain comprises the DTA amino acid sequence of Table D. In some aspects, the cell death-inducing polypeptide is granzyme B. In some aspects, the cell death-inducing domain comprises the granzyme B amino acid sequence of Table D. In some aspects, the cell death-inducing polypeptide is Bax. In some aspects, the cell death-inducing domain comprises the Bax amino acid sequence of Table D.

Also disclosed herein is an isolated cell comprising a regulatable cell survival polypeptide and a cell death-inducing polypeptide, wherein the cell-survival polypeptide comprises a ligand binding domain, wherein when expressed the cell survival polypeptide is capable of inhibiting the cell death-inducing polypeptide, and wherein upon binding to a cognate ligand, the cognate ligand inhibits the pro-survival polypeptide.

In some aspects, the cell survival polypeptide is selected from the group consisting of: XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein. In some aspects, the cell survival polypeptide is XIAP.

In some aspects, the ligand binding domain is localized at the N-terminal region of the pro-survival polypeptide or at the C-terminal region of the pro-survival polypeptide.

In some aspects, the ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER domain), heavy chain variable region (VH) of an anti-nicotine antibody, light chain variable region (VL) of an anti-nicotine antibody, a progesterone receptor domain, an FKBP domain, and an FRB domain.

In some aspects, the ABI domain comprises the ABI amino acid sequence of Table D. In some aspects, the PYL domain comprises the PYL amino acid sequence of Table D. In some aspects, the caffeine-binding single-domain antibody comprises the amino acid sequence of Table D. In some aspects, the cannabidiol binding domain comprises an amino acid sequence selected from the group consisting of sequences of CA14, DB6, DB11, DB18, and DB21 of Table D. In some aspects, the hormone-binding domain of estrogen receptor (ER) domain comprises the amino acid sequence of Table D. In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D. In some aspects, the progesterone receptor domain comprises the amino acid sequence of Table D. In some aspects, the FKBP domain comprises the amino acid sequence of Table D. In some aspects, the FRB domain comprises the amino acid sequence of Table D.

In some aspects, when the ligand binding domain comprises an ABI domain or a PYL domain, the cognate ligand is abscisic acid.

In some aspects, when the ligand binding domain comprises a caffeine-binding single-domain antibody, the cognate ligand is caffeine or a derivative thereof.

In some aspects, when the ligand binding domain comprises a cannabidiol binding domain, the cognate ligand is a cannabidiol or a phytocannabinoid. In some aspects, when the ligand binding domain comprises a hormone-binding domain of estrogen receptor (ER) domain, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, when the ligand binding domain comprises a heavy chain variable region (VH) of an anti-nicotine antibody or a light chain variable region (VL) of an anti-nicotine antibody, the cognate ligand is nicotine or a derivative thereof.

In some aspects, when the ligand binding domain is a progesterone receptor domain, the cognate ligand is mifepristone or a derivative thereof.

In some aspects, when the ligand binding domain comprises an FKBP domain, or an FRB domain, the cognate ligand is rapamycin, AP1903, AP20187, FK1012, derivatives thereof, or analogs thereof.

In some aspects, the ligand binding domain comprises a degron. In some aspects, the degron is capable of inducing degradation of the regulatable cell survival polypeptide. In some aspects, the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box. In some aspects, the degron comprises a cereblon (CRBN) polypeptide substrate domain capable of binding CRBN in response to an immunomodulatory drug (IMiD) thereby promoting ubiquitin pathway-mediated degradation of the regulatable polypeptide. In some aspects, the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some aspects, the CRBN polypeptide substrate domain is a chimeric fusion product of native CRBN polypeptide sequences. In some aspects, the CRBN polypeptide substrate domain is a IKZF3/ZFP91/IKZF3 chimeric fusion product having the amino acid sequence of

(SEQ ID NO: 87)
FNVLMVHKRSHTGERPLQCEICGFTCRQKGNLLRHIKLHTGEKPFKCHL
CNYACQRRDAL.

In some aspects, the ligand is an IMiD. In some aspects, the IMiD is an FDA-approved drug. In some aspects, the IMiD is selected from the group consisting of: thalidomide, lenalidomide, and pomalidomide.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B 1, and Purine nucleoside phosphorylase.

In some aspects, the cell death-inducing polypeptide is caspase 9 or a functional truncation thereof. In some aspects, the cell death-inducing domain comprises the caspase 9 amino acid sequence of Table D.

In some aspects, the cell death-inducing polypeptide is Diphtheria toxin fragment A (DTA). In some aspects, the cell death-inducing domain comprises the DTA amino acid sequence of Table D.

In some aspects, the cell death-inducing polypeptide is Bax. In some aspects, the cell death-inducing domain comprises the Bax amino acid sequence of Table D.

Also disclosed herein is an engineered nucleic acid comprising: an expression cassette comprising a promoter and an exogenous polynucleotide sequence encoding an inducible cell death polypeptide monomer, wherein the promoter is operably linked to the exogenous polynucleotide, wherein the inducible cell death polypeptide monomer comprises one or more ligand binding domains and a cell death-inducing domain, wherein each of the one or more ligand binding domains comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain, wherein when expressed, the cell death polypeptide monomer is oligomerizable via a cognate ligand that binds to the ligand binding domain, and wherein when the ligand oligomerizes two or more of the cell death polypeptide monomers, a cell death-inducing signal is generated in a cell.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

In some aspects, the cell death-inducing domain comprises caspase 9, or a functional truncation thereof. In some aspects, the cell death-inducing domain comprises the caspase 9 amino acid sequence of Table D.

In some aspects, the cell death-inducing domain comprises Bid, or a functional truncation thereof. In some aspects, the cell death-inducing domain comprises the Bid amino acid sequence of Table D.

In some aspects, the ABI domain comprises the amino acid sequence of Table D. In some aspects, the PYL domain comprises the amino acid sequence of Table D.

In some aspects, the caffeine-binding single-domain antibody comprises the amino acid sequence of Table D.

In some aspects, the cannabidiol binding domain comprises an amino acid sequence selected from the group consisting of the sequences for CA14, DB6, DB11, DB18, and DB21 as shown in Table D.

In some aspects, the hormone-binding domain of estrogen receptor (ER) domain comprises the amino acid sequence of Table D.

In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D.

In some aspects, the progesterone receptor domain comprises the amino acid sequence of Table D.

In some aspects, the FKBP domain comprises the amino acid sequence of Table D.

In some aspects, the FRB domain comprises the amino acid sequence of Table D.

In some aspects, each monomer comprises the same ligand binding domain. In some aspects, the inducible cell death polypeptide comprises homooligomers. In some aspects, the homooligomers comprise homodimers. In some aspects, each monomer comprises an FKBP domain. In some aspects, the ligand is FK1012, a derivative thereof, or an analog thereof.

In some aspects, the cell death-inducing domain comprises Bid, or a functional truncation thereof. In some aspects, the cell death-inducing domain comprises the Bid amino acid sequence of Table D.

In some aspects, each monomer comprises an ABI domain and a PYL domain. In some aspects, the ligand is abscisic acid. In some aspects, the cell death-inducing domain comprises caspase 9, or a functional truncation thereof. In some aspects, the cell death-inducing domain comprises the caspase 9 amino acid sequence of Table D.

In some aspects, each monomer comprises a cannabidiol binding domain comprising the CA14 amino acid sequence of Table D and a cannabidiol binding domain comprising an amino acid sequence selected from the group consisting of the sequences of DB6, DB11, DB18, and DB21 of Table D.

In some aspects, each monomer comprises a hormone-binding domain of estrogen receptor (ER) domain and an FKBP domain. In some aspects, each monomer comprises an FRB domain and a hormone-binding domain of estrogen receptor (ER) domain. In some aspects, the cell death-inducing domain comprises caspase 9, or a functional truncation thereof. In some aspects, the cell death-inducing domain comprises the caspase 9 amino acid sequence of Table D. In some aspects, the ligand is rapamycin, a derivative thereof, or an analog thereof. In some aspects, the ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, each monomer comprises two caffeine-binding single-domain antibodies. In some aspects, each caffeine-binding single-domain antibody comprises the amino acid sequence of Table D. In some aspects, the ligand is caffeine or a derivative thereof.

In some aspects, a first monomer comprises a first ligand binding domain and a second monomer comprises a second ligand binding domain. In some aspects, the inducible cell death polypeptide comprises heterooligomers. In some aspects, the heterooligomers comprise heterodimers. In some aspects, the first monomer comprises an FKBP domain and the second monomer comprises an FRB domain. In some aspects, the cell death-inducing domain comprises Bid, or a functional truncation thereof. In some aspects, the cell death-inducing domain comprises the Bid amino acid sequence of Table D.

In some aspects, the first monomer comprises a hormone-binding domain of estrogen receptor (ER) domain and the second monomer comprises an FKBP domain. In some aspects, the first monomer comprises an FRB domain and the second monomer comprises a hormone-binding domain of estrogen receptor (ER) domain. In some aspects, the first monomer comprises a hormone-binding domain of estrogen receptor (ER) domain and an FKBP domain, and the second monomer comprises an FRB domain and the second monomer comprises a hormone-binding domain of estrogen receptor (ER) domain. In some aspects, the cell death-inducing domain comprises caspase 9, or a functional truncation thereof. In some aspects, the cell death-inducing domain comprises the caspase 9 amino acid sequence of Table D. In some aspects, the ligand is rapamycin, a derivative thereof, or an analog thereof. In some aspects, the ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, the first monomer comprises an ABI domain and the second monomer comprises a PYL domain. In some aspects, the ligand is abscisic acid.

In some aspects, the first monomer comprises a heavy chain variable region (VH) of an anti-nicotine antibody and the second monomer comprises a light chain variable region (VL) of an anti-nicotine antibody. In some aspects, the anti-nicotine antibody is a Nic12 antibody. In some aspects, the VH comprises the VH amino acid sequence of Table D. In some aspects, the VL comprises the VL amino acid sequence of Table D. In some aspects, the ligand is nicotine or a derivative thereof.

In some aspects, the first monomer comprises a cannabidiol binding domain comprising an amino acid sequence selected from the group consisting of the sequences of DB6, DB11, DB18, and DB21 of Table D and the second monomer comprises a cannabidiol binding domain comprising the amino acid sequence of CA14 of Table D. In some aspects, the ligand is a cannabidiol or a phytocannabinoid.

In some aspects, each monomer further comprises a linker localized between each ligand binding domain and cell death-inducing domain. In some aspects, the linker comprises an amino acid sequence selected from the group consisting of: GGGGSGGGGSGGGGSVDGF (SEQ ID NO: 88) and ASGGGGSAS (SEQ ID NO: 89).

Also disclosed herein is an engineered nucleic acid comprising: an expression cassette comprising a promoter and an exogenous polynucleotide sequence encoding an activation-conditional control polypeptide (ACP), wherein the promoter is operably linked to the exogenous polynucleotide, wherein the ACP comprises one or more ligand binding domains and a transcription factor comprising a nucleic acid-binding domain and a transcriptional effector domain, wherein when expressed, the ACP undergoes nuclear localization upon binding of the ligand binding domain to a cognate ligand, and wherein when localized to a cell nucleus, the ACP is capable of inducing transcriptional expression of a gene of interest operably linked to an ACP-responsive promoter.

In some aspects, each ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain.

In some aspects, the ABI domain comprises the ABI amino acid sequence of Table D. In some aspects, the PYL domain comprises the PYL amino acid sequence of Table D. In some aspects, the caffeine-binding single-domain antibody comprises the amino acid sequence of Table D. In some aspects, the cannabidiol binding domain comprises an amino acid sequence selected from the group consisting of the sequences of CA14, DB6, DB11, DB18, and DB21 of Table D. In some aspects, the hormone-binding domain of estrogen receptor (ER) domain comprises the amino acid sequence of Table D. In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D. In some aspects, the progesterone receptor domain comprises the amino acid sequence of Table D. In some aspects, the FKBP domain comprises the amino acid sequence of Table D. In some aspects, the FRB domain comprises the amino acid sequence of Table D.

In some aspects, the nucleic acid-binding domain comprises a DNA-binding zinc finger protein domain (ZF protein domain). In some aspects, the ZF protein domain is modular in design and is composed of zinc finger arrays (ZFA). In some aspects, the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the chimeric polypeptide further comprises a linker localized between the nucleic acid-binding domain and the transcriptional effector domain. In some aspects, the linker comprises one or more 2A ribosome skipping tags. In some aspects, each 2A ribosome skipping tag is selected from the group consisting of: P2A, T2A, E2A, and F2A.

In some aspects, the chimeric polypeptide comprises a first ligand binding domain operably linked to the nucleic acid-binding domain and a second ligand binding domain operably linked to the transcriptional effector domain.

In some aspects, each of the first and second ligand binding domains comprises a hormone-binding domain of estrogen receptor (ER) domain. In some aspects, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, each of the first and second ligand binding domains comprises a progesterone receptor domain. In some aspects, the cognate ligand is mifepristone or a derivative thereof.

In some aspects, when the ligand binding domain comprises an ABI domain or a PYL domain, the cognate ligand is abscisic acid.

In some aspects, when the ligand binding domain comprises a caffeine-binding single-domain antibody, the cognate ligand is caffeine or a derivative thereof.

In some aspects, when the ligand binding domain comprises a cannabidiol binding domain, the cognate ligand is a cannabidiol or a phytocannabinoid. In some aspects, the cannabidiol binding domain comprises a single-domain antibody or a nanobody. In some aspects, the cannabidiol binding domain comprises an amino acid sequence selected from the group consisting of the sequence of CA14, DB6, DB11, DB18, and DB21 of Table D.

In some aspects, when the ligand binding domain comprises a hormone-binding domain of estrogen receptor (ER) domain, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, when the ligand binding domain comprises a heavy chain variable region (VH) of an anti-nicotine antibody or a light chain variable region (VL) of an anti-nicotine antibody, the cognate ligand is nicotine or a derivative thereof.

In some aspects, when the ligand binding domain is a progesterone receptor domain, the cognate ligand is mifepristone or a derivative thereof.

In some aspects, when the ligand binding domain comprises an FKBP domain or an FRB domain, the cognate ligand is rapamycin, AP1903, AP20187, FK1012, derivatives thereof, or analogs thereof.

In some aspects, the nucleic acid-binding domain comprises a DNA-binding zinc finger protein domain (ZF protein domain).

In some aspects, the ZF protein domain is modular in design and is composed of zinc finger arrays (ZFA). In some aspects, the ZF protein domain comprises one to ten ZFA.

In some aspects, the nucleic acid-binding domain binds to the ACP-responsive promoter. In some aspects, the ACP-responsive promoter comprises an ACP-binding domain sequence and a promoter sequence. In some aspects, the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof. In some aspects, the ACP-responsive promoter comprises a synthetic promoter. In some aspects, the ACP-responsive promoter comprises a minimal promoter.

In some aspects, the ACP-binding domain comprises one or more zinc finger binding sites.

In some aspects, the transcriptional activator domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

An engineered nucleic acid comprising: an expression cassette comprising a promoter and an exogenous polynucleotide sequence having the formula: C₁-L-C₂ wherein C₁ comprises a polynucleotide sequence encoding a first chimeric polypeptide comprising a first ligand binding domain and a transcriptional activation domain, L comprises a linker polynucleotide sequence, C₂ comprises a polynucleotide sequence encoding a second chimeric polypeptide comprising a second ligand binding domain and a nucleic acid-binding domain; wherein the promoter is operably linked to the exogenous polynucleotide; wherein when expressed, the first chimeric polypeptide and the second chimeric polypeptide multimerize to form an activation-conditional control polypeptide (ACP) via a cognate ligand that binds to each ligand binding domain; and wherein the multimeric ACP is capable of inducing transcriptional expression of a gene of interest operably linked to an ACP-responsive promoter.

In some aspects, each ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain.

In some aspects, the ABI domain comprises the ABI amino acid sequence of Table D. In some aspects, the PYL domain comprises the PYL amino acid sequence of Table D. In some aspects, the caffeine-binding single-domain antibody comprises the amino acid sequence of Table D. In some aspects, the cannabidiol binding domain comprises an amino acid sequence selected from the group consisting of the sequences of CA14, DB6, DB11, DB18, and DB21 of Table D. In some aspects, the hormone-binding domain of estrogen receptor (ER) domain comprises the amino acid sequence of Table D. In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D. In some aspects, the progesterone receptor domain comprises the amino acid sequence of Table D. In some aspects, the FKBP domain comprises the FKBP amino acid sequence of Table D. In some aspects, the FRB domain comprises the FRB amino acid sequence of Table D.

In some aspects, the nucleic acid-binding domain comprises a DNA-binding zinc finger protein domain (ZF protein domain). In some aspects, the ZF protein domain is modular in design and is composed of zinc finger arrays (ZFA). In some aspects, the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the chimeric polypeptide further comprises a linker localized between the nucleic acid-binding domain and the transcriptional effector domain. In some aspects, the linker comprises one or more 2A ribosome skipping tags. In some aspects, each 2A ribosome skipping tag is selected from the group consisting of: P2A, T2A, E2A, and F2A.

In some aspects, the chimeric polypeptide comprises a first ligand binding domain operably linked to the nucleic acid-binding domain and a second ligand binding domain operably linked to the transcriptional effector domain.

In some aspects, each of the first and second ligand binding domains comprises a hormone-binding domain of estrogen receptor (ER) domain. In some aspects, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, each of the first and second ligand binding domains comprises a progesterone receptor domain.

In some aspects, the cognate ligand is mifepristone or a derivative thereof.

In some aspects, when the ligand binding domain comprises an ABI domain or a PYL domain, the cognate ligand is abscisic acid.

In some aspects, when the ligand binding domain comprises a caffeine-binding single-domain antibody, the cognate ligand is caffeine or a derivative thereof.

In some aspects, when the ligand binding domain comprises a cannabidiol binding domain, the cognate ligand is a cannabidiol or a phytocannabinoid. In some aspects, the cannabidiol binding domain comprises a single-domain antibody or a nanobody. In some aspects, the cannabidiol binding domain comprises an amino acid sequence selected from the group consisting of the sequences of CA14, DB6, DB11, DB18, and DB21 of Table D.

In some aspects, when the ligand binding domain comprises a hormone-binding domain of estrogen receptor (ER) domain, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, when the ligand binding domain comprises a heavy chain variable region (VH) of an anti-nicotine antibody or a light chain variable region (VL) of an anti-nicotine antibody, the cognate ligand is nicotine or a derivative thereof.

In some aspects, when the ligand binding domain is a progesterone receptor domain, the cognate ligand is mifepristone or a derivative thereof.

In some aspects, when the ligand binding domain comprises an FKBP domain or an FRB domain, the cognate ligand is rapamycin, AP1903, AP20187, FK1012, derivatives thereof, or analogs thereof.

In some aspects, the nucleic acid-binding domain comprises a DNA-binding zinc finger protein domain (ZF protein domain). In some aspects, the ZF protein domain is modular in design and is composed of zinc finger arrays (ZFA). In some aspects, the ZF protein domain comprises one to ten ZFA.

In some aspects, the nucleic acid-binding domain binds to the ACP-responsive promoter. In some aspects, the ACP-responsive promoter comprises an ACP-binding domain sequence and a promoter sequence. In some aspects, the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof. In some aspects, the ACP-responsive promoter comprises a synthetic promoter. In some aspects, the ACP-responsive promoter comprises a minimal promoter. In some aspects, the ACP-binding domain comprises one or more zinc finger binding sites. In some aspects, the transcriptional activator domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

In some aspects, the linker polynucleotide sequence is operably associated with the translation of each chimeric polypeptide as a separate polypeptide. In some aspects, the linker polynucleotide sequence encodes a 2A ribosome skipping tag. In some aspects, the 2A ribosome skipping tag is selected from the group consisting of: P2A, T2A, E2A, and F2A. In some aspects, the linker polynucleotide sequence encodes an Internal Ribosome Entry Site (IRES). In some aspects, the linker polynucleotide sequence encodes a cleavable polypeptide. In some aspects, the cleavable polypeptide comprises a furin polypeptide sequence.

Also disclosed herein is an engineered nucleic acid comprising: an expression cassette comprising a promoter and an exogenous polynucleotide sequence encoding an activation-conditional control polypeptide (ACP) comprising a ligand binding domain and a transcriptional effector domain, wherein the promoter is operably linked to the exogenous polynucleotide, and wherein when expressed and upon binding of the ligand binding domain to a cognate ligand, the ACP is capable of modulating transcriptional expression of a gene of interest operably linked to an ACP-responsive promoter.

Also disclosed herein is an engineered nucleic acid comprising: an expression cassette comprising a promoter and an exogenous polynucleotide sequence encoding a regulatable cell survival polypeptide comprising a ligand binding domain, wherein the promoter is operably linked to the exogenous polynucleotide, wherein when expressed, the cell survival polypeptide is capable of inhibiting a cell death-inducing polypeptide, and wherein upon binding to a cognate ligand, the cognate ligand inhibits the pro-survival polypeptide.

In some aspects, the cell survival polypeptide is selected from the group consisting of: XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein. In some aspects, the cell survival polypeptide is XIAP.

In some aspects, the ligand binding domain is localized at the N-terminal region of the pro-survival polypeptide or at the C-terminal region of the pro-survival polypeptide.

In some aspects, the ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER domain), heavy chain variable region (VH) of an anti-nicotine antibody, light chain variable region (VL) of an anti-nicotine antibody, a progesterone receptor domain, an FKBP domain, and an FRB domain.

In some aspects, the ABI domain comprises the ABI amino acid sequence of Table D. In some aspects, the PYL domain comprises the PYL amino acid sequence of Table D. In some aspects, the caffeine-binding single-domain antibody comprises the amino acid sequence of Table D. In some aspects, the cannabidiol binding domain comprises an amino acid sequence selected from the group consisting of sequences of CA14, DB6, DB11, DB18, and DB21 of Table D. In some aspects, the hormone-binding domain of estrogen receptor (ER) domain comprises the amino acid sequence of Table D. In some aspects, the heavy chain variable region (VH) of an anti-nicotine antibody comprises the VH amino acid sequence of Table D. In some aspects, the light chain variable region (VL) of an anti-nicotine antibody comprises the VL amino acid sequence of Table D. In some aspects, the progesterone receptor domain comprises the amino acid sequence of Table D. In some aspects, the FKBP domain comprises the amino acid sequence of Table D. In some aspects, the FRB domain comprises the amino acid sequence of Table D.

In some aspects, when the ligand binding domain comprises an ABI domain or a PYL domain, the cognate ligand is abscisic acid.

In some aspects, when the ligand binding domain comprises a caffeine-binding single-domain antibody, the cognate ligand is caffeine or a derivative thereof.

In some aspects, when the ligand binding domain comprises a cannabidiol binding domain, the cognate ligand is a cannabidiol or a phytocannabinoid. In some aspects, when the ligand binding domain comprises a hormone-binding domain of estrogen receptor (ER) domain, the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, when the ligand binding domain comprises a heavy chain variable region (VH) of an anti-nicotine antibody or a light chain variable region (VL) of an anti-nicotine antibody, the cognate ligand is nicotine or a derivative thereof.

In some aspects, when the ligand binding domain is a progesterone receptor domain, the cognate ligand is mifepristone or a derivative thereof.

In some aspects, when the ligand binding domain comprises an FKBP domain, or an FRB domain, the cognate ligand is rapamycin, AP1903, AP20187, FK1012, derivatives thereof, or analogs thereof.

In some aspects, the ligand binding domain comprises a degron. In some aspects, the degron is capable of inducing degradation of the regulatable cell survival polypeptide. In some aspects, the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box. In some aspects, the degron comprises a cereblon (CRBN) polypeptide substrate domain capable of binding CRBN in response to an immunomodulatory drug (IMiD) thereby promoting ubiquitin pathway-mediated degradation of the regulatable polypeptide. In some aspects, the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some aspects, the CRBN polypeptide substrate domain is a chimeric fusion product of native CRBN polypeptide sequences. In some aspects, the CRBN polypeptide substrate domain is a IKZF3/ZFP91/IKZF3 chimeric fusion product having the amino acid sequence of

(SEQ ID NO: 90)
FNVLMVHKRSHTGERPLQCEICGFTCRQKGNLLRHIKLHTGEKPFKCHL
CNYACQRRDAL.

In some aspects, the ligand is an IMiD. In some aspects, the IMiD is an FDA-approved drug. In some aspects, the IMiD is selected from the group consisting of: thalidomide, lenalidomide, and pomalidomide.

In some aspects, the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B 1, and Purine nucleoside phosphorylase.

In some aspects, the cell death-inducing polypeptide is caspase 9 or a functional truncation thereof. In some aspects, the cell death-inducing domain comprises the caspase 9 amino acid sequence of Table D.

In some aspects, the cell death-inducing polypeptide is Diphtheria toxin fragment A (DTA). In some aspects, the cell death-inducing domain comprises the DTA amino acid sequence of Table D.

In some aspects, the cell death-inducing polypeptide is Bax. In some aspects, the cell death-inducing domain comprises the Bax amino acid sequence of Table D.

An engineered nucleic acid comprising: (a) a first expression cassette comprising a first promoter and a first exogenous polynucleotide sequence encoding a first chimeric polypeptide, wherein the first chimeric polypeptide comprises a first ligand binding domain and a transcriptional activation domain, wherein the first promoter is operably linked to the first exogenous polynucleotide; and (b) a second expression cassette comprising a second promoter and a second exogenous polynucleotide sequence encoding a second chimeric polypeptide, wherein the second chimeric polypeptide comprises a second ligand binding domain and a nucleic acid-binding domain, wherein the second promoter is operably linked to the second exogenous polynucleotide, wherein when expressed, the first chimeric polypeptide and the second chimeric polypeptide multimerize to form an activation-conditional control polypeptide (ACP) via a cognate ligand that binds to each ligand binding domain, and wherein the multimeric ACP is capable of inducing transcriptional expression of a gene of interest operably linked to an ACP-responsive promoter.

In some aspects, the first promoter, the second promoter, or both the first promoter and the second promoter comprise(s) a constitutive promoter, an inducible promoter, or a synthetic promoter.

In some aspects, the constitutive promoter is selected from the group consisting of: CAG, HLP, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some aspects, the inducible promoter is selected from the group consisting of: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule-responsive promoters, and tandem repeats thereof.

In some aspects, an inducible cell death polypeptide or system as described herein comprises a modified XIAP, wherein the modified XIAP comprises one or more amino acid substitutions within to positions 306-325 of SEQ ID NO: 107.

In some aspects, the one or more amino acid substitutions are at one or more positions of SEQ ID NO: 107 selected from the group consisting of: 305, 306, 308, and 325.

In some aspects, the one or more amino acid substitutions are at position 305 of SEQ ID NO: 107. In some aspects, the amino acid substitution at position 305 of SEQ ID NO: 107 is G305M.

In some aspects, the one or more amino acid substitutions are at position 306 of SEQ ID NO: 107. In some aspects, the amino acid substitution at position 306 of SEQ ID NO: 107 is G3065.

In some aspects, the one or more amino acid substitutions are at position 308 of SEQ ID NO: 107. In some aspects, the amino acid substitution at position 308 of SEQ ID NO: 107 is selected from the group consisting of T3085 and T308D. In some aspects, the amino acid substitution at position 308 of SEQ ID NO: 107 is T3085. In some aspects, the amino acid substitution at position 308 of SEQ ID NO: 107 is T308D.

In some aspects, the one or more amino acid substitutions are at position 325 of SEQ ID NO: 107. In some aspects, the amino acid substitution at position 325 of SEQ ID NO: 107 is P325S.

In some aspects, the one or more amino acid substitutions are two amino acid substitutions.

In some aspects, each of the two amino acid substitutions are at a position of SEQ ID NO: 107 selected from the group consisting of: 305, 306, 308, and 325.

In some aspects, the two amino acid substitutions are at positions 305 and 306 of SEQ ID NO: 107. In some aspects, the amino acid substitution at position 305 of SEQ ID NO: 107 is G305M and the amino acid substitution at position 306 of SEQ ID NO: 107 is G3065.

In some aspects, the two amino acid substitutions are at positions 305 and 308 of SEQ ID NO: 107. In some aspects, the amino acid substitution at position 305 of SEQ ID NO: 107 is G305M and the amino acid substitution at position 308 of SEQ ID NO: 107 is T3085.

In some aspects, the amino acid substitution at position 305 of SEQ ID NO: 107 is G305M and the amino acid substitution at position 308 of SEQ ID NO: 107 is T308D.

In some aspects, the two amino acid substitutions are at positions 305 and 325 of SEQ ID NO: 107. In some aspects, the amino acid substitution at position 305 of SEQ ID NO: 107 is G305M and the amino acid substitution at position 325 of SEQ ID NO: 107 is P325S.

In some aspects, the two amino acid substitutions are at positions 306 and 308 of SEQ ID NO: 107. In some aspects, the amino acid substitution at position 306 of SEQ ID NO: 107 is G3065 and the amino acid substitution at position 308 of SEQ ID NO: 107 is T3085.

In some aspects, the amino acid substitution at position 306 of SEQ ID NO: 107 is G3065 and the amino acid substitution at position 308 of SEQ ID NO: 107 is T308D.

In some aspects, the two amino acid substitutions are at positions 306 and 325 of SEQ ID NO: 107.

In some aspects, the amino acid substitution at position 306 of SEQ ID NO: 107 is G3065 and the amino acid substitution at position 325 of SEQ ID NO: 107 is P325S.

In some aspects, the two amino acid substitutions are at positions 308 and 325 of SEQ ID NO: 107.

In some aspects, the amino acid substitution at position 308 of SEQ ID NO: 107 is T3085 and the amino acid substitution at position 325 of SEQ ID NO: 107 is P325S.

In some aspects, the amino acid substitution at position 308 of SEQ ID NO: 107 is T308D and the amino acid substitution at position 325 of SEQ ID NO: 107 is P325S.

In some aspects, the one or more additional amino acid substitutions are three amino acid substitutions. In some aspects, each of the three amino acid substitutions are at a position of SEQ ID NO: 107 selected from the group consisting of: 305, 306, 308, and 325. In some aspects, the three amino acid substitutions are at positions 305, 306, and 308 of SEQ ID NO: 107.

In some aspects, the amino acid substitution at position 305 of SEQ ID NO: 107 is G305M, the amino acid substitution at position 306 of SEQ ID NO: 107 is G3065, and the amino acid substitution at position 308 of SEQ ID NO: 107 is T3085.

In some aspects, the amino acid substitution at position 305 of SEQ ID NO: 107 is G305M, the amino acid substitution at position 306 of SEQ ID NO: 107 is G3065, and the amino acid substitution at position 308 of SEQ ID NO: 107 is T308D.

In some aspects, the three amino acid substitutions are at positions 305, 306, and 325 of SEQ ID NO: 107. In some aspects, the amino acid substitution at position 305 of SEQ ID NO: 107 is G305M, the amino acid substitution at position 306 of SEQ ID NO: 107 is G3065, and the amino acid substitution at position 325 of SEQ ID NO: 107 is P325S.

In some aspects, the three amino acid substitutions are at positions 305, 308, and 325 of SEQ ID NO: 107. In some aspects, the amino acid substitution at position 305 of SEQ ID NO: 107 is G305M, the amino acid substitution at position 308 of SEQ ID NO: 107 is T3085, and the amino acid substitution at position 325 of SEQ ID NO: 107 is P325S. In some aspects, the amino acid substitution at position 305 of SEQ ID NO: 107 is G305M, the amino acid substitution at position 308 of SEQ ID NO: 107 is T308D, and the amino acid substitution at position 325 of SEQ ID NO: 107 is P325S.

In some aspects, the three amino acid substitutions are at positions 306, 308, and 325 of SEQ ID NO: 107. In some aspects, the amino acid substitution at position 306 of SEQ ID NO: 107 is G3065, the amino acid substitution at position 308 of SEQ ID NO: 107 is T3085, and the amino acid substitution at position 325 of SEQ ID NO: 107 is P325S. In some aspects, the amino acid substitution at position 306 of SEQ ID NO: 107 is G3065, the amino acid substitution at position 308 of SEQ ID NO: 107 is T3084, and the amino acid substitution at position 325 of SEQ ID NO: 107 is P325S.

In some aspects, the one or more additional amino acid substitutions are four amino acid substitutions.

In some aspects, the four amino acid substitutions are at positions 305, 306, 308, and 325 of SEQ ID NO: 107. In some aspects, the amino acid substitution at position 305 of SEQ ID NO: 107 is G305M, the amino acid substitution at position 306 of SEQ ID NO: 107 is G3065, the amino acid substitution at position 308 of SEQ ID NO: 107 is T3085, and the amino acid substitution at position 325 of SEQ ID NO: 107 is P325S. In some aspects, the amino acid substitution at position 305 of SEQ ID NO: 107 is G305M, the amino acid substitution at position 306 of SEQ ID NO: 107 is G3065, the amino acid substitution at position 308 of SEQ ID NO: 107 is T308D, and the amino acid substitution at position 325 of SEQ ID NO: 107 is P325S

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1A, FIG. 1B, FIG. 1C, and FIG. 1D provide examples of inducible cell death systems.

FIG. 2A shows the domains and organization of the indicated constructs for an inducible cell-death system.

FIG. 2B shows mCherry expression in cells expressing the indicated constructs following the addition of an IMiD.

FIG. 3A shows the domains and organization of the indicated constructs for an ligand-induced dimerization of an inducible cell death system using a mifepristone-based system.

FIG. 3B shows HEK293 cells engineered to express the Caspase-9/progesterone-receptor fusions and cell death upon addition of Mifepristone (both apoptotic and by live/dead).

FIG. 4A shows the domains and organization of the indicated constructs for an ligand-induced dimerization of an inducible cell death system using a mifepristone-based system.

FIG. 4B shows cell death (toxin activity) for the various inducible cell-death systems (both apoptotic and by live/dead).

FIG. 4C shows mKate reporter expression for the various inducible cell-death systems (both apoptotic and by live/dead).

FIG. 4D shows viability of cells on day 5 as a ratio of viability of cells on day 3. A no drug condition (left columns) and 1 uM Pomalidomide treatment (right columns) are shown.

FIG. 5A shows the domains and organization of the indicated constructs for an inducible cell-death system.

FIG. 5B shows confluency calculated using an Incucyte system for Smac/Diablo constructs.

FIG. 5C shows confluency calculated using an Incucyte system for tBid constructs.

FIG. 5D shows confluency calculated using an Incucyte system for Bax constructs.

FIG. 6A shows the domains and organization of the indicated constructs for a Bax inducible cell-death system.

FIG. 6B shows confluency calculated using an Incucyte system for Bax constructs.

FIG. 6C shows viability for Bax constructs.

FIG. 6D shows viability for Bax constructs.

FIG. 7 shows the domains and organization of an IMiD and tamoxifen based inducible caspase-9 dimerization (iCasp9) system.

### DETAILED DESCRIPTION

Terms used in the claims and specification are defined as set forth below unless otherwise specified.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

Engineered Nucleic Acids and Polypeptides

An engineered nucleic acid can comprise a first expression cassette comprising a first promoter and a first exogenous polynucleotide sequence. The first promoter can be operably or directly linked to the first exogenous polynucleotide sequence. The first exogenous polynucleotide sequence can encode a first polypeptide such as an activation-conditional control polypeptide (ACP). In some embodiments, a single engineered nucleic acid comprises at least one, two, three four, five, or more expression cassettes, e.g., a plurality. In general, each expression cassette can refer to a promoter operably linked to a polynucleotide sequence encoding a protein of interest.

An engineered nucleic acid can comprise an expression cassette comprising a promoter operably linked to an exogenous polynucleotide sequence that encodes at least one inducible cell death polypeptide monomer. An inducible cell death polypeptide monomer can comprise one or more ligand binding domains and at least one cell death-inducing domain. When expressed in a cell, the cell death polypeptide monomer is oligomerizable via a cognate ligand (e.g., a small molecule) that binds to the ligand binding domain(s). When the ligand oligomerizes two or more of the cell death polypeptide monomers, a cell death-inducing signal can be generated in the cell. This generally results in cell death.

An engineered nucleic acid can comprise an expression cassette comprising a promoter operably linked to an exogenous polynucleotide sequence that encodes at least one activation-conditional control polypeptide (ACP). The ACP can comprise one or more ligand binding domains and at least one transcription factor comprising at least one nucleic acid-binding domain and at least one transcriptional effector domain. When expressed in a cell, the ACP can undergo nuclear localization upon binding of the ligand binding domain(s) to a cognate ligand. When localized to the cell's nucleus, the ACP is capable of inducing transcriptional expression of a gene of interest operably linked to an ACP-responsive promoter. The gene of interest can be associated with or cause cell death such as apoptosis. This can result in cell death.

An engineered nucleic acid can comprise an expression cassette comprising a promoter operably linked to an exogenous polynucleotide sequence that encodes at least one ACP. The ACP can comprise at least one ligand binding domain and at least one transcriptional effector domain. When expressed in a cell and upon binding of the ligand binding domain(s) to a cognate ligand, the ACP is capable of modulating transcriptional expression of a gene of interest operably linked to an ACP-responsive promoter. For example, in some embodiments, when expressed in a cell and upon binding of the ligand binding domain(s) to a cognate ligand, activity of the ACP modulates transcriptional expression of a gene of interest operably linked to an ACP-responsive promoter. Alternatively, in some embodiments, binding of the ligand binding domain(s) to a cognate ligand induces degradation of the ACP, and thus ACP-based modulation of transcriptional expression of a gene of interest is abrogated by the binding to the cognate ligand. The gene of interest can be associated with or cause cell death such as apoptosis. This can result in cell death.

An engineered nucleic acid can comprise an expression cassette comprising a promoter operably linked to an exogenous polynucleotide sequence that encodes at least one regulatable cell survival polypeptide comprising at least one ligand binding domain. When expressed in a cell, the at least one cell survival polypeptide is capable of inhibiting at least one cell death-inducing polypeptide and upon binding to a cognate ligand, the cognate ligand inhibits the at least one pro-survival polypeptide. This can result in cell death.

An engineered nucleic acid can comprise an expression cassette comprising a promoter operably linked to an exogenous polynucleotide sequence having the formula: C₁-L-C₂ wherein C₁ comprises a polynucleotide sequence encoding at least a first chimeric polypeptide comprising at least a first ligand binding domain and at least a transcriptional activation domain, L comprises at least a linker polynucleotide sequence, C₂ comprises a polynucleotide sequence encoding at least a second chimeric polypeptide comprising a second ligand binding domain and at least a nucleic acid-binding domain. When expressed in a cell, the first chimeric polypeptide and the second chimeric polypeptide can multimerize to form an activation-conditional control polypeptide (ACP) via a cognate ligand that binds to each ligand binding domain. The multimeric ACP can then be capable of inducing transcriptional expression of a gene of interest operably linked to an ACP-responsive promoter. This can result in cell death.

An engineered nucleic acid can comprise an expression cassette comprising (a) a first expression cassette comprising a first promoter operably linked to a first exogenous polynucleotide sequence encoding a first chimeric polypeptide, wherein the first chimeric polypeptide comprises a first ligand binding domain and a transcriptional activation domain, (b) a second expression cassette comprising a second promoter operably linked to a second exogenous polynucleotide sequence encoding a second chimeric polypeptide, wherein the second chimeric polypeptide comprises a second ligand binding domain and a nucleic acid-binding domain. When expressed in a cell, the first chimeric polypeptide and the second chimeric polypeptide can multimerize to form an activation-conditional control polypeptide (ACP) via a cognate ligand that binds to each ligand binding domain. The multimeric ACP is then capable of inducing transcriptional expression of a gene of interest operably linked to an ACP-responsive promoter in the cell. This can result in cell death.

One or more linkers can be used between various domains of engineered nucleic acids. For example, a polypeptide linker encoded by the engineered nucleic acid(s) can comprise an amino acid sequence such as one or more of: GGGGSGGGGSGGGGSVDGF (SEQ ID NO: 91) and ASGGGGSAS (SEQ ID NO: 92). Additional exemplary linkers are shown in Table D.

In some embodiments, one or more expression cassettes can be multicistronic, i.e., more than one separate polypeptide (e.g., multiple exogenous polynucleotides or effector molecules) can be produced from a single transcript. For example, a multicistronic expression cassette can encode both a first ACP and a second ACP, e.g., both expressed from a single expression cassette driven by a constitutive promoter. In another example, a multicistronic expression cassette can encode both an effector molecule and an antigen recognizing receptor, e.g., both expressed from a single expression cassette driven by an ACP-responsive promoter. Expression cassettes can be multicistronic through the use of various linkers, e.g., a polynucleotide sequence encoding a first protein of interest can be linked to a nucleotide sequence encoding a second protein of interest, such as in a first gene:linker:second gene 5′ to 3′ orientation. Multicistronic features and options are described in the section “Multicistronic and Multiple Promoter Systems.”

In some embodiments, the engineered nucleic acid is selected from: a DNA, a cDNA, an RNA, an mRNA, and a naked plasmid (linear or circular). Also provided herein is an expression vector comprising the engineered nucleic acid.

In some embodiments, the engineered nucleic acid further comprises an insulator. The insulator can be localized between the first expression cassette and the second expression cassette. An insulator is a cis-regulatory element that has enhancer-blocking or barrier function. Enhancer-blocker insulators block enhancers from acting on the promoter of nearby genes. Barrier insulators prevent euchromatin silencing. An example of a suitable insulator of the present disclosure is the A2 insulator as described in Liu M, et al., Nat Biotechnol. 2015 Feb.; 33(2):198-203. Additional insulators are described in West et al, Genes & Dev, 002. 16: 271-288, both of which are incorporated by reference in their entirety. Other examples of suitable insulators include, without limitation, an A1 insulator, a CTCF insulator, a gypsy insulator, an HS5 insulator, and a β-globin locus insulator, such as cHS4. In some embodiments, the insulator is an A2 insulator, an A1 insulator, a CTCF insulator, an HS5 insulator, a gypsy insulator, a β-globin locus insulator, or a cHS4 insulator.

Ligand Binding Domains

A ligand binding domain can interact with a ligand such as a cognate ligand. Such an interaction can result in oligomerization, e.g., dimerization, of a plurality of ligand binding domains via ligand binding.

Exemplary ligand binding domains can include a domain, or functional fragment thereof, such as one or more of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and/or an FRB domain. Example sequences of such domains are shown in Table D.

A ligand binding domain can include a degron. The terms “degron” and “degron domain,” as used herein, refer to a protein or a part thereof that is important in regulation of protein degradation rates. Various degrons known in the art, including but not limited to short amino acid sequences, structural motifs, and exposed amino acids, can be used in various embodiments of the present disclosure. Degrons identified from a variety of organisms can be used. Degrons and degron pathways are generally known, see, e.g., Varshazsky A., PNAS 2019 Jan. 8; 116(2):358-366, hereby incorporated by reference.

The term “degradation sequence” as used herein, refers to a sequence that promotes degradation of an attached protein through either the proteasome or autophagy-lysosome pathways. Degradation sequences known in the art can be used for various embodiments of the present disclosure. In some embodiments, a degradation sequence comprises a degron identified from an organism, or a modification thereof. In some embodiments, a degradation sequence is a polypeptide that destabilize a protein such that half-life of the protein is reduced at least two fold, when fused to the protein. Many different degradation sequences/signals (e.g., of the ubiquitin-proteasome system) are known in the art, any of which may be used as provided herein. A degradation sequence may be operably linked to a cell receptor, but need not be contiguous with it as long as the degradation sequence still functions to direct degradation of the cell receptor. In some embodiments, the degradation sequence induces rapid degradation of the cell receptor. For a discussion of degradation sequences and their function in protein degradation, see, e.g., Kanemaki et al. (2013) Pflugers Arch. 465(3):419-425, Erales et al. (2014) Biochim Biophys Acta 1843(1):216-221, Schrader et al. (2009) Nat. Chem. Biol. 5(11): 815-822, Ravid et al. (2008) Nat. Rev. Mol. Cell. Biol. 9(9):679-690, Tasaki et al. (2007)Trends Biochem Sci. 32 (1 1):520-528, Meinnel et al. (2006) Biol. Chem. 387(7):839-851, Kim et al. (2013) Autophagy 9(7): 1100-1103, Varshaysky (2012) Methods Mol. Biol. 832: 1-11, and Fayadat et al. (2003) Mol Biol Cell. 14(3): 1268-1278; herein incorporated by reference.

In some embodiments, the degron or degradation sequence is selected from: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box. In some embodiments, a degron includes modifications/mutations that reduce ubiquitination relative to wild-type protein, e.g., relative to a peptide sequence or domain the degron is derived from. Modifications/mutations that reduce ubiquitination can include replacing or more lysine residues. Modifications/mutations that reduce ubiquitination can include replacing all lysine residues.

In some embodiments, the degron comprises a cereblon (CRBN) polypeptide substrate domain capable of binding CRBN in response to an immunomodulatory drug (IMiD) thereby promoting ubiquitin pathway-mediated degradation of the ACP. In some embodiments, the CRBN polypeptide substrate domain is selected from: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some embodiments, the CRBN polypeptide substrate domain is a chimeric fusion product of native CRBN polypeptide sequences. In some embodiments, the CRBN polypeptide substrate domain is a IKZF3/ZFP91/IKZF3 chimeric fusion product having the amino acid sequence of

(SEQ ID NO: 93)
FNVLMVHKRSHTGERPLQCEICGFTCRQKGNLLRHIKLHTGEKPFKCHL
CNYACQRRDAL.

In some embodiments, a degron includes a degron having the amino acid sequence of SEQ ID NO: 131. A degron can include a modified d913 degron, including amino acid substitutions relative to the amino acid sequence of SEQ ID NO: 131. In some embodiments, a degron includes a modified degron having the amino acid sequence of SEQ ID NO: 133. A d913 degron can include modifications/mutations that reduce ubiquitination relative to unmodified d913 having the amino acid sequence of SEQ ID NO: 131. A d913 degron can include replacing one or more lysine residues, e.g., relative to unmodified d913 having the amino acid sequence of SEQ ID NO: 131, such as A d913 degron can include replacing all lysine residues, e.g., relative to unmodified d913 having the amino acid sequence of SEQ ID NO: 131. A d913 degron can include replacing one or more lysine residues with arginine residues, e.g., relative to unmodified d913 including the amino acid sequence of SEQ ID NO: 131. A d913 degron can include replacing all lysine residues with arginine residues, e.g., relative to unmodified d913 having the amino acid sequence of SEQ ID NO: 131, such as a modified degron including the amino acid sequence of SEQ ID NO: 133.

In some embodiments, cereblon (CRBN) is a wild-type CRBN polypeptide, e.g., the amino acid sequence of SEQ ID NO: 127. In some embodiments, CRBN is a modified CRBN polypeptide. A modified CRBN can include mutations that reduce ubiquitination relative to wild-type CRBN. A modified CRBN can include a deletion of amino acids 194-247, which is the DDB1 interacting domain, e.g., a modified CRBN including the amino acid sequence of SEQ ID NO: 129.

Ligands and Cognate Ligand Pairs

A ligand can bind to a ligand binding domain. A given ligand that consistently binds to a given ligand binding domain can be referred to as a cognate ligand pair.

In some aspects, the ligand is FK1012, a derivative thereof, or an analog thereof.

In some aspects, the ligand is abscisic acid.

In some aspects, the ligand is rapamycin, a derivative thereof, or an analog thereof. In some aspects, the ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, the ligand is caffeine or a derivative thereof.

In some aspects, the ligand is nicotine or a derivative thereof.

In some aspects, the ligand is a cannabidiol or a phytocannabinoid.

In some aspects, the ligand is mifepristone or a derivative thereof.

In some aspects, the ligand is an IMiD. In some aspects, the IMiD is an FDA-approved drug. In some aspects, the IMiD is selected from the group consisting of: thalidomide, lenalidomide, and pomalidomide.

In some aspects, a ligand binding domain comprises a hormone-binding domain of estrogen receptor (ER) domain and the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, a ligand binding domain comprises a progesterone receptor domain and the cognate ligand is mifepristone or a derivative thereof.

In some aspects, a ligand binding domain comprises an ABI domain or a PYL domain and the cognate ligand is abscisic acid.

In some aspects, a ligand binding domain comprises a caffeine-binding single-domain antibody and the cognate ligand is caffeine or a derivative thereof.

In some aspects, a ligand binding domain comprises a cannabidiol binding domain and the cognate ligand is a cannabidiol or a phytocannabinoid. In some aspects, the cannabidiol binding domain comprises a single-domain antibody or a nanobody. In some aspects, the cannabidiol binding domain comprises an amino acid sequence selected from the group consisting of the sequence of CA14, DB6, DB11, DB18, and DB21 of Table D.

In some aspects, a ligand binding domain comprises a hormone-binding domain of estrogen receptor (ER) domain and the cognate ligand is tamoxifen or a metabolite thereof. In some aspects, the tamoxifen metabolite is selected from the group consisting of: 4-hydroxytamoxifen, N-desmethyltamoxifen, tamoxifen-N-oxide, and endoxifen.

In some aspects, a ligand binding domain comprises a heavy chain variable region (VH) of an anti-nicotine antibody or a light chain variable region (VL) of an anti-nicotine antibody and the cognate ligand is nicotine or a derivative thereof.

In some aspects, a ligand binding domain comprises a progesterone receptor domain and the cognate ligand is mifepristone or a derivative thereof.

In some aspects, a ligand binding domain comprises an FKBP domain or an FRB domain and the cognate ligand is rapamycin, AP1903, AP20187, FK1012, derivatives thereof, or analogs thereof.

Cell Death-Inducing Domains

Inducible cell death polypeptides can include one or more ligand binding domains and at least one cell death-inducing domain.

Exemplary cell death-inducing domains can be derived from a protein such as one or more of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related apoptosis-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and/or Purine nucleoside phosphorylase. Exemplary sequences can be found in Table D.

A cell death-inducing domain can include or be derived from Caspase 9, e.g., the amino acid sequence shown in SEQ ID NO: 39 or 123. A derivative of Caspase-9 includes an inducible Caspase-9 (“iCasp-9”), which is capable of inducing apoptosis due to drug-based dimerization, e.g., the amino acid sequence shown in SEQ ID NO: 48 or 125.

A cell death-inducing domain can include BAX, e.g., the amino acid sequence shown in SEQ ID NO: 32.

Regulatable Cell Survival Polypeptides

A regulatable cell survival polypeptide can comprise at least one ligand binding domain.

Exemplary cell survival polypeptides include one or more of XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein. A cell survival polypeptide can include XIAP. A cell survival polypeptide can include wild-type XIAP, e.g., having the amino acid sequence SEQ ID NO: 107. A cell survival polypeptide can include modified XIAP. A modified XIAP can include one or more amino acid substitutions with reference to SEQ ID NO: 107. A modified XIAP can include one or more amino acid substitutions within positions 305-325 with reference to SEQ ID NO: 107. A modified XIAP can include one or more amino acid substitutions including 305, 306, 308, or 325 with reference to SEQ ID NO: 107. A modified XIAP can include one or more amino acid substitutions including each of 305, 306, 308, and 325 with reference to SEQ ID NO: 107. A modified XIAP can include one or more amino acid substitutions including each of 305, 306, 308, and 325 with reference to SEQ ID NO: 107 that includes T3085, G3065, G305M, and P325S. A modified XIAP can include one or more amino acid substitutions including each of 305, 306, 308, and 325 with reference to SEQ ID NO: 107 that includes T308D, G3065, G305M, and P325S. A modified XIAP can include an amino acid substitution at position 305 of SEQ ID NO: 107. A modified XIAP can include an amino acid substitution at position 305 of SEQ ID NO: 107 that is G305M. A modified XIAP can include an amino acid substitution at position 306 of SEQ ID NO: 107. A modified XIAP can include an amino acid substitution at position 306 of SEQ ID NO: 107 that is G3065. A modified XIAP can include an amino acid substitution at position 308 of SEQ ID NO: 107. A modified XIAP can include an amino acid substitution at position 308 of SEQ ID NO: 107 that is T3085 or T308D. A modified XIAP can include an amino acid substitution at position 308 of SEQ ID NO: 107 that is T3085. A modified XIAP can include an amino acid substitution at position 308 of SEQ ID NO: 107 that is T308D. A modified XIAP can include an amino acid substitution at position 325 of SEQ ID NO: 107. A modified XIAP can include an amino acid substitution at position 325 of SEQ ID NO: 107 that is P325S.

Activation-Conditional Control Polypeptides (ACPs)

In some embodiments, the ACP includes a transcriptional modulator. In some embodiments, the ACP includes a transcriptional repressor. In some embodiments, the ACP includes a transcriptional activator. In some embodiments, the ACP includes a transcription factor. In some embodiments, the ACP comprises a DNA-binding domain and a transcriptional effector domain. In some embodiments, the transcription factor includes a zinc-finger-containing transcription factor. In some embodiments, the zinc-finger-containing transcription factor may be a synthetic transcription factor. In some embodiments, the ACP DNA-binding domain comprises a DNA-binding zinc finger protein domain (ZF protein domain). In some embodiments, the DNA-binding domain comprises a tetracycline (or derivative thereof) repressor (TetR) domain. An ACP can include one or more ligand binding domains.

Nucleic Acid Binding Domains

An engineered nucleic acid can encode least one transcription factor comprising at least one nucleic acid-binding domain. An engineered nucleic acid can encode least one transcription factor comprising at least one nucleic acid-binding domain and at least one transcriptional effector domain.

In some aspects, the nucleic acid-binding domain comprises a DNA-binding zinc finger protein domain (ZF protein domain). In some aspects, the ZF protein domain is modular in design and is composed of zinc finger arrays (ZFA). In some aspects, the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-0, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

An exemplary ZF protein domain is shown in the sequence

(SEQ ID NO: 94)
SRPGERPFQCRICMRNFSRRHGLDRHTRTHTGEKPFQCRICMRNFSDHS
SLKRHLRTHTGSQKPFQCRICMRNFSVRHNLTRHLRTHTGEKPFQCRIC
MRNFSDHSNLSRHLKTHTGSQKPFQCRICMRNFSQRSSLVRHLRTHTGE
KPFQCRICMRNFSESGHLKRHLRTHLRGS.

Transcriptional Effector Domains

An inducible cell death polypeptide or ACP as provided herein can include at least one transcriptional effector domain. For example an inducible cell death polypeptide or an ACP can encode at least one transcriptional effector domain. In addition, an inducible cell death polypeptide or an ACP can encode at least one ligand binding domain and at least one transcriptional effector domain.

In some aspects, a transcriptional effector domain includes one or more of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the transcriptional effector domain comprises a transcriptional repressor domain. In some aspects, the transcriptional repressor domain is selected from the group consisting of: a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

In some aspects, the transcriptional effector domain comprises a transcriptional activation domain. In some aspects, the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain). Transcriptional activation domains can also be referred to as transcriptional activator domains.

An inducible cell death polypeptide or ACP as provided herein can encode at least one transcription factor comprising at least one transcriptional effector domain. An inducible cell death polypeptide or ACP as provided herein can encode at least one transcription factor comprising at least one nucleic acid-binding domain and at least one transcriptional effector domain. For example, an ACP can encode at least one transcription factor comprising at least one nucleic acid-binding domain and at least one transcriptional effector domain. In addition, an ACP can encode at least one ligand binding domain and at least one transcription factor comprising at least one nucleic acid-binding domain and at least one transcriptional effector domain.

The engineered nucleic acid can encode an effector domain, such as a transcriptional effector domain. For instance, a transcriptional effector domain can be the effector domain (e.g., activator domain or repressor domain) of a transcription factor. Transcription factor effector domains are also known as transactivation domains, and act as scaffold domains for proteins such as transcription coregulators that act to activate or repress transcription of genes. Any suitable transcriptional effector domain can be used including, but not limited to, a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Kruppel associated box (KRAB) repression domain; a truncated Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, or any combination thereof.

Exemplary transcriptional effector domain protein sequences are shown in Table 1. Exemplary transcriptional effector domain nucleotide sequences are shown in Table 2.

TABLE 1
Transcriptional Effector Domain (Protein)

| Amino Acid Sequence | SEQ ID NO: | Description |
|---|---|---|
| RTLVTFKDVFVDFTREEWKLLDTAQQIVYRNV | 95 | KRAB |
| MLENYKNLVSLGYQLTKPDVILRLEKGEEPWL | | |
| V | | |
| RTLVTFKDVFVDFTREEWKLLDTAQQIVYRNV | 96 | truncated KRAB (minKRAB) |
| MLENYKNLVSLGY | | |
| EASGSGRADALDDFDLDMLGSDALDDFDLDM | 97 | VPR activation domain |
| LGSDALDDFDLDMLGSDALDDFDLDMLINSRS | | |
| SGSPKKKRKVGSQYLPDTDDRHRIEEKRKRTY | | |
| ETFKSIMKKSPFSGPTDPRPPPRRIAVPSRSSAS | | |
| VPKPAPQPYPFTSSLSTINYDEFPTMVFPSGQIS | | |
| QASALAPAPPQVLPQAPAPAPAPAMVSALAQA | | |
| PAPVPVLAPGPPQAVAPPAPKPTQAGEGTLSEA | | |
| LLQLQFDDEDLGALLGNSTDPAVFTDLASVDN | | |
| SEFQQLLNQGIPVAPHTTEPMLMEYPEAITRLV | | |
| TGAQRPPDPAPAPLGAPGLPNGLLSGDEDFSSI | | |
| ADMDFSALLGSGSGSRDSREGMFLPKPEAGSA | | |
| ISDVFEGREVCQPKRIRPFHPPGSPWANRPLPAS | | |
| LAPTPTGPVHEPVGSLTPAPVPQPLDPAPA VTP | | |
| EASHLLEDPDEETSQAVKALREMADTVIPQKE | | |
| EAAICGQMDLSHPPPRGHLDELTTTLESMTEDL | | |
| NLDSPLTPELNEILDTFLNDECLLHAMHISTGLS | | |
| IFDTSLF | | |

TABLE 2
Transcriptional Effector Domain (Nucleotide)

| Nucleic Acid Sequence | SEQ ID NO: | Description |
|---|---|---|
| AGAACCCTGGTCACCTTCAAGGACGTGTTCG | 98 | KRAB |
| TGGACTTCACCCGGGAAGAGTGGAAGCTGCT | | |
| GGATACAGCCCAGCAGATCGTGTACCGGAA | | |
| CGTGATGCTGGAAAACTACAAGAATCTGGTG | | |
| TCCCTGGGCTACCAGCTGACCAAGCCTGACG | | |
| TGATCCTGCGGCTGGAAAAGGGCGAAGAAC | | |
| CTTGGCTGGTG | | |
| AGAACCCTGGTCACCTTCAAGGACGTGTTCG | 99 | truncated KRAB (minKRAB) |
| TGGACTTCACCCGGGAAGAGTGGAAGCTGCT | | |
| GGATACAGCCCAGCAGATCGTGTACCGGAA | | |
| CGTGATGCTGGAAAACTACAAGAATCTGGTG | | |
| TCCCTGGGCTAC | | |

Promoters

In some embodiments, an engineered nucleic acid of the present disclosure comprises a first expression cassette comprising a first promoter operably linked to an exogenous polynucleotide sequence. In some embodiments, an engineered nucleic acid of the present disclosure comprises a second expression cassette comprising a promoter operably linked to a second exogenous polynucleotide sequence encoding one or more effector molecules. In some embodiments, the first expression cassette and second expression cassette are each encoded by a separate engineered nucleic acid of the present disclosure. In other embodiments, the first expression cassette and the second expression cassette are encoded by the same engineered nucleic acid of the present disclosure.

In some embodiments, an ACP-responsive promoter of the present disclosure comprises an ACP-binding domain and a promoter sequence. In some embodiments, the ACP-responsive promoter is operable linked to a nucleotide sequence encoding an effector molecule (e.g., a protein of interest).

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid of the present disclosure may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 Mar.; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 3, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Non-limiting examples of components that may be included in an inducible promoter (e.g., minimal promoters and responsive elements) are shown in Table 4.

TABLE 3
Exemplary Inducible Promoters

| | Promoter and | Transcription | Inducer | Response to inducer | |
|---|---|---|---|---|---|
| System | operator | factor (TF) | molecule | TF | T |

Transcriptional activator-responsive promoters

| | | | | | |
|---|---|---|---|---|---|
| AIR | PAIR (OalcA- | AlcR | Acetaldehyde | n.d. | A |
| | PhCMVmin) | | | | |
| ART | PART (OARG- | ArgR-VP16 | l-Arginine | B | A |
| | PhCMVmin) | | | | |
| BIT | PBIT3 (ObirA3- | BIT (BirA-VP16) | Biotin | B | A |
| | PhCMVmin) | | | | |
| Cumate - activator | PCR5 (Ocu06- | cTA (CymR-VP16) | Cumate | D | DA |
| | PhCMVmin) | | | | |
| Cumate - reverse | PCR5 (Ocu06- | rcTA (rCymR- | Cumate | B | A |
| activator | PhCMVmin) | VP16) | | | |
| E-OFF | PETR (OETR- | ET (E-VP16) | Erythromycin | D | DA |
| | PhCMVmin) | | | | |
| NICE-OFF | PNIC (ONIC- | NT (HdnoR-VP16) | 6-Hydroxy- | D | DA |
| | PhCMVmin) | | nicotine | | |
| PEACE | PTtgR1 (OttgR- | TtgA1 (TtgR- | Phloretin | D | DA |
| | PhCMVmin) | VP16) | | | |
| PIP-OFF | PPIR (OPIR- | PIT (PIP-VP16) | Pristinamycin I | D | DA |
| | Phsp70min) | | | | |
| QuoRex | PSCA (OscbR- | SCA (ScbR-VP16) | SCB1 | D | DA |
| | PhCMVmin)PSPA | | | | |
| | (OpapRI-PhCMVmin) | | | | |
| Redox | PROP (OROP- | REDOX (REX- | NADH | D | DA |
| | PhCMVmin) | VP16) | | | |
| TET-OFF | PhCMV*-1 (OtetO7- | tTA (TetR-VP16) | Tetracycline | D | DA |
| | PhCMVmin) | | | | |
| TET-ON | PhCMV*-1 (OtetO7- | rtTA (rTetR-VP16) | Doxycycline | B | A |
| | PhCMVmin) | | | | |
| TIGR | PCTA (OrheO- | CTA (RheA-VP16) | Heat | D | DA |
| | PhCMVmin) | | | | |
| TraR | O7x(tra box)- | p65-TraR | 3-Oxo-C8-HSL | B | A |
| | PhCMVmin | | | | |
| VAC-OFF | P1VanO2 (OvanO2- | VanA1 (VanR- | Vanillic acid | D | DA |
| | PhCMVmin) | VP16) | | | |

Transcriptional repressor-responsive promoters

| | | | | | |
|---|---|---|---|---|---|
| Cumate - | PcuO (PCMV5-OcuO) | CymR | Cumate | D | DR |
| repressor | | | | | |
| E-ON | PETRON8 (PSV40- | E-KRAB | Erythromycin | D | DR |
| | OETR8) | | | | |
| NICE-ON | PNIC (PSV40- | NS (HdnoR- | 6-Hydroxy- | D | DR |
| | ONIC8) | KRAB) | nicotine | | |
| PIP-ON | PPIRON (PSV40- | PIT3 (PIP-KRAB) | Pristinamycin I | D | DR |
| | OPIR3) | | | | |
| Q-ON | PSCAON8 (PSV40- | SCS (ScbR-KRAB) | SCB1 | D | DR |
| | OscbR8) | | | | |
| TET-ON<comma> | OtetO-PHPRT | tTS-H4 (TetR- | Doxycycline | D | DR |
| repressor-based | | HDAC4) | | | |
| T-REX | PtetO (PhCMV- | TetR | Tetracycline | D | DR |
| | OtetO2) | | | | |
| UREX | PUREX8 (PSV40- | mUTS (KRAB- | Uric acid | D | DR |
| | OhucO8) | HucR) | | | |
| VAC-ON | PvanON8 (PhCMV- | VanA4 (VanR- | Vanillic acid | D | DR |
| | OvanO8) | KRAB) | | | |

Hybrid promoters

| | | | | | |
|---|---|---|---|---|---|
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | TetracyclineErythromycin | DD | DADR |
| ON(NOT IF gate) | PhCMVmin | | | | |
| TET-OFFPIP- | OtetO7-OPIR3- | tTAPIT3E-KRAB | TetracyclinePristinamycin | DDD | DADRDR |
| ONE-ON | OETR8-PhCMVmin | | | | |
| | | | Ierythromycin | | |

TABLE 4
Exemplary Components of Inducible Promoters

| Name | DNA SEQUENCE | Source |
|---|---|---|
| minimal promoter; minP | AGAGGGTATATAATGGAAGCTCGACTTC | EU581860.1 |
| | CAG (SEQ ID NO: 1) | (Promega) |
| NFkB response element | GGGAATTTCCGGGGACTTTCCGGGAATT | EU581860.1 |
| protein promoter; 5x | TCCGGGGACTTTCCGGGAATTTCC (SEQ | (Promega) |
| NFkB-RE | ID NO: 2) | |
| CREB response element | CACCAGACAGTGACGTCAGCTGCCAGAT | DQ904461.1 |
| protein promoter; 4x CRE | CCCATGGCCGTCATACTGTGACGTCTTTC | (Promega) |
| | AGACACCCCATTGACGTCAATGGGAGAA | |
| | (SEQ ID NO: 3) | |
| NFAT response element | GGAGGAAAAACTGTTTCATACAGAAGGC | DQ904462.1 |
| protein promoter; 3x NFAT | GTGGAGGAAAAACTGTTTCATACAGAAG | (Promega) |
| binding sites | GCGTGGAGGAAAAACTGTTTCATACAGA | |
| | AGGCGT (SEQ ID NO: 4) | |
| SRF response element | AGGATGTCCATATTAGGACATCTAGGAT | FJ773212.1 |
| protein promoter; 5x SRE | GTCCATATTAGGACATCTAGGATGTCCA | (Promega) |
| | TATTAGGACATCTAGGATGTCCATATTA | |
| | GGACATCTAGGATGTCCATATTAGGACA | |
| | TCT (SEQ ID NO: 5) | |
| SRF response element | AGTATGTCCATATTAGGACATCTACCAT | FJ773213.1 |
| protein promoter 2; 5x | GTCCATATTAGGACATCTACTATGTCCAT | (Promega) |
| SRF-RE | ATTAGGACATCTTGTATGTCCATATTAG | |
| | GACATCTAAAATGTCCATATTAGGACAT | |
| | CT (SEQ ID NO: 6) | |
| AP1 response element | TGAGTCAGTGACTCAGTGAGTCAGTGAC | JQ858516.1 |
| protein promoter; 6x AP1- | TCAGTGAGTCAGTGACTCAG (SEQ ID NO: | (Promega) |
| RE | 7) | |
| TCF-LEF response element | AGATCAAAGGGTTTAAGATCAAAGGGCT | JX099537.1 |
| promoter; 8x TCF-LEF-RE | TAAGATCAAAGGGTATAAGATCAAAGG | (Promega) |
| | GCCTAAGATCAAAGGGACTAAGATCAA | |
| | AGGGTTTAAGATCAAAGGGCTTAAGATC | |
| | AAAGGGCCTA (SEQ ID NO: 8) | |
| SBEx4 | GTCTAGACGTCTAGACGTCTAGACGTCT | Addgene Cat No: 16495 |
| | AGAC (SEQ ID NO: 9) | |
| SMAD2/3-CAGACA x4 | CAGACACAGACACAGACACAGACA (SEQ | Jonk et al. (J Biol Chem. |
| | ID NO: 10) | 1998 Aug. 14; |
| | | 273(33):21145-52. |
| STAT3 binding site | Ggatccggtactcgagatctgcgatctaagtaag | Addgene Sequencing |
| | cttggcattccggtactgttggtaaagccac | Result #211335 |
| | (SEQ ID NO: 11) | |

Other non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter. In some embodiments, the promoter is a constitutive promoter. Exemplary constitutive promoters are shown in Table 5.

TABLE 5
Exemplary Constitutive Promoters

| Name | DNA SEQUENCE |
|---|---|
| CMV | GTTGACATTGATTATTGACTAGTTATTAATAGTAATCAATTACGGGGTCATTA |
| | GTTCATAGCCCATATATGGAGTTCCGCGTTACATAACTTACGGTAAATGGCCC |
| | GCCTGGCTGACCGCCCAACGACCCCCGCCCATTGACGTCAATAATGACGTATG |
| | TTCCCATAGTAACGCCAATAGGGACTTTCCATTGACGTCAATGGGTGGAGTAT |
| | TTACGGTAAACTGCCCACTTGGCAGTACATCAAGTGTATCATATGCCAAGTAC |
| | GCCCCCTATTGACGTCAATGACGGTAAATGGCCCGCCTGGCATTATGCCCAGT |
| | ACATGACCTTATGGGACTTTCCTACTTGGCAGTACATCTACGTATTAGTCATC |
| | GCTATTACCATGGTGATGCGGTTTTGGCAGTACATCAATGGGCGTGGATAGCG |
| | GTTTGACTCACGGGGATTTCCAAGTCTCCACCCCATTGACGTCAATGGGAGTT |
| | TGTTTTGGCACCAAAATCAACGGGACTTTCCAAAATGTCGTAACAACTCCGCC |
| | CCATTGACGCAAATGGGCGGTAGGCGTGTACGGTGGGAGGTCTATATAAGCA |
| | GAGCTC (SEQ ID NO: 12) |
| EF1a | GGCTCCGGTGCCCGTCAGTGGGCAGAGCGCACATCGCCCACAGTCCCCGAGA |
| | AGTTGGGGGGAGGGGTCGGCAATTGAACCGGTGCCTAGAGAAGGTGGCGCG |
| | GGGTAAACTGGGAAAGTGATGCCGTGTACTGGCTCCGCCTTTTTCCCGAGGGT |
| | GGGGGAGAACCGTATATAAGTGCAGTAGTCGCCGTGAACGTTCTTTTTCGCAA |
| | CGGGTTTGCCGCCAGAACACAGGTAAGTGCCGTGTGTGGTTCCCGCGGGCCT |
| | GGCCTCTTTACGGGTTATGGCCCTTGCGTGCCTTGAATTACTTCCACCTGGCTG |
| | CAGTACGTGATTCTTGATCCCGAGCTTCGGGTTGGAAGTGGGTGGGAGAGTTC |
| | GAGGCCTTGCGCTTAAGGAGCCCCTTCGCCTCGTGCTTGAGTTGAGGCCTGGC |
| | CTGGGCGCTGGGGCCGCCGCGTGCGAATCTGGTGGCACCTTCGCGCCTGTCTC |
| | GCTGCTTTCGATAAGTCTCTAGCCATTTAAAATTTTTGATGACCTGCTGCGAC |
| | GCTTTTTTTCTGGCAAGATAGTCTTGTAAATGCGGGCCAAGATCTGCACACTG |
| | GTATTTCGGTTTTTGGGGCCGCGGGCGGCGACGGGGCCCGTGCGTCCCAGCGC |
| | ACATGTTCGGCGAGGCGGGGCCTGCGAGCGCGACCACCGAGAATCGGACGGG |
| | GGTAGTCTCAAGCTGGCCGGCCTGCTCTGGTGCCTGTCCTCGCGCCGCCGTGT |
| | ATCGCCCCGCCCCGGGCGGCAAGGCTGGCCCGGTCGGCACCAGTTGCGTGAG |
| | CGGAAAGATGGCCGCTTCCCGGTCCTGCTGCAGGGAGCTCAAAATGGAGGAC |
| | GCGGCGCTCGGGAGAGCGGGCGGGTGAGTCACCCACACAAAGGAAAAGGGC |
| | CTTTCCGTCCTCAGCCGTCGCTTCATGTGACTCCACGGAGTACCGGGCGCCGT |
| | CCAGGCACCTCGATTAGTTCTCGAGCTTTTGGAGTACGTCGTCTTTAGGTTGG |
| | GGGGAGGGGTTTTATGCGATGGAGTTTCCCCACACTGAGTGGGTGGAGACTG |
| | AAGTTAGGCCAGCTTGGCACTTGATGTAATTCTCCTTGGAATTTGCCCTTTTTG |
| | AGTTTGGATCTTGGTTCATTCTCAAGCCTCAGACAGTGGTTCAAAGTTTTTTTC |
| | TTCCATTTCAGGTGTCGTGA (SEQ ID NO: 13) |
| EFS | GGATCTGCGATCGCTCCGGTGCCCGTCAGTGGGCAGAGCGCACATCGCCCAC |
| | AGTCCCCGAGAAGTTGGGGGGAGGGGTCGGCAATTGAACCGGTGCCTAGAGA |
| | AGGTGGCGCGGGGTAAACTGGGAAAGTGATGTCGTGTACTGGCTCCGCCTTTT |
| | TCCCGAGGGTGGGGGAGAACCGTATATAAGTGCAGTAGTCGCCGTGAACGTT |
| | CTTTTTCGCAACGGGTTTGCCGCCAGAACACAGCTGAAGCTTCGAGGGGCTCG |
| | CATCTCTCCTTCACGCGCCCGCCGCCCTACCTGAGGCCGCCATCCACGCCGGT |
| | TGAGTCGCGTTCTGCCGCCTCCCGCCTGTGGTGCCTCCTGAACTGCGTCCGCC |
| | GTCTAGGTAAGTTTAAAGCTCAGGTCGAGACCGGGCCTTTGTCCGGCGCTCCC |
| | TTGGAGCCTACCTAGACTCAGCCGGCTCTCCACGCTTTGCCTGACCCTGCTTG |
| | CTCAACTCTACGTCTTTGTTTCGTTTTCTGTTCTGCGCCGTTACAGATCCAAGC |
| | TGTGACCGGCGCCTAC (SEQ ID NO: 14) |
| MND | TTTATTTAGTCTCCAGAAAAAGGGGGGAATGAAAGACCCCACCTGTAGGTTT |
| | GGCAAGCTAGGATCAAGGTTAGGAACAGAGAGACAGCAGAATATGGGCCAA |
| | ACAGGATATCTGTGGTAAGCAGTTCCTGCCCCGGCTCAGGGCCAAGAACAGT |
| | TGGAACAGCAGAATATGGGCCAAACAGGATATCTGTGGTAAGCAGTTCCTGC |
| | CCCGGCTCAGGGCCAAGAACAGATGGTCCCCAGATGCGGTCCCGCCCTCAGC |
| | AGTTTCTAGAGAACCATCAGATGTTTCCAGGGTGCCCCAAGGACCTGAAATG |
| | ACCCTGTGCCTTATTTGAACTAACCAATCAGTTCGCTTCTCGCTTCTGTTCGCG |
| | CGCTTCTGCTCCCCGAGCTCAATAAAAGAGCCCA (SEQ ID NO: 15) |
| PGK | GGGGTTGGGGTTGCGCCTTTTCCAAGGCAGCCCTGGGTTTGCGCAGGGACGC |
| | GGCTGCTCTGGGCGTGGTTCCGGGAAACGCAGCGGCGCCGACCCTGGGTCTC |
| | GCACATTCTTCACGTCCGTTCGCAGCGTCACCCGGATCTTCGCCGCTACCCTT |
| | GTGGGCCCCCCGGCGACGCTTCCTGCTCCGCCCCTAAGTCGGGAAGGTTCCTT |
| | GCGGTTCGCGGCGTGCCGGACGTGACAAACGGAAGCCGCACGTCTCACTAGT |
| | ACCCTCGCAGACGGACAGCGCCAGGGAGCAATGGCAGCGCGCCGACCGCGAT |
| | GGGCTGTGGCCAATAGCGGCTGCTCAGCGGGGCGCGCCGAGAGCAGCGGCCG |
| | GGAAGGGGCGGTGCGGGAGGCGGGGTGTGGGGCGGTAGTGTGGGCCCTGTTC |
| | CTGCCCGCGCGGTGTTCCGCATTCTGCAAGCCTCCGGAGCGCACGTCGGCAGT |
| | CGGCTCCCTCGTTGACCGAATCACCGACCTCTCTCCCCAG (SEQ ID NO: 16) |
| SFFV | GTAACGCCATTTTGCAAGGCATGGAAAAATACCAAACCAAGAATAGAGAAGT |
| | TCAGATCAAGGGCGGGTACATGAAAATAGCTAACGTTGGGCCAAACAGGATA |
| | TCTGCGGTGAGCAGTTTCGGCCCCGGCCCGGGGCCAAGAACAGATGGTCACC |
| | GCAGTTTCGGCCCCGGCCCGAGGCCAAGAACAGATGGTCCCCAGATATGGCC |
| | CAACCCTCAGCAGTTTCTTAAGACCCATCAGATGTTTCCAGGCTCCCCCAAGG |
| | ACCTGAAATGACCCTGCGCCTTATTTGAATTAACCAATCAGCCTGCTTCTCGC |
| | TTCTGTTCGCGCGCTTCTGCTTCCCGAGCTCTATAAAAGAGCTCACAACCCCT |
| | CACTCGGCGCGCCAGTCCTCCGACAGACTGAGTCGCCCGGG (SEQ ID NO: 17) |
| SV40 | CTGTGGAATGTGTGTCAGTTAGGGTGTGGAAAGTCCCCAGGCTCCCCAGCAG |
| | GCAGAAGTATGCAAAGCATGCATCTCAATTAGTCAGCAACCAGGTGTGGAAA |
| | GTCCCCAGGCTCCCCAGCAGGCAGAAGTATGCAAAGCATGCATCTCAATTAG |
| | TCAGCAACCATAGTCCCGCCCCTAACTCCGCCCATCCCGCCCCTAACTCCGCC |
| | CAGTTCCGCCCATTCTCCGCCCCATGGCTGACTAATTTTTTTTATTTATGCAGA |
| | GGCCGAGGCCGCCTCTGCCTCTGAGCTATTCCAGAAGTAGTGAGGAGGCTTTT |
| | TTGGAGGCCTAGGCTTTTGCAAAAAGCT (SEQ ID NO: 18) |
| UbC | GCGCCGGGTTTTGGCGCCTCCCGCGGGCGCCCCCCTCCTCACGGCGAGCGCTG |
| | CCACGTCAGACGAAGGGCGCAGGAGCGTTCCTGATCCTTCCGCCCGGACGCT |
| | CAGGACAGCGGCCCGCTGCTCATAAGACTCGGCCTTAGAACCCCAGTATCAG |
| | CAGAAGGACATTTTAGGACGGGACTTGGGTGACTCTAGGGCACTGGTTTTCTT |
| | TCCAGAGAGCGGAACAGGCGAGGAAAAGTAGTCCCTTCTCGGCGATTCTGCG |
| | GAGGGATCTCCGTGGGGCGGTGAACGCCGATGATTATATAAGGACGCGCCGG |
| | GTGTGGCACAGCTAGTTCCGTCGCAGCCGGGATTTGGGTCGCGGTTCTTGTTT |
| | GTGGATCGCTGTGATCGTCACTTGGTGAGTTGCGGGCTGCTGGGCTGGCCGGG |
| | GCTTTCGTGGCCGCCGGGCCGCTCGGTGGGACGGAAGCGTGTGGAGAGACCG |
| | CCAAGGGCTGTAGTCTGGGTCCGCGAGCAAGGTTGCCCTGAACTGGGGGTTG |
| | GGGGGAGCGCACAAAATGGCGGCTGTTCCCGAGTCTTGAATGGAAGACGCTT |
| | GTAAGGCGGGCTGTGAGGTCGTTGAAACAAGGTGGGGGGCATGGTGGGCGGC |
| | AAGAACCCAAGGTCTTGAGGCCTTCGCTAATGCGGGAAAGCTCTTATTCGGGT |
| | GAGATGGGCTGGGGCACCATCTGGGGACCCTGACGTGAAGTTTGTCACTGAC |
| | TGGAGAACTCGGGTTTGTCGTCTGGTTGCGGGGGCGGCAGTTATGCGGTGCCG |
| | TTGGGCAGTGCACCCGTACCTTTGGGAGCGCGCGCCTCGTCGTGTCGTGACGT |
| | CACCCGTTCTGTTGGCTTATAATGCAGGGTGGGGCCACCTGCCGGTAGGTGTG |
| | CGGTAGGCTTTTCTCCGTCGCAGGACGCAGGGTTCGGGCCTAGGGTAGGCTCT |
| | CCTGAATCGACAGGCGCCGGACCTCTGGTGAGGGGAGGGATAAGTGAGGCGT |
| | CAGTTTCTTTGGTCGGTTTTATGTACCTATCTTCTTAAGTAGCTGAAGCTCCGG |
| | TTTTGAACTATGCGCTCGGGGTTGGCGAGTGTGTTTTGTGAAGTTTTTTAGGCA |
| | CCTTTTGAAATGTAATCATTTGGGTCAATATGTAATTTTCAGTGTTAGACTAGT |
| | AAAGCTTCTGCAGGTCGACTCTAGAAAATTGTCCGCTAAATTCTGGCCGTTTT |
| | TGGCTTTTTTGTTAGAC (SEQ ID NO: 19) |
| hEF1aV1 | GGCTCCGGTGCCCGTCAGTGGGCAGAGCGCACATCGCCCACAGTCCCCGAGA |
| | AGTTGGGGGGAGGGGTCGGCAATTGAACCGGTGCCTAGAGAAGGTGGCGCG |
| | GGGTAAACTGGGAAAGTGATGTCGTGTACTGGCTCCGCCTTTTTCCCGAGGGT |
| | GGGGGAGAACCGTATATAAGTGCAGTAGTCGCCGTGAACGTTCTTTTTCGCAA |
| | CGGGTTTGCCGCCAGAACACAGGTAAGTGCCGTGTGTGGTTCCCGCGGGCCT |
| | GGCCTCTTTACGGGTTATGGCCCTTGCGTGCCTTGAATTACTTCCACCTGGCTG |
| | CAGTACGTGATTCTTGATCCCGAGCTTCGGGTTGGAAGTGGGTGGGAGAGTTC |
| | GAGGCCTTGCGCTTAAGGAGCCCCTTCGCCTCGTGCTTGAGTTGAGGCCTGGC |
| | CTGGGCGCTGGGGCCGCCGCGTGCGAATCTGGTGGCACCTTCGCGCCTGTCTC |
| | GCTGCTTTCGATAAGTCTCTAGCCATTTAAAATTTTTGATGACCTGCTGCGAC |
| | GCTTTTTTTCTGGCAAGATAGTCTTGTAAATGCGGGCCAAGATCTGCACACTG |
| | GTATTTCGGTTTTTGGGGCCGCGGGCGGCGACGGGGCCCGTGCGTCCCAGCGC |
| | ACATGTTCGGCGAGGCGGGGCCTGCGAGCGCGGCCACCGAGAATCGGACGGG |
| | GGTAGTCTCAAGCTGGCCGGCCTGCTCTGGTGCCTGGTCTCGCGCCGCCGTGT |
| | ATCGCCCCGCCCTGGGCGGCAAGGCTGGCCCGGTCGGCACCAGTTGCGTGAG |
| | CGGAAAGATGGCCGCTTCCCGGCCCTGCTGCAGGGAGCTCAAAATGGAGGAC |
| | GCGGCGCTCGGGAGAGCGGGCGGGTGAGTCACCCACACAAAGGAAAAGGGC |
| | CTTTCCGTCCTCAGCCGTCGCTTCATGTGACTCCACGGAGTACCGGGCGCCGT |
| | CCAGGCACCTCGATTAGTTCTCGAGCTTTTGGAGTACGTCGTCTTTAGGTTGG |
| | GGGGAGGGGTTTTATGCGATGGAGTTTCCCCACACTGAGTGGGTGGAGACTG |
| | AAGTTAGGCCAGCTTGGCACTTGATGTAATTCTCCTTGGAATTTGCCCTTTTTG |
| | AGTTTGGATCTTGGTTCATTCTCAAGCCTCAGACAGTGGTTCAAAGTTTTTTTC |
| | TTCCATTTCAGGTGTCGTGA (SEQ ID NO: 20) |
| hCAGG | ACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCA |
| | ACGACCCCCGCCCATTGACGTCAATAATGACGTATGTTCCCATAGTAACGCCA |
| | ATAGGGACTTTCCATTGACGTCAATGGGTGGAGTATTTACGGTAAACTGCCCA |
| | CTTGGCAGTACATCAAGTGTATCATATGCCAAGTACGCCCCCTATTGACGTCA |
| | ATGACGGTAAATGGCCCGCCTGGCATTATGCCCAGTACATGACCTTATGGGAC |
| | TTTCCTACTTGGCAGTACATCTACGTATTAGTCATCGCTATTACCATGGTCGAG |
| | GTGAGCCCCACGTTCTGCTTCACTCTCCCCATCTCCCCCCCCTCCCCACCCCCA |
| | ATTTTGTATTTATTTATTTTTTAATTATTTTGTGCAGCGATGGGGGCGGGGGGG |
| | GGGGGGGGGCGCGCGCCAGGCGGGGCGGGGCGGGGCGAGGGGGGGGGGGG |
| | GGCGAGGCGGAGAGGTGCGGCGGCAGCCAATCAGAGCGGCGCGCTCCGAAA |
| | GTTTCCTTTTATGGCGAGGCGGCGGCGGCGGCGGCCCTATAAAAAGCGAAGC |
| | GCGCGGCGGGCGGGGAGTCGCTGCGACGCTGCCTTCGCCCCGTGCCCCGCTC |
| | CGCCGCCGCCTCGCGCCGCCCGCCCCGGCTCTGACTGACCGCGTTACTCCCAC |
| | AGGTGAGCGGGCGGGACGGCCCTTCTCCTCCGGGCTGTAATTAGCGCTTGGTT |
| | TAATGACGGCTTGTTTCTTTTCTGTGGCTGCGTGAAAGCCTTGAGGGGCTCCG |
| | GGAGGGCCCTTTGTGCGGGGGGAGCGGCTCGGGGGGTGCGTGCGTGTGTGTG |
| | TGCGTGGGGAGCGCCGCGTGCGGCTCCGCGCTGCCCGGCGGCTGTGAGCGCT |
| | GCGGGCGCGGCGCGGGGCTTTGTGCGCTCCGCAGTGTGCGCGAGGGGAGCGC |
| | GGCCGGGGGCGGTGCCCCGCGGTGCGGGGGGGGCTGCGAGGGGAACAAAGG |
| | CTGCGTGCGGGGTGTGTGCGTGGGGGGGTGAGCAGGGGGTGTGGGCGCGTCG |
| | GTCGGGCTGCAACCCCCCCTGCACCCCCCTCCCCGAGTTGCTGAGCACGGCCC |
| | GGCTTCGGGTGCGGGGCTCCGTACGGGGCGTGGCGCGGGGCTCGCCGTGCCG |
| | GGCGGGGGGTGGCGGCAGGTGGGGGTGCCGGGCGGGGCGGGGCCGCCTCGG |
| | GCCGGGGAGGGCTCGGGGGAGGGGCGCGGCGGCCCCCGGAGCGCCGGCGGC |
| | TGTCGAGGCGCGGCGAGCCGCAGCCATTGCCTTTTATGGTAATCGTGCGAGA |
| | GGGCGCAGGGACTTCCTTTGTCCCAAATCTGTGCGGAGCCGAAATCTGGGAG |
| | GCGCCGCCGCACCCCCTCTAGCGGGCGCGGGGCGAAGCGGTGCGGCGCCGGC |
| | AGGAAGGAAATGGGCGGGGAGGGCCTTCGTGCGTCGCCGCGCCGCCGTCCCC |
| | TTCTCCCTCTCCAGCCTCGGGGCTGTCCGCGGGGGGACGGCTGCCTTCGGGGG |
| | GGACGGGGCAGGGCGGGGTTCGGCTTCTGGCGTGTGACCGGCGGCTCTAGAG |
| | CCTCTGCTAACCATGTTCATGCCTTCTTCTTTTTCCTACAGCTCCTGGGCAACG |
| | TGCTGGTTATTGTGCTGTCTCATCATTTTGGCAAAGAATTC (SEQ ID NO: 21) |
| hEF1aV2 | GGGCAGAGCGCACATCGCCCACAGTCCCCGAGAAGTTGGGGGGAGGGGTCG |
| | GCAATTGAACCGGTGCCTAGAGAAGGTGGCGCGGGGTAAACTGGGAAAGTG |
| | ATGTCGTGTACTGGCTCCGCCTTTTTCCCGAGGGTGGGGGAGAACCGTATATA |
| | AGTGCAGTAGTCGCCGTGAACGTTCTTTTTCGCAACGGGTTTGCCGCCAGAAC |
| | ACAG (SEQ ID NO: 22) |
| hACTb | CCACTAGTTCCATGTCCTTATATGGACTCATCTTTGCCTATTGCGACACACACT |
| | CAATGAACACCTACTACGCGCTGCAAAGAGCCCCGCAGGCCTGAGGTGCCCC |
| | CACCTCACCACTCTTCCTATTTTTGTGTAAAAATCCAGCTTCTTGTCACCACCT |
| | CCAAGGAGGGGGAGGAGGAGGAAGGCAGGTTCCTCTAGGCTGAGCCGAATG |
| | CCCCTCTGTGGTCCCACGCCACTGATCGCTGCATGCCCACCACCTGGGTACAC |
| | ACAGTCTGTGATTCCCGGAGCAGAACGGACCCTGCCCACCCGGTCTTGTGTGC |
| | TACTCAGTGGACAGACCCAAGGCAAGAAAGGGTGACAAGGACAGGGTCTTCC |
| | CAGGCTGGCTTTGAGTTCCTAGCACCGCCCCGCCCCCAATCCTCTGTGGCACA |
| | TGGAGTCTTGGTCCCCAGAGTCCCCCAGCGGCCTCCAGATGGTCTGGGAGGG |
| | CAGTTCAGCTGTGGCTGCGCATAGCAGACATACAACGGACGGTGGGCCCAGA |
| | CCCAGGCTGTGTAGACCCAGCCCCCCCGCCCCGCAGTGCCTAGGTCACCCACT |
| | AACGCCCCAGGCCTGGTCTTGGCTGGGCGTGACTGTTACCCTCAAAAGCAGG |
| | CAGCTCCAGGGTAAAAGGTGCCCTGCCCTGTAGAGCCCACCTTCCTTCCCAGG |
| | GCTGCGGCTGGGTAGGTTTGTAGCCTTCATCACGGGCCACCTCCAGCCACTGG |
| | ACCGCTGGCCCCTGCCCTGTCCTGGGGAGTGTGGTCCTGCGACTTCTAAGTGG |
| | CCGCAAGCCACCTGACTCCCCCAACACCACACTCTACCTCTCAAGCCCAGGTC |
| | TCTCCCTAGTGACCCACCCAGCACATTTAGCTAGCTGAGCCCCACAGCCAGAG |
| | GTCCTCAGGCCCTGCTTTCAGGGCAGTTGCTCTGAAGTCGGCAAGGGGGAGT |
| | GACTGCCTGGCCACTCCATGCCCTCCAAGAGCTCCTTCTGCAGGAGCGTACAG |
| | AACCCAGGGCCCTGGCACCCGTGCAGACCCTGGCCCACCCCACCTGGGCGCT |
| | CAGTGCCCAAGAGATGTCCACACCTAGGATGTCCCGCGGTGGGTGGGGGGCC |
| | CGAGAGACGGGCAGGCCGGGGGCAGGCCTGGCCATGCGGGGCCGAACCGGG |
| | CACTGCCCAGCGTGGGGCGCGGGGGCCACGGCGCGCGCCCCCAGCCCCCGGG |
| | CCCAGCACCCCAAGGCGGCCAACGCCAAAACTCTCCCTCCTCCTCTTCCTCAA |
| | TCTCGCTCTCGCTCTTTTTTTTTTTCGCAAAAGGAGGGGAGAGGGGGTAAAAA |
| | AATGCTGCACTGTGCGGCGAAGCCGGTGAGTGAGCGGCGCGGGGCCAATCAG |
| | CGTGCGCCGTTCCGAAAGTTGCCTTTTATGGCTCGAGCGGCCGCGGCGGCGCC |
| | CTATAAAACCCAGCGGCGCGACGCGCCACCACCGCCGAGACCGCGTCCGCCC |
| | CGCGAGCACAGAGCCTCGCCTTTGCCGATCCGCCGCCCGTCCACACCCGCCGC |
| | CAGGTAAGCCCGGCCAGCCGACCGGGGCAGGCGGCTCACGGCCCGGCCGCAG |
| | GCGGCCGCGGCCCCTTCGCCCGTGCAGAGCCGCCGTCTGGGCCGCAGCGGGG |
| | GGCGCATGGGGGGGGAACCGGACCGCCGTGGGGGGCGCGGGAGAAGCCCCT |
| | GGGCCTCCGGAGATGGGGGACACCCCACGCCAGTTCGGAGGCGCGAGGCCGC |
| | GCTCGGGAGGCGCGCTCCGGGGGTGCCGCTCTCGGGGCGGGGGCAACCGGCG |
| | GGGTCTTTGTCTGAGCCGGGCTCTTGCCAATGGGGATCGCAGGGTGGGCGCG |
| | GCGGAGCCCCCGCCAGGCCCGGTGGGGGCTGGGGCGCCATTGCGCGTGCGCG |
| | CTGGTCCTTTGGGCGCTAACTGCGTGCGCGCTGGGAATTGGCGCTAATTGCGC |
| | GTGCGCGCTGGGACTCAAGGCGCTAACTGCGCGTGCGTTCTGGGGCCCGGGG |
| | TGCCGCGGCCTGGGCTGGGGCGAAGGCGGGCTCGGCCGGAAGGGGTGGGGTC |
| | GCCGCGGCTCCCGGGCGCTTGCGCGCACTTCCTGCCCGAGCCGCTGGCCGCCC |
| | GAGGGTGTGGCCGCTGCGTGCGCGCGCGCCGACCCGGCGCTGTTTGAACCGG |
| | GCGGAGGCGGGGCTGGCGCCCGGTTGGGAGGGGGTTGGGGCCTGGCTTCCTG |
| | CCGCGCGCCGCGGGGACGCCTCCGACCAGTGTTTGCCTTTTATGGTAATAACG |
| | CGGCCGGCCCGGCTTCCTTTGTCCCCAATCTGGGCGCGCGCCGGCGCCCCCTG |
| | GCGGCCTAAGGACTCGGCGCGCCGGAAGTGGCCAGGGCGGGGGCGACCTCG |
| | GCTCACAGCGCGCCCGGCTAT (SEQ ID NO: 23) |
| heIF4A1 | GTTGATTTCCTTCATCCCTGGCACACGTCCAGGCAGTGTCGAATCCATCTCTG |
| | CTACAGGGGAAAACAAATAACATTTGAGTCCAGTGGAGACCGGGAGCAGAA |
| | GTAAAGGGAAGTGATAACCCCCAGAGCCCGGAAGCCTCTGGAGGCTGAGACC |
| | TCGCCCCCCTTGCGTGATAGGGCCTACGGAGCCACATGACCAAGGCACTGTC |
| | GCCTCCGCACGTGTGAGAGTGCAGGGCCCCAAGATGGCTGCCAGGCCTCGAG |
| | GCCTGACTCTTCTATGTCACTTCCGTACCGGCGAGAAAGGCGGGCCCTCCAGC |
| | CAATGAGGCTGCGGGGCGGGCCTTCACCTTGATAGGCACTCGAGTTATCCAAT |
| | GGTGCCTGCGGGCCGGAGCGACTAGGAACTAACGTCATGCCGAGTTGCTGAG |
| | CGCCGGCAGGCGGGGCCGGGGCGGCCAAACCAATGCGATGGCCGGGGCGGA |
| | GTCGGGCGCTCTATAAGTTGTCGATAGGCGGGCACTCCGCCCTAGTTTCTAAG |
| | GACCATG (SEQ ID NO: 24) |
| hGAPDH | AGTTCCCCAACTTTCCCGCCTCTCAGCCTTTGAAAGAAAGAAAGGGGAGGGG |
| | GCAGGCCGCGTGCAGTCGCGAGCGGTGCTGGGCTCCGGCTCCAATTCCCCATC |
| | TCAGTCGCTCCCAAAGTCCTTCTGTTTCATCCAAGCGTGTAAGGGTCCCCGTC |
| | CTTGACTCCCTAGTGTCCTGCTGCCCACAGTCCAGTCCTGGGAACCAGCACCG |
| | ATCACCTCCCATCGGGCCAATCTCAGTCCCTTCCCCCCTACGTCGGGGCCCAC |
| | ACGCTCGGTGCGTGCCCAGTTGAACCAGGCGGCTGCGGAAAAAAAAAAGCGG |
| | GGAGAAAGTAGGGCCCGGCTACTAGCGGTTTTACGGGCGCACGTAGCTCAGG |
| | CCTCAAGACCTTGGGCTGGGACTGGCTGAGCCTGGCGGGAGGCGGGGTCCGA |
| | GTCACCGCCTGCCGCCGCGCCCCCGGTTTCTATAAATTGAGCCCGCAGCCTCC |
| | CGCTTCGCTCTCTGCTCCTCCTGTTCGACAGTCAGCCGCATCTTCTTTTGCGTC |
| | GCCAGGTGAAGACGGGCGGAGAGAAACCCGGGAGGCTAGGGACGGCCTGAA |
| | GGCGGCAGGGGGGGGCGCAGGCCGGATGTGTTCGCGCCGCTGCGGGGTGGGC |
| | CCGGGCGGCCTCCGCATTGCAGGGGGGGGCGGAGGACGTGATGCGGCGCGGG |
| | CTGGGCATGGAGGCCTGGTGGGGGAGGGGAGGGGAGGCGTGGGTGTCGGCC |
| | GGGGCCACTAGGCGCTCACTGTTCTCTCCCTCCGCGCAGCCGAGCCACATCGC |
| | TGAGACAC (SEQ ID NO: 25) |
| hGRP78 | AGTGCGGTTACCAGCGGAAATGCCTCGGGGTCAGAAGTCGCAGGAGAGATAG |
| | ACAGCTGCTGAACCAATGGGACCAGCGGATGGGGCGGATGTTATCTACCATT |
| | GGTGAACGTTAGAAACGAATAGCAGCCAATGAATCAGCTGGGGGGGCGGAG |
| | CAGTGACGTTTATTGCGGAGGGGGCCGCTTCGAATCGGCGGCGGCCAGCTTG |
| | GTGGCCTGGGCCAATGAACGGCCTCCAACGAGCAGGGCCTTCACCAATCGGC |
| | GGCCTCCACGACGGGGCTGGGGGAGGGTATATAAGCCGAGTAGGCGACGGTG |
| | AGGTCGACGCCGGCCAAGACAGCACAGACAGATTGACCTATTGGGGTGTTTC |
| | GCGAGTGTGAGAGGGAAGCGCCGCGGCCTGTATTTCTAGACCTGCCCTTCGCC |
| | TGGTTCGTGGCGCCTTGTGACCCCGGGCCCCTGCCGCCTGCAAGTCGGAAATT |
| | GCGCTGTGCTCCTGTGCTACGGCCTGTGGCTGGACTGCCTGCTGCTGCCCAAC |
| | TGGCTGGCAC (SEQ ID NO: 26) |
| hGRP94 | TAGTTTCATCACCACCGCCACCCCCCCGCCCCCCCGCCATCTGAAAGGGTTCT |
| | AGGGGATTTGCAACCTCTCTCGTGTGTTTCTTCTTTCCGAGAAGCGCCGCCAC |
| | ACGAGAAAGCTGGCCGCGAAAGTCGTGCTGGAATCACTTCCAACGAAACCCC |
| | AGGCATAGATGGGAAAGGGTGAAGAACACGTTGCCATGGCTACCGTTTCCCC |
| | GGTCACGGAATAAACGCTCTCTAGGATCCGGAAGTAGTTCCGCCGCGACCTCT |
| | CTAAAAGGATGGATGTGTTCTCTGCTTACATTCATTGGACGTTTTCCCTTAGA |
| | GGCCAAGGCCGCCCAGGCAAAGGGGCGGTCCCACGCGTGAGGGGCCCGCGG |
| | AGCCATTTGATTGGAGAAAAGCTGCAAACCCTGACCAATCGGAAGGAGCCAC |
| | GCTTCGGGCATCGGTCACCGCACCTGGACAGCTCCGATTGGTGGACTTCCGCC |
| | CCCCCTCACGAATCCTCATTGGGTGCCGTGGGTGCGTGGTGCGGCGCGATTGG |
| | TGGGTTCATGTTTCCCGTCCCCCGCCCGCGAGAAGTGGGGGTGAAAAGCGGC |
| | CCGACCTGCTTGGGGTGTAGTGGGCGGACCGCGCGGCTGGAGGTGTGAGGAT |
| | CCGAACCCAGGGGTGGGGGGTGGAGGCGGCTCCTGCGATCGAAGGGGACTTG |
| | AGACTCACCGGCCGCACGTC (SEQ ID NO: 27) |
| hHSP70 | GGGCCGCCCACTCCCCCTTCCTCTCAGGGTCCCTGTCCCCTCCAGTGAATCCC |
| | AGAAGACTCTGGAGAGTTCTGAGCAGGGGGCGGCACTCTGGCCTCTGATTGG |
| | TCCAAGGAAGGCTGGGGGGCAGGACGGGAGGCGAAAACCCTGGAATATTCC |
| | CGACCTGGCAGCCTCATCGAGCTCGGTGATTGGCTCAGAAGGGAAAAGGCGG |
| | GTCTCCGTGACGACTTATAAAAGCCCAGGGGCAAGCGGTCCGGATAACGGCT |
| | AGCCTGAGGAGCTGCTGCGACAGTCCACTACCTTTTTCGAGAGTGACTCCCGT |
| | TGTCCCAAGGCTTCCCAGAGCGAACCTGTGCGGCTGCAGGCACCGGCGCGTC |
| | GAGTTTCCGGCGTCCGGAAGGACCGAGCTCTTCTCGCGGATCCAGTGTTCCGT |
| | TTCCAGCCCCCAATCTCAGAGCGGAGCCGACAGAGAGCAGGGAACCC (SEQ |
| | ID NO: 28) |
| hKINb | GCCCCACCCCCGTCCGCGTTACAACCGGGAGGCCCGCTGGGTCCTGCACCGTC |
| | ACCCTCCTCCCTGTGACCGCCCACCTGATACCCAAACAACTTTCTCGCCCCTC |
| | CAGTCCCCAGCTCGCCGAGCGCTTGCGGGGAGCCACCCAGCCTCAGTTTCCCC |
| | AGCCCCGGGCGGGGCGAGGGGCGATGACGTCATGCCGGCGCGCGGCATTGTG |
| | GGGCGGGGCGAGGCGGGGCGCCGGGGGGAGCAACACTGAGACGCCATTTTC |
| | GGCGGCGGGAGCGGCGCAGGCGGCCGAGCGGGACTGGCTGGGTCGGCTGGG |
| | CTGCTGGTGCGAGGAGCCGCGGGGCTGTGCTCGGCGGCCAAGGGGACAGCGC |
| | GTGGGTGGCCGAGGATGCTGCGGGGCGGTAGCTCCGGCGCCCCTCGCTGGTG |
| | ACTGCTGCGCCGTGCCTCACACAGCCGAGGCGGGCTCGGCGCACAGTCGCTG |
| | CTCCGCGCTCGCGCCCGGCGGCGCTCCAGGTGCTGACAGCGCGAGAGAGCGC |
| | GGCCTCAGGAGCAACAC (SEQ ID NO: 29) |
| hUBIb | TTCCAGAGCTTTCGAGGAAGGTTTCTTCAACTCAAATTCATCCGCCTGATAAT |
| | TTTCTTATATTTTCCTAAAGAAGGAAGAGAAGCGCATAGAGGAGAAGGGAAA |
| | TAATTTTTTAGGAGCCTTTCTTACGGCTATGAGGAATTTGGGGCTCAGTTGAA |
| | AAGCCTAAACTGCCTCTCGGGAGGTTGGGCGCGGCGAACTACTTTCAGCGGC |
| | GCACGGAGACGGCGTCTACGTGAGGGGTGATAAGTGACGCAACACTCGTTGC |
| | ATAAATTTGCGCTCCGCCAGCCCGGAGCATTTAGGGGCGGTTGGCTTTGTTGG |
| | GTGAGCTTGTTTGTGTCCCTGTGGGTGGACGTGGTTGGTGATTGGCAGGATCC |
| | TGGTATCCGCTAACAGGTACTGGCCCACAGCCGTAAAGACCTGCGGGGGCGT |
| | GAGAGGGGGGAATGGGTGAGGTCAAGCTGGAGGCTTCTTGGGGTTGGGTGGG |
| | CCGCTGAGGGGAGGGGAGGGCGAGGTGACGCGACACCCGGCCTTTCTGGGAG |
| | AGTGGGCCTTGTTGACCTAAGGGGGGCGAGGGCAGTTGGCACGCGCACGCGC |
| | CGACAGAAACTAACAGACATTAACCAACAGCGATTCCGTCGCGTTTACTTGG |
| | GAGGAAGGCGGAAAAGAGGTAGTTTGTGTGGCTTCTGGAAACCCTAAATTTG |
| | GAATCCCAGTATGAGAATGGTGTCCCTTCTTGTGTTTCAATGGGATTTTTACTT |
| | CGCGAGTCTTGTGGGTTTGGTTTTGTTTTCAGTTTGCCTAACACCGTGCTTAGG |
| | TTTGAGGCAGATTGGAGTTCGGTCGGGGGAGTTTGAATATCCGGAACAGTTA |
| | GTGGGGAAAGCTGTGGACGCTTGGTAAGAGAGCGCTCTGGATTTTCCGCTGTT |
| | GACGTTGAAACCTTGAATGACGAATTTCGTATTAAGTGACTTAGCCTTGTAAA |
| | ATTGAGGGGAGGCTTGCGGAATATTAACGTATTTAAGGCATTTTGAAGGAAT |
| | AGTTGCTAATTTTGAAGAATATTAGGTGTAAAAGCAAGAAATACAATGATCCT |
| | GAGGTGACACGCTTATGTTTTACTTTTAAACTAGGTCACC (SEQ ID NO: 30) |
| MinCMV | Tccaggcgatctgacggttcactaaacgagctctgcttatataggcctcccaccgtaca |
| | cgccta (SEQ ID NO: 138) |

In some embodiments, the promoter sequence is derived from a promoter selected from: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof.

In some embodiments, the first promoter is a constitutive promoter, an inducible promoter, or a synthetic promoter. In some embodiments, the constitutive promoter is selected from: CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

In some embodiments, an ACP-responsive promoter is a synthetic promoter. In some embodiments, the ACP-responsive promoter comprises a minimal promoter. In some embodiments, the ACP-binding domain comprises one or more zinc finger binding sites. The ACP-binding domain can comprise 1, 2, 3, 4, 5, 6 7, 8, 9, 10, or more zinc finger binding sites. In some embodiments, the ACP-binding domain comprises one zinc finger binding site. In some embodiments, the ACP-binding domain comprises two zinc finger binding sites. In some embodiments, the ACP-binding domain comprises three zinc finger binding sites. In some embodiments, the ACP-binding domain comprises four zinc finger binding sites. An exemplary ACP-binding domain comprising zinc finger binding sites is shown in the sequence:

(SEQ ID NO: 100)
cgggtttcgtaacaatcgcatgaggattcgcaacgccttcGGCGTAGCCGATGTCGCGctcccgtctca
gtaaaggtcGGCGTAGCCGATGTCGCGcaatcggactgccttcgtacGGCGTAGCCGATGTCGCGcgta
tcagtcgcctcggaacGGCGTAGCCGATGTCGCGcattcgtaagaggctcactctcccttacacggagt
ggataACTAGTTCTAGAGGGTATATAATGGGGGCCA.

In some embodiments, an ACP-responsive promoter comprises an enhancer that promotes transcription when an antigen recognizing receptor engages a co cognate antigen, e. an, antigen expressed on a target cell. Enhancers can include, but are not limited to, enhancers enriched in the ATAC-seq of activated T cells (Gate et al. Nat Genet. Author manuscript; available in PMC 2019 Jan. 9; herein incorporated by reference for all purposes) or enhancers associated with upregulated genes in single-cell RNA seq data (Xhangolli et al. Genomics Proteomics Bioinformatics. 2019 April; 17(2):129-139. Doi: 10.1016/j.gpb.2019.03.002; herein incorporated by reference for all purposes). An enhancers can be a synthetic enhancer, such as a pair of transcription factors known or suspected to be upregulated in activated T cells or NK cells. Synthetic enhancers can include multiple iterations of transcription factor binding sites, such 4 iterations of two distinct transcription factor binding sites in an aaaabbbb or abababab organization. Illustrative non-limiting examples of genes from which enhancers can be derived include, but are not limited to, ATF2, ATF7, BACH1, BATF, Bcl-6, Blimp-1, BMI1, CBFB, CREB1, CREM, CTCF, E2F1, EBF1, EGR1, ETV6, FOS, FOXA1, FOXA2, GATA3, HIF1A, IKZF1, IKZF2, IRF4, JUN, JUNB, JUND, Lefl, NFAT, NFIA, NFIB, NFκB, NR2F1, Nur77, PU.1, RELA, RUNX3, SCRT1, SCRT2, SP1, STAT4, STATSA, T-Bet, Tcf7, ZBED1, ZNF143, or ZNF217.

Multicistronic and Multiple Promoter Systems

In some embodiments, engineered nucleic acids are configured to produce multiple polypeptides. For example, nucleic acids may be configured to produce 2-20 different polypeptides. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 polypeptides. In some embodiments, nucleic acids are configured to produce 1, 2, 3, 4, 5, 6, 7, 8, 9, 10, 11, 12, 13, 14, 15, 16, 17, 18, 19, or 20 polypeptides.

In some embodiments, engineered nucleic acids can be multicistronic, i.e., more than one separate polypeptide (e.g., multiple exogenous polynucleotides or effector molecules) can be produced from a single transcript. Engineered nucleic acids can be multicistronic through the use of various linkers, e.g., a polynucleotide sequence encoding a first exogenous polynucleotide or effector molecule can be linked to a nucleotide sequence encoding a second exogenous polynucleotide or effector molecule, such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

“Linkers,” as used herein can refer to polypeptides that link a first polypeptide sequence and a second polypeptide sequence or the multicistronic linkers described above.

Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

Engineered Cells

Also provided herein are cells, and methods of producing cells, that comprise one or more engineered nucleic acids of the present disclosure. These cells are referred to herein as “engineered cells.” These cells, which typically contain one or more engineered nucleic acids, do not occur in nature. In some embodiments, the cells are isolated cells that recombinantly express the one or more engineered nucleic acids. In some embodiments, the engineered one or more nucleic acids are expressed from one or more vectors or a selected locus from the genome of the cell. In some embodiments, the cells are engineered to include a first nucleic acid comprising a promoter operably linked to a nucleotide sequence.

An engineered cell of the present disclosure can comprise an engineered nucleic acid integrated into the cell's genome. An engineered cell can comprise an engineered nucleic acid capable of expression without integrating into the cell's genome, for example, engineered with a transient expression system such as a plasmid or mRNA.

Engineered Cell Types

An engineered cell or isolated cell of the present disclosure can be a human cell. An engineered cell or isolated cell can be a human primary cell. An engineered primary cell can be any somatic cell. An engineered primary cell can be any stem cell. An engineered primary cell can be an induced pluripotent stem cell (iPSC). In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered or isolated cell of the present disclosure is selected from: a T cell (e.g., a CD8+ T cell, a CD4+ T cell, or a gamma-delta T cell), a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage (e.g., an M1 macrophage or an M2 macrophage), a monocyte, a dendritic cell, an erythrocyte, a platelet cell, a neuron, an oligodendrocyte, an astrocyte, a placode-derived cell, a Schwann cell, a cardiomyocyte, an endothelial cell, a nodal cell, a microglial cell, a hepatocyte, a cholangiocyte, a beta cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, an engineered or isolated cell of the present disclosure is a T cell (e.g., a, a CD8+ T cell, a CD4+ T cell, or a gamma-delta T cell). In some embodiments, an engineered or isolated cell of the present disclosure is a cytotoxic T lymphocyte (CTL). In some embodiments, an engineered or isolated cell of the present disclosure is a regulatory T cell. In some embodiments, an engineered or isolated cell of the present disclosure is a Natural Killer T (NKT) cell. In some embodiments, an engineered or isolated cell of the present disclosure is a Natural Killer (NK) cell. In some embodiments, an engineered or isolated cell of the present disclosure is a B cell. In some embodiments, an engineered or isolated cell of the present disclosure is a tumor-infiltrating lymphocyte (TIL). In some embodiments, an engineered or isolated cell of the present disclosure is an innate lymphoid cell. In some embodiments, an engineered or isolated cell of the present disclosure is a mast cell. In some embodiments, an engineered or isolated cell of the present disclosure is an eosinophil. In some embodiments, an engineered or isolated cell of the present disclosure is a basophil. In some embodiments, an engineered or isolated cell of the present disclosure is a neutrophil. In some embodiments, an engineered or isolated cell of the present disclosure is a myeloid cell. In some embodiments, an engineered or isolated cell of the present disclosure is a macrophage e.g., an M1 macrophage or an M2 macrophage). In some embodiments, an engineered or isolated cell of the present disclosure is a monocyte. In some embodiments, an engineered or isolated cell of the present disclosure is a dendritic cell. In some embodiments, an engineered or isolated cell of the present disclosure is an erythrocyte. In some embodiments, an engineered or isolated cell of the present disclosure is a platelet cell. In some embodiments, an engineered or isolated cell of the present disclosure is a neuron. In some embodiments, an engineered or isolated cell of the present disclosure is an oligodendrocyte. In some embodiments, an engineered or isolated cell of the present disclosure is an astrocyte. In some embodiments, an engineered or isolated cell of the present disclosure is a placode-derived cell. In some embodiments, an engineered or isolated cell of the present disclosure is a Schwann cell. In some embodiments, an engineered or isolated cell of the present disclosure is a cardiomyocyte. In some embodiments, an engineered or isolated cell of the present disclosure is an endothelial cell. In some embodiments, an engineered or isolated cell of the present disclosure is a nodal cell. In some embodiments, an engineered or isolated cell of the present disclosure is a microglial cell. In some embodiments, an engineered or isolated cell of the present disclosure is a hepatocyte. In some embodiments, an engineered or isolated cell of the present disclosure is a cholangiocyte. In some embodiments, an engineered or isolated cell of the present disclosure is a beta cell. In some embodiments, an engineered or isolated cell of the present disclosure is a human embryonic stem cell (ESC). In some embodiments, an engineered or isolated cell of the present disclosure is an ESC-derived cell. In some embodiments, an engineered or isolated cell of the present disclosure is a pluripotent stem cell. In some embodiments, an engineered or isolated cell of the present disclosure is a mesenchymal stromal cell (MSC). In some embodiments, an engineered or isolated cell of the present disclosure is an induced pluripotent stem cell (iPSC). In some embodiments, an engineered or isolated cell of the present disclosure is an iPSC-derived cell. In some embodiments, an engineered cell is autologous. In some embodiments, an engineered cell is allogeneic. In some embodiments, an engineered or isolated cell of the present disclosure is a CD34+ cell, a CD3+ cell, a CD8+ cell, a CD16+ cell, and/or a CD4+ cell.

In some embodiments, an engineered cell of the present disclosure is a tumor cell selected from: an adenocarcinoma cell, a bladder tumor cell, a brain tumor cell, a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a prostate tumor cell, a skin tumor cell, a thyroid tumor cell, and a uterine tumor cell.

In some embodiments, an engineered cell of the present disclosure is a bacterial cell selected from: Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Methods of Engineering Cells

Also provided herein are compositions and methods for engineering cells with any nucleic acid as described herein.

In general, cells are engineered through introduction (i.e., delivery) of one or more polynucleotides of the present disclosure. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

In some embodiments, the engineered cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some embodiments, the oncolytic virus is a recombinant oncolytic virus comprising the first expression cassette and the second expression cassette. In some embodiments, the oncolytic virus further comprises the third expression cassette.

The virus, including any of the oncolytic viruses described herein, can be a recombinant virus that encodes one more transgenes encoding one or more effector molecules, such as any of the engineered nucleic acids described herein. The virus, including any of the oncolytic viruses described herein, can be a recombinant virus that encodes one more transgenes encoding one or more of the two or more effector molecules, such as any of the engineered nucleic acids described herein. In some embodiments, the cell is engineered via transduction with an oncolytic virus.

Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more effector molecules. The transgenes encoding the one or more effector molecules can be configured to express the one or more effector molecules.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immunodeficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

Lipid Structure Delivery Systems

Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery-A Review. Nanomaterials 2017, 7(5), 94), herein incorporated by reference for all purposes.

Genomic Editing Systems

A genomic editing systems can be used to engineer a host genome to encode an engineered nucleic acid, such as an engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tcl/mariner transposon superfamily, such as a Sleeping Beauty transposon system, described in more detail in Hudecek et al. (Crit Rev Biochem Mol Biol. 2017 Aug.52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA—binding domain of TALE (transcription activator-like effectors) and the catalytic domain of restriction endonuclease FokI. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; U.S. Pat. No. 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

Methods of Use

Methods for treatment of diseases are also encompassed by this disclosure. Said methods include administering a therapeutically effective amount of an engineered nucleic acid, engineered cell, or isolated cell as described above. In some aspects, provided herein are methods of treating a subject in need thereof, the method comprising administering a therapeutically effective dose of any of the engineered cells, isolated cells, or compositions disclosed herein.

In vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the effector molecules described herein. The methods provided herein also include delivering a composition in vivo capable of producing two or more of the effector molecules described herein. Compositions capable of in vivo production of effector molecules include, but are not limited to, any of the engineered nucleic acids described herein. Compositions capable of in vivo production of effector molecules can be a naked mRNA or a naked plasmid.

Pharmaceutical Compositions

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Whether it is a cell, polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16^{th} edition, Osol, A. (ed), 1980.

A composition can be administered alone or in combination with other treatments, either simultaneously or sequentially dependent upon the condition to be treated.

### EMBODIMENTS

1. An isolated cell comprising an inducible cell death polypeptide comprising two or more monomers, wherein each monomer comprises one or more ligand binding domains and an cell death-inducing domain,
wherein each of the one or more ligand binding domains comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, an FRB domain, a cereblon domain, optionally comprising the amino acid sequence set forth in one of SEQ ID NOs: 127 and 129, a degron, optionally comprising the amino acid sequence set forth in one of SEQ ID NOs: 131 and 133, a progesterone receptor domain, optionally comprising the amino acid sequence of SEQ ID NO: 52,
wherein each monomer is oligomerizable via a cognate ligand that binds to the ligand binding domain, and
wherein when the ligand oligomerizes each monomer, an cell death-inducing signal is generated in the cell.

2. The isolated cell of embodiment 1, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and Purine nucleoside phosphorylase.

3. The isolated cell of embodiment 1, wherein the cell death-inducing domain comprises caspase 9, or a functional truncation thereof.

4. The isolated cell of embodiment 3, wherein the cell death-inducing domain comprises the amino acid sequence of SEQ ID NO:39.

5. The isolated cell of embodiment 1, wherein the cell death-inducing domain comprises Bid, or a functional truncation thereof.

6. The isolated cell of embodiment 5, wherein the cell death-inducing domain comprises the amino acid sequence of SEQ ID NO: 54.

7. The isolated cell of embodiment 1, wherein the ABI domain comprises the amino acid sequence of SEQ ID NO: 31.

8. The isolated cell of embodiment 1, wherein the PYL domain comprises the amino acid sequence of SEQ ID NO: 53.

9. The isolated cell of embodiment 1, wherein the caffeine-binding single-domain antibody comprises the amino acid sequence of SEQ ID NO: 33.

10. The isolated cell of embodiment 1, wherein the cannabidiol binding domain comprises an amino acid sequence selected from the group consisting of SEQ ID NO: 34, 35, 36, 37, and 38.

11. The isolated cell of embodiment 1, wherein the hormone-binding domain of estrogen receptor (ER) domain comprises the amino acid sequence of SEQ ID NO: 42.

12. The isolated cell of embodiment 1, wherein the heavy chain variable region (VH) of an anti-nicotine antibody comprises the amino acid sequence of SEQ ID NO: 50.

13. The isolated cell of embodiment 1, wherein the light chain variable region (VL) of an anti-nicotine antibody comprises the amino acid sequence of SEQ ID NO: 51.

14. The isolated cell of embodiment 1, wherein the progesterone receptor domain comprises the amino acid sequence of SEQ ID NO: 52.

15. The isolated cell of embodiment 1, wherein the FKBP domain comprises the amino acid sequence of SEQ ID NO: 43.

16. The isolated cell of embodiment 1, wherein the FRB domain comprises the amino acid sequence of SEQ ID NO: 44.

17. The isolated cell of any one of embodiments 1-16, wherein each monomer comprises the same ligand binding domain.

18. The isolated cell of embodiment 17, wherein the inducible cell death polypeptide comprises homooligomers.

19. The isolated cell of embodiment 18, wherein the homooligomers comprise homodimers.

20. The isolated cell of any one of embodiments 17-19, wherein each monomer comprises an FKBP domain.

21. The isolated cell of embodiment 20, wherein the ligand is FK1012, a derivative thereof, or an analog thereof.

22. The isolated cell of embodiment 20 or embodiment 21, wherein the cell death-inducing domain comprises Bid, or a functional truncation thereof.

23. The isolated cell of embodiment 22, wherein the cell death-inducing domain comprises the amino acid sequence of SEQ ID NO: 54.

24. The isolated cell of any one of embodiments 1-6, wherein each monomer comprises an ABI domain and a PYL domain.

25. The isolated cell of embodiment 24, wherein the ligand is abscisic acid.

26. The isolated cell of embodiment 24 or embodiment 25, wherein the cell death-inducing domain comprises caspase 9, or a functional truncation thereof.

27. The isolated cell of embodiment 26, wherein the cell death-inducing domain comprises the amino acid sequence of SEQ ID NO: 39.

28. The isolated cell of any one of embodiments 1-6, wherein each monomer comprises a cannabidiol binding domain comprising the amino acid sequence of SEQ ID NO: 34 and a cannabidiol binding domain comprising an amino acid sequence selected from the group consisting of SEQ ID NO: 35, 36, 37, and 38.

29. The isolated cell of any one of embodiments 1-6, wherein each monomer comprises a hormone-binding domain of estrogen receptor (ER) domain and an FKBP domain.

30. The isolated cell of any one of embodiments 1-6, wherein each monomer comprises an FRB domain and a hormone-binding domain of estrogen receptor (ER) domain.

31. The isolated cell embodiment 29 or embodiment 30, wherein the cell death-inducing domain comprises caspase 9, or a functional truncation thereof.

32. The isolated cell of embodiment 31, wherein the cell death-inducing domain comprises the amino acid sequence of SEQ ID NO: 39.

33. The isolated cell of any one of embodiments 29-32, wherein the ligand is rapamycin, a derivative thereof, or an analog thereof.

34. The isolated cell of any one of embodiments 29-33, wherein the ligand is tamoxifen or a metabolite thereof.

35. The isolated cell of embodiment 34, wherein the tamoxifen metabolite is selected from the group consisting of: 4-hydroxytamoxifen, N-desmethyltamoxifen, tamoxifen-N-oxide, and endoxifen.

36. The isolated cell of any one of embodiments 1-6, wherein each monomer comprises two caffeine-binding single-domain antibodies.

37. The isolated cell of embodiment 36, wherein each caffeine-binding single-domain antibody comprises the amino acid sequence of SEQ ID NO: 33.

38. The isolated cell of embodiment 36 or embodiment 37, wherein the ligand is caffeine or a derivative thereof.

39. The isolated cell of any one of embodiments 1-38, wherein each monomer comprises a progesterone receptor domain comprising the amino acid sequence of SEQ ID NO: 52.

40. The isolated cell of embodiment 39, wherein the ligand is mifepristone or a derivative thereof

41. The isolated cell of any one of embodiments 1-38, wherein a first monomer comprises a first ligand binding domain and a second monomer comprises a second ligand binding domain.

42. The isolated cell of embodiment 41, wherein the inducible cell death polypeptide comprises heterooligomers.

43. The isolated cell of embodiment 42, wherein the heterooligomers comprise heterodimers.

44. The isolated cell of any one of embodiments 41-43, wherein the first monomer comprises an FKBP domain and the second monomer comprises an FRB domain.

45. The isolated cell of embodiment 44, wherein the cell death-inducing domain comprises Bid, or a functional truncation thereof.

46. The isolated cell of embodiment 45, wherein the cell death-inducing domain comprises the amino acid sequence of SEQ ID NO: 54.

47. The isolated cell of any one of embodiments 41-43, wherein the first monomer comprises a hormone-binding domain of estrogen receptor (ER) domain and the second monomer comprises an FKBP domain.

48. The isolated cell of any one of embodiments 41-43, wherein the first monomer comprises an FRB domain and the second monomer comprises a hormone-binding domain of estrogen receptor (ER) domain.

49. The isolated cell of any one of embodiments 41-43, wherein the first monomer comprises a hormone-binding domain of estrogen receptor (ER) domain and an FKBP domain, and the second monomer comprises an FRB domain and the second monomer comprises a hormone-binding domain of estrogen receptor (ER) domain.

50. The isolated cell of any one of embodiments 47-49, wherein the cell death-inducing domain comprises caspase 9, or a functional truncation thereof.

51. The isolated cell of embodiment 50, wherein the cell death-inducing domain comprises the amino acid sequence of SEQ ID NO: 39.

52. The isolated cell of any one of embodiments 44-51, wherein the ligand is rapamycin, a derivative thereof, or an analog thereof.

53. The isolated cell of any one of embodiments 47-52, wherein the ligand is tamoxifen or a metabolite thereof.

54. The isolated cell of embodiment 53, wherein the tamoxifen metabolite is selected from the group consisting of: 4-hydroxytamoxifen, N-desmethyltamoxifen, tamoxifen-N-oxide, and endoxifen.

55. The isolated cell of any one of embodiments 41-43, wherein the first monomer comprises an ABI domain and the second monomer comprises a PYL domain.

56. The isolated cell of embodiment 55, wherein the ligand is abscisic acid.

57. The isolated cell of any one of embodiments 41-43, wherein the first monomer comprises a heavy chain variable region (VH) of an anti-nicotine antibody and the second monomer comprises a light chain variable region (VL) of an anti-nicotine antibody.

58. The isolated cell of embodiment 57, wherein the anti-nicotine antibody is a Nic12 antibody.

59. The isolated cell of embodiment 57 or embodiment 58, wherein the VH comprises the amino acid sequence of SEQ ID NO: 50.

60. The isolated cell of any one of embodiments 57-59, wherein the VL comprises the amino acid sequence of SEQ ID NO: 51.

61. The isolated cell of any one of embodiments 57-60, wherein the ligand is nicotine or a derivative thereof.

62. The isolated cell of any one of embodiments 41-43, wherein the first monomer comprises a cannabidiol binding domain comprising an amino acid sequence selected from the group consisting of SEQ ID NO: 35, 36, 37, and 38 and the second monomer comprises a cannabidiol binding domain comprising the amino acid sequence of SEQ ID NO: 34.

63. The isolated cell of embodiment 62, wherein the ligand is a cannabidiol or a phytocannabinoid.

64. The isolated cell of any one of embodiments 41-43, wherein the first monomer comprises a cereblon domain comprising the amino acid sequence set forth in one of SEQ ID NOs: 127 and 129, and the second monomer comprises a degron comprising the amino acid sequence set forth in one of SEQ ID NOs: 131 and 133.

65. The isolated cell of embodiment 64, wherein the ligand is an IMiD.

66. The isolated cell of embodiment 65, wherein the IMiD is an FDA-approved drug.

67. The isolated cell of embodiment 65 or embodiment 66, wherein the IMiD is selected from the group consisting of: thalidomide, lenalidomide, and pomalidomide.
68. The isolated cell of any one of embodiments 1-67, wherein each monomer further comprises a linker localized between each ligand binding domain and cell death-inducing domain.

69. The isolated cell of embodiment 68, where the linker comprises an amino acid sequence selected from the group consisting of: GGGGSGGGGSGGGGSVDGF (SEQ ID NO: 101) and ASGGGGSAS (SEQ ID NO: 102).

70. An isolated cell comprising an activation-conditional control polypeptide (ACP),
wherein the ACP comprises one or more ligand binding domains and a transcription factor comprising a nucleic acid-binding domain and a transcriptional effector domain,
wherein the ACP undergoes nuclear localization upon binding of the ligand binding domain to a cognate ligand, and
wherein when localized to a cell nucleus, the ACP is capable of inducing transcriptional expression of a gene of interest operably linked to an ACP-responsive promoter.

71. An isolated cell comprising a multimeric activation-conditional control polypeptide (ACP), wherein the multimeric ACP comprises:
a) a first chimeric polypeptide, wherein the first chimeric polypeptide comprises a first ligand binding domain and a transcriptional activation domain; and
b) a second chimeric polypeptide, wherein the second chimeric polypeptide comprises a second ligand binding domain and a nucleic acid-binding domain,
wherein the first chimeric polypeptide and the second chimeric polypeptide multimerize to form the multimeric ACP via a cognate ligand that binds to each ligand binding domain, and
wherein the multimeric ACP is capable of inducing transcriptional expression of a gene of interest operably linked to an ACP-responsive promoter.

72. The isolated cell of embodiment 70 or embodiment 71, wherein each ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain.

73. The isolated cell of embodiment 72, wherein the ABI domain comprises the amino acid sequence of SEQ ID NO: 31.

74. The isolated cell of embodiment 72, wherein the PYL domain comprises the amino acid sequence of SEQ ID NO: 53.

75. The isolated cell of embodiment 72, wherein the caffeine-binding single-domain antibody comprises the amino acid sequence of SEQ ID NO: 33.

76. The isolated cell of embodiment 72, wherein the cannabidiol binding domain comprises an amino acid sequence selected from the group consisting of SEQ ID NO: 34, 35, 36, 37, and 38.

77. The isolated cell of embodiment 72, wherein the hormone-binding domain of estrogen receptor (ER) domain comprises the amino acid sequence of SEQ ID NO: 42.

78. The isolated cell of embodiment 72, wherein the heavy chain variable region (VH) of an anti-nicotine antibody comprises the amino acid sequence of SEQ ID NO: 50.

79. The isolated cell of embodiment 72, wherein the light chain variable region (VL) of an anti-nicotine antibody comprises the amino acid sequence of SEQ ID NO: 51.

80. The isolated cell of embodiment 72, wherein the progesterone receptor domain comprises the amino acid sequence of SEQ ID NO: 52.

81. The isolated cell of embodiment 72, wherein the FKBP domain comprises the amino acid sequence of SEQ ID NO: 43.

82. The isolated cell of embodiment 72, wherein the FRB domain comprises the amino acid sequence of SEQ ID NO: 44.

83. The isolated cell of embodiment 71, wherein the nucleic acid-binding domain comprises a DNA-binding zinc finger protein domain (ZF protein domain).

84. The isolated cell of embodiment 83, wherein the ZF protein domain is modular in design and is composed of zinc finger arrays (ZFA).

85. The isolated cell of any one of embodiments 70-84, wherein the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

86. The isolated cell of any one of embodiments 70 and 72-85, wherein the chimeric polypeptide further comprises a linker localized between the nucleic acid-binding domain and the transcriptional effector domain.

87. The isolated cell of embodiment 86, wherein the linker comprises one or more 2A ribosome skipping tags.

88. The isolated cell of embodiment 87, wherein each 2A ribosome skipping tag is selected from the group consisting of: P2A, T2A, E2A, and F2A.

89. The isolated cell of any one of embodiments 71-88, wherein the chimeric polypeptide comprises a first ligand binding domain operably linked to the nucleic acid-binding domain and a second ligand binding domain operably linked to the transcriptional effector domain.

90. The isolated cell of any one of embodiments 71-89, wherein each of the first and second ligand binding domains comprises a hormone-binding domain of estrogen receptor (ER) domain.

91. The isolated cell of embodiment 90, wherein the cognate ligand is tamoxifen or a metabolite thereof.

92. The isolated cell of embodiment 91, wherein the tamoxifen metabolite is selected from the group consisting of: 4-hydroxytamoxifen, N-desmethyltamoxifen, tamoxifen-N-oxide, and endoxifen.

93. The isolated cell of any one of embodiments 71-89, wherein each of the first and second ligand binding domains comprises a progesterone receptor domain.

94. The isolated cell of embodiment 93, wherein the cognate ligand is mifepristone or a derivative thereof.

95. The isolated cell of any one of embodiments 1-89, wherein when the ligand binding domain comprises an ABI domain or a PYL domain, the cognate ligand is abscisic acid.

96. The isolated cell of any one of embodiments 1-89, wherein when the ligand binding domain comprises a caffeine-binding single-domain antibody, the cognate ligand is caffeine or a derivative thereof.

97. The isolated cell of any one of embodiments 1-89, wherein when the ligand binding domain comprises a cannabidiol binding domain, the cognate ligand is a cannabidiol or a phytocannabinoid.

98. The isolated cell of embodiment 97, wherein the cannabidiol binding domain comprises a single-domain antibody or a nanobody.

99. The isolated cell of embodiment 98, wherein the cannabidiol binding domain comprises an amino acid sequence selected from the group consisting of SEQ ID NO: 34, 35, 36, 37, and 38.

100. The isolated cell of any one of embodiments 1-89, wherein when the ligand binding domain comprises a hormone-binding domain of estrogen receptor (ER) domain, the cognate ligand is tamoxifen or a metabolite thereof.

101. The isolated cell of embodiment 100, wherein the tamoxifen metabolite is selected from the group consisting of: 4-hydroxytamoxifen, N-desmethyltamoxifen, tamoxifen-N-oxide, and endoxifen.

102. The isolated cell of any one of embodiments 1-89, wherein when the ligand binding domain comprises a heavy chain variable region (VH) of an anti-nicotine antibody or a light chain variable region (VL) of an anti-nicotine antibody, the cognate ligand is nicotine or a derivative thereof.

103. The isolated cell of any one of embodiments 1-89, wherein when the ligand binding domain is a progesterone receptor domain, the cognate ligand is mifepristone or a derivative thereof.

104. The isolated cell of any one of embodiments 1-89, wherein when the ligand binding domain comprises an FKBP domain or an FRB domain, the cognate ligand is rapamycin, AP1903, AP20187, FK1012, derivatives thereof, or analogs thereof.

105. The isolated cell of any one of embodiments 70-104, wherein the nucleic acid-binding domain comprises a DNA-binding zinc finger protein domain (ZF protein domain).

106. The isolated cell of embodiment 105, wherein the ZF protein domain is modular in design and is composed of zinc finger arrays (ZFA).

107. The isolated cell of embodiment 106, wherein the ZF protein domain comprises one to ten ZFA.

108. The isolated cell of any one of embodiments 105-107, wherein the nucleic acid-binding domain binds to the ACP-responsive promoter.

109. The isolated cell of any one of embodiments 70-108, wherein the ACP-responsive promoter comprises an ACP-binding domain sequence and a promoter sequence.

110. The isolated cell of embodiment 109, wherein the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof.

111. The isolated cell of embodiment 109 or embodiment 110, wherein the ACP-responsive promoter comprises a synthetic promoter.

112. The isolated cell of any one of embodiments 105-111, wherein the ACP-responsive promoter comprises a minimal promoter.

113. The isolated cell of any one of embodiments 105-111, wherein the ACP-binding domain comprises one or more zinc finger binding sites.

114. The isolated cell of any one of embodiments 71-113, wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

115. An isolated cell comprising an activation-conditional control polypeptide (ACP),
wherein the ACP comprises a ligand binding domain and a transcriptional effector domain, and
wherein upon binding of the ligand binding domain to a cognate ligand, the ACP is capable of modulating transcriptional expression of a gene of interest operably linked to an ACP-responsive promoter.

116. The isolated cell of embodiment 115, wherein the ligand binding domain is localized 5′ of the transcriptional effector domain or 3′ of the transcriptional effector domain.

117. The isolated cell of embodiment 115 or embodiment 116, wherein the transcriptional effector domain comprises a transcriptional repressor.

118. The isolated cell of embodiment 115, wherein the transcriptional repressor comprises a transcriptional repressor domain is selected from the group consisting of: a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

119. The isolated cell of embodiment 115 or embodiment 116, wherein the transcriptional effector domain comprises a transcriptional activator.

120. The isolated cell of embodiment 119, wherein the transcriptional activator comprises a transcriptional activation domain selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

121. The isolated cell of any one of embodiments 115-120, wherein the ACP is a transcription factor.

122. The isolated cell of any one of embodiments 115-121, wherein the ACP is a zinc-finger-containing transcription factor.

123. The isolated cell of embodiment 122, wherein the zinc finger-containing transcription factor comprises a DNA-binding zinc finger protein domain (ZF protein domain) and the transcriptional repressor domain or the transcriptional activation domain.

124. The isolated cell of embodiment 123, wherein the ZF protein domain is modular in design and is composed of zinc finger arrays (ZFA).

125. The isolated cell of embodiment 124, wherein the ZF protein domain comprises one to ten ZFA.

126. The isolated cell of any one of embodiments 123-125, wherein the DNA binding zinc finger protein domain binds to the ACP-responsive promoter.

127. The isolated cell of any one of embodiments 115-126, wherein the ACP-responsive promoter comprises an ACP-binding domain and a promoter sequence.

128. The isolated cell of embodiment 127, wherein the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof.

129. The isolated cell of any one of embodiments 115-128, wherein the ACP-responsive promoter is a synthetic promoter.

130. The isolated cell of any one of embodiments 115-129 wherein the ACP-responsive promoter comprises a minimal promoter.

131. The isolated cell of any one of embodiments 127-130, wherein the ACP-binding domain comprises one or more zinc finger binding sites.

132. The isolated cell of any one of embodiments 115-131, wherein the gene of interest is an cell death-inducing polypeptide.

133. The isolated cell of embodiment 132, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and Purine nucleoside phosphorylase.

134. The isolated cell of embodiment 132, wherein the cell death-inducing polypeptide is caspase 9 or a functional truncation thereof.

135. The isolated cell of embodiment 134, wherein the cell death-inducing domain comprises the amino acid sequence of SEQ ID NO: 39.

136. The isolated cell of embodiment 132, wherein the cell death-inducing polypeptide is Diphtheria toxin fragment A (DTA).

137. The isolated cell of embodiment 136, wherein the cell death-inducing domain comprises the amino acid sequence of SEQ ID NO: 41.

138. The isolated cell of embodiment 132, wherein the cell death-inducing polypeptide is granzyme B.

139. The isolated cell of embodiment 138, wherein the cell death-inducing domain comprises the amino acid sequence of SEQ ID NO: 47.

140. The isolated cell of embodiment 132, wherein the cell death-inducing polypeptide is Bax.

141. The isolated cell of embodiment 140, wherein the cell death-inducing domain comprises the amino acid sequence of SEQ ID NO: 32.

142. An isolated cell comprising a regulatable cell survival polypeptide and an cell death-inducing polypeptide,
wherein the cell-survival polypeptide comprises a ligand binding domain,
wherein when expressed the cell survival polypeptide is capable of inhibiting the cell death-inducing polypeptide, and
wherein upon binding to a cognate ligand, the cognate ligand inhibits the pro-survival polypeptide.

143. The isolated cell of embodiment 142, wherein the cell survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein.

144. The isolated cell of embodiment 142, wherein the cell survival polypeptide is XIAP or a modified XIAP.

145. The isolated cell of any one of embodiments 142-144, wherein the ligand binding domain is localized at the N-terminal region of the pro-survival polypeptide or at the C-terminal region of the pro-survival polypeptide.

146. The isolated cell of any one of embodiments 115-145, wherein the ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER domain), heavy chain variable region (VH) of an anti-nicotine antibody, light chain variable region (VL) of an anti-nicotine antibody, a progesterone receptor domain, an FKBP domain, and an FRB domain.

147. The isolated cell of embodiment 146, wherein the ABI domain comprises the amino acid sequence of SEQ ID NO: 31.

148. The isolated cell of embodiment 146, wherein the PYL domain comprises the amino acid sequence of SEQ ID NO: 53.

149. The isolated cell of embodiment 146, wherein the caffeine-binding single-domain antibody comprises the amino acid sequence of SEQ ID NO: 33.
150. The isolated cell of embodiment 146, wherein the cannabidiol binding domain comprises an amino acid sequence selected from the group consisting of SEQ ID NO: 34, 35, 36, 37, and 38.

151. The isolated cell of embodiment 146, wherein the hormone-binding domain of estrogen receptor (ER) domain comprises the amino acid sequence of SEQ ID NO: 42.

152. The isolated cell of embodiment 146, wherein the heavy chain variable region (VH) of an anti-nicotine antibody comprises the amino acid sequence of SEQ ID NO: 50.

153. The isolated cell of embodiment 146, wherein the light chain variable region (VL) of an anti-nicotine antibody comprises the amino acid sequence of SEQ ID NO: 51.

154. The isolated cell of embodiment 146, wherein the progesterone receptor domain comprises the amino acid sequence of SEQ ID NO: 52.

155. The isolated cell of embodiment 146, wherein the FKBP domain comprises the amino acid sequence of SEQ ID NO: 43.

156. The isolated cell of embodiment 146, wherein the FRB domain comprises the amino acid sequence of SEQ ID NO: 44.

157. The isolated cell of any one of embodiments 115-156, wherein when the ligand binding domain comprises an ABI domain or a PYL domain, the cognate ligand is abscisic acid.

158. The isolated cell of any one of embodiments 115-156, wherein when the ligand binding domain comprises a caffeine-binding single-domain antibody, the cognate ligand is caffeine or a derivative thereof.

159. The isolated cell of any one of embodiments 115-156, wherein when the ligand binding domain comprises a cannabidiol binding domain, the cognate ligand is a cannabidiol or a phytocannabinoid.

160. The isolated cell of any one of embodiments 115-156, wherein when the ligand binding domain comprises a hormone-binding domain of estrogen receptor (ER) domain, the cognate ligand is tamoxifen or a metabolite thereof.

161. The isolated cell of embodiment 160, wherein the tamoxifen metabolite is selected from the group consisting of: 4-hydroxytamoxifen, N-desmethyltamoxifen, tamoxifen-N-oxide, and endoxifen.

162. The isolated cell of any one of embodiments 115-156, wherein when the ligand binding domain comprises a heavy chain variable region (VH) of an anti-nicotine antibody or a light chain variable region (VL) of an anti-nicotine antibody, the cognate ligand is nicotine or a derivative thereof.

163. The isolated cell of any one of embodiments 115-156, wherein when the ligand binding domain is a progesterone receptor domain, the cognate ligand is mifepristone or a derivative thereof.

164. The isolated cell of any one of embodiments 115-156, wherein when the ligand binding domain comprises an FKBP domain, or an FRB domain, the cognate ligand is rapamycin, AP1903, AP20187, FK1012, derivatives thereof, or analogs thereof.

165. The isolated cell of any one of embodiments 115-164, wherein the ligand binding domain comprises a degron.

166. The isolated cell of embodiment 165, wherein the degron is capable of inducing degradation of the regulatable cell survival polypeptide.

167. The isolated cell of embodiment 165 or embodiment 166, wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), Spmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

168. The isolated cell of any one of embodiments 165-167, wherein the degron comprises a cereblon (CRBN) polypeptide substrate domain capable of binding CRBN in response to an immunomodulatory drug (IMiD) thereby promoting ubiquitin pathway-mediated degradation of the regulatable polypeptide.

169. The isolated cell of embodiment 168, wherein the CRBN polypeptide substrate domain is selected from the group consisting of: IKZF1, IKZF3, Ckla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

170. The isolated cell of embodiment 168 or embodiment 169, wherein the CRBN polypeptide substrate domain is a chimeric fusion product of native CRBN polypeptide sequences.

171. The isolated cell of any one of embodiments 168-170, wherein the CRBN polypeptide substrate domain is a IKZF3/ZFP91/IKZF3 chimeric fusion product having the amino acid sequence of

(SEQ ID NO: 103)
FNVLMVHKRSHTGERPLQCEICGFTCRQKGNLLRHIKLHTGEKPFKCHL
CNYACQRRDAL.

172. The isolated cell of any one of embodiments 115-171, wherein the ligand is an IMiD.

173. The isolated cell of embodiment 172, wherein the IMiD is an FDA-approved drug.

174. The isolated cell of embodiment 172 or embodiment 173, wherein the IMiD is selected from the group consisting of: thalidomide, lenalidomide, and pomalidomide.

175. The isolated cell of any one of embodiments 142-174, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B1, and Purine nucleoside phosphorylase.

176. The isolated cell of any one of embodiments 142-174, wherein the cell death-inducing polypeptide is caspase 9 or a functional truncation thereof.

177. The isolated cell of embodiment 176, wherein the cell death-inducing domain comprises the amino acid sequence of SEQ ID NO: 39.

178. The isolated cell of any one of embodiments 142-174, wherein the cell death-inducing polypeptide is Diphtheria toxin fragment A (DTA).

179. The isolated cell of embodiment 178, wherein the cell death-inducing domain comprises the amino acid sequence of SEQ ID NO: 41.

180. The isolated cell of any one of embodiments 142-174, wherein the cell death-inducing polypeptide is Bax.

181. The isolated cell of embodiment 180, wherein the cell death-inducing domain comprises the amino acid sequence of SEQ ID NO: 32.

182. An engineered nucleic acid comprising:
an expression cassette comprising a promoter and an exogenous polynucleotide sequence encoding an inducible cell death polypeptide monomer, wherein the promoter is operably linked to the exogenous polynucleotide,
wherein the inducible cell death polypeptide monomer comprises one or more ligand binding domains and an cell death-inducing domain,
wherein each of the one or more ligand binding domains comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, an FRB domain, a cereblon domain, optionally comprising the amino acid sequence set forth in one of SEQ ID NOs: 127 and 129, a degron, optionally comprising the amino acid sequence set forth in one of SEQ ID NOs: 131 and 133,
wherein when expressed, the cell death polypeptide monomer is oligomerizable via a cognate ligand that binds to the ligand binding domain, and
wherein when the ligand oligomerizes two or more of the cell death polypeptide monomers, an cell death-inducing signal is generated in a cell.

183. The engineered nucleic acid of embodiment 182, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

184. The engineered nucleic acid of embodiment 182, wherein the cell death-inducing domain comprises caspase 9, or a functional truncation thereof.

185. The engineered nucleic acid of embodiment 184, wherein the cell death-inducing domain comprises the amino acid sequence of SEQ ID NO: 39.

186. The engineered nucleic acid of embodiment 182, wherein the cell death-inducing domain comprises Bid, or a functional truncation thereof.

187. The engineered nucleic acid of embodiment 186, wherein the cell death-inducing domain comprises the amino acid sequence of SEQ ID NO: 54.

188. The engineered nucleic acid of embodiment 182, wherein the ABI domain comprises the amino acid sequence of SEQ ID NO: 31.

189. The engineered nucleic acid of embodiment 182, wherein the PYL domain comprises the amino acid sequence of SEQ ID NO: 53.

190. The engineered nucleic acid of embodiment 182, wherein the caffeine-binding single-domain antibody comprises the amino acid sequence of SEQ ID NO: 33.

191. The engineered nucleic acid of embodiment 182, wherein the cannabidiol binding domain comprises an amino acid sequence selected from the group consisting of SEQ ID NO: 34, 35, 36, 37, and 38.

192. The engineered nucleic acid of embodiment 182, wherein the hormone-binding domain of estrogen receptor (ER) domain comprises the amino acid sequence of SEQ ID NO: 42.

193. The engineered nucleic acid of embodiment 182, wherein the heavy chain variable region (VH) of an anti-nicotine antibody comprises the amino acid sequence of SEQ ID NO: 50.

194. The engineered nucleic acid of embodiment 182, wherein the light chain variable region (VL) of an anti-nicotine antibody comprises the amino acid sequence of SEQ ID NO: 51.

195. The engineered nucleic acid of embodiment 182, wherein the progesterone receptor domain comprises the amino acid sequence of SEQ ID NO: 52.

196. The engineered nucleic acid of embodiment 182, wherein the FKBP domain comprises the amino acid sequence of SEQ ID NO: 43.

197. The engineered nucleic acid of embodiment 182, wherein the FRB domain comprises the amino acid sequence of SEQ ID NO: 44.

198. The engineered nucleic acid of any one of embodiments 182-197, wherein each monomer comprises the same ligand binding domain.

199. The engineered nucleic acid of embodiment 198, wherein the inducible cell death polypeptide comprises homooligomers.

200. The engineered nucleic acid of embodiment 199, wherein the homooligomers comprise homodimers.

201. The engineered nucleic acid of any one of embodiments 198-200, wherein each monomer comprises an FKBP domain.

202. The engineered nucleic acid of embodiment 201, wherein the ligand is FK1012, a derivative thereof, or an analog thereof.

203. The engineered nucleic acid of embodiment 201 or embodiment 202, wherein the cell death-inducing domain comprises Bid, or a functional truncation thereof.

204. The engineered nucleic acid of embodiment 203, wherein the cell death-inducing domain comprises the amino acid sequence of SEQ ID NO: 54.

205. The engineered nucleic acid of any one of embodiments 182-188, wherein each monomer comprises an ABI domain and a PYL domain.

206. The engineered nucleic acid of embodiment 205, wherein the ligand is abscisic acid.

207. The engineered nucleic acid of embodiment 205 or embodiment 206, wherein the cell death-inducing domain comprises caspase 9, or a functional truncation thereof.

208. The engineered nucleic acid of embodiment 207, wherein the cell death-inducing domain comprises the amino acid sequence of SEQ ID NO: 39.

209. The engineered nucleic acid of any one of embodiments 182-188, wherein each monomer comprises a cannabidiol binding domain comprising the amino acid sequence of SEQ ID NO: 34 and a cannabidiol binding domain comprising an amino acid sequence selected from the group consisting of SEQ ID NO: 35, 36, 37, and 38.

210. The engineered nucleic acid of any one of embodiments 182-188, wherein each monomer comprises a hormone-binding domain of estrogen receptor (ER) domain and an FKBP domain.

211. The engineered nucleic acid of any one of embodiments 182-188, wherein each monomer comprises an FRB domain and a hormone-binding domain of estrogen receptor (ER) domain.

212. The engineered nucleic acid embodiment 210 or embodiment 211, wherein the cell death-inducing domain comprises caspase 9, or a functional truncation thereof.

213. The engineered nucleic acid of embodiment 212, wherein the cell death-inducing domain comprises the amino acid sequence of SEQ ID NO: 39.

214. The engineered nucleic acid of any one of embodiments 210-215, wherein the ligand is rapamycin, a derivative thereof, or an analog thereof.

215. The engineered nucleic acid of any one of embodiments 210-214, wherein the ligand is tamoxifen or a metabolite thereof.

216. The engineered nucleic acid of embodiment 215, wherein the tamoxifen metabolite is selected from the group consisting of: 4-hydroxytamoxifen, N-desmethyltamoxifen, tamoxifen-N-oxide, and endoxifen.

217. The engineered nucleic acid of any one of embodiments 182-188, wherein each monomer comprises two caffeine-binding single-domain antibodies.

218. The engineered nucleic acid of embodiment 217, wherein each caffeine-binding single-domain antibody comprises the amino acid sequence of SEQ ID NO: 33.

219. The engineered nucleic acid of embodiment 217 or embodiment 218, wherein the ligand is caffeine or a derivative thereof.

220. The engineered nucleic acid of any one of embodiments 182-219, wherein each monomer comprises a progesterone receptor domain comprising the amino acid sequence of SEQ ID NO: 52.

221. The engineered nucleic of embodiment 220, wherein the ligand is mifepristone or a derivative thereof

222. The engineered nucleic acid of any one of embodiments 182-188, wherein a first monomer comprises a first ligand binding domain and a second monomer comprises a second ligand binding domain.

223. The engineered nucleic acid of embodiment 222, wherein the inducible cell death polypeptide comprises heterooligomers.

224. The engineered nucleic acid of embodiment 223, wherein the heterooligomers comprise heterodimers.

225. The engineered nucleic acid of any one of embodiments 222-224, wherein the first monomer comprises an FKBP domain and the second monomer comprises an FRB domain.

226. The engineered nucleic acid of embodiment 225, wherein the cell death-inducing domain comprises Bid, or a functional truncation thereof.

227. The engineered nucleic acid of embodiment 226, wherein the cell death-inducing domain comprises the amino acid sequence of SEQ ID NO: 54.

228. The engineered nucleic acid of any one of embodiments 222-224, wherein the first monomer comprises a hormone-binding domain of estrogen receptor (ER) domain and the second monomer comprises an FKBP domain.

229. The engineered nucleic acid of any one of embodiments 222-224, wherein the first monomer comprises an FRB domain and the second monomer comprises a hormone-binding domain of estrogen receptor (ER) domain.

230. The engineered nucleic acid of any one of embodiments 222-224, wherein the first monomer comprises a hormone-binding domain of estrogen receptor (ER) domain and an FKBP domain, and the second monomer comprises an FRB domain and the second monomer comprises a hormone-binding domain of estrogen receptor (ER) domain.

231. The engineered nucleic acid of any one of embodiments 228-230, wherein the cell death-inducing domain comprises caspase 9, or a functional truncation thereof.

232. The engineered nucleic acid of embodiment 231, wherein the cell death-inducing domain comprises the amino acid sequence of SEQ ID NO: 39.

233. The engineered nucleic acid of any one of embodiments 225-232, wherein the ligand is rapamycin, a derivative thereof, or an analog thereof.

234. The engineered nucleic acid of any one of embodiments 228-232, wherein the ligand is tamoxifen or a metabolite thereof.

235. The engineered nucleic acid of embodiment 234, wherein the tamoxifen metabolite is selected from the group consisting of: 4-hydroxytamoxifen, N-desmethyltamoxifen, tamoxifen-N-oxide, and endoxifen.

236. The engineered nucleic acid of any one of embodiments 222-224, wherein the first monomer comprises an ABI domain and the second monomer comprises a PYL domain.

237. The engineered nucleic acid of embodiment 236, wherein the ligand is abscisic acid.

238. The engineered nucleic acid of any one of embodiments 222-224, wherein the first monomer comprises a heavy chain variable region (VH) of an anti-nicotine antibody and the second monomer comprises a light chain variable region (VL) of an anti-nicotine antibody.

239. The engineered nucleic acid of embodiment 238, wherein the anti-nicotine antibody is a Nic 12 antibody.

240. The engineered nucleic acid of embodiment 238 or embodiment 239, wherein the VH comprises the amino acid sequence of SEQ ID NO: 50.

241. The engineered nucleic acid of any one of embodiments 238-240, wherein the VL comprises the amino acid sequence of SEQ ID NO: 51.

242. The engineered nucleic acid of any one of embodiments 238-241, wherein the ligand is nicotine or a derivative thereof.

243. The engineered nucleic acid of any one of embodiments 222-224, wherein the first monomer comprises a cannabidiol binding domain comprising an amino acid sequence selected from the group consisting of SEQ ID NO: 35, 36, 37, and 38 and the second monomer comprises a cannabidiol binding domain comprising the amino acid sequence of SEQ ID NO: 34.

244. The engineered nucleic acid of embodiment 243, wherein the ligand is a cannabidiol or a phytocannabinoid.

245. The engineered nucleic acid of any one of embodiments 222-224, wherein the first monomer comprises a cereblon domain comprising the amino acid sequence set forth in one of SEQ ID NOs: 127 and 129, and the second monomer comprises a degron comprising the amino acid sequence set forth in one of SEQ ID NOs: 131 and 133.

246. The engineered nucleic acid of embodiment 245, wherein the ligand is an IMiD.

247. The engineered nucleic acid of embodiment 246, wherein the IMiD is an FDA-approved drug.

248. The engineered nucleic acid of embodiment 245 or embodiment 246, wherein the IMiD is selected from the group consisting of: thalidomide, lenalidomide, and pomalidomide.

249. The engineered nucleic acid of any one of embodiments 176-248, wherein each monomer further comprises a linker localized between each ligand binding domain and cell death-inducing domain.

250. The engineered nucleic acid of embodiment 249, where the linker comprises an amino acid sequence selected from the group consisting of: GGGGSGGGGSGGGGSVDGF (SEQ ID NO: 104) and ASGGGGSAS (SEQ ID NO: 105).

251. An engineered nucleic acid comprising:
an expression cassette comprising a promoter and an exogenous polynucleotide sequence encoding an activation-conditional control polypeptide (ACP), wherein the promoter is operably linked to the exogenous polynucleotide,
wherein the ACP comprises one or more ligand binding domains and a transcription factor comprising a nucleic acid-binding domain and a transcriptional effector domain,
wherein when expressed, the ACP undergoes nuclear localization upon binding of the ligand binding domain to a cognate ligand, and
wherein when localized to a cell nucleus, the ACP is capable of inducing transcriptional expression of a gene of interest operably linked to an ACP-responsive promoter.

252. An engineered nucleic acid comprising:
an expression cassette comprising a promoter and an exogenous polynucleotide sequence having the formula:

C₁-L-C₂
wherein
C₁ comprises a polynucleotide sequence encoding a first chimeric polypeptide comprising a first ligand binding domain and a transcriptional activation domain,
L comprises a linker polynucleotide sequence,
C₂ comprises a polynucleotide sequence encoding a second chimeric polypeptide comprising a second ligand binding domain and a nucleic acid-binding domain;
wherein the promoter is operably linked to the exogenous polynucleotide;
wherein when expressed, the first chimeric polypeptide and the second chimeric polypeptide multimerize to form an activation-conditional control polypeptide (ACP) via a cognate ligand that binds to each ligand binding domain; and
wherein the multimeric ACP is capable of inducing transcriptional expression of a gene of interest operably linked to an ACP-responsive promoter. The engineered nucleic acid of embodiment 251 or embodiment 252, wherein each ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER) domain, heavy chain variable region (VH) of an anti-nicotine antibody, light chain variable region (VL) of an anti-nicotine antibody, a progesterone receptor domain, an FKBP domain, and an FRB domain.

253. The engineered nucleic acid of embodiment 252, wherein the ABI domain comprises the amino acid sequence of SEQ ID NO: 31.

254. The engineered nucleic acid of embodiment 252, wherein the PYL domain comprises the amino acid sequence of SEQ ID NO: 53.

255. The engineered nucleic acid of embodiment 252, wherein the caffeine-binding single-domain antibody comprises the amino acid sequence of SEQ ID NO: 33.

256. The engineered nucleic acid of embodiment 252, wherein the cannabidiol binding domain comprises an amino acid sequence selected from the group consisting of SEQ ID NO: 35, 36, 37, and 38.

257. The engineered nucleic acid of embodiment 252, wherein the hormone-binding domain of estrogen receptor (ER) domain comprises the amino acid sequence of SEQ ID NO: 42.

258. The engineered nucleic acid of embodiment 252, wherein the heavy chain variable region (VH) of an anti-nicotine antibody comprises the amino acid sequence of SEQ ID NO: 50.

259. The engineered nucleic acid of embodiment 252, wherein the light chain variable region (VL) of an anti-nicotine antibody comprises the amino acid sequence of SEQ ID NO: 51.

260. The engineered nucleic acid of embodiment 252, wherein the progesterone receptor domain comprises the amino acid sequence of SEQ ID NO: 52.

261. The engineered nucleic acid of embodiment 252, wherein the FKBP domain comprises the amino acid sequence of SEQ ID NO: 43.

262. The engineered nucleic acid of embodiment 252, wherein the FRB domain comprises the amino acid sequence of SEQ ID NO: 44.

263. The engineered nucleic acid of embodiment 250, wherein the nucleic acid-binding domain comprises a DNA-binding zinc finger protein domain (ZF protein domain).

264. The engineered nucleic acid of embodiment 263, wherein the ZF protein domain is modular in design and is composed of zinc finger arrays (ZFA).

265. The engineered nucleic acid of embodiment 263 or embodiment 264, wherein the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

266. The engineered nucleic acid of any one of embodiments 250 and 263-265, wherein the chimeric polypeptide further comprises a linker localized between the nucleic acid-binding domain and the transcriptional effector domain.

267. The engineered nucleic acid of embodiment 266, wherein the linker comprises one or more 2A ribosome skipping tags.

268. The engineered nucleic acid of embodiment 267, wherein each 2A ribosome skipping tag is selected from the group consisting of: P2A, T2A, E2A, and F2A.

269. The engineered nucleic acid of any one of embodiments 250 and 263-268, wherein the chimeric polypeptide comprises a first ligand binding domain operably linked to the nucleic acid-binding domain and a second ligand binding domain operably linked to the transcriptional effector domain.

270. The engineered nucleic acid of any one of embodiments 259 and 263-269, wherein each of the first and second ligand binding domains comprises a hormone-binding domain of estrogen receptor (ER) domain.

271. The engineered nucleic acid of embodiment 270, wherein the cognate ligand is tamoxifen or a metabolite thereof.

272. The engineered nucleic acid of embodiment 271, wherein the tamoxifen metabolite is selected from the group consisting of: 4-hydroxytamoxifen, N-desmethyltamoxifen, tamoxifen-N-oxide, and endoxifen.

273. The engineered nucleic acid of any one of embodiments 250-269, wherein each of the first and second ligand binding domains comprises a progesterone receptor domain.

274. The engineered nucleic acid of embodiment 273, wherein the cognate ligand is mifepristone or a derivative thereof.

275. The engineered nucleic acid of any one of embodiments 182-249 and 251-269, wherein when the ligand binding domain comprises an ABI domain or a PYL domain, the cognate ligand is abscisic acid.

276. The engineered nucleic acid of any one of embodiments 182-249 and 251-269, wherein when the ligand binding domain comprises a caffeine-binding single-domain antibody, the cognate ligand is caffeine or a derivative thereof.

277. The engineered nucleic acid of any one of embodiments 182-249 and 251-269, wherein when the ligand binding domain comprises a cannabidiol binding domain, the cognate ligand is a cannabidiol or a phytocannabinoid.

278. The engineered nucleic acid of embodiment 277, wherein the cannabidiol binding domain comprises a single-domain antibody or a nanobody.

279. The engineered nucleic acid of embodiment 278, wherein the cannabidiol binding domain comprises an amino acid sequence selected from the group consisting of SEQ ID NO: 34, 35, 36, 37, and 38.

280. The engineered nucleic acid of any one of embodiments 182-249 and 251-269, wherein when the ligand binding domain comprises a hormone-binding domain of estrogen receptor (ER) domain, the cognate ligand is tamoxifen or a metabolite thereof.

281. The engineered nucleic acid of embodiment 280, wherein the tamoxifen metabolite is selected from the group consisting of: 4-hydroxytamoxifen, N-desmethyltamoxifen, tamoxifen-N-oxide, and endoxifen.

282. The engineered nucleic acid of any one of embodiments 182-249 and 251-269, wherein when the ligand binding domain comprises a heavy chain variable region (VH) of an anti-nicotine antibody or a light chain variable region (VL) of an anti-nicotine antibody, the cognate ligand is nicotine or a derivative thereof.

283. The engineered nucleic acid of any one of embodiments 182-249 and 251-269, wherein when the ligand binding domain is a progesterone receptor domain, the cognate ligand is mifepristone or a derivative thereof.

284. The engineered nucleic acid of any one of embodiments 182-249 and 251-269, wherein when the ligand binding domain comprises an FKBP domain or an FRB domain, the cognate ligand is rapamycin, AP1903, AP20187, FK1012, derivatives thereof, or analogs thereof.

285. The engineered nucleic acid of any one of embodiments 250-284, wherein the nucleic acid-binding domain comprises a DNA-binding zinc finger protein domain (ZF protein domain).

286. The engineered nucleic acid of embodiment 285, wherein the ZF protein domain is modular in design and is composed of zinc finger arrays (ZFA).

287. The engineered nucleic acid of embodiment 286, wherein the ZF protein domain comprises one to ten ZF motifs.

288. The engineered nucleic acid of any one of embodiments 285-287, wherein the nucleic acid-binding domain binds to the ACP-responsive promoter.

289. The engineered nucleic acid of any one of embodiments 250-288, wherein the ACP-responsive promoter comprises an ACP-binding domain sequence and a promoter sequence.

290. The engineered nucleic acid of embodiment 289, wherein the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof.

291. The engineered nucleic acid of embodiment 289 or embodiment 290, wherein the ACP-responsive promoter comprises a synthetic promoter.

292. The engineered nucleic acid of any one of embodiments 285-291, wherein the ACP-responsive promoter comprises a minimal promoter.

293. The engineered nucleic acid of any one of embodiments 285-292, wherein the ACP-binding domain comprises one or more zinc finger binding sites.

294. The engineered nucleic acid of any one of embodiments 250-293, wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

295. The engineered nucleic acid of embodiment 252, wherein the linker polynucleotide sequence is operably associated with the translation of each chimeric polypeptide as a separate polypeptide.

296. The engineered nucleic acid of embodiment 252 or embodiment 295, wherein the linker polynucleotide sequence encodes a 2A ribosome skipping tag.

297. The engineered nucleic acid of embodiment 296, wherein the 2A ribosome skipping tag is selected from the group consisting of: P2A, T2A, E2A, and F2A.

298. The engineered nucleic acid of embodiment 252 or embodiment 295, wherein the linker polynucleotide sequence encodes an Internal Ribosome Entry Site (IRES).

299. The engineered nucleic acid of any one of embodiments 252-298, wherein the linker polynucleotide sequence encodes a cleavable polypeptide.

300. The engineered nucleic acid of embodiment 299, wherein the cleavable polypeptide comprises a furin polypeptide sequence.

301. An engineered nucleic acid comprising:
an expression cassette comprising a promoter and an exogenous polynucleotide sequence encoding an activation-conditional control polypeptide (ACP) comprising a ligand binding domain and a transcriptional effector domain,
wherein the promoter is operably linked to the exogenous polynucleotide, and
wherein when expressed and upon binding of the ligand binding domain to a cognate ligand, the ACP is capable of modulating transcriptional expression of a gene of interest operably linked to an ACP-responsive promoter.

302. The engineered nucleic acid of embodiment 301, wherein the ligand binding domain is localized 5′ of the transcriptional effector domainor 3′ of the transcriptional effector domain.

303. The engineered nucleic acid of embodiment 301 or embodiment 302, wherein the transcriptional effector domain comprises a transcriptional repressor.

304. The engineered nucleic acid of embodiment 303, wherein the transcriptional repressor comprises a transcriptional repressor domain is selected from the group consisting of: a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

305. The engineered nucleic acid of embodiment 301 or embodiment 302, wherein the transcriptional effector domain comprises a transcriptional activator.

306. The engineered nucleic acid of embodiment 305, wherein the transcriptional activator comprises a transcriptional activation domain selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

307. The engineered nucleic acid of any one of embodiments 301-306, wherein the ACP is a transcription factor.

308. The engineered nucleic acid of any one of embodiments 301-307, wherein the ACP is a zinc-finger-containing transcription factor.

309. The engineered nucleic acid of embodiment 308, wherein the zinc finger-containing transcription factor comprises a DNA-binding zinc finger protein domain (ZF protein domain) and the transcriptional repressor domain or the transcriptional activation domain.

310. The engineered nucleic acid of embodiment 309, wherein the ZF protein domain is modular in design and is composed of zinc finger arrays (ZFA).

311. The engineered nucleic acid of embodiment 210, wherein the ZF protein domain comprises one to ten ZFA.

312. The engineered nucleic acid of any one of embodiments 309-311, wherein the DNA binding zinc finger protein domain binds to the ACP-responsive promoter.

313. The engineered nucleic acid of any one of embodiments 301-312, wherein the ACP-responsive promoter comprises an ACP-binding domain and a promoter sequence.

314. The engineered nucleic acid of embodiment 313, wherein the promoter sequence is derived from a promoter selected from the group consisting of minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTATA, minTK, inducer molecule-responsive promoters, and tandem repeats thereof.

315. The engineered nucleic acid of any one of embodiments 301-314, wherein the ACP-responsive promoter is a synthetic promoter.

316. The engineered nucleic acid of any one of embodiments 301-315 wherein the ACP-responsive promoter comprises a minimal promoter.

317. The engineered nucleic acid of any one of embodiments 313-316, wherein the ACP-binding domain comprises one or more zinc finger binding sites.

318. The engineered nucleic acid of any one of embodiments 301-317, wherein the gene of interest is an cell death-inducing polypeptide.

319. The engineered nucleic acid of embodiment 318, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

320. The engineered nucleic acid of embodiment 318, wherein the cell death-inducing polypeptide is caspase 9 or a functional truncation thereof.

321. The engineered nucleic acid of embodiment 320, wherein the cell death-inducing domain comprises the amino acid sequence of SEQ ID NO: 39.

322. The engineered nucleic acid of embodiment 318, wherein the cell death-inducing polypeptide is Diphtheria toxin fragment A (DTA).

323. The engineered nucleic acid of embodiment 322, wherein the cell death-inducing domain comprises the amino acid sequence of SEQ ID NO: 41.

324. The engineered nucleic acid of embodiment 318, wherein the cell death-inducing polypeptide is granzyme B.

325. The engineered nucleic acid of embodiment 324, wherein the cell death-inducing domain comprises the amino acid sequence of SEQ ID NO: 47.

326. The engineered nucleic acid of embodiment 318, wherein the cell death-inducing polypeptide is Bax.

327. The engineered nucleic acid of embodiment 326, wherein the cell death-inducing domain comprises the amino acid sequence of SEQ ID NO: 32.

328. An engineered nucleic acid comprising:
an expression cassette comprising a promoter and an exogenous polynucleotide sequence encoding a regulatable cell survival polypeptide comprising a ligand binding domain, wherein the promoter is operably linked to the exogenous polynucleotide,
wherein when expressed, the cell survival polypeptide is capable of inhibiting an cell death-inducing polypeptide, and
wherein upon binding to a cognate ligand, the cognate ligand inhibits the pro-survival polypeptide.

329. The engineered nucleic acid of embodiment 328, wherein the cell survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein.

330. The engineered nucleic acid of embodiment 328, wherein the cell survival polypeptide is XIAP or a modified XIAP.

331. The engineered nucleic acid of any one of embodiments 328-330, wherein the ligand binding domain is localized at the N-terminal region of the pro-survival polypeptide or at the C-terminal region of the pro-survival polypeptide.

332. The engineered nucleic acid of any one of embodiments 301-331, wherein the ligand binding domain comprises a domain, or functional fragment thereof, selected from the group consisting of: an ABI domain, a PYL domain, a caffeine-binding single-domain antibody, a cannabidiol binding domain, a hormone-binding domain of estrogen receptor (ER domain), heavy chain variable region (VH) of an anti-nicotine antibody, light chain variable region (VL) of an anti-nicotine antibody, a progesterone receptor domain, an FKBP domain, and an FRB domain.

333. The engineered nucleic acid of embodiment 332, wherein the ABI domain comprises the amino acid sequence of SEQ ID NO: 31.

334. The engineered nucleic acid of embodiment 332, wherein the PYL domain comprises the amino acid sequence of SEQ ID NO: 53.

335. The engineered nucleic acid of embodiment 332, wherein the caffeine-binding single-domain antibody comprises the amino acid sequence of SEQ ID NO: 33.

336. The engineered nucleic acid of embodiment 332, wherein the cannabidiol binding domain comprises an amino acid sequence selected from the group consisting of SEQ ID NO: 34, 35, 36, 37, and 38.

337. The engineered nucleic acid of embodiment 332, wherein the hormone-binding domain of estrogen receptor (ER) domain comprises the amino acid sequence of SEQ ID NO: 42.

338. The engineered nucleic acid of embodiment 332, wherein the heavy chain variable region (VH) of an anti-nicotine antibody comprises the amino acid sequence of SEQ ID NO: 50.

339. The engineered nucleic acid of embodiment 332, wherein the light chain variable region (VL) of an anti-nicotine antibody comprises the amino acid sequence of SEQ ID NO: 51.

340. The engineered nucleic acid of embodiment 332, wherein the progesterone receptor domain comprises the amino acid sequence of SEQ ID NO: 52.

341. The engineered nucleic acid of embodiment 332, wherein the FKBP domain comprises the amino acid sequence of SEQ ID NO: 43.

342. The engineered nucleic acid of embodiment 332, wherein the FRB domain comprises the amino acid sequence of SEQ ID NO: 44.

343. The engineered nucleic acid of any one of embodiments 301-342, wherein when the ligand binding domain comprises an ABI domain or a PYL domain, the cognate ligand is abscisic acid.

344. The engineered nucleic acid of any one of embodiments 301-342, wherein when the ligand binding domain comprises a caffeine-binding single-domain antibody, the cognate ligand is caffeine or a derivative thereof.

345. The engineered nucleic acid of any one of embodiments 301-342, wherein when the ligand binding domain comprises a cannabidiol binding domain, the cognate ligand is a cannabidiol or a phytocannabinoid.

346. The engineered nucleic acid of any one of embodiments 301-342, wherein when the ligand binding domain comprises a hormone-binding domain of estrogen receptor (ER) domain, the cognate ligand is tamoxifen or a metabolite thereof.

347. The engineered nucleic acid of embodiment 346, wherein the tamoxifen metabolite is selected from the group consisting of: 4-hydroxytamoxifen, N-desmethyltamoxifen, tamoxifen-N-oxide, and endoxifen.

348. The engineered nucleic acid of any one of embodiments 301-342, wherein when the ligand binding domain comprises a heavy chain variable region (VH) of an anti-nicotine antibody or a light chain variable region (VL) of an anti-nicotine antibody, the cognate ligand is nicotine or a derivative thereof.

349. The engineered nucleic acid of any one of embodiments 301-342, wherein when the ligand binding domain is a progesterone receptor domain, the cognate ligand is mifepristone or a derivative thereof.

350. The engineered nucleic acid of any one of embodiments 301-342, wherein when the ligand binding domain comprises an FKBP domain, or an FRB domain, the cognate ligand is rapamycin, AP1903, AP20187, FK1012, derivatives thereof, or analogs thereof.

351. The engineered nucleic acid of any one of embodiments 301-342, wherein the ligand binding domain comprises a degron.

352. The engineered nucleic acid of embodiment 351, wherein the degron is capable of inducing degradation of the regulatable cell survival polypeptide.

353. The engineered nucleic acid of embodiment 351 or embodiment 352, wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), Spmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.

354. The engineered nucleic acid of any one of embodiments 351-353, wherein the degron comprises a cereblon (CRBN) polypeptide substrate domain capable of binding CRBN in response to an immunomodulatory drug (IMiD) thereby promoting ubiquitin pathway-mediated degradation of the regulatable polypeptide.

355. The engineered nucleic acid of embodiment 354, wherein the CRBN polypeptide substrate domain is selected from the group consisting of: IKZF1, IKZF3, Ckla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

356. The engineered nucleic acid of embodiment 354 or embodiment 355, wherein the CRBN polypeptide substrate domain is a chimeric fusion product of native CRBN polypeptide sequences.

357. The engineered nucleic acid of any one of embodiments 354-356, wherein the CRBN polypeptide substrate domain is a IKZF3/ZFP91/IKZF3 chimeric fusion product having the amino acid sequence of

(SEQ ID NO: 106)
FNVLMVHKRSHTGERPLQCEICGFTCRQKGNLLRHIKLHTGEKPFKCHL
CNYACQRRDAL.

358. The engineered nucleic acid of any one of embodiments 301-357, wherein the ligand is an IMiD.

359. The engineered nucleic acid of embodiment 358, wherein the IMiD is an FDA-approved drug.

360. The engineered nucleic acid of embodiment 357 or embodiment 358, wherein the IMiD is selected from the group consisting of: thalidomide, lenalidomide, and pomalidomide.

361. The engineered nucleic acid of any one of embodiments 328-360, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B1, and Purine nucleoside phosphorylase.

362. The engineered nucleic acid of any one of embodiments 328-360, wherein the cell death-inducing polypeptide is caspase 9 or a functional truncation thereof.

363. The engineered nucleic acid of embodiment 362, wherein the cell death-inducing domain comprises the amino acid sequence of SEQ ID NO: 39.

364. The engineered nucleic acid of any one of embodiments 328-360, wherein the cell death-inducing polypeptide is Diphtheria toxin fragment A (DTA).

365. The engineered nucleic acid of embodiment 364, wherein the cell death-inducing domain comprises the amino acid sequence of SEQ ID NO: 41.

366. The engineered nucleic acid of any one of embodiments 328-360, wherein the cell death-inducing polypeptide is Bax.

367. The engineered nucleic acid of embodiment 366, wherein the cell death-inducing domain comprises the amino acid sequence of SEQ ID NO: 32.

368. The engineered nucleic acid of embodiment 182-367, wherein the promoter comprises a constitutive promoter, an inducible promoter, or a synthetic promoter.

369. The engineered nucleic acid of embodiment 368, wherein the constitutive promoter is selected from the group consisting of: CAG, HLP, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

370. The engineered nucleic acid of embodiment 369, wherein the inducible promoter is selected from the group consisting of: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof. An engineered nucleic acid comprising:
a) a first expression cassette comprising a first promoter and a first exogenous polynucleotide sequence encoding a first chimeric polypeptide, wherein the first chimeric polypeptide comprises a first ligand binding domain and a transcriptional activation domain, wherein the first promoter is operably linked to the first exogenous polynucleotide; and
b) a second expression cassette comprising a second promoter and a second exogenous polynucleotide sequence encoding a second chimeric polypeptide, wherein the second chimeric polypetide comprises a second ligand binding domain and a nucleic acid-binding domain, wherein the second promoter is operably linked to the second exogenous polynucleotide,
wherein when expressed, the first chimeric polypeptide and the second chimeric polypeptide multimerize to form an activation-conditional control polypeptide (ACP) via a cognate ligand that binds to each ligand binding domain, and
wherein the multimeric ACP is capable of inducing transcriptional expression of a gene of interest operably linked to an ACP-responsive promoter.

371. The engineered nucleic acid of embodiment 370, wherein the first promoter, the second promoter, or both the first promoter and the second promoter comprise(s) a constitutive promoter, an inducible promoter, or a synthetic promoter.

372. The engineered nucleic acid of embodiment 371, wherein the constitutive promoter is selected from the group consisting of: CAG, HLP, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb.

373. The engineered nucleic acid of embodiment 372, wherein the inducible promoter is selected from the group consisting of: minP, NFκB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule-responsive promoters, and tandem repeats thereof.

374. An inducible cell death polypeptide comprising two or more monomers,
wherein each monomer comprises one or more ligand binding domains and a cell death-inducing domain,
wherein each of the one or more ligand binding domains comprises a domain, or functional fragment thereof, selected from the group consisting of:
an ABI domain, optionally comprising the amino acid sequence of SEQ ID NO: 31; or
a PYL domain, optionally comprising the amino acid sequence of SEQ ID NO: 53; or
a caffeine-binding single-domain antibody, optionally comprising the amino acid sequence of SEQ ID NO: 33; or
a cannabidiol binding domain, optionally comprising an amino acid sequence selected from the group consisting of SEQ ID NO: 34, 35, 36, 37, and 38; or
a hormone-binding domain of estrogen receptor (ER) domain, optionally comprising the amino acid sequence of SEQ ID NO: 42; or
a heavy chain variable region (VH) of an anti-nicotine antibody, optionally comprising the amino acid sequence of SEQ ID NO: 50, and/or the light chain variable region (VL) of an anti-nicotine antibody, optionally comprising the amino acid sequence of SEQ ID NO: 51; or
a progesterone receptor domain, optionally comprising the amino acid sequence of SEQ ID NO: 52; or
a FKBP domain, optionally comprising the amino acid sequence of SEQ ID NO: 43,
a FRB domain, optionally comprising the amino acid sequence of SEQ ID NO: 44,
wherein each monomer is oligomerizable via a cognate ligand that binds to the ligand binding domain,
a cereblon domain, optionally comprising the amino acid sequence set forth in one of SEQ ID NOs: 127 and 129, and
a degron, optionally comprising the amino acid sequence set forth in one of SEQ ID NOs: 131 and 133 and
wherein when the ligand oligomerizes each monomer, a cell death-inducing signal is generated in the cell.

375. The inducible cell death polypeptide of embodiment 374, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

376. The inducible cell death polypeptide of embodiment 374, wherein the cell death-inducing domain comprises:
caspase 9, or a functional truncation thereof, optionally wherein the cell death-inducing domain comprises the amino acid sequence of SEQ ID NO:39; or
Bid, or a functional truncation thereof, optionally, wherein the cell death-inducing domain comprises the amino acid sequence of SEQ ID NO: 54.

377. The inducible cell death polypeptide of any one of embodiment 3743 or embodiment 375, wherein each monomer comprises the same ligand binding domain, optionally wherein each monomer comprises:
an FKBP domain, optionally wherein the ligand is FK1012, a derivative thereof, or an analog thereof; or
an ABI domain and a PYL domain, optionally wherein the ligand is abscisic acid; or
a first cannabidiol binding domain optionally comprising the amino acid sequence of SEQ ID NO: 34, and a second cannabidiol binding domain comprising an amino acid sequence selected from the group consisting of SEQ ID NO: 35, 36, 37, and 38, optionally wherein the ligand is a phytocannabinoid, optionally the phytocannabinoid is cannabidiol; or
a hormone-binding domain of estrogen receptor (ER) domain and an FKBP domain, optionally wherein the ligand is rapamycin or a derivative thereof or an analog thereof and/or tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen; or
two caffeine-binding single-domain antibodies, optionally wherein each caffeine-binding single-domain antibody comprises the amino acid sequence of SEQ ID NO: 33, optionally wherein the ligand is caffeine or a derivative thereof; and
optionally wherein the inducible cell death polypeptide comprises homooligomers, optionally wherein the homooligomers comprise heterodimers.

378. The inducible cell death polypeptide of any one of embodiments 374-377, wherein a first monomer comprises a first ligand binding domain and a second monomer comprises a second ligand binding domain, optionally wherein:
the first monomer comprises an FKBP domain and the second monomer comprises an FRB domain, optionally wherein the ligand is rapamycin or a derivative thereof; or
the first monomer comprises a hormone-binding domain of estrogen receptor (ER) domain and the second monomer comprises an FKBP domain, optionally wherein the ligand is rapamycin or a derivative thereof and/or tamoxifen or a metabolite thereof; or
the first monomer comprises an FRB domain and the second monomer comprises a hormone-binding domain of estrogen receptor (ER) domain, optionally wherein the ligand is rapamycin or a derivative thereof and/or tamoxifen or a metabolite thereof; or
wherein the first monomer comprises a hormone-binding domain of estrogen receptor (ER) domain and an FKBP domain, and the second monomer comprises an FRB domain and a hormone-binding domain of estrogen receptor (ER) domain, optionally wherein the ligand is rapamycin or a derivative thereof and/or tamoxifen or a metabolite thereof; or
the first monomer comprises an ABI domain and the second monomer comprises a PYL domain, optionally wherein the ligand comprises abscisic acid; or
the first monomer comprises a heavy chain variable region (VH) of an anti-nicotine antibody and the second monomer comprises a light chain variable region (VL) of an anti-nicotine antibody, optionally wherein the anti-nicotine antibody is a Nic12 antibody, optionally wherein the VH comprises the amino acid sequence of SEQ ID NO: 50, and optionally wherein the VL comprises the amino acid sequence of SEQ ID NO: 51, and optionally wherein the ligand is nicotine or a derivative thereof;
the first monomer comprises a cannabidiol binding domain comprising an amino acid sequence selected from the group consisting of SEQ ID NO: 35, 36, 37, and 38 and the second monomer comprises a cannabidiol binding domain comprising the amino acid sequence of SEQ ID NO: 34. Optionally wherein the ligand is a phytocannabinoid, optionally wherein the phytocannabinoid is cannabidiol;
the first monomer comprises a cereblon domain comprising the amino acid sequence set forth in one of SEQ ID NOs: 127 and 129, and the second monomer comprises a degron comprising the
amino acid sequence set forth in one of SEQ ID NOs: 131 and 133, optionally wherein the ligand is an IMiD, optionally wherein the IMiD is an FDA-approved drug, optionally wherein the IMiD is selected from the group consisting of: thalidomide, lenalidomide, and pomalidomide,
optionally wherein the inducible cell death polypeptide comprises heterooligomers, optionally wherein the heterooligomers comprise heterodimers; and optionally wherein the tamoxifen metabolite is selected from the group consisting of: 4-hydroxytamoxifen, N-desmethyltamoxifen, tamoxifen-N-oxide, and endoxifen.

379. The inducible cell death polypeptide of any one of embodiments 374-378, wherein each monomer further comprises a linker localized between each ligand binding domain and cell death-inducing domain, optionally wherein the linker comprises an amino acid sequence selected from the group consisting of: GGGGSGGGGSGGGGSVDGF (SEQ ID NO: 101) and ASGGGGSAS (SEQ ID NO: 102).

380. An inducible cell death polypeptide comprising an activation-conditional control polypeptide (ACP),
wherein the ACP comprises a ligand binding domain and a transcriptional effector domain, and
wherein upon binding of the ligand binding domain to a cognate ligand, the ACP is capable of modulating transcriptional expression of a gene of interest operably linked to an ACP-responsive promoter.

381. An activation-conditional control polypeptide (ACP), comprising:
one or more ligand binding domains and a transcription factor comprising a nucleic acid-binding domain and a transcriptional effector domain,
wherein the ACP undergoes nuclear localization upon binding of the ligand binding domain to a cognate ligand, and
wherein when localized to a cell nucleus, the ACP is capable of inducing transcriptional expression of a gene of interest operably linked to an ACP-responsive promoter,
optionally wherein the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain, and
optionally wherein the ligand binding domain comprises:
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen; or
a progesterone receptor domain optionally comprising the amino acid sequence of SEQ ID NO: 52, and optionally wherein the cognate ligand is mifepristone or a derivative thereof.

382. An activation-conditional control polypeptide (ACP) comprising:
a) a first chimeric polypeptide, wherein the first chimeric polypeptide comprises a first ligand binding domain and a transcriptional activation domain; and
b) a second chimeric polypeptide, wherein the second chimeric polypeptide comprises a second ligand binding domain and a nucleic acid-binding domain,
wherein the first chimeric polypeptide and the second chimeric polypeptide multimerize to form the multimeric ACP via a cognate ligand that binds to each ligand binding domain, and
wherein the multimeric ACP is capable of inducing transcriptional expression of a gene of interest operably linked to an ACP-responsive promoter, and
optionally wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

383. The ACP of embodiment 380 or embodiment 382, wherein each ligand binding domain comprises a domain, or functional fragment thereof, selected from the group consisting of:
an ABI domain, optionally comprising the amino acid sequence of SEQ ID NO: 31, and optionally wherein the cognate ligand is abscisic acid;
a PYL domain, optionally comprising the amino acid sequence of SEQ ID NO: 53, and optionally wherein the cognate ligand is abscisic acid;
a caffeine-binding single-domain antibody optionally comprising the amino acid sequence of SEQ ID NO: 33, and optionally wherein the cognate ligand is caffeine or a derivative thereof;
a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol;
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen;
a heavy chain variable region (VH) of an anti-nicotine antibody optionally comprising the amino acid sequence of SEQ ID NO: 50, and optionally wherein the cognate ligand is nicotine or a derivative thereof;
a light chain variable region (VL) of an anti-nicotine antibody optionally comprising the amino acid sequence of SEQ ID NO: 51, and optionally wherein the cognate ligand is nicotine or a derivative thereof;
a progesterone receptor domain optionally comprising the amino acid sequence of SEQ ID NO: 52, and optionally wherein the cognate ligand is mifepristone or a derivative thereof;
an FKBP domain optionally comprising the amino acid sequence of SEQ ID NO: 43, and optionally wherein the cognate ligand is rapamycin, AP1903, AP20187, FK1012, derivatives thereof, or analogs thereof; and
an FRB domain optionally comprising the amino acid sequence of SEQ ID NO: 44, and optionally wherein the cognate ligand is rapamycin, AP1903, AP20187, FK1012, derivatives thereof, or analogs thereof.

384. The ACP of any one of embodiments 381-383, wherein the nucleic acid-binding domain comprises a DNA-binding zinc finger protein domain (ZF protein domain), optionally wherein the ZF protein domain is modular in design and is composed of an array of zinc finger motifs, optionally wherein the ZF-protein domain comprises one to ten zinc finger motifs.

385. The ACP of any one of embodiments 381, 383, and 384, wherein the chimeric polypeptide further comprises a linker localized between the nucleic acid-binding domain and the transcriptional effector domain, optionally wherein the linker comprises one or more 2A ribosome skipping tags, optionally wherein each 2A ribosome skipping tag is selected from the group consisting of: P2A, T2A, E2A, and F2A.

386. The ACP of any one of embodiments 381 and 383-385, wherein the chimeric polypeptide comprises a first ligand binding domain operably linked to the nucleic acid-binding domain and a second ligand binding domain operably linked to the transcriptional effector domain; optionally wherein:
each of the first and second ligand binding domains comprises a hormone-binding domain of estrogen receptor (ER) domain, optionally wherein the cognate ligand is tamoxifen or a metabolite thereof, optionally wherein the tamoxifen metabolite is selected from the group consisting of: 4-hydroxytamoxifen, N-desmethyltamoxifen, tamoxifen-N-oxide, and endoxifen; or
each of the first and second ligand binding domains comprises a progesterone receptor domain., optionally wherein the cognate ligand is mifepristone or a derivative thereof, and optionally wherein when the ligand binding domain comprises an ABI domain or a PYL domain, the cognate ligand is abscisic acid; or
each of the first and second ligand binding domains comprises a caffeine-binding single-domain antibody, optionally wherein the cognate ligand is caffeine or a derivative thereof; or
each of the first and the second ligand binding domains comprises a cannabidiol binding domain, optionally wherein the cognate ligand is a cannabidiol or a phytocannabinoid, optionally wherein the cannabidiol binding domain comprises a single-domain antibody or a nanobody, and optionally wherein the cannabidiol binding domain comprises an amino acid sequence selected from the group consisting of SEQ ID NO: 34, 35, 36, 37, and 38.

387. The ACP of any one of embodiments 381-386, wherein the nucleic acid-binding domain binds to the ACP-responsive promoter, optionally wherein the ACP-responsive promoter comprises an ACP-binding domain sequence and a promoter sequence, optionally wherein the promoter sequence comprises a minimal promoter, optionally wherein the promoter sequence is an inducible promoter and further comprises a responsive element selected from the group consisting of: NFκB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule-responsive promoters, and tandem repeats thereof, and optionally wherein the ACP-responsive promoter comprises a synthetic promoter, and optionally wherein the ACP-binding domain comprises one or more zinc finger binding sites.

388. The ACP of embodiment 386, wherein the ligand binding domain is localized N-terminal to the transcriptional effector domain or C-terminal to the transcriptional effector domain.

389. The ACP of embodiment 380 or embodiment 381, wherein the transcriptional effector domain comprises:
a transcriptional repressor, optionally wherein the transcriptional repressor comprises a transcriptional repressor domain is selected from the group consisting of: a Kruppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain; or
a transcriptional activator, optionally wherein the transcriptional activator comprises a transcriptional activation domain selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

390. The ACP of any one of embodiments 380-389, wherein the gene of interest is a cell death-inducing polypeptide, optionally wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase.

391. The ACP of embodiment 380, wherein the cell death-inducing polypeptide is:
caspase 9 or a functional truncation thereof, optionally comprising the amino acid sequence of SEQ ID NO: 39; or
Diphtheria toxin fragment A (DTA), optionally comprising the amino acid sequence of SEQ ID NO: 41; or
granzyme B, optionally comprising the amino acid sequence of SEQ ID NO: 47; or
Bax, optionally comprising the amino acid sequence of SEQ ID NO: 32.

392. An inducible cell death system comprising an engineered regulatable cell survival polypeptide, the cell survival polypeptide comprising:
a pro-survival polypeptide and a heterologous ligand binding domain,
wherein upon binding of the ligand binding domain to a cognate ligand, the cognate ligand inhibits the pro-survival polypeptide, optionally wherein the pro-survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein.

393. An inducible cell death system comprising a regulatable cell survival polypeptide and a cell death-inducing polypeptide,
wherein the cell-survival polypeptide comprises a pro-survival polypeptide and a heterologous
ligand binding domain,
wherein when expressed the cell survival polypeptide is capable of inhibiting the cell death-inducing polypeptide, and
wherein upon binding to a cognate ligand, the cognate ligand inhibits the pro-survival polypeptide, optionally wherein the cell survival polypeptide is selected from the group consisting of: XIAP, a modified XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein.

394. The inducible cell death system of embodiment 392 or 393, wherein the pro-survival polypeptide is XIAP or a modified XIAP.

395. The inducible cell death system of any one of embodiment 392-394, wherein the ligand binding domain is localized at the N-terminal region of the pro-survival polypeptide or at the C-terminal region of the pro-survival polypeptide.

396. The inducible cell death system of any one of embodiments 392-395, wherein the ligand binding domain comprises a domain, or functional fragment thereof, selected from the group consisting of:
an ABI domain, optionally comprising the amino acid sequence of SEQ ID NO: 31, and optionally wherein the cognate ligand is abscisic acid;
a PYL domain, optionally comprising the amino acid sequence of SEQ ID NO: 53, and optionally wherein the cognate ligand is abscisic acid;
a caffeine-binding single-domain antibody optionally comprising the amino acid sequence of SEQ ID NO: 33, and optionally wherein the cognate ligand is caffeine or a derivative thereof;
a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol;
a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen;
a heavy chain variable region (VH) of an anti-nicotine antibody optionally comprising the amino acid sequence of SEQ ID NO: 50, and optionally wherein the cognate ligand is nicotine or a derivative thereof;
a light chain variable region (VL) of an anti-nicotine antibody optionally comprising the amino acid sequence of SEQ ID NO: 51, and optionally wherein the cognate ligand is nicotine or a derivative thereof;
a progesterone receptor domain optionally comprising the amino acid sequence of SEQ ID NO: 52, and optionally wherein the cognate ligand is mifepristone or a derivative thereof;
an FKBP domain optionally comprising the amino acid sequence of SEQ ID NO: 43, and optionally wherein the cognate ligand is rapamycin, AP1903, AP20187, FK1012, derivatives thereof, or analogs thereof; and
an FRB domain optionally comprising the amino acid sequence of SEQ ID NO: 44, and optionally wherein the cognate ligand is rapamycin, AP1903, AP20187, FK1012, derivatives thereof, or analogs thereof.

397. The inducible cell death system of any one of embodiments 392-393, wherein the ligand binding domain comprises a degron, optionally wherein the degron is capable of inducing degradation of the regulatable cell survival polypeptide, and optionally wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), Spmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box.
398. The inducible cell death system of embodiment 397, wherein the degron comprises a cereblon (CRBN) polypeptide substrate domain capable of binding CRBN in response to an immunomodulatory drug (IMiD) thereby promoting ubiquitin pathway-mediated degradation of the regulatable polypeptide, optionally wherein the CRBN polypeptide substrate domain is selected from the group consisting of: IKZF1, IKZF3, Ckla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

399. The inducible cell death system of embodiment 398, wherein the CRBN polypeptide substrate domain is a chimeric fusion product of native CRBN polypeptide sequences, optionally wherein the CRBN polypeptide substrate domain is a IKZF3/ZFP91/IKZF3 chimeric fusion product having the amino acid sequence of

(SEQ ID NO: 103)
FNVLMVHKRSHTGERPLQCEICGFTCRQKGNLLRHIKLHTGEKPFKCHLC
NYACQRRDAL.

400. The inducible cell death system of any one of embodiments 397-399, wherein the ligand is an IMiD, optionally wherein the IMiD is an FDA-approved drug, and optionally wherein the IMiD is selected from the group consisting of: thalidomide, lenalidomide, and pomalidomide.

401. The inducible cell death system of any one of embodiments 393-400, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic chytochrom P450-2B 1, and Purine nucleoside phosphorylase.

402. The inducible cell death system of embodiment 401, wherein the cell death-inducing polypeptide is selected from the group consisting of:
caspase 9 or a functional truncation thereof, optionally comprising the amino acid sequence of SEQ ID NO: 39;
Diphtheria toxin fragment A (DTA), optionally comprising the amino acid sequence of SEQ ID NO: 41; and
Bax, optionally comprising the amino acid sequence of SEQ ID NO: 32.

403. An isolated cell comprising the inducible cell death polypeptide of any one of embodiments 374-379, the ACP of any one of embodiments 380-391, or the inducible cell death system of any one of embodiments 392-402.

404. An engineered nucleic acid encoding the inducible cell death polypeptide of any one of embodiments 374-377 and 379, the engineered nucleic acid comprising:
an expression cassette comprising a promoter and an exogenous polynucleotide sequence encoding the inducible cell death polypeptide monomer, wherein the first ligand binding domain and the second ligand binding domain are the same, wherein the promoter is operably linked to the exogenous polynucleotide.

405. An engineered nucleic acid encoding the inducible cell death polypeptide of any one of embodiments 374, 375, and 378, the engineered nucleic acid comprising:
an expression cassette comprising a promoter and an exogenous polynucleotide sequence encoding the first inducible cell death polypeptide monomer and the second inducible cell death polypeptide monomer, wherein the promoter is operably linked to the exogenous polynucleotide.

406. An engineered nucleic acid encoding the inducible cell death polypeptide of any one of embodiments 374, 375, and 378, the engineered nucleic acid comprising:
a) a first expression cassette comprising a first promoter and a first exogenous polynucleotide sequence encoding the first inducible cell death polypeptide monomer, wherein the first promoter is operably linked to the first exogenous polynucleotide; and
b) a second expression cassette comprising a second promoter and a second exogenous polynucleotide sequence encoding the second inducible cell death polypeptide monomer, wherein the second promoter is operably linked to the second exogenous polynucleotide.

407. An engineered nucleic acid encoding the activation-conditional control polypeptide (ACP) of embodiment 380 or 381, the engineered nucleic acid comprising:
an expression cassette comprising a promoter and an exogenous polynucleotide sequence encoding the ACP, wherein the promoter is operably linked to the exogenous polynucleotide.

408. An engineered nucleic acid encoding the ACP of embodiment 382, the engineered nucleic acid comprising:
an expression cassette comprising a promoter and an exogenous polynucleotide sequence having the formula:

C₁-L-C₂
wherein
C₁ comprises a polynucleotide sequence encoding the first chimeric polypeptide,
L comprises a linker polynucleotide sequence,
C₂ comprises a polynucleotide sequence encoding the second chimeric polypeptide;
wherein the promoter is operably linked to the exogenous polynucleotide.

409. The engineered nucleic acid of embodiment 408, wherein the linker polynucleotide sequence is operably associated with the translation of each chimeric polypeptide as a separate polypeptide, optionally wherein the linker polynucleotide sequence encodes:
a 2A ribosome skipping tag, optionally wherein the 2A ribosome skipping tag is selected from the group consisting of: P2A, T2A, E2A, and F2A; or
an Internal Ribosome Entry Site (IRES); or
a cleavable polypeptide, optionally comprising a furin polypeptide sequence.

410. An engineered nucleic acid encoding the ACP of embodiment 382, the engineered nucleic acid comprising:
a) a first expression cassette comprising a first promoter and a first exogenous polynucleotide sequence encoding the first chimeric polypeptide, wherein the first promoter is operably linked to the first exogenous polynucleotide; and
b) a second expression cassette comprising a second promoter and a second exogenous polynucleotide sequence encoding the second chimeric polypeptide, wherein the second promoter is operably linked to the second exogenous polynucleotide.

411. An engineered nucleic acid encoding the inducible cell death system of embodiment 392, the engineered nucleic acid comprising:
an expression cassette comprising a promoter and an exogenous polynucleotide sequence encoding the engineered regulatable cell survival polypeptide, wherein the promoter is operably linked to the exogenous polynucleotide.

412. An engineered nucleic acid encoding the inducible cell death system of embodiment 393, the engineered nucleic acid comprising:
a) a first expression cassette comprising a first promoter and a first exogenous polynucleotide sequence encoding the cell survival polypeptide, wherein the first promoter is operably linked to the first exogenous polynucleotide; and
b) a second expression cassette comprising a second promoter and a second exogenous polynucleotide sequence encoding the cell death polypeptide, wherein the second promoter is operably linked to the second exogenous polynucleotide.

413. The engineered nucleic acid of any one of embodiments 404, 405, 407, 409, and 411,
wherein the promoter comprises a constitutive promoter or an inducible promoter, and optionally is a synthetic promoter,
optionally wherein the constitutive promoter is selected from the group consisting of: CAG, HLP, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb, and
optionally wherein the inducible promoter comprises a minimal promoter and a responsive element selected from the group consisting of: NFκB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule-responsive promoters, and tandem repeats thereof.

414. The engineered nucleic acid of any one of embodiments 406, 410, and 412, wherein the first promoter, the second promoter, or both the first promoter and the second promoter comprise(s) a constitutive promoter or an inducible promoter, and optionally is a synthetic promoter,
optionally wherein the constitutive promoter is selected from the group consisting of: CAG, HLP, CMV, EFS, SFFV, SV40, MND, PGK, UbC, hEF1aV1, hCAGG, hEF1aV2, hACTb, heIF4A1, hGAPDH, hGRP78, hGRP94, hHSP70, hKINb, and hUBIb, and
optionally wherein the inducible promoter comprises a minimal promoter and a responsive element selected from the group consisting of: NFκB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule-responsive promoters, and tandem repeats thereof.

415. The inducible cell death system of any one of embodiment 392-402, wherein the XIAP comprises the amino acid sequence of SEQ ID NO: 107, wherein the modified XIAP comprises one or more amino acid substitutions within to positions 306-325 of SEQ ID NO:107.

416. The inducible cell death system of embodiment 415, wherein the one or more amino acid substitutions are at one or more positions of SEQ ID NO: 107 selected from the group consisting of: 305, 306, 308, and 325.

417. The inducible cell death system of embodiment 416, wherein the one or more amino acid substitutions are at position 305 of SEQ ID NO: 107.

418. The inducible cell death system of embodiment 417, wherein the amino acid substitution at position 305 of SEQ ID NO: 107 is G305M.

419. The inducible cell death system of any one of claims 416-418, wherein the one or more amino acid substitutions are at position 306 of SEQ ID NO: 107.

420. The inducible cell death system of embodiment 419, wherein the amino acid substitution at position 306 of SEQ ID NO: 107 is G3065.

421. The inducible cell death system of any one of claims 416-420, wherein the one or more amino acid substitutions are at position 308 of SEQ ID NO: 107.

422. The inducible cell death system of embodiment 421, wherein the amino acid substitution at position 308 of SEQ ID NO: 107 is selected from the group consisting of T3085 and T308D.

423. The inducible cell death system of embodiment 422, wherein the amino acid substitution at position 308 of SEQ ID NO: 107 is T3085.

424. The inducible cell death system of embodiment 422, wherein the amino acid substitution at position 308 of SEQ ID NO: 107 is T308D.

425. The inducible cell death system of any one of claims 416-424, wherein the one or more amino acid substitutions are at position 325 of SEQ ID NO: 107.

426. The inducible cell death system of embodiment 425, wherein the amino acid substitution at position 325 of SEQ ID NO: 107 is P325S.

427. The inducible cell death system of any one of claims 415-426, wherein the one or more amino acid substitutions are two amino acid substitutions.

428. The inducible cell death system of embodiment 427 wherein each of the two amino acid substitutions are at a position of SEQ ID NO: 107 selected from the group consisting of: 305, 306, 308, and 325.

429. The inducible cell death system of embodiment 428, wherein the two amino acid substitutions are at positions 305 and 306 of SEQ ID NO: 107.

430. The inducible cell death system of embodiment 429, wherein the amino acid substitution at position 305 of SEQ ID NO: 107 is G305M and the amino acid substitution at position 306 of SEQ ID NO: 107 is G3065.

431. The inducible cell death system of embodiment 427, wherein the two amino acid substitutions are at positions 305 and 308 of SEQ ID NO: 107.

432. The inducible cell death system of embodiment 431, wherein the amino acid substitution at position 305 of SEQ ID NO: 107 is G305M and the amino acid substitution at position 308 of SEQ ID NO: 107 is T3085.

433. The inducible cell death system of embodiment 431, wherein the amino acid substitution at position 305 of SEQ ID NO: 107 is G305M and the amino acid substitution at position 308 of SEQ ID NO: 107 is T308D.

434. The inducible cell death system of embodiment 427, wherein the two amino acid substitutions are at positions 305 and 325 of SEQ ID NO: 107.

435. The inducible cell death system of embodiment 434, wherein the amino acid substitution at position 305 of SEQ ID NO: 107 is G305M and the amino acid substitution at position 325 of SEQ ID NO: 107 is P325S.

436. The inducible cell death system of embodiment 427, wherein the two amino acid substitutions are at positions 306 and 308 of SEQ ID NO: 107.

437. The inducible cell death system of embodiment 436, wherein the amino acid substitution at position 306 of SEQ ID NO: 107 is G3065 and the amino acid substitution at position 308 of SEQ ID NO: 107 is T3085.

438. The inducible cell death system of embodiment 436, wherein the amino acid substitution at position 306 of SEQ ID NO: 107 is G3065 and the amino acid substitution at position 308 of SEQ ID NO: 107 is T308D.

439. The inducible cell death system of embodiment 437, wherein the two amino acid substitutions are at positions 306 and 325 of SEQ ID NO: 107.

440. The inducible cell death system of embodiment 439, wherein the amino acid substitution at position 306 of SEQ ID NO: 107 is G3065 and the amino acid substitution at position 325 of SEQ ID NO: 107 is P325S.

441. The inducible cell death system of embodiment 427, wherein the two amino acid substitutions are at positions 308 and 325 of SEQ ID NO: 107.

442. The inducible cell death system of embodiment 441, wherein the amino acid substitution at position 308 of SEQ ID NO: 107 is T3085 and the amino acid substitution at position 325 of SEQ ID NO: 107 is P325S.

443. The inducible cell death system of embodiment 441, wherein the amino acid substitution at position 308 of SEQ ID NO: 107 is T308D and the amino acid substitution at position 325 of SEQ ID NO: 107 is P325S.

444. The inducible cell death system of any one of claims 415 to 443, wherein the one or more additional amino acid substitutions are three amino acid substitutions.

445. The inducible cell death system of embodiment 444, wherein each of the three amino acid substitutions are at a position of SEQ ID NO: 107 selected from the group consisting of: 305, 306, 308, and 325.

446. The inducible cell death system of embodiment 445, wherein the three amino acid substitutions are at positions 305, 306, and 308 of SEQ ID NO: 107.

447. The inducible cell death system of embodiment 446, wherein the amino acid substitution at position 305 of SEQ ID NO: 107 is G305M, the amino acid substitution at position 306 of SEQ ID NO: 107 is G3065, and the amino acid substitution at position 308 of SEQ ID NO: 107 is T3085.

448. The inducible cell death system of embodiment 447, wherein the amino acid substitution at position 305 of SEQ ID NO: 107 is G305M, the amino acid substitution at position 306 of SEQ ID NO: 107 is G3065, and the amino acid substitution at position 308 of SEQ ID NO: 107 is T308D.

449. The inducible cell death system of embodiment 444, wherein the three amino acid substitutions are at positions 305, 306, and 325 of SEQ ID NO: 107.

450. The inducible cell death system of embodiment 449, wherein the amino acid substitution at position 305 of SEQ ID NO: 107 is G305M, the amino acid substitution at position 306 of SEQ ID NO: 107 is G3065, and the amino acid substitution at position 325 of SEQ ID NO: 107 is P325S.

451. The inducible cell death system of embodiment 444, wherein the three amino acid substitutions are at positions 305, 308, and 325 of SEQ ID NO: 107.

452. The inducible cell death system of embodiment 451, wherein the amino acid substitution at position 305 of SEQ ID NO: 107 is G305M, the amino acid substitution at position 308 of SEQ ID NO: 107 is T3085, and the amino acid substitution at position 325 of SEQ ID NO: 107 is P325S.

453. The inducible cell death system of embodiment 451, wherein the amino acid substitution at position 305 of SEQ ID NO: 107 is G305M, the amino acid substitution at position 308 of SEQ ID NO: 107 is T308D, and the amino acid substitution at position 325 of SEQ ID NO: 107 is P325S.

454. The inducible cell death system of embodiment 444, wherein the three amino acid substitutions are at positions 306, 308, and 325 of SEQ ID NO: 107.

455. The inducible cell death system of embodiment 454, wherein the amino acid substitution at position 306 of SEQ ID NO: 107 is G3065, the amino acid substitution at position 308 of SEQ ID NO: 107 is T3085, and the amino acid substitution at position 325 of SEQ ID NO: 107 is P325S.

456. The inducible cell death system of embodiment 454, wherein the amino acid substitution at position 306 of SEQ ID NO: 107 is G3065, the amino acid substitution at position 308 of SEQ ID NO: 107 is T3084, and the amino acid substitution at position 325 of SEQ ID NO: 107 is P325S.

457. The modified XIAP polypeptide of any one of claims 415 to 456, wherein the one or more additional amino acid substitutions are four amino acid substitutions.

458. The inducible cell death system of embodiment 457, wherein the four amino acid substitutions are at positions 305, 306, 308, and 325 of SEQ ID NO: 107.

459. The inducible cell death system of embodiment 458, wherein the amino acid substitution at position 305 of SEQ ID NO: 107 is G305M, the amino acid substitution at position 306 of SEQ ID NO: 107 is G3065, the amino acid substitution at position 308 of SEQ ID NO: 107 is T3085, and the amino acid substitution at position 325 of SEQ ID NO: 107 is P325S.

460. The inducible cell death system of embodiment 458, wherein the amino acid substitution at position 305 of SEQ ID NO: 107 is G305M, the amino acid substitution at position 306 of SEQ ID NO: 107 is G3065, the amino acid substitution at position 308 of SEQ ID NO: 107 is T308D, and the amino acid substitution at position 325 of SEQ ID NO: 107 is P325S.

### EXAMPLES

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2^{nd} Edition, 1989); Methods In Enzymology (S. Colowick and N. Kaplan eds., Academic Press, Inc.); Remington's Pharmaceutical Sciences, 18^{th} Edition (Easton, Pennsylvania: Mack Publishing Company, 1990); Carey and Sundberg Advanced Organic Chemistry 3^{rd} Ed. (Plenum Press) Vols A and B(1992).

### Example 1: Inducible Cell Death Systems and Methods

Materials, Methods, and Assays

Early passage 293s or primary cell types are transduced with either a lentiviral, retroviral, or adenoviral vector. The vector(s) encode an inducible form of caspase-9, inducible form of cell death gene product (such as Bax), or a chemically-inducible cell death gene circuit. Table A shows exemplary constructs for each of Systems 1, 2, 3, and 4 (described further below). Table B shows exemplary cell death-inducing genes that can be used in engineered cells. Table C shows exemplary survival genes that can be used in engineered cells. Table D shows exemplary sequences used in the constructs of Table A.

Cells expressing the cell death gene products or circuits are either tagged either with one or more fluorescent proteins or selected using selection markers, such as puromycin.

Addition of chemical inducer of dimerization/oligomerization/proximity to 293s or primary cell types after transduction with cell death-inducing circuit results in the apoptotic death of cells expressing the cell death-inducing gene circuits, while cells that do not contain the gene circuit do not undergo cell death similar to that of non-transduced cell controls.

Cell death is analyzed using cell-based assays for cell death detection such as TUNEL assay or cell staining with Annexin V and 7-Aminoactinomycin D using FACS analysis.

Systems

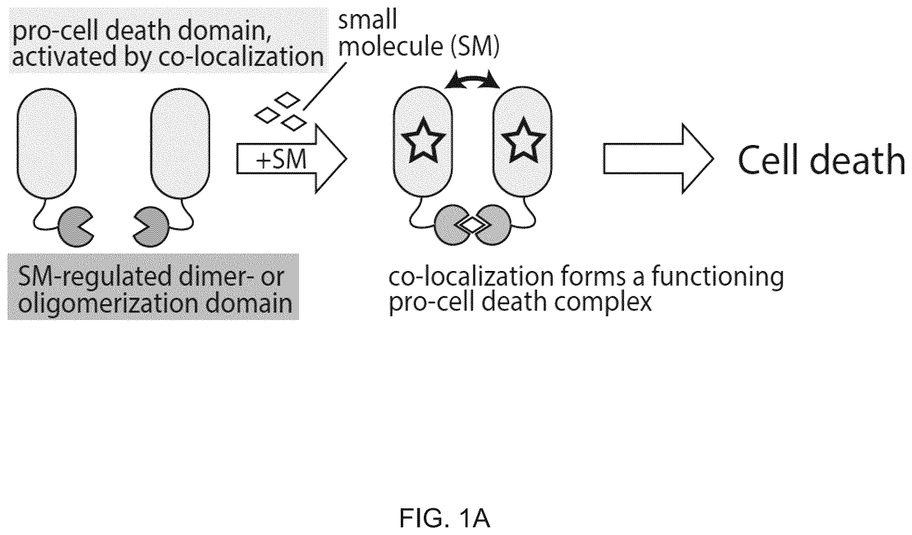

System 1: Cell death can be activated by a chemical inducer of dimerization (CID)/oligomerization/proximity or multiple chemical inducers, which activates the pro-cell death-inducing gene product by binding to specific ligand binding domain(s), causing homo-dimerization or hetero-dimerization of said domains, thereby activating the cell death-inducing pathways. An example of this system is shown in FIG. 1A.

System 2: A specific chemical, through binding to its corresponding specific ligand-binding domain(s), induces either the nuclear translocation and/or oligomerization (homo-dimerization or hetero-dimerization) or a combination of both processes (i.e. nuclear translocation and oligomerization), resulting in transcriptional activation of cell death-inducing gene product(s), such as Caspase-9, truncated BID (tBID) or Granzyme B. An example of this system is shown in FIG. 1B.

System 3: Degradation of a transcriptional repressor (such as KRAB) results in relief of transcriptional inhibition, resulting in transcriptional activation of cell death-inducing payload through combinatorial actions of specific zinc-finger pairing of transcriptional activation domains to respective cell death-inducing gene products. An example of this system is shown in FIG. 1C.

System 4: Cell death-inducing gene circuit(s) can be regulated by a chemical or a combination of chemicals that synergistically regulates the relative expression of anti-cell death and cell death-inducing gene products. The anti-cell death gene product is regulated by a chemical-regulated degron system, such that addition of said chemical (such as pomalidomide or lenalidomide) triggers the degradation of the anti-cell death gene product (such as XIAP) and causes cell death. An example of this system is shown in FIG. 1D.

TABLE A

| | | | Cell | | | |
|---|---|---|---|---|---|---|
| | | Ligand | death | Pro- | | |
| | | Binding | Inducing | Survival | | |
| System | Gene(s) | Domain | Domain | Domain | Drug/Ligand | Promoter |
| 1 | ABI_PYL-GS linker 1- | ABI_PYL | iCasp9 | NA | Abscisic acid | SFFV |
| | iCasp9 | | | | | |
| 1 | ABI-GS linker 1- | ABI | iCasp9 | NA | Abscisic acid | SFFV |
| | iCasp9 | | | | | |
| 1 | PYL-GS linker 1- | PYL | iCasp9 | NA | Abscisic acid | SFFV |
| | iCasp9 | | | | | |
| 1 | FKBP_FRB_tBID | FKBP and | tBID | NA | rapamycin | SFFV |
| | | FRB | | | | |
| 1 | aCaffVHH/acVHH-GS | acVHH | iCasp9 | NA | Caffeine | SFFV |
| | linker 1-iCasp9 | | | | or derivatives | |
| 1 | cannabidiol binding | cannabidiol | iCasp9 | NA | Cannabidiol | SFFV |
| | domain 1 (CA14)-GS | binding | | | or phytocannabinoids | |
| | linker 1--iCasp9 | domain | | | | |
| 1 | cannabidiol binding | cannabidiol | iCasp9 | NA | Cannabidiol | SFFV |
| | domain 2 | binding | | | or phytocannabinoids | |
| | (DB6/DB11/DB18/DB21)- | domain | | | | |
| | GS linker 1-iCasp9 | | | | | |
| 1 | cannabidiol binding | cannabidiol | iCasp9 | NA | Cannabidiol | SFFV |
| | domain 1 | binding | | | or phytocannabinoids | |
| | (CA14)_cannabidiol | domain | | | | |
| | binding domain 2 | | | | | |
| | (DB6/DB11/DB18/DB21)- | | | | | |
| | iCasp9 | | | | | |
| 1 | ER-FKBP-GS linker 1- | ER-FKBP | iCasp9 | NA | Tamoxifen/rapamycin | SFFV |
| | iCasp9 | | | | or derivatives | |
| 1 | FRB-ER-GS linker 1- | FRB-ER | iCasp9 | NA | Tamoxifen/rapamycin | SFFV |
| | iCasp9 | | | | or derivatives | |
| 1 | ER-GS linker 1-iCasp9 | ER | iCasp9 | NA | Tamoxifen | SFFV |
| | | | | | or derivatives | |
| 1 | NicVH-GS linker 1- | NicVH | iCasp9 | NA | Nicotine | SFFV |
| | iCasp9 | | | | or derivatives | |
| 1 | NicVL-GS linker 1- | NicVL | iCasp9 | NA | Nicotine | SFFV |
| | iCasp9 | | | | or derivatives | |
| 1 | PR-GS linker 1-iCasp9 | PR | iCasp9 | NA | Mifepristone | SFFV |
| | | (progesterone | | | (RU-486/Mifeprex) | |
| | | receptor) | | | | |
| 1 | ER-GS linker 2-iCasp9 | ER (estrogen | iCasp9 | NA | Tamoxifen | SFFV |
| | | receptor) | | | or derivatives | |
| 1 | NicVH-GS linker 2- | NicVH | iCasp9 | NA | Nicotine | SFFV |
| | iCasp9 | | | | or derivatives | |
| 1 | NicVL-GS linker 1- | NicVL | iCasp9 | NA | Nicotine | SFFV |
| | iCasp9 | | | | or derivatives | |
| 1 | iCasp9-GS linker 1-ER | ERT2 | iCasp9 | NA | Tamoxifen | SFFV |
| 1 | iCasp9-GS linker 1- | NicVH | iCasp9 | NA | Nicotine | SFFV |
| | NicVH | | | | or derivatives | |
| 1 | iCasp9-GS linker 1- | NicVH | iCasp9 | NA | Nicotine | |
| | NicVL | | | | or derivatives | |
| 1 | iCasp9-GS linker 1- | acVHH | iCasp9 | NA | Caffeine | SFFV |
| | acVHH | | | | or derivatives | |
| 1 | iCasp9-GS linker 2-ER | ER | iCasp9 | NA | Tamoxifen | SFFV |
| | | | | | or derivatives | |
| 1 | iCasp9-GS linker 2- | acVHH | iCasp9 | NA | Caffeine | SFFV |
| | acVHH | | | | or derivatives | |
| 2 | ZF10-1 VPR ER | ER | NA | NA | Tamoxifen | SFFV |
| | | | | | or derivatives | |
| 2 | ERT2_ZF10-1-2A-VPR- | ER | NA | NA | Tamoxifen | SFFV |
| | ER | | | | or derivatives | |
| 2 | ZF10-1 VPR NicVH | NicVH | NA | NA | Nicotine | minP |
| | | | | | or derivatives | |
| 2 | ZF10-1 VPR NicVL | NicVL | NA | NA | Nicotine | minP |
| | | | | | or derivatives | |
| 2 | NicVH-ZF10-1-2A-VPR- | NicVH/NicVL | NA | NA | Nicotine | minP |
| | NicVL | | | | or derivatives | |
| 2 | acVHH_ZF10-1-2A-VPR- | acVHH | NA | NA | Caffeine | minP |
| | acVHH | | | | or derivatives | |
| 2 | cannabidiol binding | cannabidiol | NA | NA | Cannabidiol | minP |
| | domain 1 (CA14)-ZF10-1- | binding | | | or phytocannabinoids | |
| | 2A-VPR-cannabidiol | domain | | | | |
| | binding domain 2 | | | | | |
| | (DB6/DB11/DB18/DB21) | | | | | |
| 2 | FKBP-ZF10-1-2ª-VPR- | FKBP-FRB | NA | NA | Rapamycin | minP |
| | FRB | | | | | |
| 2 | ZF10-1 VPR PR | PR | NA | NA | Mifepristone | minP |
| | | | | | (RU-486/Mifeprex) | |
| 2 | PR_ZF10-1-2ª-VPR_PR | PR | NA | NA | Mifepristone | minP |
| | | | | | (RU-486/Mifeprex) | |
| 2 | ABI-ZF10-1-2A-VPR- | ABI_PYL | NA | NA | Abscisic acid | minP |
| | PYL | | | | | |
| 2 | 4 × ZF10-1 binding site | NA | DTA | NA | NA | YB |
| | minP Diphtheria toxin A | | | | | TATA |
| | (DTA) | | | | | |
| 2 | 4 × ZF10-1 binding site | super degron | DTA | NA | IMiDs | YB |
| | minP Diphtheria toxin A | | | | | TATA |
| | (DTA) | | | | | |
| | d913 degron | | | | | |
| 2 | 4 × ZF10-1 binding site | NA | Caspase | NA | NA | YB |
| | minP Caspase 9 | | 9 | | | TATA |
| 2 | 4 × ZF10-1 binding site | super degron | Caspase | NA | IMiDs | YB |
| | minP Caspase 9 | | 9 | | | TATA |
| | d913 degron | | | | | |
| 2 | 4 × ZF10-1 binding site | acVHH | iCasp9 | NA | Caffeine | YB |
| | minP acVHH-iCasp9 | | | | or derivatives | TATA |
| 2 | 4 × ZF10-1 binding site | NicVH | iCasp9 | NA | Nicotine | YB |
| | minP NicVH_NicVL- | | | | or derivatives | TATA |
| | iCasp9 | | | | | |
| 2 | 4 × ZF10-1 binding site | super degron | Caspase | NA | IMiDs | YB |
| | minP | | 9 | | | TATA |
| | Caspase 9 (C287A) | | | | | |
| | d913 degron | | | | | |
| 2 | 4 × ZF10-1 binding site | | Caspase | NA | NA | YB |
| | minP Caspase 9 (C287A) | | 9 | | | TATA |
| 3 | NLS ZF10-1 KRAB | super degron | NA | NA | IMiDs | SFFV |
| | d913 degron | | | | | |
| 3 | 4 × ZF10-1 binding site | NA | NA | NA | NA | YB |
| | minP Diphtheria toxin A | | | | | TATA |
| | (DTA) | | | | | |
| 3 | 4 × ZF10-1 binding site | super degron | NA | NA | IMiDs | YB |
| | minP Diphtheria toxin A | | | | | TATA |
| | (DTA) | | | | | |
| | d913 degron | | | | | |
| 3 | 4 × ZF10-1 binding site | NA | NA | NA | NA | YB |
| | minP Granzyme B | | | | | TATA |
| 3 | 4 × ZF10-1 binding site | NA | NA | NA | IMiDs | YB |
| | minP Granzyme B super | | | | | TATA |
| | degron | | | | | |
| 3 | 4 × ZF10-1 binding site | NA | Caspase | NA | NA | YB |
| | minP Caspase 9 | | 9 | | | TATA |
| 3 | 4 × ZF10-1 binding site | super degron | Caspase | NA | IMiDs | YB |
| | minP Caspase 9 | | 9 | | | TATA |
| | d913 degron | | | | | |
| 3 | 4 × ZF10-1 binding site | acVHH | iCasp9 | NA | Caffeine | YB |
| | minPacVHH-iCasp9 | | | | or derivatives | TATA |
| 3 | 4 × ZF10-1 binding site | NicVH | iCasp9 | NA | Nicotine | YB |
| | minPNicVH_NicVL- | | | | or derivatives | TATA |
| | iCasp9 | | | | | |
| 3 | 4 × ZF10-1 binding site | super degron | Caspase | NA | IMiDs | YB |
| | minP | | 9 | | | TATA |
| | Caspase 9 (C287A) | | | | | |
| | d913 degron | | | | | |
| 3 | 4 × ZF10-1 binding site | Caspase 9 | Caspase | NA | NA | YB |
| | minP | | 9 | | | TATA |
| | Caspase 9 (C287A) | | | | | |
| 4 | human BAX (hBAX) | NA | hBAX | NA | NA | PGK_1 |
| 4 | human BAX (hBAX) | NA | hBAX | NA | NA | SFFV |
| 4 | XIAP-myc-d913 degron | NA | NA | XIAP | NA | SFFV |
| 4 | d913 degron-XIAP-myc | NA | NA | XIAP | NA | SFFV |

TABLE B
Cell death inducing proteins

DTA
Bax
Bad
Bcl-xS
Bak
Bik
Caspase 3
Caspase 8
Caspase 9
Fas or fragment thereof
Fas-associated death domain-containing
protein (FADD), TRADD or fragment thereof
homotypic interactions through death domain (DD),
such as that of TNF receptors (TNF-R)
homotypic interactions through death effector
domain (DED), such as that of Fas and Fas-associated
death domain-containing protein (FADD) homotypic interactions
through caspase recruitment domain (CARD) domains,
such as APAF-1
Granzyme B
Smac/DIABLO
OMI
BMF
Bid/truncated BID (tBID)
Bim
PUMA (p53-upregulated modulator of apoptosis)
Noxa/PMA-induced protein 1
Hrk
Cytochrome c
Caspase 6 or a fragment thereof
Caspase 7 or a fragment thereof
ARTS/Sept4
TNF-related apoptosis-inducing ligand (TRAIL)
Herpes Simplex Virus Thymidine Kinase
Varicella Zoster Virus Thymidine Kinase
Carboxyl Esterase
Cytosine Deaminase
Nitroreductase Fksb
Carboxypeptidase G2
Carboxypeptidase A
Horsearadish peroxidase
Linamarase
Hepatic chytochrom P450-2B1
Purine nucleoside phosphorylase

TABLE C
Pro-survival proteins

XIAP
Bcl-2
Bcl-xL
Mcl-1
E1B-19K
Bcl-w
Bfl1/BCL-2A1/A1
FLIP (FLICE-inhibitory protein)

TABLE D

| | SEQ | | SEQ | |
|---|---|---|---|---|
| Component | ID | | ID | |
| Name | NO | Amino acid sequence | NO | Nucleic acid sequence |
| ABI | 31 | VPLYGFTSICGRRPEMEAAVSTIPRFLQSSS | 58 | GTGCCCCTGTATGGCTTCACTTCCATTTGTG |
| | | GSMLDGRFDPQSAAHFFGVYDGHGGSQV | | GCCGACGGCCTGAAATGGAAGCCGCGGTGT |
| | | ANYCRERMHLALAEEIAKEKPMLCDGDT | | CAACCATACCACGGTTTCTGCAGAGCTCATC |
| | | WLEKWKKALFNSFLRVDSEIESVAPETVG | | AGGCTCCATGCTGGACGGACGCTTTGATCCA |
| | | STSVVAVVFPSHIFVANCGDSRAVLCRGK | | CAGTCTGCCGCACATTTCTTTGGAGTCTACG |
| | | TALPLSVDHKPDREDEAARIEAAGGKVIQ | | ACGGCCACGGGGGCAGCCAGGTCGCCAACT |
| | | WNGARVFGVLAMSRSIGDRYLKPSIIPDPE | | ACTGCAGGGAAAGGATGCATTTGGCACTTG |
| | | VTAVKRVKEDDCLILASDGVWDVMTDEE | | CCGAAGAGATCGCCAAAGAGAAGCCCATGT |
| | | ACEMARKRILLWHKKNAVAGDASLLADE | | TGTGTGATGGGGATACCTGGCTGGAGAAGT |
| | | RRKEGKDPAAMSAAEYLSKLAIQRGSKDN | | GGAAGAAAGCGCTTTTTAACTCTTTTCTGAG |
| | | ISVVVVDLK | | AGTGGATTCTGAGATAGAATCTGTCGCACCC |
| | | | | GAGACCGTGGGCAGCACATCCGTCGTAGCC |
| | | | | GTAGTGTTTCCCTCCCACATATTCGTCGCCA |
| | | | | ACTGCGGCGACAGTCGAGCCGTCCTCTGCC |
| | | | | GAGGTAAGACCGCCCTGCCTCTGAGTGTTG |
| | | | | ACCATAAGCCCGACCGGGAGGATGAGGCCG |
| | | | | CCCGAATCGAGGCCGCCGGTGGAAAAGTCA |
| | | | | TCCAATGGAACGGCGCAAGAGTGTTCGGCG |
| | | | | TGCTGGCGATGTCCAGGAGCATTGGAGACC |
| | | | | GGTACCTGAAGCCCAGCATAATCCCAGATC |
| | | | | CCGAAGTGACCGCAGTCAAGAGGGTGAAAG |
| | | | | AGGACGATTGTCTGATCCTGGCTAGCGATG |
| | | | | GCGTATGGGACGTGATGACTGATGAGGAGG |
| | | | | CGTGTGAAATGGCCCGCAAGCGAATCCTGC |
| | | | | TGTGGCATAAAAAAAACGCAGTCGCGGGGG |
| | | | | ACGCTTCTCTTCTGGCAGACGAAAGGCGCA |
| | | | | AAGAAGGTAAAGACCCGGCTGCTATGAGCG |
| | | | | CCGCCGAATATCTCAGTAAGCTGGCAATTCA |
| | | | | GCGAGGGTCCAAAGACAACATTTCCGTGGT |
| | | | | CGTGGTAGACCTCAAA |
| 4x ZF10-1 | | NA | 59 | CGGGTTTCGTAACAATCGCATGAGGATTCGC |
| binding site | | | | AACGCCTTCGGCGTAGCCGATGTCGCGCTCC |
| (BS) | | | | CGTCTCAGTAAAGGTCGGCGTAGCCGATGT |
| | | | | CGCGCAATCGGACTGCCTTCGTACGGCGTA |
| | | | | GCCGATGTCGCGCGTATCAGTCGCCTCGGA |
| | | | | ACGGCGTAGCCGATGTCGCGCATTCGTAAG |
| | | | | AGGCTCACTCTCCCTTACACGGAGTGGATA |
| Human Bax | 32 | DGSGEQPRGGGPTSSEQIMKTGALLLQGFI | 60 | GACGGGTCCGGGGAGCAGCCCAGAGGCGGG |
| | | QDRAGRMGGEAPELALDPVPQDASTKKL | | GGGCCCACCAGCTCTGAGCAGATCATGAAG |
| | | SECLKRIGDELDSNMELQRMIAAVDTDSP | | ACAGGGGCCCTTTTGCTTCAGGGTTTCATCC |
| | | REVFFRVAADMFSDGNFNWGRVVALFYF | | AGGATCGAGCAGGGCGAATGGGTGGAGAG |
| | | ASKLVLKALCTKVPELIRTIMGWTLDFLRE | | GCACCCGAGCTGGCCCTGGACCCGGTGCCT |
| | | RLLGWIQDQGGWDGLLSYFGTPTWQTVTI | | CAGGATGCGTCCACCAAGAAGCTGAGCGAG |
| | | FVAGVLTASLTIWKKMG | | TGTCTCAAGCGCATCGGGGACGAACTGGAC |
| | | | | AGTAACATGGAGCTGCAGAGGATGATTGCC |
| | | | | GCCGTGGACACAGACTCCCCCCGAGAGGTC |
| | | | | TTTTTCCGAGTGGCAGCTGACATGTTTTCTG |
| | | | | ACGGCAACTTCAACTGGGGCCGGGTTGTCG |
| | | | | CCCTTTTCTACTTTGCCAGCAAACTGGTGCT |
| | | | | CAAGGCCCTGTGCACCAAGGTGCCGGAACT |
| | | | | GATCAGAACCATCATGGGCTGGACATTGGA |
| | | | | CTTCCTCCGGGAGCGGCTGTTGGGCTGGATC |
| | | | | CAAGACCAGGGTGGTTGGGACGGCCTCCTC |
| | | | | TCCTACTTTGGGACGCCCACGTGGCAGACCG |
| | | | | TGACCATCTTTGTGGCGGGAGTGCTCACCGC |
| | | | | CTCACTCACCATCTGGAAGAAGATGGGC |
| caffeine- | 33 | GSQVQLVESGGGLVQAGGSLRLSCTASGR | 61 | GGCTCTCAGGTTCAATTGGTGGAATCTGGAG |
| binding | | TGTIYSMAWFRQAPGKEREFLATVGWSSG | | GGGGTCTCGTACAGGCAGGCGGTTCTCTCCG |
| domain | | ITYYMDSVKGRFTISRDKGKNTVYLQMDS | | ACTGAGTTGCACAGCCTCCGGTAGGACTGG |
| (acVHH/ | | LKPEDTAVYYCTATRAYSVGYDYWGQGT | | GACCATCTACTCAATGGCCTGGTTTCGCCAG |
| aCaffVHH) | | QVTVSS | | GCCCCAGGCAAAGAAAGAGAGTTTCTTGCC |
| | | | | ACTGTAGGTTGGAGTTCTGGGATCACATACT |
| | | | | ACATGGATTCAGTTAAAGGAAGATTCACTA |
| | | | | TCAGCCGAGATAAAGGGAAAAATACTGTGT |
| | | | | ACCTCCAGATGGACTCTCTGAAACCGGAGG |
| | | | | ACACGGCTGTCTACTACTGTACAGCCACCCG |
| | | | | CGCCTACTCCGTAGGGTATGACTACTGGGG |
| | | | | GCAAGGAACACAGGTAACCGTCTCTAGC |
| cannabidiol | 34 | EVQLQASGGGFVQPGGSLRLSCAASGSTS | 62 | GAGGTGCAGCTGCAGGCCAGCGGTGGCGGA |
| binding | | RQYDMGWFRQAPGKEREFVSAISSNQDQP | | TTCGTGCAGCCCGGAGGTTCACTGAGGCTG |
| domain 1 | | PYYADSVKGRFTISRDNSKNTVYLQMNSL | | AGTTGCGCCGCCAGCGGCTCTACGAGTCGA |
| (CA14) | | RAEDTATYYCAFKQHHANGAYWGQGTQ | | CAATATGACATGGGCTGGTTCAGGCAGGCC |
| | | VTVSS | | CCCGGCAAGGAGAGGGAGTTCGTGAGCGCC |
| | | | | ATCAGCTCTAACCAAGATCAGCCTCCCTACT |
| | | | | ATGCCGACTCAGTGAAGGGCAGGTTCACCA |
| | | | | TCAGCAGGGACAACAGCAAGAACACCGTGT |
| | | | | ACCTGCAGATGAACTCTCTGAGGGCCGAGG |
| | | | | ACACCGCCACCTATTACTGCGCCTTCAAGCA |
| | | | | GCACCATGCAAATGGCGCATACTGGGGACA |
| | | | | GGGAACCCAGGTGACCGTGTCTAGC |
| cannabidiol | 35 | EVQLQASGGGFVQPGGSLRLSCAASGRFS | 63 | GAGGTGCAGCTGCAGGCCAGCGGTGGCGGA |
| binding | | WGEEMGWFRQAPGKEREFVSAISWAATP | | TTCGTGCAGCCCGGAGGTTCACTGAGGCTG |
| domain 2 | | WQYYADSVKGRFTISRDNSKNTVYLQMN | | AGTTGCGCCGCCAGCGGCCGGTTCTCCTGGG |
| (DB6) | | SLRAEDTATYYCADEWHIGHVSYWGQGT | | GTGAAGAGATGGGCTGGTTCAGGCAGGCCC |
| | | QVTVSS | | CCGGCAAGGAGAGGGAGTTCGTGAGCGCCA |
| | | | | TCAGCTGGGCCGCTACCCCCTGGCAGTACTA |
| | | | | TGCCGACTCAGTGAAGGGCAGGTTCACCAT |
| | | | | CAGCAGGGACAACAGCAAGAACACCGTGTA |
| | | | | CCTGCAGATGAACTCTCTGAGGGCCGAGGA |
| | | | | CACCGCCACCTATTACTGCGCCGATGAGTGG |
| | | | | CACATAGGCCACGTCAGTTACTGGGGACAG |
| | | | | GGAACCCAGGTGACCGTGTCTAGC |
| cannabidiol | 36 | EVQLQASGGGFVQPGGSLRLSCAASGTTS | 64 | GAGGTGCAGCTGCAGGCCAGCGGTGGCGGA |
| binding | | DNDTMGWFRQAPGKEREFVSAISWNGGR | | TTCGTGCAGCCCGGAGGTTCACTGAGGCTG |
| domain 2 | | DEYYADSVKGRFTISRDNSKNTVYLQMNS | | AGTTGCGCCGCCAGCGGCACCACTTCAGAT |
| (DB11) | | LRAEDTATYYCAYQDNRSWQEYWGQGT | | AATGATACCATGGGCTGGTTCAGGCAGGCC |
| | | QVTVSS | | CCCGGCAAGGAGAGGGAGTTCGTGAGCGCC |
| | | | | ATCAGCTGGAACGGCGGGGGGACGAATAC |
| | | | | TATGCCGACTCAGTGAAGGGCAGGTTCACC |
| | | | | ATCAGCAGGGACAACAGCAAGAACACCGTG |
| | | | | TACCTGCAGATGAACTCTCTGAGGGCCGAG |
| | | | | GACACCGCCACCTATTACTGCGCCTACCAAG |
| | | | | ACAACAGGAGCTGGCAAGAATACTGGGGAC |
| | | | | AGGGAACCCAGGTGACCGTGTCTAGC |
| cannabidiol | 37 | EVQLQASGGGFVQPGGSLRLSCAASGGYS | 65 | GAGGTGCAGCTGCAGGCCAGCGGTGGCGGA |
| binding | | RADDMGWFRQAPGKEREFVSAISFGETDS | | TTCGTGCAGCCCGGAGGTTCACTGAGGCTG |
| domain 2 | | FYYADSVKGRFTISRDNSKNTVYLQMNSL | | AGTTGCGCCGCCAGCGGCGGTTATTCTCGCG |
| (DB18) | | RAEDTATYYCAYHNYTNMFEYWGQGTQ | | CCGATGATATGGGCTGGTTCAGGCAGGCCC |
| | | VTVSS | | CCGGCAAGGAGAGGGAGTTCGTGAGCGCCA |
| | | | | TCAGCTTCGGAGAGACGGACAGCTTTTACTA |
| | | | | TGCCGACTCAGTGAAGGGCAGGTTCACCAT |
| | | | | CAGCAGGGACAACAGCAAGAACACCGTGTA |
| | | | | CCTGCAGATGAACTCTCTGAGGGCCGAGGA |
| | | | | CACCGCCACCTATTACTGCGCCTACCACAAT |
| | | | | TACACTAATATGTTTGAGTACTGGGGACAG |
| | | | | GGAACCCAGGTGACCGTGTCTAGC |
| cannabidiol | 38 | EVQLQASGGGFVQPGGSLRLSCAASGTTY | 66 | GAGGTGCAGCTGCAGGCCAGCGGTGGCGGA |
| binding | | GQTNMGWFRQAPGKEREFVSAISGLQGR | | TTCGTGCAGCCCGGAGGTTCACTGAGGCTG |
| domain 2 | | DLYYADSVKGRFTISRDNSKNTVYLQMNS | | AGTTGCGCCGCCAGCGGCACCACTTACGGA |
| (DB21) | | LRAEDTATYYCAFHDFLRMWEYWGQGT | | CAAACCAATATGGGCTGGTTCAGGCAGGCC |
| | | QVTVSS | | CCCGGCAAGGAGAGGGAGTTCGTGAGCGCC |
| | | | | ATCAGCGGACTTCAAGGCAGGGATCTTTACT |
| | | | | ATGCCGACTCAGTGAAGGGCAGGTTCACCA |
| | | | | TCAGCAGGGACAACAGCAAGAACACCGTGT |
| | | | | ACCTGCAGATGAACTCTCTGAGGGCCGAGG |
| | | | | ACACCGCCACCTATTACTGCGCCTTCCACGA |
| | | | | TTTCCTTAGGATGTGGGAATACTGGGGACA |
| | | | | GGGAACCCAGGTGACCGTGTCTAGC |
| Caspase 9 | 39 | DEADRRLLRRCRLRLVEELQVDQLWDVL | 67 | GACGAAGCGGATCGGCGGCTCCTGCGGCGG |
| | | LSRELFRPHMIEDIQRAGSGSRRDQARQLII | | TGCCGGCTGCGGCTGGTGGAAGAGCTGCAG |
| | | DLETRGSQALPLFISCLEDTGQDMLASFLR | | GTGGACCAGCTCTGGGACGTCCTGCTGAGC |
| | | TNRQAAKLSKPTLENLTPVVLRPEIRKPEV | | CGCGAGCTGTTCAGGCCCCATATGATCGAG |
| | | LRPETPRPVDIGSGGFGDVGALESLRGNAD | | GACATCCAGCGGGCAGGCTCTGGATCTCGG |
| | | LAYILSMEPCGHCLIINNVNFCRESGLRTR | | CGGGATCAGGCCAGGCAGCTGATCATAGAT |
| | | TGSNIDCEKLRRRFSSLHFMVEVKGDLTA | | CTGGAGACTCGAGGGAGTCAGGCTCTTCCTT |
| | | KKMVLALLELARQDHGALDCCVVVILSH | | TGTTCATCTCCTGCTTAGAGGACACAGGCCA |
| | | GCQASHLQFPGAVYGTDGCPVSVEKIVNIF | | GGACATGCTGGCTTCGTTTCTGCGAACTAAC |
| | | NGTSCPSLGGKPKLFFIQACGGEQKDHGFE | | AGGCAAGCAGCAAAGTTGTCGAAGCCAACC |
| | | VASTSPEDESPGSNPEPDATPFQEGLRTFD | | CTAGAAAACCTTACCCCAGTGGTGCTCAGA |
| | | QLDAISSLPTPSDIFVSYSTFPGFVSWRDPK | | CCAGAGATTCGCAAACCAGAGGTTCTCAGA |
| | | SGSWYVETLDDIFEQWAHSEDLQSLLLRV | | CCGGAAACACCCAGACCAGTGGACATTGGT |
| | | ANAVSVKGIYKQMPGCFNFLRKKLFFKTS | | TCTGGAGGATTCGGTGATGTCGGTGCTCTTG |
| | | | | AGAGTTTGAGGGGAAATGCAGATTTGGCTT |
| | | | | ACATCCTGAGCATGGAGCCCTGTGGCCACT |
| | | | | GCCTCATTATCAACAATGTGAACTTCTGCCG |
| | | | | TGAGTCCGGGCTCCGCACCCGCACTGGCTCC |
| | | | | AACATCGACTGTGAGAAGTTGCGGCGTCGC |
| | | | | TTCTCCTCGCTGCATTTCATGGTGGAGGTGA |
| | | | | AGGGCGACCTGACTGCCAAGAAAATGGTGC |
| | | | | TGGCTTTGCTGGAGCTGGCGCGGCAGGACC |
| | | | | ACGGTGCTCTGGACTGCTGCGTGGTGGTCAT |
| | | | | TCTCTCTCACGGCTGTCAGGCCAGCCACCTG |
| | | | | CAGTTCCCAGGGGCTGTCTACGGCACAGAT |
| | | | | GGATGCCCTGTGTCGGTCGAGAAGATTGTG |
| | | | | AACATCTTCAATGGGACCAGCTGCCCCAGC |
| | | | | CTGGGAGGGAAGCCCAAGCTCTTTTTCATCC |
| | | | | AGGCCTGTGGTGGGGAGCAGAAAGACCATG |
| | | | | GGTTTGAGGTGGCCTCCACTTCCCCTGAAGA |
| | | | | CGAGTCCCCTGGCAGTAACCCCGAGCCAGA |
| | | | | TGCCACCCCGTTCCAGGAAGGTTTGAGGAC |
| | | | | CTTCGACCAGCTGGACGCCATATCTAGTTTG |
| | | | | CCCACACCCAGTGACATCTTTGTGTCCTACT |
| | | | | CTACTTTCCCAGGTTTTGTTTCCTGGAGGGA |
| | | | | CCCCAAGAGTGGCTCCTGGTACGTTGAGAC |
| | | | | CCTGGACGACATCTTTGAGCAGTGGGCTCAC |
| | | | | TCTGAAGACCTGCAGTCCCTCCTGCTTAGGG |
| | | | | TCGCTAATGCTGTTTCGGTGAAAGGGATTTA |
| | | | | TAAACAGATGCCTGGTTGCTTTAATTTCCTC |
| | | | | CGGAAAAAACTTTTCTTTAAAACATCA |
| d913 degron | 40 | FNVLMVHKRSHTGERPLQCEICGFTCRQK | 68 | TTCAATGTCTTAATGGTTCATAAGCGAAGCC |
| (without start | | GNLLRHIKLHTGEKPFKCHLCNYACQRRD | | ATACTGGTGAACGCCCATTGCAGTGCGAAA |
| codon; codon | | AL | | TATGCGGCTTTACCTGCCGCCAGAAAGGTA |
| sequence | | | | ACCTCCTCCGCCACATTAAACTGCACACAGG |
| version 1) | | | | GGAAAAACCTTTTAAGTGTCACCTCTGCAAC |
| | | | | TATGCATGCCAAAGAAGAGATGCGCTC |
| Diphtheria | 41 | DPDDVVDSSKSFVMENFSSYHGTKPGYVD | 69 | GACCCTGATGATGTTGTTGATTCTTCTAAAT |
| toxin A | | SIQKGIQKPKSGTQGNYDDDWKGFYSTDN | | CTTTTGTGATGGAAAACTTTTCTTCGTACCA |
| (DTA) | | KYDAAGYSVDNENPLSGKAGGVVKVTYP | | CGGGACTAAACCTGGTTATGTAGATTCCATT |
| | | GLTKVLALKVDNAETIKKELGLSLTEPLM | | CAAAAAGGTATACAAAAGCCAAAATCTGGT |
| | | EQVGTEEFIKRFGDGASRVVLSLPFAEGSS | | ACACAAGGAAATTATGACGATGATTGGAAA |
| | | SVEYINNWEQAKALSVELEINFETRGKRG | | GGGTTTTATAGTACCGACAATAAATACGAC |
| | | QDAMYEYMAQACAGNRVRRSLCEGTLLL | | GCTGCGGGATACTCTGTAGATAATGAAAAC |
| | | WCDIIGQTTYRDLKL | | CCGCTCTCTGGAAAAGCTGGAGGCGTGGTC |
| | | | | AAAGTGACGTATCCAGGACTGACGAAGGTT |
| | | | | CTCGCACTAAAAGTGGATAATGCCGAAACT |
| | | | | ATTAAGAAAGAGTTAGGTTTAAGTCTCACTG |
| | | | | AACCGTTGATGGAGCAAGTCGGAACGGAAG |
| | | | | AGTTTATCAAAAGGTTCGGTGATGGTGCTTC |
| | | | | GCGTGTAGTGCTCAGCCTTCCCTTCGCTGAG |
| | | | | GGGAGTTCTAGCGTTGAATATATTAATAACT |
| | | | | GGGAACAGGCGAAAGCGTTAAGCGTAGAAC |
| | | | | TTGAGATTAATTTTGAAACCCGTGGAAAAC |
| | | | | GTGGCCAAGATGCGATGTATGAGTATATGG |
| | | | | CTCAAGCCTGTGCAGGAAATCGTGTCAGGC |
| | | | | GATCTCTTTGTGAAGGAACCTTACTTCTGTG |
| | | | | GTGTGACATAATTGGACAAACTACCTACAG |
| | | | | AGATTTAAAGCTC |
| ER (ERT2 | 42 | AGDMRAANLWPSPLMIKRSKKNSLALSLT | 70 | GCCGGCGATATGAGAGCTGCTAACCTGTGG |
| mutant) | | ADQMVSALLDAEPPILYSEYDPTRPFSEAS | | CCTTCTCCACTGATGATCAAGCGGTCCAAGA |
| | | MMGLLTNLADRELVHMINWAKRVPGFVD | | AGAACAGTCTGGCCCTGAGCCTGACCGCCG |
| | | LTLHDQVHLLECAWLEILMIGLVWRSMEH | | ACCAGATGGTTTCAGCACTGCTGGATGCCG |
| | | PVKLLFAPNLLLDRNQGKCVEGMVEIFDM | | AGCCTCCTATCCTGTACAGCGAGTACGACCC |
| | | LLATSSRFRMMNLQGEEFVCLKSIILLNSG | | CACCAGACCTTTTAGCGAGGCCAGCATGAT |
| | | VYTFLSSTLKSLEEKDHIHRVLDKITDTLIH | | GGGCCTGCTGACCAATCTGGCCGACAGAGA |
| | | LMAKAGLTLQQQHQRLAQLLLILSHIRHM | | ACTGGTGCACATGATCAACTGGGCCAAGCG |
| | | SNKGMEHLYSMKCKNVVPLYDLLLEAAD | | CGTGCCCGGCTTTGTGGATCTGACACTGCAC |
| | | AHRLHAPTSRGGASVEETDQSHLATAGST | | GACCAAGTGCATCTGCTCGAGTGCGCCTGG |
| | | SSHSLQKYYITGEAEGFPAT | | CTGGAAATCCTGATGATCGGACTCGTGTGGC |
| | | | | GGAGCATGGAACACCCTGTGAAGCTGCTGT |
| | | | | TCGCCCCTAACCTGCTGCTGGACAGAAACC |
| | | | | AGGGCAAATGCGTGGAAGGCATGGTGGAAA |
| | | | | TCTTCGATATGCTGCTGGCCACCTCCAGCCG |
| | | | | GTTCCGGATGATGAATCTGCAGGGCGAAGA |
| | | | | GTTCGTGTGCCTGAAGTCCATTATCCTGCTG |
| | | | | AACAGCGGCGTGTACACCTTTCTGAGCAGC |
| | | | | ACCCTGAAGTCTCTGGAAGAGAAGGACCAC |
| | | | | ATCCACAGAGTGCTGGACAAGATCACCGAC |
| | | | | ACACTGATCCACCTGATGGCCAAGGCCGGA |
| | | | | CTGACTCTGCAGCAGCAGCATCAAAGACTG |
| | | | | GCCCAGCTGCTGCTCATCCTGAGCCACATCA |
| | | | | GACACATGAGCAACAAAGGCATGGAACATC |
| | | | | TGTACAGCATGAAGTGCAAGAACGTGGTGC |
| | | | | CCCTGTACGATCTGCTGCTCGAAGCCGCTGA |
| | | | | TGCCCACAGACTGCATGCCCCTACATCTAGA |
| | | | | GGCGGAGCCTCCGTGGAAGAGACAGATCAG |
| | | | | TCTCATCTGGCCACCGCCGGCAGCACAAGCT |
| | | | | CTCATTCTCTGCAGAAGTACTACATCACCGG |
| | | | | CGAGGCCGAGGGATTTCCTGCCACA |
| FKBP | 43 | GVQVETISPGDGRTFPKRGQTCVVHYTGM | 71 | GGAGTGCAGGTGGAAACCATCTCCCCAGGA |
| | | LEDGKKFDSSRDRNKPFKFMLGKQEVIRG | | GACGGGCGCACCTTCCCCAAGCGCGGCCAG |
| | | WEEGVAQMSVGQRAKLTISPDYAYGATG | | ACCTGCGTGGTGCACTACACCGGGATGCTTG |
| | | HPGIIPPHATLVFDVELLKLE | | AAGATGGAAAGAAATTTGATTCCTCCCGGG |
| | | | | ACAGAAACAAGCCCTTTAAGTTTATGCTAG |
| | | | | GCAAGCAGGAGGTGATCCGAGGCTGGGAAG |
| | | | | AAGGGGTTGCCCAGATGAGTGTGGGTCAGA |
| | | | | GAGCCAAACTGACTATATCTCCAGATTATGC |
| | | | | CTATGGTGCCACTGGGCACCCAGGCATCATC |
| | | | | CCACCACATGCCACTCTCGTCTTCGATGTGG |
| | | | | AGCTTCTAAAACTGGAA |
| FRB | 44 | ILWHEMWHEGLEEASRLYFGERNVKGMF | 72 | ATCCTCTGGCATGAGATGTGGCATGAAGGC |
| | | EVLEPLHAMMERGPQTLKETSFNQAYGR | | CTGGAAGAGGCATCTCGTTTGTACTTTGGGG |
| | | DLMEAQEWCRKYMKSGNVKDLTQAWDL | | AAAGGAACGTGAAAGGCATGTTTGAGGTGC |
| | | YYHVFRRISK | | TGGAGCCCTTGCATGCTATGATGGAACGGG |
| | | | | GCCCCCAGACTCTGAAGGAAACATCCTTTA |
| | | | | ATCAGGCCTATGGTCGAGATTTAATGGAGG |
| | | | | CCCAAGAGTGGTGCAGGAAGTACATGAAAT |
| | | | | CAGGGAATGTCAAGGACCTCACCCAAGCCT |
| | | | | GGGACCTCTATTATCATGTGTTCCGACGAAT |
| | | | | CTCAAAG |
| GS linker 1 | 45 | GGGGSGGGGSGGGGSVDGF | 73 | GGGGGTGGAGGTTCAGGGGGTGGAGGTTCA |
| | | | | GGTGGTGGCGGTAGTGTCGATGGCTTC |
| GS linker 2 | 46 | ASGGGGSAS | 74 | GCTAGTGGCGGCGGCGGCAGTGCTAGT |
| Granzyme B | 47 | QPILLLLAFLLLPRADAGEIIGGHEAKPHSR | 75 | CAACCAATCCTGCTTCTGCTGGCCTTCCTCC |
| | | PYMAYLMIWDQKSLKRCGGFLIQDDFVLT | | TGCTGCCCAGGGCAGATGCAGGGGAGATCA |
| | | AAHCWGSSINVTLGAHNIKEQEPTQQFIPV | | TCGGGGGACATGAGGCCAAGCCCCACTCCC |
| | | KRPIPHPAYNPKNFSNDIMLLQLERKAKRT | | GCCCCTACATGGCTTATCTTATGATCTGGGA |
| | | RAVQPLRLPSNKAQVKPGQTCSVAGWGQ | | TCAGAAGTCTCTGAAGAGGTGCGGTGGCTT |
| | | TAPLGKHSHTLQEVKMTVQEDRKCESDLR | | CCTGATACAAGACGACTTCGTGCTGACAGCT |
| | | HYYDSTIELCVGDPEIKKTSFKGDSGGPLV | | GCTCACTGTTGGGGAAGCTCCATAAATGTCA |
| | | CNKVAQGIVSYGRNNGMPPRACTKVSSFV | | CCTTGGGGGCCCACAATATCAAAGAACAGG |
| | | HWIKKTMKRH | | AGCCGACCCAGCAGTTTATCCCTGTGAAAA |
| | | | | GACCCATCCCCCATCCAGCCTATAATCCTAA |
| | | | | GAACTTCTCCAACGACATCATGCTACTGCAG |
| | | | | CTGGAGAGAAAGGCCAAGCGGACCAGAGCT |
| | | | | GTGCAGCCCCTCAGGCTACCTAGCAACAAG |
| | | | | GCCCAGGTGAAGCCAGGGCAGACATGCAGT |
| | | | | GTGGCCGGCTGGGGGCAGACGGCCCCCCTG |
| | | | | GGAAAACACTCACACACACTACAAGAGGTG |
| | | | | AAGATGACAGTGCAGGAAGATCGAAAGTGC |
| | | | | GAATCTGACTTACGCCATTATTACGACAGTA |
| | | | | CCATTGAGTTGTGCGTGGGGGACCCAGAGA |
| | | | | TTAAAAAGACTTCCTTTAAGGGGGACTCTGG |
| | | | | AGGCCCTCTTGTGTGTAACAAGGTGGCCCA |
| | | | | GGGCATTGTCTCCTATGGACGAAACAATGG |
| | | | | CATGCCTCCACGAGCCTGCACCAAAGTCTCA |
| | | | | AGCTTTGTACACTGGATAAAGAAAACCATG |
| | | | | AAACGCCAC |
| iCasp9 | 48 | DVGALESLRGNADLAYILSMEPCGHCLIIN | 76 | GATGTCGGTGCTCTTGAGAGTTTGAGGGGA |
| | | NVNFCRESGLRTRTGSNIDCEKLRRRFSSL | | AATGCAGATTTGGCTTACATCCTGAGCATGG |
| | | HFMVEVKGDLTAKKMVLALLELARQDHG | | AGCCCTGTGGCCACTGCCTCATTATCAACAA |
| | | ALDCCVVVILSHGCQASHLQFPGAVYGTD | | TGTGAACTTCTGCCGTGAGTCCGGGCTCCGC |
| | | GCPVSVEKIVNIFNGTSCPSLGGKPKLFFIQ | | ACCCGCACTGGCTCCAACATCGACTGTGAG |
| | | ACGGEQKDHGFEVASTSPEDESPGSNPEPD | | AAGTTGCGGCGTCGCTTCTCCTCGCTGCATT |
| | | ATPFQEGLRTFDQLDAISSLPTPSDIFVSYS | | TCATGGTGGAGGTGAAGGGCGACCTGACTG |
| | | TFPGFVSWRDPKSGSWYVETLDDIFEQWA | | CCAAGAAAATGGTGCTGGCTTTGCTGGAGC |
| | | HSEDLQSLLLRVANAVSVKGIYKQMPGCF | | TGGCGCGGCAGGACCACGGTGCTCTGGACT |
| | | NFLRKKLFFKTS | | GCTGCGTGGTGGTCATTCTCTCTCACGGCTG |
| | | | | TCAGGCCAGCCACCTGCAGTTCCCAGGGGC |
| | | | | TGTCTACGGCACAGATGGATGCCCTGTGTCG |
| | | | | GTCGAGAAGATTGTGAACATCTTCAATGGG |
| | | | | ACCAGCTGCCCCAGCCTGGGAGGGAAGCCC |
| | | | | AAGCTCTTTTTCATCCAGGCCTGTGGTGGGG |
| | | | | AGCAGAAAGACCATGGGTTTGAGGTGGCCT |
| | | | | CCACTTCCCCTGAAGACGAGTCCCCTGGCAG |
| | | | | TAACCCCGAGCCAGATGCCACCCCGTTCCA |
| | | | | GGAAGGTTTGAGGACCTTCGACCAGCTGGA |
| | | | | CGCCATATCTAGTTTGCCCACACCCAGTGAC |
| | | | | ATCTTTGTGTCCTACTCTACTTTCCCAGGTTT |
| | | | | TGTTTCCTGGAGGGACCCCAAGAGTGGCTCC |
| | | | | TGGTACGTTGAGACCCTGGACGACATCTTTG |
| | | | | AGCAGTGGGCTCACTCTGAAGACCTGCAGT |
| | | | | CCCTCCTGCTTAGGGTCGCTAATGCTGTTTC |
| | | | | GGTGAAAGGGATTTATAAACAGATGCCTGG |
| | | | | TTGCTTTAATTTCCTCCGGAAAAAACTTTTC |
| | | | | TTTAAAACATCA |
| KRAB | 49 | RTLVTFKDVFVDFTREEWKLLDTAQQIVY | 77 | AGAACACTGGTTACGTTCAAGGACGTGTTTG |
| | | RNVMLENYKNLVSLGYQLTKPDVILRLEK | | TGGACTTTACACGTGAGGAGTGGAAATTGC |
| | | GEEPWLV | | TGGATACTGCGCAACAAATTGTGTATCGAA |
| | | | | ATGTCATGCTTGAGAATTACAAGAACCTCGT |
| | | | | CAGTCTCGGATACCAGTTGACGAAACCGGA |
| | | | | TGTGATCCTTAGGCTCGAAAAGGGGGAAGA |
| | | | | ACCTTGGCTGGTA |
| minKRAB | 137 | RTLVTFKDVFVDFTREEWKLLDTAQQIVY | | |
| | | RNVMLENYKNLVSLGY | | |
| NicVH | 50 | QMQLLESGPGLVKPSETLSLTCTVSGGSIW | 78 | CAGATGCAACTGTTGGAATCGGGACCGGGT |
| | | GWIRQPPGKGLEWIGSIYSSGSTYYNPSLK | | TTGGTCAAGCCGAGCGAGACATTGAGTTTA |
| | | SRVTTSVDTSKNQFSLRLSSVTAADTAVY | | ACGTGCACGGTTTCAGGCGGATCAATTTGG |
| | | YCVAWFGDLLSLKGVELWGQGTLVTVS | | GGTTGGATTCGCCAGCCGCCAGGAAAGGGA |
| | | | | CTGGAGTGGATTGGATCTATTTACTCCTCCG |
| | | | | GTTCCACCTACTATAATCCGAGCTTGAAGTC |
| | | | | CCGTGTGACAACAAGCGTGGATACATCCAA |
| | | | | AAACCAGTTTTCATTGCGCCTGTCCTCAGTA |
| | | | | ACCGCAGCCGACACAGCCGTATATTATTGC |
| | | | | GTTGCTTGGTTTGGCGACTTATTAAGTCTTA |
| | | | | AAGGGGTAGAGCTTTGGGGCCAGGGAACTC |
| | | | | TTGTGACGGTATCG |
| NicVL | 51 | QSELTQPPSASGTPGQRVTISCSGSSSNIGS | 79 | CAATCCGAGTTGACCCAGCCGCCTAGTGCTA |
| | | NYVYWYQQLPGTAPKLLIYRNNQRPSGVP | | GTGGAACACCGGGACAGCGCGTGACAATTT |
| | | DRFSGSKSGTSASLAISGLRSEDEADYYCA | | CATGCTCCGGTTCAAGCTCAAATATCGGCTC |
| | | AWDDSLSAWVFGGGTQLDILG | | TAATTATGTTTACTGGTATCAGCAGCTTCCA |
| | | | | GGTACTGCGCCTAAGCTCTTAATCTACCGTA |
| | | | | ACAATCAACGTCCGTCCGGTGTGCCCGACC |
| | | | | GCTTCTCAGGCAGTAAAAGTGGTACTTCCGC |
| | | | | ATCCCTTGCAATTTCGGGACTTCGTAGCGAA |
| | | | | GATGAGGCAGACTATTACTGTGCAGCATGG |
| | | | | GATGATTCCTTATCAGCTTGGGTATTCGGTG |
| | | | | GCGGAACTCAATTAGACATTTTGGGG |
| PR domain | 52 | GIRKFKKFNKVRVVRALDAVALPQPLGVP | 80 | GGGATCCGAAAATTTAAAAAGTTCAATAAA |
| | | NESQALSQRFTFSPGQDIQLIPPLINLLMSIE | | GTCAGAGTTGTGAGAGCACTGGATGCTGTT |
| | | PDVIYAGHDNTKPDTSSSLLTSLNQLGERQ | | GCTCTCCCACAGCCATTGGGCGTTCCAAATG |
| | | LLSVVKWSKSLPGFRNLHIDDQITLIQYSW | | AAAGCCAAGCCCTAAGCCAGAGATTCACTT |
| | | MSLMVFGLGWRSYKHVSGQMLYFAPDLI | | TTTCACCAGGTCAAGACATACAGTTGATTCC |
| | | LNEQRMKESSFYSLCLTMWQIPQEFVKLQ | | ACCACTGATCAACCTGTTAATGAGCATTGAA |
| | | VSQEEFLCMKVLLLLNTIPLEGLRSQTQFE | | CCAGATGTGATCTATGCAGGACATGACAAC |
| | | EMRSSYIRELIKAIGLRQKGVVSSSQRFYQ | | ACAAAACCTGACACCTCCAGTTCTTTGCTGA |
| | | LTKLLDNLHDLVKQLHLYCLNTFIQSRALS | | CAAGTCTTAATCAACTAGGCGAGAGGCAAC |
| | | VEFPEMMSEVIAAQLPKILAGMVKPLLFH | | TTCTTTCAGTAGTCAAGTGGTCTAAATCATT |
| | | KK | | GCCAGGTTTTCGAAACTTACATATTGATGAC |
| | | | | CAGATAACTCTCATTCAGTATTCTTGGATGA |
| | | | | GCTTAATGGTGTTTGGTCTAGGATGGAGATC |
| | | | | CTACAAACATGTCAGTGGGCAGATGCTGTA |
| | | | | TTTTGCACCTGATCTAATACTAAATGAACAG |
| | | | | CGGATGAAAGAATCATCATTCTATTCATTAT |
| | | | | GCCTTACCATGTGGCAGATCCCACAGGAGTT |
| | | | | TGTCAAGCTTCAAGTTAGCCAAGAAGAGTT |
| | | | | CCTCTGTATGAAAGTATTGTTACTTCTTAAT |
| | | | | ACAATTCCTTTGGAAGGGCTACGAAGTCAA |
| | | | | ACCCAGTTTGAGGAGATGAGGTCAAGCTAC |
| | | | | ATTAGAGAGCTCATCAAGGCAATTGGTTTG |
| | | | | AGGCAAAAAGGAGTTGTGTCGAGCTCACAG |
| | | | | CGTTTCTATCAACTTACAAAACTTCTTGATA |
| | | | | ACTTGCATGATCTTGTCAAACAGCTTCATCT |
| | | | | GTACTGCTTGAATACATTTATCCAGTCCCGG |
| | | | | GCACTGAGTGTTGAATTTCCAGAAATGATGT |
| | | | | CTGAAGTTATTGCTGCACAATTACCCAAGAT |
| | | | | ATTGGCAGGGATGGTGAAACCCCTTCTCTTT |
| | | | | CATAAAAAG |
| PYL | 53 | TQDEFTQLSQSIAEFHTYQLGNGRCSSLLA | 81 | ACTCAAGACGAATTCACCCAACTCTCCCAAT |
| | | QRIHAPPETVWSVVRRFDRPQIYKHFIKSC | | CAATCGCCGAGTTCCACACGTACCAACTCG |
| | | NVSEDFEMRVGCTRDVNVISGLPANTSRE | | GTAACGGCCGTTGCTCATCTCTCCTAGCTCA |
| | | RLDLLDDDRRVTGFSITGGEHRLRNYKSV | | GCGAATCCACGCGCCGCCGGAAACAGTATG |
| | | TTVHRFEKEEEEERIWTVVLESYVVDVPE | | GTCCGTGGTGAGACGTTTCGATAGGCCACA |
| | | GNSEEDTRLFADTVIRLNLQKLASITEAMN | | GATTTACAAACACTTCATCAAAAGCTGTAAC |
| | | | | GTGAGTGAAGATTTCGAGATGCGAGTGGGA |
| | | | | TGCACGCGCGACGTGAACGTGATAAGTGGA |
| | | | | TTACCGGCGAATACGTCTCGAGAGAGATTA |
| | | | | GATCTGTTGGACGATGATCGGAGAGTGACT |
| | | | | GGGTTTAGTATAACCGGTGGTGAACATAGG |
| | | | | CTGAGGAATTATAAATCGGTTACGACGGTTC |
| | | | | ATAGATTTGAGAAAGAAGAAGAAGAAGAA |
| | | | | AGGATCTGGACCGTTGTTTTGGAATCTTATG |
| | | | | TTGTTGATGTACCGGAAGGTAATTCGGAGG |
| | | | | AAGATACGAGATTGTTTGCTGATACGGTTAT |
| | | | | TAGATTGAATCTTCAGAAACTTGCTTCGATC |
| | | | | ACTGAAGCTATGAAC |
| tBID | 54 | DCEVNNGSSLRDECITNLLVFGFLQSCSDN | | GACTGTGAGGTCAACAACGGTTCCAGCCTC |
| | | SFRRELDALGHELPVLAPQWEGYDELQTD | | AGGGATGAGTGCATCACAAACCTACTGGTG |
| | | GNRSSHSRLGRIEADSESQEDIIRNIARHLA | | TTTGGCTTCCTCCAAAGCTGTTCTGACAACA |
| | | QVGDSMDRSIPPGLVNGLALQLRNTSRSE | | GCTTCCGCAGAGAGCTGGACGCACTGGGCC |
| | | EDRNRDLATALEQLLQAYPRDMEKEKTM | | ACGAGCTGCCAGTGCTGGCTCCCCAGTGGG |
| | | LVLALLLAKKVASHTPSLLRDVFHTTVNFI | | AGGGCTACGATGAGCTGCAGACTGATGGCA |
| | | NQNLRTYVRSLARNGMD | | ACCGCAGCAGCCACTCCCGCTTGGGAAGAA |
| | | | | TAGAGGCAGATTCTGAAAGTCAAGAAGACA |
| | | | | TCATCCGGAATATTGCCAGGCACCTCGCCCA |
| | | | | GGTCGGGGACAGCATGGACCGTAGCATCCC |
| | | | | TCCGGGCCTGGTGAACGGCCTGGCCCTGCA |
| | | | | GCTCAGGAACACCAGCCGGTCGGAGGAGGA |
| | | | | CCGGAACAGGGACCTGGCCACTGCCCTGGA |
| | | | | GCAGCTGCTGCAGGCCTACCCTAGAGACAT |
| | | | | GGAGAAGGAGAAGACCATGCTGGTGCTGGC |
| | | | | CCTGCTGCTGGCCAAGAAGGTGGCCAGTCA |
| | | | | CACGCCGTCCTTGCTCCGTGATGTCTTTCAC |
| | | | | ACAACAGTGAATTTTATTAACCAGAACCTAC |
| | | | | GCACCTACGTGAGGAGCTTAGCCAGAAATG |
| | | | | GGATGGAC |
| VPR | 55 | EASGSGRADALDDFDLDMLGSDALDDFD | 82 | GAAGCCTCTGGAAGCGGCAGAGCTGACGCC |
| | | LDMLGSDALDDFDLDMLGSDALDDFDLD | | CTGGATGACTTCGACCTGGATATGCTGGGCA |
| | | MLINSRSSGSPKKKRKVGSQYLPDTDDRH | | GCGACGCTCTGGACGATTTTGACCTCGACAT |
| | | RIEEKRKRTYETFKSIMKKSPFSGPTDPRPP | | GCTGGGATCTGATGCACTCGACGATTTCGAT |
| | | PRRIAVPSRSSASVPKPAPQPYPFTSSLSTIN | | TTGGACATGCTCGGCAGTGATGCCTTGGACG |
| | | YDEFPTMVFPSGQISQASALAPAPPQVLPQ | | ACTTTGATCTTGATATGCTCATCAACAGCCG |
| | | APAPAPAPAMVSALAQAPAPVPVLAPGPP | | GTCCAGCGGCAGCCCCAAGAAAAAAAGAAA |
| | | QAVAPPAPKPTQAGEGTLSEALLQLQFDD | | AGTGGGCTCCCAGTACCTGCCTGACACCGA |
| | | EDLGALLGNSTDPAVFTDLASVDNSEFQQ | | CGACAGACACCGGATCGAGGAAAAGCGGA |
| | | LLNQGIPVAPHTTEPMLMEYPEAITRLVTG | | AGCGGACCTACGAGACATTCAAGAGCATCA |
| | | AQRPPDPAPAPLGAPGLPNGLLSGDEDFSS | | TGAAGAAGTCCCCATTCAGCGGCCCCACCG |
| | | IADMDFSALLGSGSGSRDSREGMFLPKPEA | | ATCCTAGACCTCCACCTAGAAGAATCGCCGT |
| | | GSAISDVFEGREVCQPKRIRPFHPPGSPWA | | GCCTAGCAGATCTAGCGCCTCCGTGCCTAAA |
| | | NRPLPASLAPTPTGPVHEPVGSLTPAPVPQ | | CCTGCTCCTCAGCCTTATCCTTTCACCAGCA |
| | | PLDPAPAVTPEASHLLEDPDEETSQAVKAL | | GCCTGAGCACCATCAACTACGACGAGTTCC |
| | | REMADTVIPQKEEAAICGQMDLSHPPPRG | | CTACCATGGTGTTCCCCAGCGGCCAGATCTC |
| | | HLDELTTTLESMTEDLNLDSPLTPELNEIL | | TCAGGCTTCTGCTCTTGCTCCAGCTCCTCCTC |
| | | DTFLNDECLLHAMHISTGLSIFDTSLF | | AGGTTCTGCCTCAAGCTCCTGCACCAGCACC |
| | | | | GGCTCCAGCTATGGTTTCTGCTTTGGCTCAG |
| | | | | GCCCCTGCTCCTGTGCCTGTTCTTGCTCCTG |
| | | | | GACCACCTCAGGCTGTTGCTCCTCCTGCTCC |
| | | | | AAAACCTACACAGGCCGGCGAAGGCACACT |
| | | | | GTCTGAAGCTCTGCTGCAGCTCCAGTTCGAT |
| | | | | GACGAAGATCTGGGCGCCCTGCTGGGCAAT |
| | | | | TCTACAGATCCTGCCGTGTTTACCGATCTGG |
| | | | | CCAGCGTGGACAACAGCGAGTTTCAGCAGC |
| | | | | TCCTGAATCAGGGCATCCCTGTGGCTCCTCA |
| | | | | CACCACCGAACCTATGCTGATGGAATACCC |
| | | | | CGAGGCCATCACCAGACTGGTCACCGGTGC |
| | | | | TCAAAGACCACCTGATCCAGCTCCAGCACC |
| | | | | ACTGGGAGCACCTGGACTGCCTAATGGACT |
| | | | | GCTGTCTGGCGACGAGGACTTCAGCTCTATC |
| | | | | GCCGACATGGATTTCTCTGCCCTGCTCGGCT |
| | | | | CTGGCAGCGGCTCTAGAGATAGCAGAGAAG |
| | | | | GCATGTTCCTGCCTAAGCCTGAGGCCGGCTC |
| | | | | TGCCATCTCCGATGTGTTCGAGGGAAGAGA |
| | | | | AGTGTGCCAGCCTAAGCGGATCCGGCCTTTT |
| | | | | CACCCTCCTGGAAGCCCTTGGGCCAACAGA |
| | | | | CCTCTGCCTGCTTCTCTGGCCCCTACACCAA |
| | | | | CAGGACCTGTGCACGAACCTGTGGGCAGTC |
| | | | | TGACCCCAGCTCCTGTTCCTCAACCTCTGGA |
| | | | | TCCCGCTCCTGCTGTGACACCTGAAGCCTCT |
| | | | | CATCTGCTGGAAGATCCCGACGAAGAGACA |
| | | | | AGCCAGGCCGTGAAGGCCCTGAGAGAAATG |
| | | | | GCCGACACAGTGATCCCTCAGAAAGAGGAA |
| | | | | GCCGCCATCTGCGGACAGATGGACCTGTCTC |
| | | | | ATCCTCCACCAAGAGGCCACCTGGACGAGC |
| | | | | TGACAACCACACTGGAATCCATGACCGAGG |
| | | | | ACCTGAACCTGGACAGCCCTCTGACACCCG |
| | | | | AGCTGAACGAGATCCTGGACACCTTCCTGA |
| | | | | ACGACGAGTGTCTGCTGCACGCCATGCACA |
| | | | | TCTCTACCGGCCTGAGCATCTTCGACACCAG |
| | | | | CCTGTTT |
| XIAP (with | 56 | SRGSEFMTFNSFEGSKTCVPADINKEEEFV | 83 | TCTAGAGGATCCGAATTCATGACTTTTAACA |
| N-terminal | | EEFNRLKTFANFPSGSPVSASTLARAGFLY | | GTTTTGAAGGATCTAAAACTTGTGTACCTGC |
| modification) | | TGEGDTVRCFSCHAAVDRWQYGDSAVGR | | AGACATCAATAAGGAAGAAGAATTTGTAGA |
| | | HRKVSPNCRFINGFYLENSATQSTNSGIQN | | AGAGTTTAATAGATTAAAAACTTTTGCTAAT |
| | | GQYKVENYLGSRDHFALDRPSETHADYLL | | TTTCCAAGTGGTAGTCCTGTTTCAGCATCAA |
| | | RTGQVVDISDTIYPRNPAMYSEEARLKSFQ | | CACTGGCACGAGCAGGGTTTCTTTATACTGG |
| | | NWPDYAHLTPRELASAGLYYTGIGDQVQ | | TGAAGGAGATACCGTGCGGTGCTTTAGTTGT |
| | | CFCCGGKLKNWEPCDRAWSEHRRHFPNC | | CATGCAGCTGTAGATAGGTGGCAATATGGA |
| | | FFVLGRNLNIRSESDAVSSDRNFPNSTNLP | | GACTCAGCAGTTGGAAGACACAGGAAAGTA |
| | | RNPSMADYEARIFTFGTWIYSVNKEQLAR | | TCCCCAAATTGCAGATTTATCAACGGCTTTT |
| | | AGFYALGEGDKVKCFHCGGGLTDWKPSE | | ATCTTGAAAATAGTGCCACGCAGTCTACAA |
| | | DPWEQHAKWYPGCKYLLEQKGQEYINNI | | ATTCTGGTATCCAGAATGGTCAGTACAAAGT |
| | | HLTHSLEECLVRTTEKTPSLTRRIDDTIFQN | | TGAAAACTATCTGGGAAGCAGAGATCATTT |
| | | PMVQEAIRMGFSFKDIKKIMEEKIQISGSN | | TGCCTTAGACAGGCCATCTGAGACACATGC |
| | | YKSLEVLVADLVNAQKDSMQDESSQTSL | | AGACTATCTTTTGAGAACTGGGCAGGTTGTA |
| | | QKEISTEEQLRRLQEEKLCKICMDRNIAIVF | | GATATATCAGACACCATATACCCGAGGAAC |
| | | VPCGHLVTCKQCAEAVDKCPMCYTVITFK | | CCTGCCATGTATAGTGAAGAAGCTAGATTA |
| | | QKIFMS | | AAGTCCTTTCAGAACTGGCCAGACTATGCTC |
| | | | | ACCTAACCCCAAGAGAGTTAGCAAGTGCTG |
| | | | | GACTCTACTACACAGGTATTGGTGACCAAGT |
| | | | | GCAGTGCTTTTGTTGTGGTGGAAAACTGAAA |
| | | | | AATTGGGAACCTTGTGATCGTGCCTGGTCAG |
| | | | | AACACAGGCGACACTTTCCTAATTGCTTCTT |
| | | | | TGTTTTGGGCCGGAATCTTAATATTCGAAGT |
| | | | | GAATCTGATGCTGTGAGTTCTGATAGGAATT |
| | | | | TCCCAAATTCAACAAATCTTCCAAGAAATCC |
| | | | | ATCCATGGCAGATTATGAAGCACGGATCTTT |
| | | | | ACTTTTGGGACATGGATATACTCAGTTAACA |
| | | | | AGGAGCAGCTTGCAAGAGCTGGATTTTATG |
| | | | | CTTTAGGTGAAGGTGATAAAGTAAAGTGCT |
| | | | | TTCACTGTGGAGGAGGGCTAACTGATTGGA |
| | | | | AGCCCAGTGAAGACCCTTGGGAACAACATG |
| | | | | CTAAATGGTATCCAGGGTGCAAATATCTGTT |
| | | | | AGAACAGAAGGGACAAGAATATATAAACA |
| | | | | ATATTCATTTAACTCATTCACTTGAGGAGTG |
| | | | | TCTGGTAAGAACTACTGAGAAAACACCATC |
| | | | | ACTAACTAGAAGAATTGATGATACCATCTTC |
| | | | | CAAAATCCTATGGTACAAGAAGCTATACGA |
| | | | | ATGGGGTTCAGTTTCAAGGACATTAAGAAA |
| | | | | ATAATGGAGGAAAAAATTCAGATATCTGGG |
| | | | | AGCAACTATAAATCACTTGAGGTTCTGGTTG |
| | | | | CAGATCTAGTGAATGCTCAGAAAGACAGTA |
| | | | | TGCAAGATGAGTCAAGTCAGACTTCATTAC |
| | | | | AGAAAGAGATTAGTACTGAAGAGCAGCTAA |
| | | | | GGCGCCTGCAAGAGGAGAAGCTTTGCAAAA |
| | | | | TCTGTATGGATAGAAATATTGCTATCGTTTT |
| | | | | TGTTCCTTGTGGACATCTAGTCACTTGTAAA |
| | | | | CAATGTGCTGAAGCAGTTGACAAGTGTCCC |
| | | | | ATGTGCTACACAGTCATTACTTTCAAGCAAA |
| | | | | AAATTTTTATGTCT |
| ZF10-1 | 57 | SRPGERPFQCRICMRNFSRRHGLDRHTRTH | 84 | TCTAGACCCGGCGAAAGACCCTTCCAGTGC |
| | | TGEKPFQCRICMRNFSDHSSLKRHLRTHTG | | CGGATCTGCATGCGGAACTTCAGCAGAAGG |
| | | SQKPFQCRICMRNFSVRHNLTRHLRTHTG | | CACGGCCTGGACAGACACACCAGAACACAC |
| | | EKPFQCRICMRNFSDHSNLSRHLKTHTGSQ | | ACAGGCGAGAAGCCTTTCCAGTGTAGAATC |
| | | KPFQCRICMRNFSQRSSLVRHLRTHTGEKP | | TGTATGCGCAATTTCAGCGACCACAGCAGC |
| | | FQCRICMRNFSESGHLKRHLRTHLRGS | | CTGAAGCGGCACCTGAGAACCCATACCGGC |
| | | | | AGCCAGAAACCATTTCAATGCCGCATCTGTA |
| | | | | TGAGAAACTTCTCCGTGCGGCACAACCTGA |
| | | | | CCAGACACCTGAGGACACACACCGGGGAGA |
| | | | | AACCCTTTCAGTGCAGAATATGCATGAGGA |
| | | | | ATTTCTCCGACCACTCCAACCTGAGCCGCCA |
| | | | | CCTGAAAACTCACACCGGCTCTCAAAAGCC |
| | | | | ATTTCAGTGTCGTATATGTATGCGGAATTTT |
| | | | | TCCCAGCGGAGCAGCCTCGTGCGCCATCTG |
| | | | | AGGACTCATACTGGCGAAAAGCCCTTCCAA |
| | | | | TGTCGCATATGCATGCGCAACTTTAGCGAGT |
| | | | | CCGGCCACCTGAAGAGACATCTGCGGACAC |
| | | | | ACCTGAGAGGCTCT |
| XIAP | 107 | MTFNSFEGSKTCVPADINKEEEFVEEFNRL | 108 | ATGACTTTTAACAGTTTTGAAGGATCTAAAA |
| | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGGGLTDWKPSEDPWEQH | | CACGCAGTCTACAAATTCTGGTATCCAGAAT |
| | | AKWYPGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAGGAGG |
| | | | | GCTAACTGATTGGAAGCCCAGTGAAGACCC |
| | | | | TTGGGAACAACATGCTAAATGGTATCCAGG |
| | | | | GTGCAAATATCTGTTAGAACAGAAGGGACA |
| | | | | AGAATATATAAACAATATTCATTTAACTCAT |
| | | | | TCACTTGAGGAGTGTCTGGTAAGAACTACTG |
| | | | | AGAAAACACCATCACTAACTAGAAGAATTG |
| | | | | ATGATACCATCTTCCAAAATCCTATGGTACA |
| | | | | AGAAGCTATACGAATGGGGTTCAGTTTCAA |
| | | | | GGACATTAAGAAAATAATGGAGGAAAAAAT |
| | | | | TCAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| XIAP (T308S, | 109 | MTFNSFEGSKTCVPADINKEEEFVEEFNRL | 110 | ATGACTTTTAACAGTTTTGAAGGATCTAAAA |
| G306S, | | KTFANFPSGSPVSASTLARAGFLYTGEGDT | | CTTGTGTACCTGCAGACATCAATAAGGAAG |
| G305M, | | VRCFSCHAAVDRWQYGDSAVGRHRKVSP | | AAGAATTTGTAGAAGAGTTTAATAGATTAA |
| P325S) | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGMSLSDWKPSEDPWEQH | | CACGCAGTCTACAAATTCTGGTATCCAGAAT |
| | | AKWYSGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAatgagcCT |
| | | | | AagcGATTGGAAGCCCAGTGAAGACCCTTGG |
| | | | | GAACAACATGCTAAATGGTATagcGGGTGCA |
| | | | | AATATCTGTTAGAACAGAAGGGACAAGAAT |
| | | | | ATATAAACAATATTCATTTAACTCATTCACT |
| | | | | TGAGGAGTGTCTGGTAAGAACTACTGAGAA |
| | | | | AACACCATCACTAACTAGAAGAATTGATGA |
| | | | | TACCATCTTCCAAAATCCTATGGTACAAGAA |
| | | | | GCTATACGAATGGGGTTCAGTTTCAAGGAC |
| | | | | ATTAAGAAAATAATGGAGGAAAAAATTCAG |
| | | | | ATATCTGGGAGCAACTATAAATCACTTGAG |
| | | | | GTTCTGGTTGCAGATCTAGTGAATGCTCAGA |
| | | | | AAGACAGTATGCAAGATGAGTCAAGTCAGA |
| | | | | CTTCATTACAGAAAGAGATTAGTACTGAAG |
| | | | | AGCAGCTAAGGCGCCTGCAAGAGGAGAAGC |
| | | | | TTTGCAAAATCTGTATGGATAGAAATATTGC |
| | | | | TATCGTTTTTGTTCCTTGTGGACATCTAGTCA |
| | | | | CTTGTAAACAATGTGCTGAAGCAGTTGACA |
| | | | | AGTGTCCCATGTGCTACACAGTCATTACTTT |
| | | | | CAAGCAAAAAATTTTTATGTCT |
| XIAP | 111 | MTFNSFEGSKTCVPADINKEEEFVEEFNRL | 112 | ATGACTTTTAACAGTTTTGAAGGATCTAAAA |
| (T308D, | | KTFANFPSGSPVSASTLARAGFLYTGEGDT | | CTTGTGTACCTGCAGACATCAATAAGGAAG |
| G306S, | | VRCFSCHAAVDRWQYGDSAVGRHRKVSP | | AAGAATTTGTAGAAGAGTTTAATAGATTAA |
| G305M, | | NCRFINGFYLENSATQSTNSGIQNGQYKVE | | AAACTTTTGCTAATTTTCCAAGTGGTAGTCC |
| P325S) | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGMSLDDWKPSEDPWEQH | | CACGCAGTCTACAAATTCTGGTATCCAGAAT |
| | | AKWYSGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAatgagcCT |
| | | | | AgacGATTGGAAGCCCAGTGAAGACCCTTGG |
| | | | | GAACAACATGCTAAATGGTATagcGGGTGCA |
| | | | | AATATCTGTTAGAACAGAAGGGACAAGAAT |
| | | | | ATATAAACAATATTCATTTAACTCATTCACT |
| | | | | TGAGGAGTGTCTGGTAAGAACTACTGAGAA |
| | | | | AACACCATCACTAACTAGAAGAATTGATGA |
| | | | | TACCATCTTCCAAAATCCTATGGTACAAGAA |
| | | | | GCTATACGAATGGGGTTCAGTTTCAAGGAC |
| | | | | ATTAAGAAAATAATGGAGGAAAAAATTCAG |
| | | | | ATATCTGGGAGCAACTATAAATCACTTGAG |
| | | | | GTTCTGGTTGCAGATCTAGTGAATGCTCAGA |
| | | | | AAGACAGTATGCAAGATGAGTCAAGTCAGA |
| | | | | CTTCATTACAGAAAGAGATTAGTACTGAAG |
| | | | | AGCAGCTAAGGCGCCTGCAAGAGGAGAAGC |
| | | | | TTTGCAAAATCTGTATGGATAGAAATATTGC |
| | | | | TATCGTTTTTGTTCCTTGTGGACATCTAGTCA |
| | | | | CTTGTAAACAATGTGCTGAAGCAGTTGACA |
| | | | | AGTGTCCCATGTGCTACACAGTCATTACTTT |
| | | | | CAAGCAAAAAATTTTTATGTCT |
| XIAP (T308S) | 113 | MTFNSFEGSKTCVPADINKEEEFVEEFNRL | 114 | ATGACTTTTAACAGTTTTGAAGGATCTAAAA |
| | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGGGLSDWKPSEDPWEQH | | CACGCAGTCTACAAATTCTGGTATCCAGAAT |
| | | AKWYPGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAGGAGG |
| | | | | GCTAagcGATTGGAAGCCCAGTGAAGACCCT |
| | | | | TGGGAACAACATGCTAAATGGTATCCAGGG |
| | | | | TGCAAATATCTGTTAGAACAGAAGGGACAA |
| | | | | GAATATATAAACAATATTCATTTAACTCATT |
| | | | | CACTTGAGGAGTGTCTGGTAAGAACTACTG |
| | | | | AGAAAACACCATCACTAACTAGAAGAATTG |
| | | | | ATGATACCATCTTCCAAAATCCTATGGTACA |
| | | | | AGAAGCTATACGAATGGGGTTCAGTTTCAA |
| | | | | GGACATTAAGAAAATAATGGAGGAAAAAAT |
| | | | | TCAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| XIAP | 115 | MTFNSFEGSKTCVPADINKEEEFVEEFNRL | 116 | ATGACTTTTAACAGTTTTGAAGGATCTAAAA |
| (T308D) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGGGLDDWKPSEDPWEQH | | CACGCAGTCTACAAATTCTGGTATCCAGAAT |
| | | AKWYPGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAGGAGG |
| | | | | GCTAgacGATTGGAAGCCCAGTGAAGACCCT |
| | | | | TGGGAACAACATGCTAAATGGTATCCAGGG |
| | | | | TGCAAATATCTGTTAGAACAGAAGGGACAA |
| | | | | GAATATATAAACAATATTCATTTAACTCATT |
| | | | | CACTTGAGGAGTGTCTGGTAAGAACTACTG |
| | | | | AGAAAACACCATCACTAACTAGAAGAATTG |
| | | | | ATGATACCATCTTCCAAAATCCTATGGTACA |
| | | | | AGAAGCTATACGAATGGGGTTCAGTTTCAA |
| | | | | GGACATTAAGAAAATAATGGAGGAAAAAAT |
| | | | | TCAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| XIAP | 117 | MTFNSFEGSKTCVPADINKEEEFVEEFNRL | 118 | ATGACTTTTAACAGTTTTGAAGGATCTAAAA |
| (G306S) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGGSLTDWKPSEDPWEQH | | CACGCAGTCTACAAATTCTGGTATCCAGAAT |
| | | AKWYPGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAGGAagc |
| | | | | CTAACTGATTGGAAGCCCAGTGAAGACCCT |
| | | | | TGGGAACAACATGCTAAATGGTATCCAGGG |
| | | | | TGCAAATATCTGTTAGAACAGAAGGGACAA |
| | | | | GAATATATAAACAATATTCATTTAACTCATT |
| | | | | CACTTGAGGAGTGTCTGGTAAGAACTACTG |
| | | | | AGAAAACACCATCACTAACTAGAAGAATTG |
| | | | | ATGATACCATCTTCCAAAATCCTATGGTACA |
| | | | | AGAAGCTATACGAATGGGGTTCAGTTTCAA |
| | | | | GGACATTAAGAAAATAATGGAGGAAAAAAT |
| | | | | TCAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| XIAP | 119 | MTFNSFEGSKTCVPADINKEEEFVEEFNRL | 120 | ATGACTTTTAACAGTTTTGAAGGATCTAAAA |
| (G305M) | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGMGLTDWKPSEDPWEQ | | CACGCAGTCTACAAATTCTGGTATCCAGAAT |
| | | HAKWYPGCKYLLEQKGQEYINNIHLTHSL | | GGTCAGTACAAAGTTGAAAACTATCTGGGA |
| | | EECLVRTTEKTPSLTRRIDDTIFQNPMVQE | | AGCAGAGATCATTTTGCCTTAGACAGGCCAT |
| | | AIRMGFSFKDIKKIMEEKIQISGSNYKSLEV | | CTGAGACACATGCAGACTATCTTTTGAGAAC |
| | | LVADLVNAQKDSMQDESSQTSLQKEISTE | | TGGGCAGGTTGTAGATATATCAGACACCAT |
| | | EQLRRLQEEKLCKICMDRNIAIVFVPCGHL | | ATACCCGAGGAACCCTGCCATGTATAGTGA |
| | | VTCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAatgGGGC |
| | | | | TAACTGATTGGAAGCCCAGTGAAGACCCTT |
| | | | | GGGAACAACATGCTAAATGGTATCCAGGGT |
| | | | | GCAAATATCTGTTAGAACAGAAGGGACAAG |
| | | | | AATATATAAACAATATTCATTTAACTCATTC |
| | | | | ACTTGAGGAGTGTCTGGTAAGAACTACTGA |
| | | | | GAAAACACCATCACTAACTAGAAGAATTGA |
| | | | | TGATACCATCTTCCAAAATCCTATGGTACAA |
| | | | | GAAGCTATACGAATGGGGTTCAGTTTCAAG |
| | | | | GACATTAAGAAAATAATGGAGGAAAAAATT |
| | | | | CAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| XIAP (P325S) | 121 | MTFNSFEGSKTCVPADINKEEEFVEEFNRL | 122 | ATGACTTTTAACAGTTTTGAAGGATCTAAAA |
| | | KTFANFPSGSPVSASTLARAGFLYTGEGDT | | CTTGTGTACCTGCAGACATCAATAAGGAAG |
| | | VRCFSCHAAVDRWQYGDSAVGRHRKVSP | | AAGAATTTGTAGAAGAGTTTAATAGATTAA |
| | | NCRFINGFYLENSATQSTNSGIQNGQYKVE | | AAACTTTTGCTAATTTTCCAAGTGGTAGTCC |
| | | NYLGSRDHFALDRPSETHADYLLRTGQVV | | TGTTTCAGCATCAACACTGGCACGAGCAGG |
| | | DISDTIYPRNPAMYSEEARLKSFQNWPDY | | GTTTCTTTATACTGGTGAAGGAGATACCGTG |
| | | AHLTPRELASAGLYYTGIGDQVQCFCCGG | | CGGTGCTTTAGTTGTCATGCAGCTGTAGATA |
| | | KLKNWEPCDRAWSEHRRHFPNCFFVLGR | | GGTGGCAATATGGAGACTCAGCAGTTGGAA |
| | | NLNIRSESDAVSSDRNFPNSTNLPRNPSMA | | GACACAGGAAAGTATCCCCAAATTGCAGAT |
| | | DYEARIFTFGTWIYSVNKEQLARAGFYAL | | TTATCAACGGCTTTTATCTTGAAAATAGTGC |
| | | GEGDKVKCFHCGGGLTDWKPSEDPWEQH | | CACGCAGTCTACAAATTCTGGTATCCAGAAT |
| | | AKWYSGCKYLLEQKGQEYINNIHLTHSLE | | GGTCAGTACAAAGTTGAAAACTATCTGGGA |
| | | ECLVRTTEKTPSLTRRIDDTIFQNPMVQEAI | | AGCAGAGATCATTTTGCCTTAGACAGGCCAT |
| | | RMGFSFKDIKKIMEEKIQISGSNYKSLEVL | | CTGAGACACATGCAGACTATCTTTTGAGAAC |
| | | VADLVNAQKDSMQDESSQTSLQKEISTEE | | TGGGCAGGTTGTAGATATATCAGACACCAT |
| | | QLRRLQEEKLCKICMDRNIAIVFVPCGHLV | | ATACCCGAGGAACCCTGCCATGTATAGTGA |
| | | TCKQCAEAVDKCPMCYTVITFKQKIFMS | | AGAAGCTAGATTAAAGTCCTTTCAGAACTG |
| | | | | GCCAGACTATGCTCACCTAACCCCAAGAGA |
| | | | | GTTAGCAAGTGCTGGACTCTACTACACAGGT |
| | | | | ATTGGTGACCAAGTGCAGTGCTTTTGTTGTG |
| | | | | GTGGAAAACTGAAAAATTGGGAACCTTGTG |
| | | | | ATCGTGCCTGGTCAGAACACAGGCGACACT |
| | | | | TTCCTAATTGCTTCTTTGTTTTGGGCCGGAAT |
| | | | | CTTAATATTCGAAGTGAATCTGATGCTGTGA |
| | | | | GTTCTGATAGGAATTTCCCAAATTCAACAAA |
| | | | | TCTTCCAAGAAATCCATCCATGGCAGATTAT |
| | | | | GAAGCACGGATCTTTACTTTTGGGACATGGA |
| | | | | TATACTCAGTTAACAAGGAGCAGCTTGCAA |
| | | | | GAGCTGGATTTTATGCTTTAGGTGAAGGTGA |
| | | | | TAAAGTAAAGTGCTTTCACTGTGGAGGAGG |
| | | | | GCTAACTGATTGGAAGCCCAGTGAAGACCC |
| | | | | TTGGGAACAACATGCTAAATGGTATagcGGG |
| | | | | TGCAAATATCTGTTAGAACAGAAGGGACAA |
| | | | | GAATATATAAACAATATTCATTTAACTCATT |
| | | | | CACTTGAGGAGTGTCTGGTAAGAACTACTG |
| | | | | AGAAAACACCATCACTAACTAGAAGAATTG |
| | | | | ATGATACCATCTTCCAAAATCCTATGGTACA |
| | | | | AGAAGCTATACGAATGGGGTTCAGTTTCAA |
| | | | | GGACATTAAGAAAATAATGGAGGAAAAAAT |
| | | | | TCAGATATCTGGGAGCAACTATAAATCACTT |
| | | | | GAGGTTCTGGTTGCAGATCTAGTGAATGCTC |
| | | | | AGAAAGACAGTATGCAAGATGAGTCAAGTC |
| | | | | AGACTTCATTACAGAAAGAGATTAGTACTG |
| | | | | AAGAGCAGCTAAGGCGCCTGCAAGAGGAGA |
| | | | | AGCTTTGCAAAATCTGTATGGATAGAAATAT |
| | | | | TGCTATCGTTTTTGTTCCTTGTGGACATCTAG |
| | | | | TCACTTGTAAACAATGTGCTGAAGCAGTTGA |
| | | | | CAAGTGTCCCATGTGCTACACAGTCATTACT |
| | | | | TTCAAGCAAAAAATTTTTATGTCT |
| Caspase-9 | 123 | MDEADRRLLRRCRLRLVEELQVDQLWDV | 124 | ATGGACGAAGCGGATCGGCGGCTCCTGCGG |
| (inclusive of | | LLSRELFRPHMIEDIQRAGSGSRRDQARQL | | CGGTGCCGGCTGCGGCTGGTGGAAGAGCTG |
| start codon | | IIDLETRGSQALPLFISCLEDTGQDMLASFL | | CAGGTGGACCAGCTCTGGGACGTCCTGCTG |
| and C- | | RTNRQAAKLSKPTLENLTPVVLRPEIRKPE | | AGCCGCGAGCTGTTCAGGCCCCATATGATC |
| terminal 6X | | VLRPETPRPVDIGSGGFGDVGALESLRGNA | | GAGGACATCCAGCGGGCAGGCTCTGGATCT |
| histidine tag) | | DLAYILSMEPCGHCLIINNVNFCRESGLRT | | CGGCGGGATCAGGCCAGGCAGCTGATCATA |
| | | RTGSNIDCEKLRRRFSSLHFMVEVKGDLT | | GATCTGGAGACTCGAGGGAGTCAGGCTCTT |
| | | AKKMVLALLELARQDHGALDCCVVVILS | | CCTTTGTTCATCTCCTGCTTAGAGGACACAG |
| | | HGCQASHLQFPGAVYGTDGCPVSVEKIVN | | GCCAGGACATGCTGGCTTCGTTTCTGCGAAC |
| | | IFNGTSCPSLGGKPKLFFIQACGGEQKDHG | | TAACAGGCAAGCAGCAAAGTTGTCGAAGCC |
| | | FEVASTSPEDESPGSNPEPDATPFQEGLRTF | | AACCCTAGAAAACCTTACCCCAGTGGTGCTC |
| | | DOLDAISSLPTPSDIFVSYSTFPGFVSWRDP | | AGACCAGAGATTCGCAAACCAGAGGTTCTC |
| | | KSGSWYVETLDDIFEQWAHSEDLQSLLLR | | AGACCGGAAACACCCAGACCAGTGGACATT |
| | | VANAVSVKGIYKQMPGCFNFLRKKLFFKT | | GGTTCTGGAGGATTCGGTGATGTCGGTGCTC |
| | | SHHHHHH | | TTGAGAGTTTGAGGGGAAATGCAGATTTGG |
| | | | | CTTACATCCTGAGCATGGAGCCCTGTGGCCA |
| | | | | CTGCCTCATTATCAACAATGTGAACTTCTGC |
| | | | | CGTGAGTCCGGGCTCCGCACCCGCACTGGCT |
| | | | | CCAACATCGACTGTGAGAAGTTGCGGCGTC |
| | | | | GCTTCTCCTCGCTGCATTTCATGGTGGAGGT |
| | | | | GAAGGGCGACCTGACTGCCAAGAAAATGGT |
| | | | | GCTGGCTTTGCTGGAGCTGGCGCGGCAGGA |
| | | | | CCACGGTGCTCTGGACTGCTGCGTGGTGGTC |
| | | | | ATTCTCTCTCACGGCTGTCAGGCCAGCCACC |
| | | | | TGCAGTTCCCAGGGGCTGTCTACGGCACAG |
| | | | | ATGGATGCCCTGTGTCGGTCGAGAAGATTGT |
| | | | | GAACATCTTCAATGGGACCAGCTGCCCCAG |
| | | | | CCTGGGAGGGAAGCCCAAGCTCTTTTTCATC |
| | | | | CAGGCCTGTGGTGGGGAGCAGAAAGACCAT |
| | | | | GGGTTTGAGGTGGCCTCCACTTCCCCTGAAG |
| | | | | ACGAGTCCCCTGGCAGTAACCCCGAGCCAG |
| | | | | ATGCCACCCCGTTCCAGGAAGGTTTGAGGA |
| | | | | CCTTCGACCAGCTGGACGCCATATCTAGTTT |
| | | | | GCCCACACCCAGTGACATCTTTGTGTCCTAC |
| | | | | TCTACTTTCCCAGGTTTTGTTTCCTGGAGGG |
| | | | | ACCCCAAGAGTGGCTCCTGGTACGTTGAGA |
| | | | | CCCTGGACGACATCTTTGAGCAGTGGGCTCA |
| | | | | CTCTGAAGACCTGCAGTCCCTCCTGCTTAGG |
| | | | | GTCGCTAATGCTGTTTCGGTGAAAGGGATTT |
| | | | | ATAAACAGATGCCTGGTTGCTTTAATTTCCT |
| | | | | CCGGAAAAAACTTTTCTTTAAAACATCACAC |
| | | | | CACCACCACCACCAC |
| iCasp-9 | 125 | MDVGALESLRGNADLAYILSMEPCGHCLII | 126 | ATGGATGTCGGTGCTCTTGAGAGTTTGAGGG |
| (inclusive of | | NNVNFCRESGLRTRTGSNIDCEKLRRRFSS | | GAAATGCAGATTTGGCTTACATCCTGAGCAT |
| start codon) | | LHFMVEVKGDLTAKKMVLALLELARQDH | | GGAGCCCTGTGGCCACTGCCTCATTATCAAC |
| | | GALDCCVVVILSHGCQASHLQFPGAVYGT | | AATGTGAACTTCTGCCGTGAGTCCGGGCTCC |
| | | DGCPVSVEKIVNIFNGTSCPSLGGKPKLFFI | | GCACCCGCACTGGCTCCAACATCGACTGTG |
| | | QACGGEQKDHGFEVASTSPEDESPGSNPEP | | AGAAGTTGCGGCGTCGCTTCTCCTCGCTGCA |
| | | DATPFQEGLRTFDQLDAISSLPTPSDIFVSY | | TTTCATGGTGGAGGTGAAGGGCGACCTGAC |
| | | STFPGFVSWRDPKSGSWYVETLDDIFEQW | | TGCCAAGAAAATGGTGCTGGCTTTGCTGGA |
| | | AHSEDLQSLLLRVANAVSVKGIYKQMPGC | | GCTGGCGCGGCAGGACCACGGTGCTCTGGA |
| | | FNFLRKKLFFKTS | | CTGCTGCGTGGTGGTCATTCTCTCTCACGGC |
| | | | | TGTCAGGCCAGCCACCTGCAGTTCCCAGGG |
| | | | | GCTGTCTACGGCACAGATGGATGCCCTGTGT |
| | | | | CGGTCGAGAAGATTGTGAACATCTTCAATG |
| | | | | GGACCAGCTGCCCCAGCCTGGGAGGGAAGC |
| | | | | CCAAGCTCTTTTTCATCCAGGCCTGTGGTGG |
| | | | | GGAGCAGAAAGACCATGGGTTTGAGGTGGC |
| | | | | CTCCACTTCCCCTGAAGACGAGTCCCCTGGC |
| | | | | AGTAACCCCGAGCCAGATGCCACCCCGTTC |
| | | | | CAGGAAGGTTTGAGGACCTTCGACCAGCTG |
| | | | | GACGCCATATCTAGTTTGCCCACACCCAGTG |
| | | | | ACATCTTTGTGTCCTACTCTACTTTCCCAGGT |
| | | | | TTTGTTTCCTGGAGGGACCCCAAGAGTGGCT |
| | | | | CCTGGTACGTTGAGACCCTGGACGACATCTT |
| | | | | TGAGCAGTGGGCTCACTCTGAAGACCTGCA |
| | | | | GTCCCTCCTGCTTAGGGTCGCTAATGCTGTT |
| | | | | TCGGTGAAAGGGATTTATAAACAGATGCCT |
| | | | | GGTTGCTTTAATTTCCTCCGGAAAAAACTTT |
| | | | | TCTTTAAAACATCA |
| CRBN WT | 127 | MAGEGDQQDAAHNMGNHLPLLPAESEEE | 128 | ATGGCTGGGGAGGGAGATCAACAGGACGCA |
| | | DEMEVEDQDSKEAKKPNIINFDTSLPTSHT | | GCGCACAACATGGGTAACCATCTTCCACTCC |
| | | YLGADMEEFHGRTLHDDDSCQVIPVLPQV | | TCCCGGCTGAGAGCGAGGAGGAGGATGAAA |
| | | MMILIPGQTLPLQLFHPQEVSMVRNLIQKD | | TGGAGGTTGAGGATCAGGATTCCAAAGAGG |
| | | RTFAVLAYSNVQEREAQFGTTAEIYAYRE | | CAAAGAAGCCAAATATCATCAACTTTGACA |
| | | EQDFGIEIVKVKAIGRQRFKVLELRTQSDG | | CATCCCTCCCCACTTCACATACATACCTTGG |
| | | IQQAKVQILPECVLPSTMSAVQLESLNKCQ | | GGCTGATATGGAGGAGTTTCATGGCCGCAC |
| | | IFPSKPVSREDQCSYKWWQKYQKRKFHC | | TCTCCACGACGATGACTCATGTCAAGTAATC |
| | | ANLTSWPRWLYSLYDAETLMDRIKKQLR | | CCGGTCCTCCCACAAGTGATGATGATTCTCA |
| | | EWDENLKDDSLPSNPIDFSYRVAACLPIDD | | TCCCCGGGCAAACACTCCCGCTCCAGCTGTT |
| | | VLRIQLLKIGSAIQRLRCELDIMNKCTSLCC | | CCACCCCCAAGAAGTAAGTATGGTCCGAAA |
| | | KQCQETEITTKNEIFSLSLCGPMAAYVNPH | | CCTTATCCAAAAAGATCGGACGTTCGCCGTT |
| | | GYVHETLTVYKACNLNLIGRPSTEHSWFP | | CTGGCGTACTCCAACGTACAAGAACGCGAA |
| | | GYAWTVAQCKICASHIGWKFTATKKDMS | | GCTCAGTTTGGCACGACCGCAGAAATATAC |
| | | PQKFWGLTRSALLPTIPDTEDEISPDKVILC | | GCCTACCGGGAGGAGCAAGATTTCGGAATA |
| | | L | | GAAATCGTGAAAGTGAAGGCAATTGGGAGA |
| | | | | CAGCGATTTAAAGTATTGGAATTGCGAACG |
| | | | | CAGAGCGATGGTATTCAACAGGCCAAAGTC |
| | | | | CAAATACTGCCGGAATGCGTGCTTCCGTCTA |
| | | | | CTATGAGCGCAGTGCAGTTGGAATCTTTGAA |
| | | | | CAAGTGCCAGATATTTCCAAGCAAGCCAGT |
| | | | | ATCTCGGGAGGACCAATGTAGCTATAAGTG |
| | | | | GTGGCAAAAGTACCAAAAGCGCAAATTCCA |
| | | | | CTGTGCAAACCTGACTTCTTGGCCCAGATGG |
| | | | | CTGTATTCCTTGTACGATGCGGAGACTTTGA |
| | | | | TGGATAGAATTAAAAAGCAACTGCGAGAAT |
| | | | | GGGACGAAAACCTGAAAGACGACTCACTTC |
| | | | | CGAGTAATCCAATCGATTTCTCATACCGGGT |
| | | | | GGCTGCTTGTTTGCCCATAGATGACGTGTTG |
| | | | | CGGATCCAGCTGCTTAAAATAGGATCCGCC |
| | | | | ATACAACGGCTTCGGTGTGAGCTGGATATA |
| | | | | ATGAATAAGTGTACAAGCCTCTGTTGTAAGC |
| | | | | AATGTCAAGAGACCGAAATTACTACTAAGA |
| | | | | ATGAGATATTCTCTTTGTCACTTTGCGGACC |
| | | | | TATGGCTGCCTACGTTAATCCACATGGGTAC |
| | | | | GTGCACGAGACTCTTACCGTGTATAAAGCCT |
| | | | | GTAATCTTAACCTTATAGGCAGGCCATCCAC |
| | | | | TGAACACAGTTGGTTCCCTGGTTATGCGTGG |
| | | | | ACGGTAGCACAGTGTAAAATTTGTGCATCTC |
| | | | | ACATCGGCTGGAAGTTTACGGCAACTAAAA |
| | | | | AGGACATGAGTCCCCAGAAGTTTTGGGGTC |
| | | | | TCACCCGATCCGCCCTTCTGCCGACCATCCC |
| | | | | AGATACCGAAGACGAAATCTCTCCAGATAA |
| | | | | AGTGATACTGTGTTTG |
| CRBN (del | 129 | MAGEGDQQDAAHNMGNHLPLLPAESEEE | 130 | ATGGCGGGCGAGGGCGATCAGCAAGACGCG |
| 194-247) | | DEMEVEDQDSKEAKKPNIINFDTSLPTSHT | | GCTCACAACATGGGAAATCACTTACCCTTAC |
| | | YLGADMEEFHGRTLHDDDSCQVIPVLPQV | | TTCCCGCAGAATCTGAAGAGGAAGATGAGA |
| | | MMILIPGQTLPLQLFHPQEVSMVRNLIQKD | | TGGAGGTAGAGGATCAGGACTCTAAAGAAG |
| | | RTFAVLAYSNVQEREAQFGTTAEIYAYRE | | CGAAAAAGCCTAATATCATCAACTTTGACA |
| | | EQDFGIEIVKVKAIGRQRFKVLELRTQSDG | | CCTCTCTTCCAACCAGTCACACTTACCTGGG |
| | | IQQAKVQILPECVLPSTYDAETLMDRIKKQ | | GGCAGACATGGAGGAATTCCATGGTCGAAC |
| | | LREWDENLKDDSLPSNPIDFSYRVAACLPI | | TCTCCACGACGACGATAGCTGCCAAGTGAT |
| | | DDVLRIQLLKIGSAIQRLRCELDIMNKCTS | | ACCAGTGCTCCCTCAGGTAATGATGATTCTT |
| | | LCCKQCQETEITTKNEIFSLSLCGPMAAYV | | ATTCCTGGACAGACCCTGCCTCTACAGCTGT |
| | | NPHGYVHETLTVYKACNLNLIGRPSTEHS | | TCCACCCTCAAGAGGTGAGCATGGTCAGGA |
| | | WFPGYAWTVAQCKICASHIGWKFTATKK | | ATTTGATCCAGAAGGACAGAACATTTGCGG |
| | | DMSPQKFWGLTRSALLPTIPDTEDEISPDK | | TCCTGGCCTACTCAAACGTACAGGAGCGAG |
| | | VILCL | | AGGCTCAGTTCGGGACCACTGCCGAAATAT |
| | | | | ACGCGTACCGGGAGGAGCAAGACTTCGGGA |
| | | | | TCGAAATCGTGAAGGTAAAGGCCATTGGCA |
| | | | | GACAACGGTTTAAGGTCCTTGAGCTCCGGA |
| | | | | CGCAGAGTGATGGGATACAACAGGCTAAAG |
| | | | | TGCAGATCCTACCAGAGTGTGTATTACCATC |
| | | | | TACCTATGATGCAGAGACTCTCATGGACCGC |
| | | | | ATAAAAAAGCAATTAAGGGAATGGGACGAA |
| | | | | AACCTCAAGGACGATTCACTCCCATCCAACC |
| | | | | CCATTGACTTCTCATATAGGGTCGCTGCTTG |
| | | | | TTTGCCTATCGACGACGTCCTTAGGATACAG |
| | | | | CTCCTGAAAATCGGAAGCGCAATACAAAGA |
| | | | | TTGCGCTGTGAGCTGGACATTATGAATAAGT |
| | | | | GCACTTCACTGTGTTGTAAGCAGTGTCAAGA |
| | | | | GACCGAAATCACTACGAAGAACGAGATCTT |
| | | | | CTCTCTCTCCCTCTGCGGGCCAATGGCAGCA |
| | | | | TATGTAAATCCGCATGGGTATGTTCATGAGA |
| | | | | CACTAACTGTGTACAAGGCCTGCAATTTAAA |
| | | | | CTTGATCGGCCGGCCATCTACAGAACACAG |
| | | | | TTGGTTCCCGGGTTATGCCTGGACAGTGGCA |
| | | | | CAGTGCAAAATTTGTGCTTCCCACATTGGAT |
| | | | | GGAAATTTACAGCCACAAAGAAGGATATGA |
| | | | | GTCCCCAAAAGTTCTGGGGACTGACCAGGA |
| | | | | GCGCTTTATTGCCCACCATCCCGGACACAGA |
| | | | | GGACGAAATCTCTCCGGACAAGGTGATTCT |
| | | | | CTGTCTG |
| d913 WT | 131 | MFNVLMVHKRSHTGERPLQCEICGFTCRQ | 132 | atgTTCAACGTGCTGATGGTGCACAAGCGGA |
| (inclusive of | | KGNLLRHIKLHTGEKPFKCHLCNYACQRR | | GCCACACCGGCGAAAGACCTCTGCAGTGTG |
| start codon; | | DAL | | AAATCTGCGGCTTCACCTGTCGGCAGAAGG |
| codon | | | | GCAACCTGCTGCGGCACATCAAACTGCACA |
| sequence | | | | CAGGCGAGAAGCCCTTCAAGTGCCACCTGT |
| version 2) | | | | GCAATTACGCCTGCCAGAGAAGAGATGCCC |
| | | | | TG |
| d913 (all Lys- | 133 | MFNVLMVHRRSHTGERPLQCEICGFTCRQ | 134 | atgTTCAACGTGCTGATGGTGCACAGGCGGA |
| Arg) | | RGNLLRHIRLHTGERPFRCHLCNYACQRR | | GCCACACCGGCGAAAGACCTCTGCAGTGTG |
| | | DAL | | AAATCTGCGGCTTCACCTGTCGGCAGAGGG |
| | | | | GCAACCTGCTGCGGCACATCAGACTGCACA |
| | | | | CAGGCGAGAGGCCCTTCAGGTGCCACCTGT |
| | | | | GCAATTACGCCTGCCAGAGAAGAGATGCCC |
| | | | | TG |
| Smac/ | 135 | MAALKSWLSRSVTSFFRYRQCLCVPVVAN | 136 | ATGGCCGCTCTGAAGTCCTGGCTGAGCAGA |
| DIABLO | | FKKRCFSELIRPWHKTVTIGFGVTLCAVPI | | AGCGTGACCAGCTTCTTCCGGTACAGACAGT |
| | | AQKSEPHSLSSEALMRRAVSLVTDSTSTFL | | GCCTGTGCGTGCCCGTGGTGGCCAACTTCAA |
| | | SQTTYALIEAITEYTKAVYTLTSLYRQYTS | | GAAGAGATGCTTCAGCGAGCTGATCAGACC |
| | | LLGKMNSEEEDEVWQVIIGARAEMTSKHQ | | CTGGCACAAGACCGTGACCATCGGCTTTGG |
| | | EYLKLETTWMTAVGLSEMAAEAAYQTGA | | CGTGACCCTGTGTGCCGTGCCTATCGCTCAG |
| | | DQASITARNHIQLVKLQVEEVHQLSRKAE | | AAGTCTGAGCCTCACAGCCTGTCTAGCGAG |
| | | TKLAEAQIEELRQKTQEEGEERAESEQEAY | | GCCCTTATGAGAAGGGCCGTGTCTCTGGTCA |
| | | LRED | | CCGACAGCACCAGCACATTTCTGAGCCAGA |
| | | | | CCACATACGCCCTGATCGAGGCCATCACCG |
| | | | | AGTACACCAAGGCCGTGTACACCCTGACCA |
| | | | | GCCTGTACCGGCAGTACACATCTCTGCTGGG |
| | | | | CAAGATGAACAGCGAGGAAGAGGACGAAG |
| | | | | TCTGGCAAGTGATCATCGGCGCCAGAGCCG |
| | | | | AGATGACCAGCAAGCACCAAGAGTACCTGA |
| | | | | AGCTGGAAACCACCTGGATGACAGCCGTGG |
| | | | | GCCTGTCTGAAATGGCCGCCGAAGCTGCTTA |
| | | | | TCAGACCGGCGCTGATCAGGCCAGCATCAC |
| | | | | CGCCAGAAATCACATCCAGCTGGTCAAGCT |
| | | | | GCAGGTCGAGGAAGTGCACCAGCTGTCCAG |
| | | | | AAAGGCCGAGACAAAGCTGGCTGAGGCCCA |
| | | | | GATCGAGGAACTGCGGCAGAAAACCCAAGA |
| | | | | GGAAGGCGAGGAAAGAGCCGAGTCTGAGC |
| | | | | AAGAGGCCTACCTGAGAGAGGAC |

### Example 2: IMiD Based Regulation of an Apoptotic Factor (Caspase-9)

Materials, Methods, and Assays

Cell engineering and assessment: 24 hours before transduction 50,000 U87MG cells (alone or a specified stable cell line expressing an anti-toxin construct “SB03213” or synthetic transcription factor (SynTF) repressor construct “SB03936” generated by lentiviral transduction) were plated on a 24-well dish. Next day, cells were transduced with 50,000 pg of virus (based on p24 titer) of construct encoding the cell death-inducing toxin Caspase-9 under the control of an activation-conditional control polypeptide (ACP)—responsive promoter (also referred to as SynTF-responsive promoter) with a P2A-linked mCherry. Transduced cells were split into drug free media or media with an immunomodulatory drug (IMiD) 1 uM of pomalidomide or 1 uM of iberdomide 48 hours after transduction. Expression of mCherry-tagged toxin was quantified by flow cytometry 48 hours after splitting into no drug/+ drug conditions. Lentivirus was generated in a modified LentiX cell line expressing a constitutive anti-toxin (XIAP) and transcription factor repressor to prevent toxin-payload induced death of virus production cells.

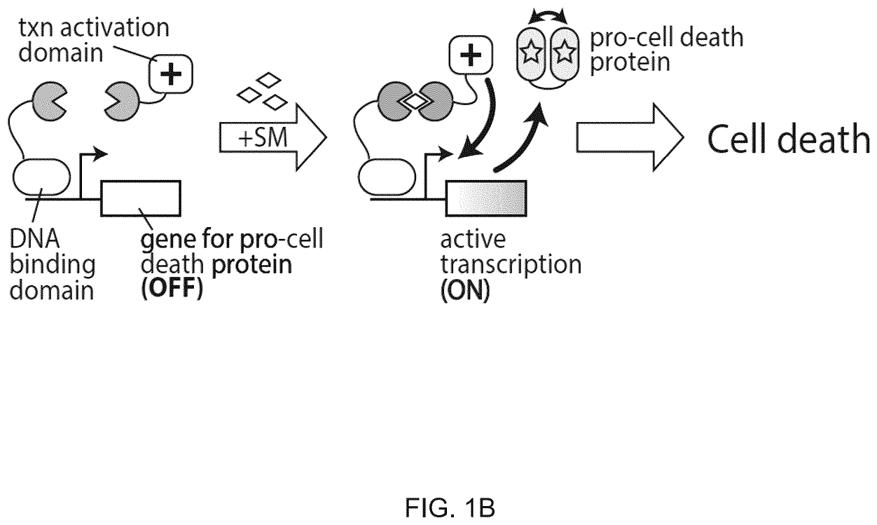

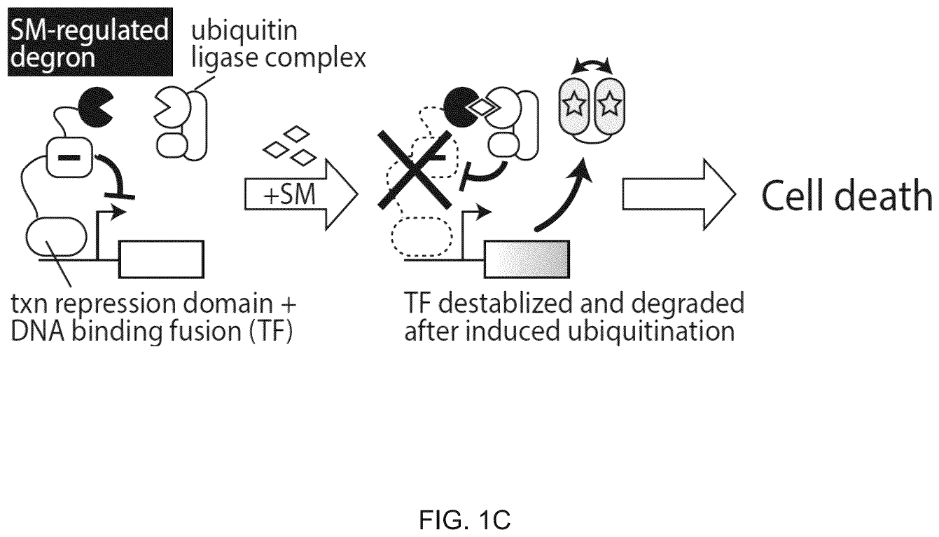

FIG. 2A shows the domains and organization of the constructs tested.

Results

Cells were generated (either as a stable cell-line or co-transduced) to express an ACP including a transcriptional repressor and/or the anti-toxin XIAP. Cells were then lentivirally transduced to express the cell-death inducing toxin Caspase-9. Both the ACP transcriptional repressor and the anti-toxin XIAP include a degron that in response to addition of an IMiD promotes ubiquitin pathway-mediated degradation of the peptides. Upon IMiD addition, degradation of the repressor leads to expression of pro-apoptotic Caspase-9 (see System 3; degron-tagged transcriptional repressor) and degradation of the pro-survival protein XIAP (System 4; degron-tagged pro-survival), as described in Example 1.

As shown in FIG. 2B, a U7MG cell line stably expressing the ACP (SB03936) has lower mCherry expression in all conditions indicating potent repression by the ACP; however, in the presence of drug a small increase in mCherry expression was observed, indicating that the ACP can regulate expression of an apoptotic factor.

### Example 3: Ligand-Induced Dimerization of an Inducible Cell Death System Using a Mifepristone-Based System

Materials, Methods, and Assays

Cell engineering and assessment: 24 hours before transduction 50,000 HEK293T cells were plated on a 24-well dish. Next day, cells were transduced with 50,000 pg of SB03080 (Progesterone receptor domain-Gly-Ser linker-iCasp9-IRES-red fluorescent protein) (based on p24 titer). Transduced cells were split into drug free media or media with 10 uM of Mifepristone 48 hours after transduction. 24 hours later, samples were stained for annexin V, a marker of apoptosis, and Sytox Red a live/dead stain and quantified by flow cytometry.

Results

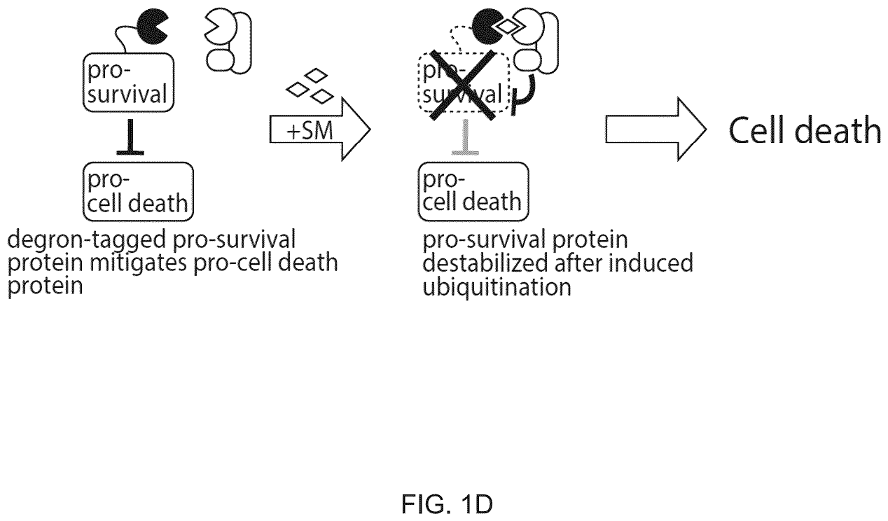

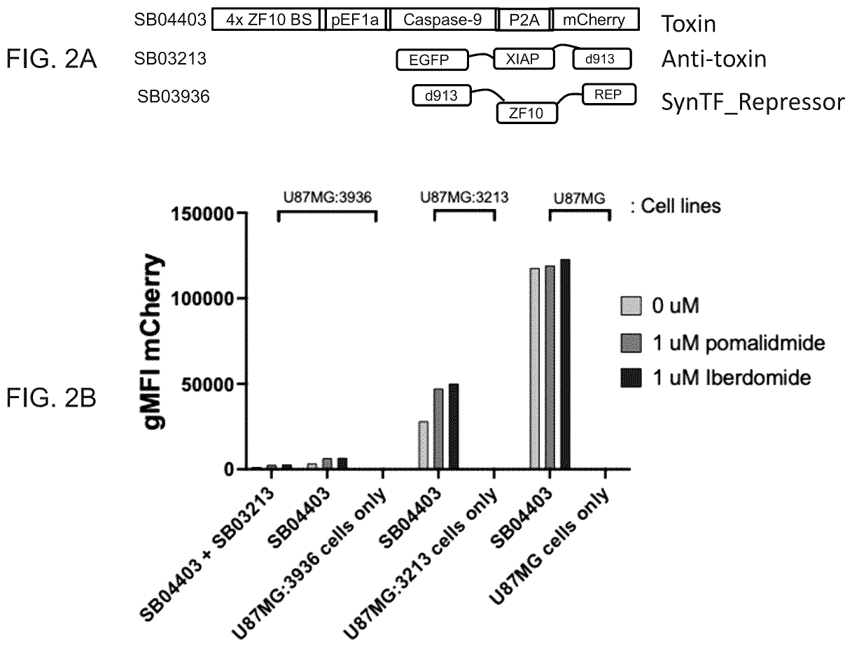

Cells were lentivirally transduced to express the toxin Caspase-9 with an IRES-red fluorescent protein (mKate) expressed under control of an SFFV promoter. The Caspase-9 protein includes a progesterone receptor domain. Upon Mifepristone addition, monomers of pro-apoptotic Caspase-9 oligomerize through binding of the progesterone receptor domain to Mifepristone (see FIG. 3A and System 1; chemical inducer of dimerization that activates the pro-cell death-inducing protein), as described in Example 1.

As shown in FIG. 3B, HEK293 cells engineered to express the Caspase-9/progesterone-receptor fusion demonstrated increased cell death upon addition of Mifepristone (both apoptotic and by live/dead). Accordingly, the results demonstrate control of cell-death through use of a chemical inducer of dimerization that activates the pro-cell death-inducing protein Caspase9.

### Example 4: Transcriptional Regulation of Constitutively Expressed Caspase-9 in HEK293T Cells

Materials, Methods, and Assays

Cell engineering and assessment: HEK293T cell lines stably expressing a synTF repressor or antitoxin, previously generated through lentiviral transduction, were seeded at 50,000 cells/well in 24-well plate and transfected with 50,000 pg of specified toxin constructs 24 hours later (see Table F). Cells were split into media only or 1 uM Pomalidomide conditions 2 days post transduction. Day 3 and Day 5 growth in media or media with 1 uM Pomalidomide, cells were harvested and stained with SytoxRed and Annexin V dye. Cell viability and apoptosis were quantified by flow cytometry.

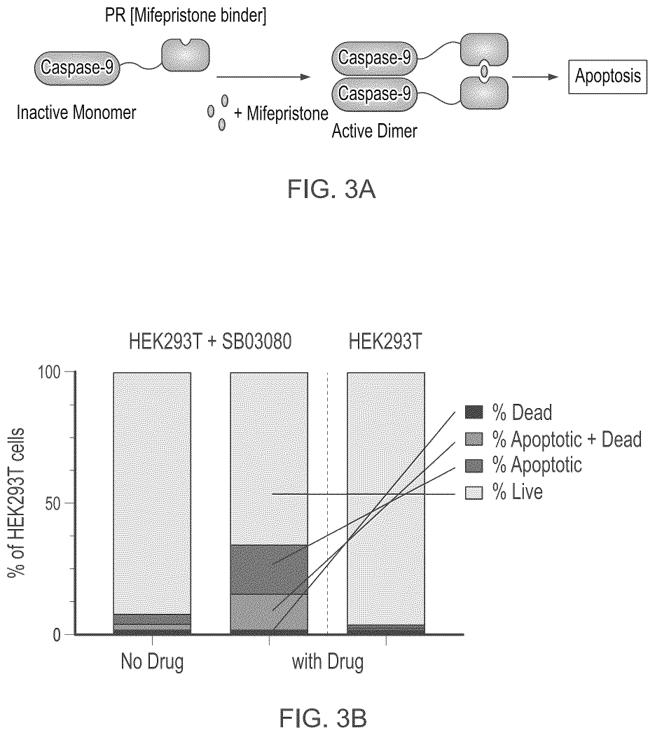

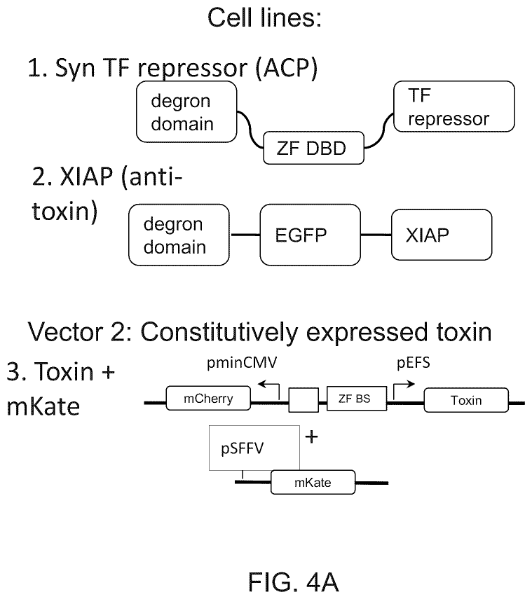

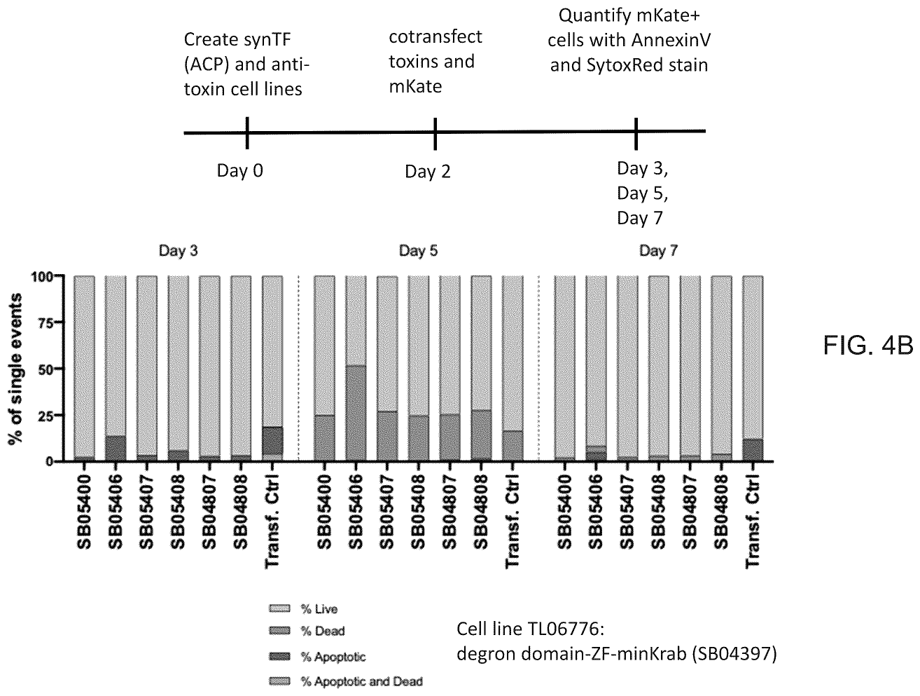

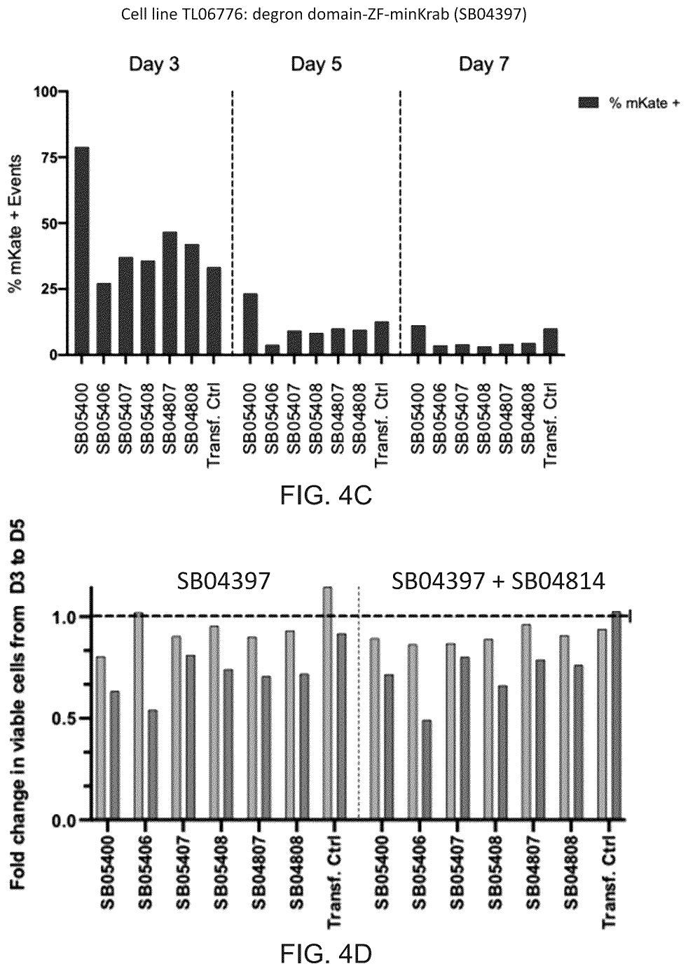

The HEK293T cell lines generated are shown in Table E below. The various cell-death inducing toxins are shown in Table F below. FIG. 4A shows the domains and organization of the constructs tested.

TABLE E
HEK293T cell lines expressing ACP (SynTF)
repressors and/or anti-toxins

| Cell Line | Construct | Description |
|---|---|---|
| TL06775 | SB04809 | degron domain-ZF-HDAC4 |
| TL06776 | SB04397 | degron domain-ZF-minKrab |
| TL06777 | SB04809 + | degron domain-ZF-HDAC4 + |
| | SB04814 | degron domain-XIAP |
| TL06778 | SB04397 + | degron domain-ZF-minKrab + |
| | SB04814 | degron domain-XIAP |

TABLE F
Cell-death inducing toxin constructs

| Construct | Description |
|---|---|
| SB05400 | 4xBS pEF1a: Casp9-P2A-mCherry |
| SB05406 | 4xBS pEF1a: hBax insulator inv pMinCMV: mCherry |
| SB05407 | 4xBS pEF1a: tBid insulator inv pMinCMV: mCherry |
| SB05408 | 4xBS pEF1a: Smac/DIABLO insulator inv |
| | pMinCMV: mCherry |
| SB04807 | 4xBS pEF1a: Casp9 insulator inv pMinCMV: mCherry |
| SB04808 | 4xBS pEF1a: Casp9 insulator inv pMinCMV: mCherry |

Results

Stable cell lines were generated to express an ACP including a transcriptional repressor and/or the anti-toxin XIAP. Cells were then lentivirally transduced to express the cell-death inducing toxin Caspase-9. Cells were also engineered to express mKate in order to quantify Casp9+ transduced cells. Both the ACP transcriptional repressor and the anti-toxin XIAP include a degron that in response to addition of an IMiD promotes ubiquitin pathway-mediated degradation of the peptides. Upon IMiD addition, degradation of the repressor leads to expression of pro-apoptotic Caspase-9 (see System 3; degron-tagged transcriptional repressor) and degradation of the pro-survival protein XIAP (System 4; degron-tagged pro-survival), as described in Example 1.

The cell line TL06776 was assessed following expression of degron domain-ZF-minKrab (SB04397). As shown in FIG. 4B, toxin activity appeared to peak at Day 5 and by Day 7 drop out of population. Further, the assay demonstrated the ability to assess the potential of a toxin as a suicide switch by comparing cell viability on Day 3 to Day 5. However, as shown in FIG. 4C, mKate expression was too transient to use as a way to gate on toxin+ population. Further, addition of XIAP had no observed impact on preventing progression of apoptosis. HDAC4 was also assessed in place of minKrab (data not shown).

Additional toxin constructs were assessed through calculating the switch function by quantifying viability of cells on day 5 as a ratio of viability of cells on day 3. Functionality of the suicide switches is indicated by the no drug condition being close to 1.0 fold change (FIG. 4D left columns), and 1 uM Pomalidomide treatment will result in decline in fraction of viable cells (FIG. 4D right columns). As shown in FIG. 4D, SB05406 (Bax) resulted in a 50% decline in the viable cell population demonstrating potential as a toxin in a suicide switch.

### Example 5: Screening Pro-Apoptotic Members of the Apoptotic Pathway for Toxicity in HEK293T Cells

Materials, Methods, and Assays

Cell engineering and assessment: 5,000 HEK293T cells were plated in a 96-well flat bottom TC treated plate. Cells were transduced same day with 5,000 pg of specified constructs (based on p24 titer). Plates were transferred to the Incucyte where images of the cell layer were captured at 4 images per well every 2 hours over the course of 11 days with 10× objective. Confluency was calculated using the Incucyte basic pipeline software to map phase overlay. Cells were split down to 5,000 cells per well on day 4.

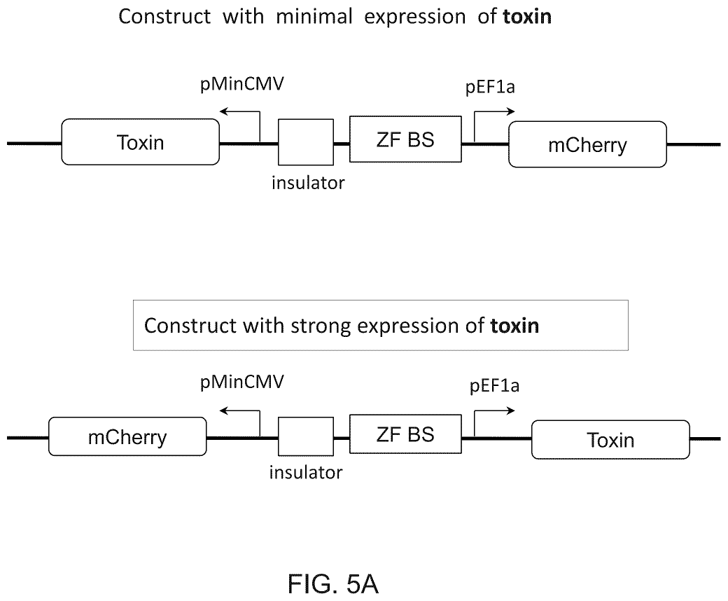

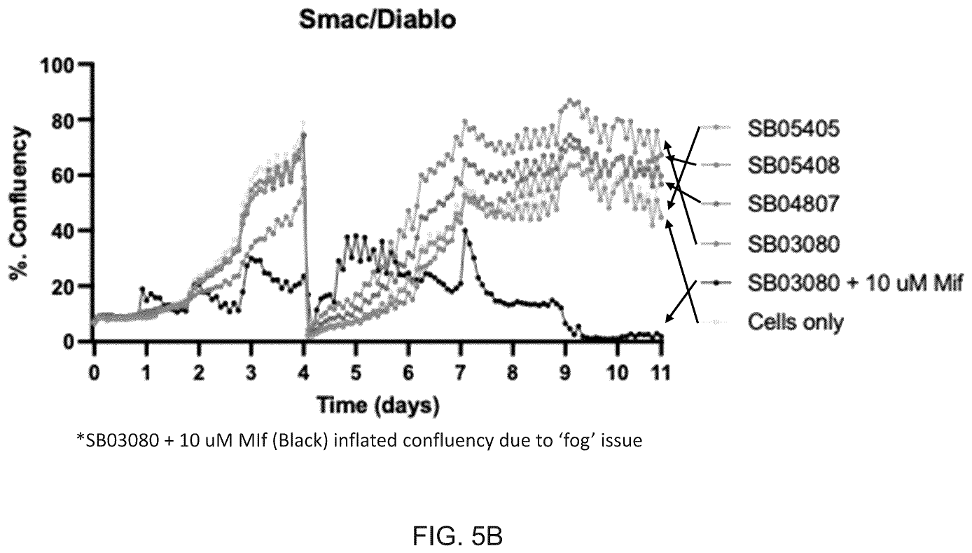

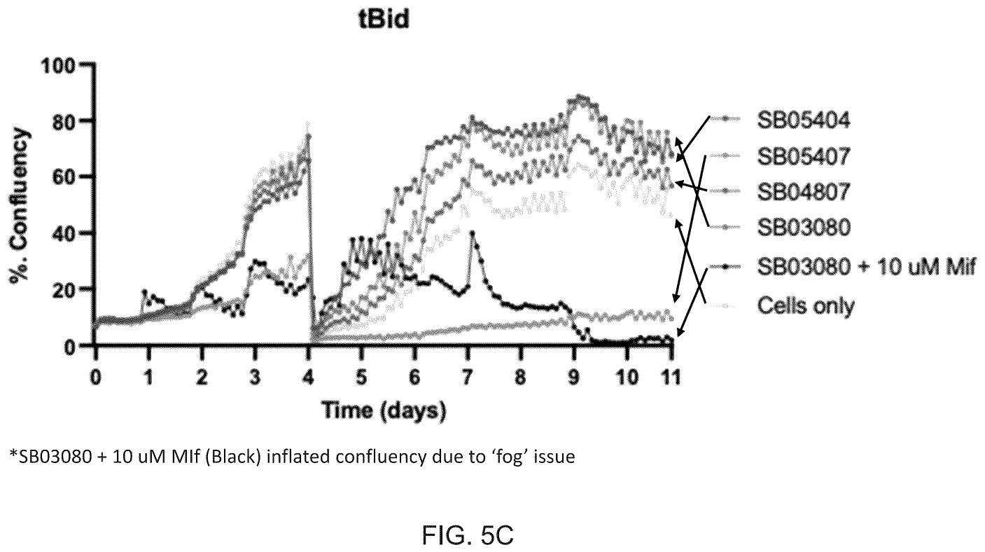

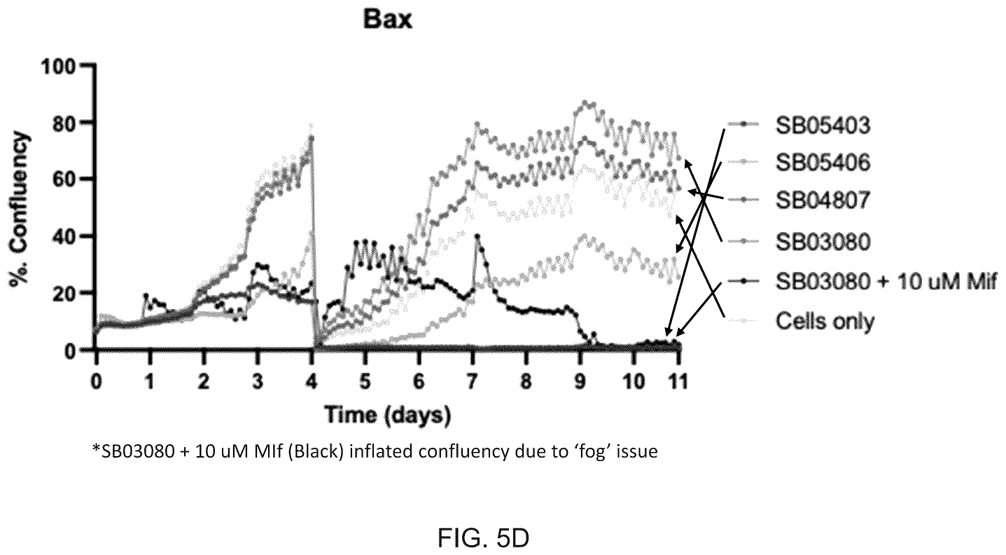

The various cell-death inducing toxins are shown in Table G below. FIG. 5A shows the domains and organization of the constructs tested.

TABLE G
Cell-death inducing toxin constructs

| Construct | Description |
|---|---|
| SB05403 | pMinCMV: BAX |
| SB05404 | pMinCMV: tBID |
| SB05405 | pMinCMV: Smac/DIABLO |
| SB05406 | pEF1a: BAX |
| SB05407 | pEF1a: tBID |
| SB05408 | pEF1a: Smac/DIABLO |
| SB04807 | pEF1a: Caspase-9 (inactive monomer) |
| SB03080 | iCasp9 inducible with Mifepristone |

Results

Various cell-death inducing peptides were assessed to determine which pro-apoptotic members of the apoptotic pathway resulted in cell death over a course of 10 days. Cells were then lentivirally transduced to express the indicated cell-death inducing toxin.

As shown in FIG. 5B, Smac/Diablo (SB05408) did not have an impact on cell viability compared to the negative control (“Cells only”) suggesting Smac/Diablo may not be a viable toxin for a suicide switch.

As shown in FIG. 5C, tBid expression from a strong promoter impacted cell viability (SB05407) while expression from a minimal promoter did not (SB05404) compared to negative control (“Cells only”) indicating potential use in a suicide switch.

As shown in FIG. 5D, expression of Bax from a minimal and strong promoter impacted cell viability (SB05403/SB05406) compared to non-transduced control (“Cells only”) indicating potential use in a suicide switch. Expression from the minimal promoter (SB05403) appeared to be more toxic.

Overall, the results demonstrate that Bax and tBid impacted cell growth with up to a 70% and 87% decline in viability of a heterogeneous population, respectively, indicating potential use in suicide switches, while Smac/Diablo did not.

### Example 6: Transcriptional Regulation of Constitutively Expressed Caspase-9 in HEK293T Cells

Materials, Methods, and Assays

Cell engineering and assessment: 5,000 HEK293T cells were plated in a 96-well flat bottom TC treated plate. Cells were transduced same day with 5,000 pg of specified constructs (based on p24 titer). Plate transferred to the Incucyte where images of the cell layer were captured at 4 images per well every 2 hours over the course of 7 days with 10× objective. Confluency was calculated using the Incucyte basic pipeline software to map phase overlay in each well.

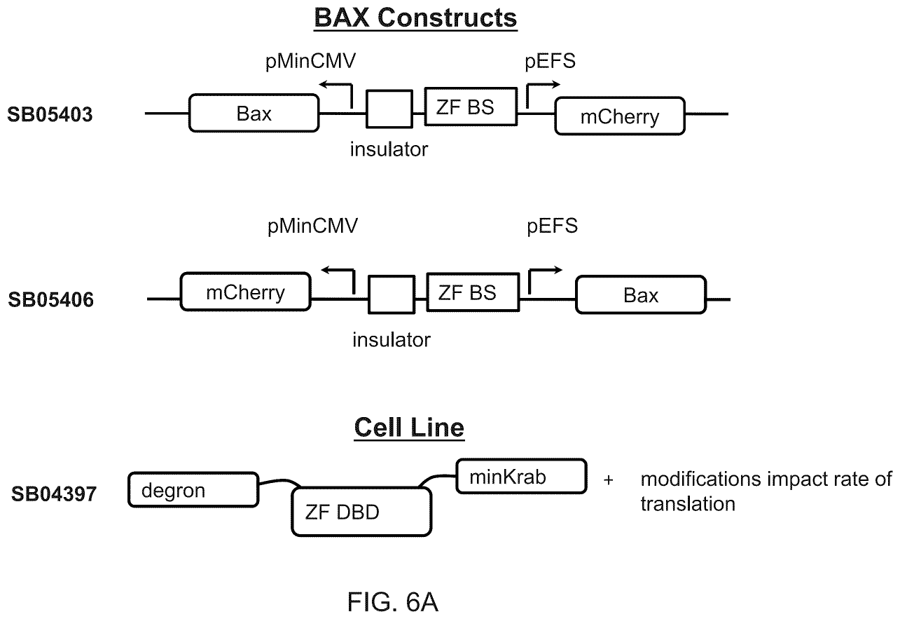

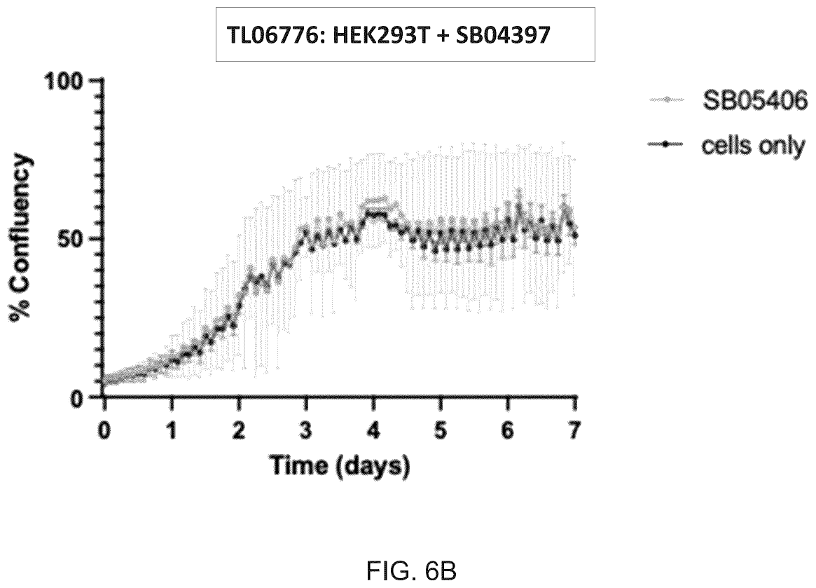

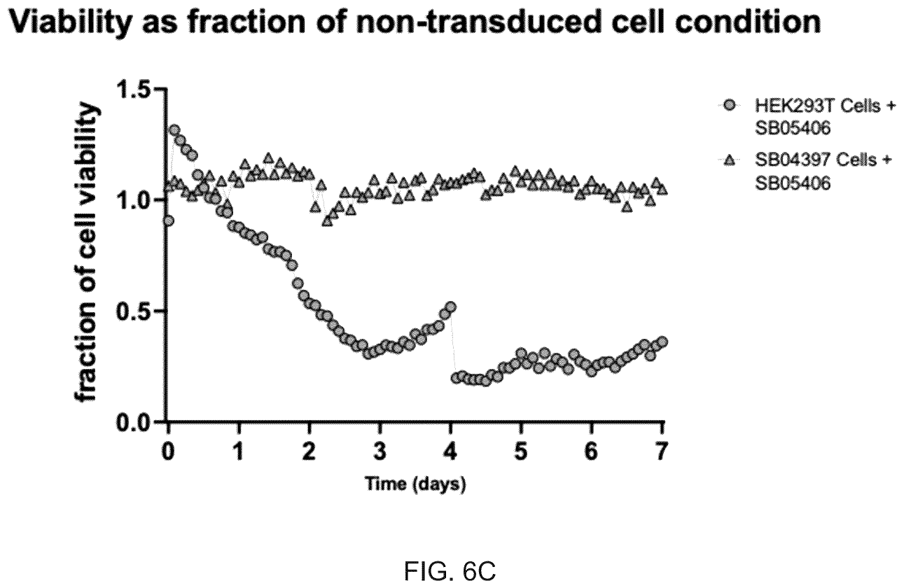

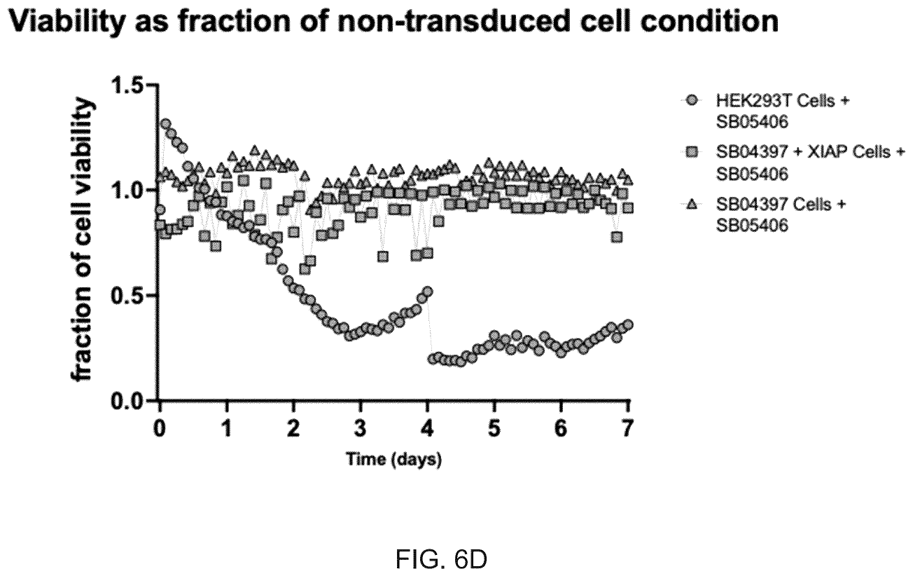

The HEK293T cell lines generated are shown in Table H below. The various cell-death inducing toxins are shown in Table I below. FIG. 6A shows the domains and organization of the constructs tested.

TABLE H
HEK293T cell lines expressing ACP (SynTF)
repressors and/or anti-toxins

| Cell Line | Construct | Description |
|---|---|---|
| TL06776 | SB04397 | degron domain-ZF-minKrab |

TABLE I
Cell-death inducing toxin constructs

| Construct | Description |
|---|---|
| SB05403 | pMinCMV: BAX |
| SB05406 | pEF1a: BAX |
| SB03080 | iCasp9 inducible with Mifepristone |

Results

Stable cell lines were generated to express an ACP including a transcriptional repressor. Cells were then lentivirally transduced to express the cell-death inducing toxin BAX or iCasp-9 under control of an ACP responsive promoter. The ACP transcriptional repressor includes a degron that in response to addition of an IMiD promotes ubiquitin pathway-mediated degradation of the peptides. Upon IMiD addition, degradation of the repressor leads to expression of the cell-death inducing peptide (see System 3; degron-tagged transcriptional repressor), as described in Example 1. In the absence of IMiD, the ACP represses transcription of the cell-death inducing toxins.

The cell line TL06776 was assessed following expression of degron domain-ZF-minKrab (SB04397) and percent confluency was quantified from pictures taken every 2 hours over course of 7 days in triplicate. As shown in FIG. 6B, cell expansion and percent confluency appeared comparable between transduced and non-transduced conditions. Viability was further assessed as the ratio of confluency of a given sample relative to the confluency of non-transduced samples. As shown in FIGS. 6C and 6D, viability of the cells was increased in cell lines expressing the ACP repressor with XIAP expression not providing a detectable benefit. Expression of Bax in HEK293T:SB04397 cell line restores cell viability and wild-type morphology (compared to a 70% decline in viability when BAX is expressed in HEK293T cells that do not express the ACP. SB05403 could not be assessed because confluency could not be quantified due to ‘fog’ issue. In summary, the results demonstrate that the ACP-expressing SB04397 cell line was able to repress SB05406 toxicity.

### Example 7: Assesment of IMiD and Tamoxifen Based Inducible Caspase-9 Dimerization (iCasp9) System

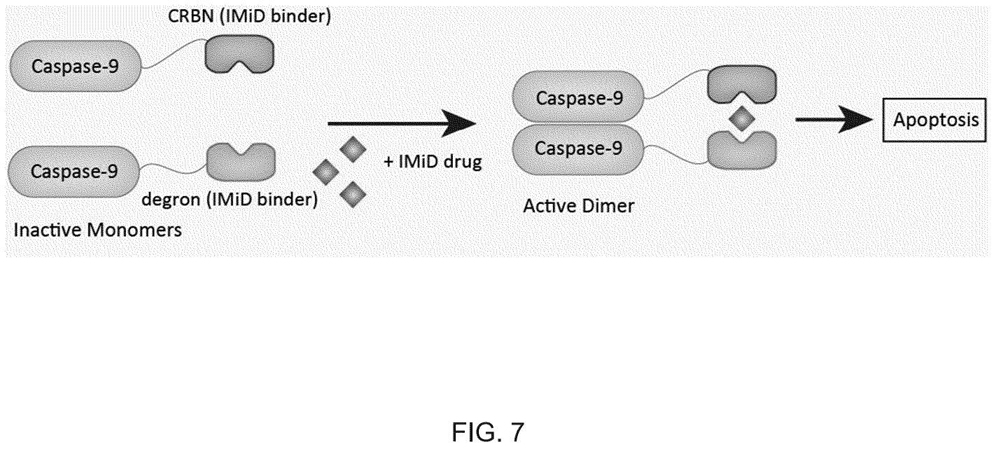

Cells are engineered as described above. The various components of the cell-death inducing toxins are shown in Table J below. FIG. 7 shows the domains and organization of the constructs. Assessed are: a dual-vector suicide switch where toxicity is regulated by drug-dependent dimerization; a CRBN modified to prevent complexing to the E3 Ubiquitin ligase complex in presence of IMiDs; and additional modifications to the degron and Caspase-9 to prevent ubiquitination.

TABLE J
Cell-death inducing toxin constructs

| Construct | Description |
|---|---|
| SB04887 | pSFFV: d913-EGFP-XIAP (T308S, G306S, G305M, P325S) |
| SB04888 | pSFFV: d913-EGFP-XIAP (T308D, G306S, G305M,P325S) |
| SB04889 | pSFFV: d913-EGFP-XIAP (T308S) |
| SB04890 | pSFFV: d913-EGFP-XIAP (T308D) |
| SB04891 | pSFFV: d913-EGFP-XIAP (G306S) |
| SB04892 | pSFFV: d913-EGFP-XIAP (G305M) |
| SB04893 | pSFFV: d913-EGFP-XIAP (P325S) |
| SB06094 | pEF1a: CRBN (del194-247)-iCasp9 |
| SB06095 | pEF1a: d913-iCasp9 |
| SB06096 | pEF1a: d913-iCasp9 (all Lys−>Arg) |
| SB06097 | pEF1a: iCasp-9-CRBN (del194-247) |
| SB06098 | pEF1a: iCasp9-d913 |
| SB06099 | pEF1a: iCasp9-d913 (all Lys−>Arg) |

TABLE K
Cell-death inducing toxin construct sequences

| | SEQ ID | |
|---|---|---|
| Construct | NO: | DNA Sequence (5′-3′) |
| SB04887 | 139 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAatgagcCTAagcGATTGGAAGCCCAGTGAAGACCCTTGGGAAC |
| | | AACATGCTAAATGGTATagcGGGTGCAAATATCTGTTAGAACAGAAGG |
| | | GACAAGAATATATAAACAATATTCATTTAACTCATTCACTTGAGGAGTG |
| | | TCTGGTAAGAACTACTGAGAAAACACCATCACTAACTAGAAGAATTGA |
| | | TGATACCATCTTCCAAAATCCTATGGTACAAGAAGCTATACGAATGGG |
| | | GTTCAGTTTCAAGGACATTAAGAAAATAATGGAGGAAAAAATTCAGAT |
| | | ATCTGGGAGCAACTATAAATCACTTGAGGTTCTGGTTGCAGATCTAGT |
| | | GAATGCTCAGAAAGACAGTATGCAAGATGAGTCAAGTCAGACTTCATT |
| | | ACAGAAAGAGATTAGTACTGAAGAGCAGCTAAGGCGCCTGCAAGAGG |
| | | AGAAGCTTTGCAAAATCTGTATGGATAGAAATATTGCTATCGTTTTTGT |
| | | TCCTTGTGGACATCTAGTCACTTGTAAACAATGTGCTGAAGCAGTTGA |
| | | CAAGTGTCCCATGTGCTACACAGTCATTACTTTCAAGCAAAAAATTTTT |
| | | ATGTCT |
| SB04888 | 140 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAatgagcCTAgacGATTGGAAGCCCAGTGAAGACCCTTGGGAAC |
| | | AACATGCTAAATGGTATagcGGGTGCAAATATCTGTTAGAACAGAAGG |
| | | GACAAGAATATATAAACAATATTCATTTAACTCATTCACTTGAGGAGTG |
| | | TCTGGTAAGAACTACTGAGAAAACACCATCACTAACTAGAAGAATTGA |
| | | TGATACCATCTTCCAAAATCCTATGGTACAAGAAGCTATACGAATGGG |
| | | GTTCAGTTTCAAGGACATTAAGAAAATAATGGAGGAAAAAATTCAGAT |
| | | ATCTGGGAGCAACTATAAATCACTTGAGGTTCTGGTTGCAGATCTAGT |
| | | GAATGCTCAGAAAGACAGTATGCAAGATGAGTCAAGTCAGACTTCATT |
| | | ACAGAAAGAGATTAGTACTGAAGAGCAGCTAAGGCGCCTGCAAGAGG |
| | | AGAAGCTTTGCAAAATCTGTATGGATAGAAATATTGCTATCGTTTTTGT |
| | | TCCTTGTGGACATCTAGTCACTTGTAAACAATGTGCTGAAGCAGTTGA |
| | | CAAGTGTCCCATGTGCTACACAGTCATTACTTTCAAGCAAAAAATTTTT |
| | | ATGTCT |
| SB04889 | 141 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAGGAGGGCTAagcGATTGGAAGCCCAGTGAAGACCCTTGGGA |
| | | ACAACATGCTAAATGGTATCCAGGGTGCAAATATCTGTTAGAACAGAA |
| | | GGGACAAGAATATATAAACAATATTCATTTAACTCATTCACTTGAGGA |
| | | GTGTCTGGTAAGAACTACTGAGAAAACACCATCACTAACTAGAAGAAT |
| | | TGATGATACCATCTTCCAAAATCCTATGGTACAAGAAGCTATACGAAT |
| | | GGGGTTCAGTTTCAAGGACATTAAGAAAATAATGGAGGAAAAAATTCA |
| | | GATATCTGGGAGCAACTATAAATCACTTGAGGTTCTGGTTGCAGATCT |
| | | AGTGAATGCTCAGAAAGACAGTATGCAAGATGAGTCAAGTCAGACTT |
| | | CATTACAGAAAGAGATTAGTACTGAAGAGCAGCTAAGGCGCCTGCAA |
| | | GAGGAGAAGCTTTGCAAAATCTGTATGGATAGAAATATTGCTATCGTT |
| | | TTTGTTCCTTGTGGACATCTAGTCACTTGTAAACAATGTGCTGAAGCA |
| | | GTTGACAAGTGTCCCATGTGCTACACAGTCATTACTTTCAAGCAAAAA |
| | | ATTTTTATGTCT |
| SB04890 | 142 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAGGAGGGCTAgacGATTGGAAGCCCAGTGAAGACCCTTGGGA |
| | | ACAACATGCTAAATGGTATCCAGGGTGCAAATATCTGTTAGAACAGAA |
| | | GGGACAAGAATATATAAACAATATTCATTTAACTCATTCACTTGAGGA |
| | | GTGTCTGGTAAGAACTACTGAGAAAACACCATCACTAACTAGAAGAAT |
| | | TGATGATACCATCTTCCAAAATCCTATGGTACAAGAAGCTATACGAAT |
| | | GGGGTTCAGTTTCAAGGACATTAAGAAAATAATGGAGGAAAAAATTCA |
| | | GATATCTGGGAGCAACTATAAATCACTTGAGGTTCTGGTTGCAGATCT |
| | | AGTGAATGCTCAGAAAGACAGTATGCAAGATGAGTCAAGTCAGACTT |
| | | CATTACAGAAAGAGATTAGTACTGAAGAGCAGCTAAGGCGCCTGCAA |
| | | GAGGAGAAGCTTTGCAAAATCTGTATGGATAGAAATATTGCTATCGTT |
| | | TTTGTTCCTTGTGGACATCTAGTCACTTGTAAACAATGTGCTGAAGCA |
| | | GTTGACAAGTGTCCCATGTGCTACACAGTCATTACTTTCAAGCAAAAA |
| | | ATTTTTATGTCT |
| SB04891 | 143 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAGGAagcCTAACTGATTGGAAGCCCAGTGAAGACCCTTGGGA |
| | | ACAACATGCTAAATGGTATCCAGGGTGCAAATATCTGTTAGAACAGAA |
| | | GGGACAAGAATATATAAACAATATTCATTTAACTCATTCACTTGAGGA |
| | | GTGTCTGGTAAGAACTACTGAGAAAACACCATCACTAACTAGAAGAAT |
| | | TGATGATACCATCTTCCAAAATCCTATGGTACAAGAAGCTATACGAAT |
| | | GGGGTTCAGTTTCAAGGACATTAAGAAAATAATGGAGGAAAAAATTCA |
| | | GATATCTGGGAGCAACTATAAATCACTTGAGGTTCTGGTTGCAGATCT |
| | | AGTGAATGCTCAGAAAGACAGTATGCAAGATGAGTCAAGTCAGACTT |
| | | CATTACAGAAAGAGATTAGTACTGAAGAGCAGCTAAGGCGCCTGCAA |
| | | GAGGAGAAGCTTTGCAAAATCTGTATGGATAGAAATATTGCTATCGTT |
| | | TTTGTTCCTTGTGGACATCTAGTCACTTGTAAACAATGTGCTGAAGCA |
| | | GTTGACAAGTGTCCCATGTGCTACACAGTCATTACTTTCAAGCAAAAA |
| | | ATTTTTATGTCT |
| SB04892 | 144 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAatgGGGCTAACTGATTGGAAGCCCAGTGAAGACCCTTGGGAA |
| | | CAACATGCTAAATGGTATCCAGGGTGCAAATATCTGTTAGAACAGAAG |
| | | GGACAAGAATATATAAACAATATTCATTTAACTCATTCACTTGAGGAGT |
| | | GTCTGGTAAGAACTACTGAGAAAACACCATCACTAACTAGAAGAATTG |
| | | ATGATACCATCTTCCAAAATCCTATGGTACAAGAAGCTATACGAATGG |
| | | GGTTCAGTTTCAAGGACATTAAGAAAATAATGGAGGAAAAAATTCAGA |
| | | TATCTGGGAGCAACTATAAATCACTTGAGGTTCTGGTTGCAGATCTAG |
| | | TGAATGCTCAGAAAGACAGTATGCAAGATGAGTCAAGTCAGACTTCAT |
| | | TACAGAAAGAGATTAGTACTGAAGAGCAGCTAAGGCGCCTGCAAGAG |
| | | GAGAAGCTTTGCAAAATCTGTATGGATAGAAATATTGCTATCGTTTTT |
| | | GTTCCTTGTGGACATCTAGTCACTTGTAAACAATGTGCTGAAGCAGTT |
| | | GACAAGTGTCCCATGTGCTACACAGTCATTACTTTCAAGCAAAAAATT |
| | | TTTATGTCT |
| SB04893 | 145 | ATGACTTTTAACAGTTTTGAAGGATCTAAAACTTGTGTACCTGCAGAC |
| | | ATCAATAAGGAAGAAGAATTTGTAGAAGAGTTTAATAGATTAAAAACTT |
| | | TTGCTAATTTTCCAAGTGGTAGTCCTGTTTCAGCATCAACACTGGCAC |
| | | GAGCAGGGTTTCTTTATACTGGTGAAGGAGATACCGTGCGGTGCTTT |
| | | AGTTGTCATGCAGCTGTAGATAGGTGGCAATATGGAGACTCAGCAGT |
| | | TGGAAGACACAGGAAAGTATCCCCAAATTGCAGATTTATCAACGGCTT |
| | | TTATCTTGAAAATAGTGCCACGCAGTCTACAAATTCTGGTATCCAGAA |
| | | TGGTCAGTACAAAGTTGAAAACTATCTGGGAAGCAGAGATCATTTTGC |
| | | CTTAGACAGGCCATCTGAGACACATGCAGACTATCTTTTGAGAACTG |
| | | GGCAGGTTGTAGATATATCAGACACCATATACCCGAGGAACCCTGCC |
| | | ATGTATAGTGAAGAAGCTAGATTAAAGTCCTTTCAGAACTGGCCAGAC |
| | | TATGCTCACCTAACCCCAAGAGAGTTAGCAAGTGCTGGACTCTACTA |
| | | CACAGGTATTGGTGACCAAGTGCAGTGCTTTTGTTGTGGTGGAAAAC |
| | | TGAAAAATTGGGAACCTTGTGATCGTGCCTGGTCAGAACACAGGCGA |
| | | CACTTTCCTAATTGCTTCTTTGTTTTGGGCCGGAATCTTAATATTCGAA |
| | | GTGAATCTGATGCTGTGAGTTCTGATAGGAATTTCCCAAATTCAACAA |
| | | ATCTTCCAAGAAATCCATCCATGGCAGATTATGAAGCACGGATCTTTA |
| | | CTTTTGGGACATGGATATACTCAGTTAACAAGGAGCAGCTTGCAAGA |
| | | GCTGGATTTTATGCTTTAGGTGAAGGTGATAAAGTAAAGTGCTTTCAC |
| | | TGTGGAGGAGGGCTAACTGATTGGAAGCCCAGTGAAGACCCTTGGG |
| | | AACAACATGCTAAATGGTATagcGGGTGCAAATATCTGTTAGAACAGAA |
| | | GGGACAAGAATATATAAACAATATTCATTTAACTCATTCACTTGAGGA |
| | | GTGTCTGGTAAGAACTACTGAGAAAACACCATCACTAACTAGAAGAAT |
| | | TGATGATACCATCTTCCAAAATCCTATGGTACAAGAAGCTATACGAAT |
| | | GGGGTTCAGTTTCAAGGACATTAAGAAAATAATGGAGGAAAAAATTCA |
| | | GATATCTGGGAGCAACTATAAATCACTTGAGGTTCTGGTTGCAGATCT |
| | | AGTGAATGCTCAGAAAGACAGTATGCAAGATGAGTCAAGTCAGACTT |
| | | CATTACAGAAAGAGATTAGTACTGAAGAGCAGCTAAGGCGCCTGCAA |
| | | GAGGAGAAGCTTTGCAAAATCTGTATGGATAGAAATATTGCTATCGTT |
| | | TTTGTTCCTTGTGGACATCTAGTCACTTGTAAACAATGTGCTGAAGCA |
| | | GTTGACAAGTGTCCCATGTGCTACACAGTCATTACTTTCAAGCAAAAA |
| | | ATTTTTATGTCT |
| SB06094 | 146 | ATGGCGGGCGAGGGCGATCAGCAAGACGCGGCTCACAACATGGGAA |
| | | ATCACTTACCCTTACTTCCCGCAGAATCTGAAGAGGAAGATGAGATG |
| | | GAGGTAGAGGATCAGGACTCTAAAGAAGCGAAAAAGCCTAATATCAT |
| | | CAACTTTGACACCTCTCTTCCAACCAGTCACACTTACCTGGGGGCAG |
| | | ACATGGAGGAATTCCATGGTCGAACTCTCCACGACGACGATAGCTGC |
| | | CAAGTGATACCAGTGCTCCCTCAGGTAATGATGATTCTTATTCCTGGA |
| | | CAGACCCTGCCTCTACAGCTGTTCCACCCTCAAGAGGTGAGCATGGT |
| | | CAGGAATTTGATCCAGAAGGACAGAACATTTGCGGTCCTGGCCTACT |
| | | CAAACGTACAGGAGCGAGAGGCTCAGTTCGGGACCACTGCCGAAAT |
| | | ATACGCGTACCGGGAGGAGCAAGACTTCGGGATCGAAATCGTGAAG |
| | | GTAAAGGCCATTGGCAGACAACGGTTTAAGGTCCTTGAGCTCCGGAC |
| | | GCAGAGTGATGGGATACAACAGGCTAAAGTGCAGATCCTACCAGAGT |
| | | GTGTATTACCATCTACCTATGATGCAGAGACTCTCATGGACCGCATAA |
| | | AAAAGCAATTAAGGGAATGGGACGAAAACCTCAAGGACGATTCACTC |
| | | CCATCCAACCCCATTGACTTCTCATATAGGGTCGCTGCTTGTTTGCCT |
| | | ATCGACGACGTCCTTAGGATACAGCTCCTGAAAATCGGAAGCGCAAT |
| | | ACAAAGATTGCGCTGTGAGCTGGACATTATGAATAAGTGCACTTCACT |
| | | GTGTTGTAAGCAGTGTCAAGAGACCGAAATCACTACGAAGAACGAGA |
| | | TCTTCTCTCTCTCCCTCTGCGGGCCAATGGCAGCATATGTAAATCCG |
| | | CATGGGTATGTTCATGAGACACTAACTGTGTACAAGGCCTGCAATTTA |
| | | AACTTGATCGGCCGGCCATCTACAGAACACAGTTGGTTCCCGGGTTA |
| | | TGCCTGGACAGTGGCACAGTGCAAAATTTGTGCTTCCCACATTGGAT |
| | | GGAAATTTACAGCCACAAAGAAGGATATGAGTCCCCAAAAGTTCTGG |
| | | GGACTGACCAGGAGCGCTTTATTGCCCACCATCCCGGACACAGAGG |
| | | ACGAAATCTCTCCGGACAAGGTGATTCTCTGTCTGgggggggaggttcagg |
| | | gggtggaggttcaggtggtggcggtagtgtcgatggcttcatggatgtcggtgctcttgagagtttgagg |
| | | ggaaatgcagatttggcttacatcctgagcatggagccctgtggccactgcctcattatcaacaatgtga |
| | | acttctgccgtgagtccgggctccgcacccgcactggctccaacatcgactgtgagaagttgcggcgtc |
| | | gcttctcctcgctgcatttcatggtggaggtgaagggcgacctgactgccaagaaaatggtgctggcttt |
| | | gctggagctggcgcggcaggaccacggtgctctggactgctgcgtggtggtcattctctctcacggctgt |
| | | caggccagccacctgcagttcccaggggctgtctacggcacagatggatgccctgtgtcggtcgaga |
| | | agattgtgaacatcttcaatgggaccagctgccccagcctgggagggaagcccaagctctttttcatcc |
| | | aggcctgtggtggggagcagaaagaccatgggtttgaggtggcctccacttcccctgaagacgagtc |
| | | ccctggcagtaaccccgagccagatgccaccccgttccaggaaggtttgaggaccttcgaccagctg |
| | | gacgccatatctagtttgcccacacccagtgacatctttgtgtcctactctactttcccaggttttgtttcctgg |
| | | agggaccccaagagtggctcctggtacgttgagaccctggacgacatctttgagcagtgggctcactct |
| | | gaagacctgcagtccctcctgcttagggtcgctaatgctgtttcggtgaaagggatttataaacagatgc |
| | | ctggttgctttaatttcctccggaaaaaacttttctttaaaacatca |
| SB06095 | 147 | atgTTCAACGTGCTGATGGTGCACAAGCGGAGCCACACCGGCGAAAG |
| | | ACCTCTGCAGTGTGAAATCTGCGGCTTCACCTGTCGGCAGAAGGGCA |
| | | ACCTGCTGCGGCACATCAAACTGCACACAGGCGAGAAGCCCTTCAAG |
| | | TGCCACCTGTGCAATTACGCCTGCCAGAGAAGAGATGCCCTGgggggtg |
| | | gaggttcagggggtggaggttcaggtggtggcggtagtgtcgatggcttcatggatgtcggtgctcttga |
| | | gagtttgaggggaaatgcagatttggcttacatcctgagcatggagccctgtggccactgcctcattatc |
| | | aacaatgtgaacttctgccgtgagtccgggctccgcacccgcactggctccaacatcgactgtgagaa |
| | | gttgcggcgtcgcttctcctcgctgcatttcatggtggaggtgaagggcgacctgactgccaagaaaat |
| | | ggtgctggctttgctggagctggcgcggcaggaccacggtgctctggactgctgcgtggtggtcattctc |
| | | tctcacggctgtcaggccagccacctgcagttcccaggggctgtctacggcacagatggatgccctgt |
| | | gtcggtcgagaagattgtgaacatcttcaatgggaccagctgccccagcctgggagggaagcccaa |
| | | gctctttttcatccaggcctgtggtggggagcagaaagaccatgggtttgaggtggcctccacttcccctg |
| | | aagacgagtcccctggcagtaaccccgagccagatgccaccccgttccaggaaggtttgaggacctt |
| | | cgaccagctggacgccatatctagtttgcccacacccagtgacatctttgtgtcctactctactttcccagg |
| | | ttttgtttcctggagggaccccaagagtggctcctggtacgttgagaccctggacgacatctttgagcagt |
| | | gggctcactctgaagacctgcagtccctcctgcttagggtcgctaatgctgtttcggtgaaagggatttat |
| | | aaacagatgcctggttgctttaatttcctccggaaaaaacttttctttaaaacatca |
| SB06096 | 148 | atgTTCAACGTGCTGATGGTGCACAGGCGGAGCCACACCGGCGAAAG |
| | | ACCTCTGCAGTGTGAAATCTGCGGCTTCACCTGTCGGCAGAGGGGC |
| | | AACCTGCTGCGGCACATCAGACTGCACACAGGCGAGAGGCCCTTCA |
| | | GGTGCCACCTGTGCAATTACGCCTGCCAGAGAAGAGATGCCCTGggg |
| | | ggtggaggttcagggggggaggttcaggtggtggcggtagtgtcgatggCTTcgatgtcggtgctC |
| | | TTgagagTTTgaggggAAAtgcagaTTTggCTTacatcctgagcatggagccctgtggcca |
| | | ctgcctcattatcaacaatgtgaaCTTctgccgtgagtccgggctccgcacccgcactggctccaaca |
| | | tcgactgtgagAGGttgcggcgtcgCTTctcctcgctgcaTTTcatggtggaggtgAGGggcga |
| | | cctgactgccAGGAGAatggtgctggcTTTgctggagctggcgcggcaggaccacggtgctctg |
| | | gactgctgcgtggtggtcattctctctcacggctgtcaggccagccacctgcagttcccaggggctgtct |
| | | acggcacagatggatgccctgtgtcggtcgagAGGattgtgaacatCtTcaatgggaccagctgcc |
| | | ccagcctgggagggAGGcccAGGctcTttTtcatccaggcctgtggtggggagcagAGAgac |
| | | catgggTtTgaggtggcctccaCtTcccctgAaGacgagtcccctggcagtaaccccgagccaga |
| | | tgccaccccgttccaggAaGgTtTgaggacCtTcgaccagctggacgccatatctagTtTgccca |
| | | cacccagtgacatcTtTgtgtcctactctacTtTcccaggTttTgTtTcctggagggaccccAGGa |
| | | gtggctcctggtacgttgagaccctggacgacatcTtTgagcagtgggctcactctgAaGacctgca |
| | | gtccctcctgCtTagggtcgctaatgctgTtTcggtgAGAgggaTtTatAGAcagatgcctggttg |
| | | cTtTaaTtTcctccggAGAGGAcTttTcTtTAgAacatca |
| SB06097 | 149 | atggatgtcggtgctcttgagagtttgaggggaaatgcagatttggcttacatcctgagcatggagccct |
| | | gtggccactgcctcattatcaacaatgtgaacttctgccgtgagtccgggctccgcacccgcactggctc |
| | | caacatcgactgtgagaagttgcggcgtcgcttctcctcgctgcatttcatggtggaggtgaagggcga |
| | | cctgactgccaagaaaatggtgctggctttgctggagctggcgcggcaggaccacggtgctctggact |
| | | gctgcgtggtggtcattctctctcacggctgtcaggccagccacctgcagttcccaggggctgtctacgg |
| | | cacagatggatgccctgtgtcggtcgagaagattgtgaacatcttcaatgggaccagctgccccagcct |
| | | gggagggaagcccaagctctttttcatccaggcctgtggtggggagcagaaagaccatgggtttgag |
| | | gtggcctccacttcccctgaagacgagtcccctggcagtaaccccgagccagatgccaccccgttcca |
| | | ggaaggtttgaggaccttcgaccagctggacgccatatctagtttgcccacacccagtgacatctttgtgt |
| | | cctactctactttcccaggttttgtttcctggagggaccccaagagtggctcctggtacgttgagaccctgg |
| | | acgacatctttgagcagtgggctcactctgaagacctgcagtccctcctgcttagggtcgctaatgctgttt |
| | | cggtgaaagggatttataaacagatgcctggttgctttaatttcctccggaaaaaacttttctttaaaacat |
| | | cagggggtggaggttcagggggtggaggttcaggtggtggcggtagtgtcgatggcttcATGGCG |
| | | GGCGAGGGCGATCAGCAAGACGCGGCTCACAACATGGGAAATCACT |
| | | TACCCTTACTTCCCGCAGAATCTGAAGAGGAAGATGAGATGGAGGTA |
| | | GAGGATCAGGACTCTAAAGAAGCGAAAAAGCCTAATATCATCAACTTT |
| | | GACACCTCTCTTCCAACCAGTCACACTTACCTGGGGGCAGACATGGA |
| | | GGAATTCCATGGTCGAACTCTCCACGACGACGATAGCTGCCAAGTGA |
| | | TACCAGTGCTCCCTCAGGTAATGATGATTCTTATTCCTGGACAGACCC |
| | | TGCCTCTACAGCTGTTCCACCCTCAAGAGGTGAGCATGGTCAGGAAT |
| | | TTGATCCAGAAGGACAGAACATTTGCGGTCCTGGCCTACTCAAACGT |
| | | ACAGGAGCGAGAGGCTCAGTTCGGGACCACTGCCGAAATATACGCG |
| | | TACCGGGAGGAGCAAGACTTCGGGATCGAAATCGTGAAGGTAAAGG |
| | | CCATTGGCAGACAACGGTTTAAGGTCCTTGAGCTCCGGACGCAGAGT |
| | | GATGGGATACAACAGGCTAAAGTGCAGATCCTACCAGAGTGTGTATT |
| | | ACCATCTACCTATGATGCAGAGACTCTCATGGACCGCATAAAAAAGCA |
| | | ATTAAGGGAATGGGACGAAAACCTCAAGGACGATTCACTCCCATCCA |
| | | ACCCCATTGACTTCTCATATAGGGTCGCTGCTTGTTTGCCTATCGACG |
| | | ACGTCCTTAGGATACAGCTCCTGAAAATCGGAAGCGCAATACAAAGA |
| | | TTGCGCTGTGAGCTGGACATTATGAATAAGTGCACTTCACTGTGTTGT |
| | | AAGCAGTGTCAAGAGACCGAAATCACTACGAAGAACGAGATCTTCTC |
| | | TCTCTCCCTCTGCGGGCCAATGGCAGCATATGTAAATCCGCATGGGT |
| | | ATGTTCATGAGACACTAACTGTGTACAAGGCCTGCAATTTAAACTTGA |
| | | TCGGCCGGCCATCTACAGAACACAGTTGGTTCCCGGGTTATGCCTGG |
| | | ACAGTGGCACAGTGCAAAATTTGTGCTTCCCACATTGGATGGAAATTT |
| | | ACAGCCACAAAGAAGGATATGAGTCCCCAAAAGTTCTGGGGACTGAC |
| | | CAGGAGCGCTTTATTGCCCACCATCCCGGACACAGAGGACGAAATCT |
| | | CTCCGGACAAGGTGATTCTCTGTCTG |
| SB06098 | 150 | atgTTCAACGTGCTGATGGTGCACAGGCGGAGCCACACCGGCGAAAG |
| | | ACCTCTGCAGTGTGAAATCTGCGGCTTCACCTGTCGGCAGAGGGGC |
| | | AACCTGCTGCGGCACATCAGACTGCACACAGGCGAGAGGCCCTTCA |
| | | GGTGCCACCTGTGCAATTACGCCTGCCAGAGAAGAGATGCCCTGggg |
| | | ggtggaggttcagggggtggaggttcaggtggtggcggtagtgtcgatggCTTcgatgtcggtgctC |
| | | TTgagagTTTgaggggAAAtgcagaTTTggCTTacatcctgagcatggagccctgtggcca |
| | | ctgcctcattatcaacaatgtgaaCTTctgccgtgagtccgggctccgcacccgcactggctccaaca |
| | | tcgactgtgagAGGttgcggcgtcgCTTctcctcgctgcaTTTcatggtggaggtgAGGggcga |
| | | cctgactgccAGGAGAatggtgctggcTTTgctggagctggcgcggcaggaccacggtgctctg |
| | | gactgctgcgtggtggtcattctctctcacggctgtcaggccagccacctgcagttcccaggggctgtct |
| | | acggcacagatggatgccctgtgtcggtcgagAGGattgtgaacatCtTcaatgggaccagctgcc |
| | | ccagcctgggagggAGGcccAGGctcTttTtcatccaggcctgtggtggggagcagAGAgac |
| | | catgggTtTgaggtggcctccaCtTcccctgAaGacgagtcccctggcagtaaccccgagccaga |
| | | tgccaccccgttccaggAaGgTtTgaggacCtTcgaccagctggacgccatatctagTtTgccca |
| | | cacccagtgacatcTtTgtgtcctactctacTtTcccaggTttTgTtTcctggagggaccccAGGa |
| | | gtggctcctggtacgttgagaccctggacgacatcTtTgagcagtgggctcactctgAaGacctgca |
| ` | | gtccctcctgCtTagggtcgctaatgctgTtTcggtgAGAgggaTtTatAGAcagatgcctggttg |
| | | cTtTaaTtTcctccggAGAGGAcTttTcTtTAgAacatca |
| SB06099 | 151 | atggatgtcggtgctCTTgagagTTTgaggggAAAtgcagaTTTggCTTacatcctgagcat |
| | | ggagccctgtggccactgcctcattatcaacaatgtgaaCTTctgccgtgagtccgggctccgcacc |
| | | cgcactggctccaacatcgactgtgagAGGttgcggcgtcgCTTctcctcgctgcaTTTcatggtg |
| | | gaggtgAGGggcgacctgactgccAGGAGAatggtgctggcTTTgctggagctggcgcggc |
| | | aggaccacggtgctctggactgctgcgtggtggtcattctctctcacggctgtcaggccagccacctgc |
| | | agttcccaggggctgtctacggcacagatggatgccctgtgtcggtcgagAGGattgtgaacatCtT |
| | | caatgggaccagctgccccagcctgggagggAGGcccAGGctcTttTtcatccaggcctgtggt |
| | | ggggagcagAGAgaccatgggTtTgaggtggcctccaCtTcccctgAaGacgagtcccctggc |
| | | agtaaccccgagccagatgccaccccgttccaggAaGgTtTgaggacCtTcgaccagctggac |
| | | gccatatctagTtTgcccacacccagtgacatcTtTgtgtcctactctac TtTcccaggTttTgTtTc |
| | | ctggagggaccccAGGagtggctcctggtacgttgagaccctggacgacatcTtTgagcagtggg |
| | | ctcactctgAaGacctgcagtccctcctgCtTagggtcgctaatgctgTtTcggtgAGAgggaTtT |
| | | atAGAcagatgcctggttgcTtTaaTtTcctccggAGAGGAcTttTcTtTAgAacatcagggg |
| | | gtggaggttcagggggtggaggttcaggtggtggcggtagtgtcgatggCTTcTTCAACGTGC |
| | | TGATGGTGCACAGGCGGAGCCACACCGGCGAAAGACCTCTGCAGTG |
| | | TGAAATCTGCGGCTTCACCTGTCGGCAGAGGGGCAACCTGCTGCGG |
| | | CACATCAGACTGCACACAGGCGAGAGGCCCTTCAGGTGCCACCTGT |
| | | GCAATTACGCCTGCCAGAGAAGAGATGCCCTG |

Results

Various cell-death inducing peptides are assessed to determine which pro-apoptotic members of the apoptotic pathway resulted in cell death over a course of 10 days. Cells are then lentivirally transduced to express the indicated cell-death inducing toxin. The results demonstrate a functional IMiD and tamoxifen based inducible caspase-9 dimerization (iCasp9) system.

### OTHER EMBODIMENTS

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

1-20. (canceled)

21. A method of treating a subject in need thereof, comprising:
delivering to the subject in need thereof a therapeutically effective amount of an engineered cell comprising an inducible cell death system or an engineered nucleic acid comprising the inducible cell death system,
wherein the inducible cell death system comprises two or more polypeptide monomers, wherein each polypeptide monomer comprises one or more ligand binding domains and a cell death-inducing domain, wherein the polypeptide monomers are configured to oligomerize upon contacting the polypeptide monomers with a cognate ligand of the one or more ligand binding domains and generate a cell-death inducing signal in a cell in which the polypeptide monomers are expressed, and wherein:
i. each polypeptide monomer of the two or more polypeptide monomers comprises the same ligand binding domain, wherein the same ligand binding domain comprises a domain or functional fragment thereof selected from the group consisting of: a hormone-binding domain of estrogen receptor (ER) domain, optionally comprising the amino acid sequence of SEQ ID NO: 42; an ABI domain, optionally comprising the amino sequence of SEQ ID NO: 31; a PYL domain, optionally comprising the amino acid sequence of SEQ ID NO: 53; a caffeine-binding single-domain antibody, optionally comprising the amino acid sequence of SEQ ID NO: 33; a cannabidiol binding domain, optionally comprising an amino acid sequence selected from the group consisting of SEQ ID NO: 34, 35, 36, 37, and 38; a heavy chain variable region (VH) of an anti-nicotine antibody, optionally comprising the amino acid sequence of SEQ ID NO: 50, and/or the light chain variable region (VL) of an anti-nicotine antibody, optionally comprising the amino acid sequence of SEQ ID NO: 51; optionally comprising the amino acid sequence of SEQ ID NO: 43; a progesterone receptor domain, optionally comprising the amino acid sequence of SEQ ID NO: 52; and combinations thereof; or
ii. a first polypeptide monomer of the two or more polypeptide monomers comprises a first ligand binding domain, a second polypeptide monomer of the two or more polypeptide monomers comprises a second ligand binding domain, and wherein the second ligand binding domain is different from the first ligand binding domain.

22. The method of claim 21, wherein:
the first ligand binding domain of (ii) comprises a cereblon domain, optionally comprising the amino acid sequence set forth in one of SEQ ID NOs: 127 and 129, and wherein the second ligand binding domain of (ii) comprises a degron, optionally comprising the amino acid sequence set forth in one of SEQ ID NOs: 131 and 133, optionally wherein the cognate ligand is an IMiD, optionally wherein the IMiD is an FDA-approved drug, and optionally wherein the IMiD is selected from the group consisting of: thalidomide, lenalidomide, and pomalidomide; or
the first polypeptide monomer comprises a hormone-binding domain of estrogen receptor (ER) domain and the second polypeptide monomer comprises an FKBP domain, optionally wherein the cognate ligand is rapamycin or a derivative thereof and/or tamoxifen or a metabolite thereof; or
the first polypeptide monomer comprises an FRB domain and the second polypeptide monomer comprises a hormone-binding domain of estrogen receptor (ER) domain, optionally wherein the cognate ligand is rapamycin or a derivative thereof and/or tamoxifen or a metabolite thereof; or
wherein the first polypeptide monomer comprises a hormone-binding domain of estrogen receptor (ER) domain and an FKBP domain, and the second polypeptide monomer comprises an FRB domain and a hormone-binding domain of estrogen receptor (ER) domain, optionally wherein the cognate ligand is rapamycin or a derivative thereof and/or tamoxifen or a metabolite thereof; or
the first polypeptide monomer comprises an ABI domain and the second polypeptide monomer comprises a PYL domain, optionally wherein the cognate ligand comprises abscisic acid; or
the first polypeptide monomer comprises a heavy chain variable region (VH) of an anti-nicotine antibody and the second polypeptide monomer comprises a light chain variable region (VL) of an anti-nicotine antibody, optionally wherein the anti-nicotine antibody is a Nic12 antibody, optionally wherein the VH comprises the amino acid sequence of SEQ ID NO: 50, and optionally wherein the VL comprises the amino acid sequence of SEQ ID NO: 51, and optionally wherein the cognate ligand is nicotine or a derivative thereof; or
the first polypeptide monomer comprises a cannabidiol binding domain comprising an amino acid sequence selected from the group consisting of SEQ ID NO: 35, 36, 37, and 38 and the second polypeptide monomer comprises a cannabidiol binding domain comprising the amino acid sequence of SEQ ID NO: 34. optionally wherein the cognate ligand is a phytocannabinoid, optionally wherein the phytocannabinoid is cannabidiol.

23. The method of claim 21, wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32.

24. A pharmaceutical composition comprising:
a) an inducible cell death system, an engineered nucleic acid encoding the inducible cell death system, or an engineered cell comprising the inducible cell death system, wherein the inducible cell death system comprises two or more polypeptide monomers, wherein each polypeptide monomer comprises one or more ligand binding domains and a cell death-inducing domain, wherein the polypeptide monomers are configured to oligomerize upon contacting the polypeptide monomers with a cognate ligand of the one or more ligand binding domains and generate a cell-death inducing signal in a cell in which the polypeptide monomers are expressed, and wherein:
i) each polypeptide monomer of the two or more polypeptide monomers comprises the same ligand binding domain, wherein the same ligand binding domain comprises a domain or functional fragment thereof selected from the group consisting of: a hormone-binding domain of estrogen receptor (ER) domain, optionally comprising the amino acid sequence of SEQ ID NO: 42; an ABI domain, optionally comprising the amino sequence of SEQ ID NO: 31; a PYL domain, optionally comprising the amino acid sequence of SEQ ID NO: 53; a caffeine-binding single-domain antibody, optionally comprising the amino acid sequence of SEQ ID NO: 33; a cannabidiol binding domain, optionally comprising an amino acid sequence selected from the group consisting of SEQ ID NO: 34, 35, 36, 37, and 38; a heavy chain variable region (VH) of an anti-nicotine antibody, optionally comprising the amino acid sequence of SEQ ID NO: 50, and/or the light chain variable region (VL) of an anti-nicotine antibody, optionally comprising the amino acid sequence of SEQ ID NO: 51; optionally comprising the amino acid sequence of SEQ ID NO: 43; a progesterone receptor domain, optionally comprising the amino acid sequence of SEQ ID NO: 52; and combinations thereof; or
ii) a first polypeptide monomer of the two or more polypeptide monomers comprises a first ligand binding domain, a second polypeptide monomer of the two or more polypeptide monomers comprises a second ligand binding domain, and wherein the second ligand binding domain is different from the first ligand binding domain; and
b) a pharmaceutically acceptable carrier.

25. An inducible cell death system comprising an activation-conditional control polypeptide (ACP),
wherein the ACP comprises a ligand binding domain and a transcriptional effector domain, and
wherein upon binding of the ligand binding domain to a cognate ligand, the ACP is capable of modulating transcriptional expression of a gene of interest operably linked to an ACP-responsive promoter,
wherein the gene of interest comprises a cell death inducing polypeptide.

26. The inducible cell death system comprising an activation-conditional control polypeptide (ACP) of claim 25, wherein the ligand binding domain comprises a degron, optionally wherein the degron is capable of inducing degradation of the ACP,
optionally wherein the degron is selected from the group consisting of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron, an Siah binding motif, an SPOP SBC docking motif, and a PCNA binding PIP box, or the degron comprises a cereblon (CRBN) polypeptide substrate domain capable of binding CRBN in response to an immunomodulatory drug (IMiD) thereby promoting ubiquitin pathway-mediated degradation of the regulatable polypeptide, optionally wherein the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN, optionally wherein the CRBN polypeptide substrate domain is a chimeric fusion product of native CRBN polypeptide sequences, optionally wherein the CRBN polypeptide substrate domain is a IKZF3/ZFP91/IKZF3 chimeric fusion product having the amino acid sequence of
(SEQ ID NO: 103)
FNVLMVHKRSHTGERPLQCEICGFTCRQKGNLLRHIKLHTGEKPFKCHLC
NYACQRRDAL.

27. The inducible cell death system comprising an activation-conditional control polypeptide (ACP) of claim 25, wherein the transcriptional effector domain is comprised in a transcription factor that further comprises a nucleic acid-binding domain.

28. The inducible cell death system comprising an activation-conditional control polypeptide (ACP) of claim 25, wherein the transcriptional effector domain is selected from the group consisting of: a Herpes Simplex Virus Protein 16 (VP16) activation domain; an activation domain comprising four tandem copies of VP16, a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); a histone acetyltransferase (HAT) core domain of the human E1A-associated protein p300 (p300 HAT core activation domain); a Krüppel associated box (KRAB) repression domain; a Repressor Element Silencing Transcription Factor (REST) repression domain; a WRPW motif of the hairy-related basic helix-loop-helix repressor proteins, the motif is known as a WRPW repression domain; a DNA (cytosine-5)-methyltransferase 3B (DNMT3B) repression domain; and an HP1 alpha chromoshadow repression domain.

29. The inducible cell death system comprising an activation-conditional control polypeptide (ACP) of claim 25, wherein:
the ligand binding domain comprises: a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen; or
the ligand binding domain comprises: a progesterone receptor domain optionally comprising the amino acid sequence of SEQ ID NO: 52, and optionally wherein the cognate ligand is mifepristone or a derivative thereof, or
the ligand binding domain comprises: a domain or functional fragment thereof selected from the group consisting of: an ABI domain, optionally comprising the amino acid sequence of SEQ ID NO: 31, and optionally wherein the cognate ligand is abscisic acid; a PYL domain, optionally comprising the amino acid sequence of SEQ ID NO: 53, and optionally wherein the cognate ligand is abscisic acid; a caffeine-binding single-domain antibody optionally comprising the amino acid sequence of SEQ ID NO: 33, and optionally wherein the cognate ligand is caffeine or a derivative thereof; a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol; a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen; a heavy chain variable region (VH) of an anti-nicotine antibody optionally comprising the amino acid sequence of SEQ ID NO: 50, and optionally wherein the cognate ligand is nicotine or a derivative thereof; a light chain variable region (VL) of an anti-nicotine antibody optionally comprising the amino acid sequence of SEQ ID NO: 51, and optionally wherein the cognate ligand is nicotine or a derivative thereof; a progesterone receptor domain optionally comprising the amino acid sequence of SEQ ID NO: 52, and optionally wherein the cognate ligand is mifepristone or a derivative thereof; an FRB domain optionally comprising the amino acid sequence of SEQ ID NO: 44, and optionally wherein the cognate ligand is rapamycin, AP1903, AP20187, FK1012, derivatives thereof, or analogs thereof.

30. The inducible cell death system comprising an activation-conditional control polypeptide (ACP) of claim 25, wherein:
the nucleic acid-binding domain comprises a DNA-binding zinc finger protein domain (ZF protein domain), optionally wherein the ZF protein domain is modular in design and is composed of an array of zinc finger motifs, optionally wherein the ZF-protein domain comprises one to ten zinc finger motifs.

31. The inducible cell death system comprising an activation-conditional control polypeptide (ACP) of claim 25, wherein: the gene of interest is a cell death-inducing polypeptide, optionally wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32.

32. A pharmaceutical composition comprising:
a. the inducible cell death system comprising an activation-conditional control polypeptide (ACP) of claim 25; or an isolated cell comprising the inducible cell death system comprising an activation-conditional control polypeptide (ACP) of claim 25; or an engineered nucleic acid encoding the inducible cell death system comprising an activation-conditional control polypeptide (ACP) of claim 25; and
b. a pharmaceutically acceptable carrier.

33. A method of treating a subject in need thereof, comprising administering to the subject a therapeutically effective dose of a pharmaceutical composition of claim 32.

34. A multimeric activation-conditional control polypeptide (ACP) comprising:
a) a first chimeric polypeptide, wherein the first chimeric polypeptide comprises a first ligand binding domain and a transcriptional activation domain; and
b) a second chimeric polypeptide, wherein the second chimeric polypeptide comprises a second ligand binding domain and a nucleic acid-binding domain,
wherein the first chimeric polypeptide and the second chimeric polypeptide oligomerize to form the multimeric ACP via a cognate ligand that binds to each ligand binding domain, and wherein the multimeric ACP is capable of inducing transcriptional expression of a gene of interest operably linked to an ACP-responsive promoter,
wherein the gene of interest is a cell death-inducing polypeptide, optionally wherein the cell death-inducing domain is derived from a protein selected from the group consisting of: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-R), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B 1, and Purine nucleoside phosphorylase, optionally wherein the caspase 9 or a functional truncation thereof, comprises the amino acid sequence of SEQ ID NO: 39, optionally wherein the DTA comprises the amino acid sequence of SEQ ID NO: 41, optionally wherein the granzyme B comprises the amino acid sequence of SEQ ID NO: 47, optionally wherein the Bax comprises the amino acid sequence of SEQ ID NO: 32, and wherein the transcriptional activation domain is selected from the group consisting of: a Herpes Simplex Virus Protein 16 (VP16) activation domain; an activation domain comprising four tandem copies of VP16; a VP64 activation domain; a p65 activation domain of NFκB; an Epstein-Barr virus R transactivator (Rta) activation domain; a tripartite activator comprising the VP64, the p65, and the Rta activation domains (VPR activation domain); a tripartite activator comprising the VP64, the p65, and the HSF1 activation domains (VPH activation domain); and a histone acetyltransferase (HAT) core domain of the human E1A-associated protein p300 (p300 HAT core activation domain).

35. The multimeric activation-conditional control polypeptide (ACP) of claim 34, wherein the one or more ligand binding domains of a first of the two or more polypeptide monomers comprise a cereblon domain, optionally comprising the amino acid sequence set forth in one of SEQ ID NOs: 127 and 129, and the one or more ligand binding domains of a second of the two or more polypeptide monomers comprise a degron, optionally comprising the amino acid sequence set forth in one of SEQ ID NOs: 131 and 133, optionally wherein the cognate ligand is an IMiD, optionally wherein the IMiD is an FDA-approved drug, and optionally wherein the IMiD is selected from the group consisting of: thalidomide, lenalidomide, and pomalidomide.

36. The multimeric activation-conditional control polypeptide (ACP) of claim 34, wherein the nucleic acid-binding domain comprises a DNA-binding zinc finger protein domain (ZF protein domain), optionally wherein the ZF protein domain is modular in design and is composed of an array of zinc finger motifs, optionally wherein the ZF-protein domain comprises one to ten zinc finger motifs.

37. The multimeric activation-conditional control polypeptide (ACP) of claim 34, wherein the nucleic acid-binding domain binds to the ACP-responsive promoter, optionally wherein the ACP-responsive promoter comprises an ACP-binding domain sequence and a promoter sequence, optionally wherein the promoter sequence comprises a minimal promoter, optionally wherein the promoter sequence is an inducible promoter and further comprises a responsive element selected from the group consisting of: NFκB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule-responsive promoters, and tandem repeats thereof, and optionally wherein the ACP-responsive promoter comprises a synthetic promoter, and optionally wherein the ACP-binding domain comprises one or more zinc finger binding sites.

38. The multimeric activation-conditional control polypeptide (ACP) of claim 34, wherein the one or more ligand binding domains of each of the two or more polypeptide monomers comprise a domain or functional fragment thereof selected from the group consisting of: an ABI domain, optionally comprising the amino acid sequence of SEQ ID NO: 31, and optionally wherein the cognate ligand is abscisic acid; a PYL domain, optionally comprising the amino acid sequence of SEQ ID NO: 53, and optionally wherein the cognate ligand is abscisic acid; a caffeine-binding single-domain antibody optionally comprising the amino acid sequence of SEQ ID NO: 33, and optionally wherein the cognate ligand is caffeine or a derivative thereof; a cannabidiol binding domain, optionally comprising an amino acid sequence selected from the group consisting of SEQ ID NO: 34, 35, 36, 37, and 38, optionally wherein the cognate ligand is a phytocannabinoid, optionally wherein the phytocannabinoid is cannabidiol; a hormone-binding domain of estrogen receptor (ER) domain optionally comprising the amino acid sequence of SEQ ID NO: 42, optionally wherein the cognate ligand is tamoxifen or a metabolite thereof, and optionally wherein the tamoxifen metabolite is selected from the group consisting of: 4-hydroxytamoxifen, N-desmethyltamoxifen, tamoxifen-N-oxide, and endoxifen; a heavy chain variable region (VH) of an anti-nicotine antibody optionally comprising the amino acid sequence of SEQ ID NO: 50, and optionally wherein the cognate ligand is nicotine or a derivative thereof; a light chain variable region (VL) of an anti-nicotine antibody optionally comprising the amino acid sequence of SEQ ID NO: 51, and optionally wherein the cognate ligand is nicotine or a derivative thereof; a progesterone receptor domain optionally comprising the amino acid sequence of SEQ ID NO: 52, and optionally wherein the cognate ligand is mifepristone or a derivative thereof; an FRB domain optionally comprising the amino acid sequence of SEQ ID NO: 44, and optionally wherein the cognate ligand is rapamycin, AP1903, AP20187, FK1012, derivatives thereof, or analogs thereof; or
the one or more ligand binding domains of a first of the two or more polypeptide monomers comprise an FKBP domain, optionally comprising the amino acid sequence of SEQ ID NO: 43, and the one or more ligand binding domains of a second of the two or more polypeptide monomers comprise an FRB domain, optionally comprising the amino acid sequence of SEQ ID NO: 44, optionally wherein the cognate ligand is rapamycin, AP1903, AP20187, FK1012, derivatives thereof, or analogs thereof.

39. A pharmaceutical composition comprising:
a. the multimeric activation-conditional control polypeptide (ACP) of claim 34; or an isolated cell comprising the multimeric activation-conditional control polypeptide (ACP) of claim 34; or an engineered nucleic acid encoding the multimeric activation-conditional control polypeptide (ACP) of claim 34; and
b. a pharmaceutically acceptable carrier.

40. A method of treating a subject in need thereof, comprising administering to the subject a therapeutically effective dose of a pharmaceutical composition of claim 39.
